(19) United States
(12) Patent Application Publication
(10) Pub. No.: US 2024/0043833 A1
(43) Pub. Date: Feb. 8, 2024

(54) SYSTEMS AND METHODS FOR SPATIAL REFERENCE SEQUENCING

(71) Applicant: ULTIMA GENOMICS, INC., Newark, CA (US)

(72) Inventors: Florian OBERSTRASS, Redwood City, CA (US); Ron Saar DOVER, Palo Alto, CA (US); Zohar SHIPONY, Rehovot (IL); Gilad ALMOGY, Palo Alto, CA (US); Daniel MAZUR, San Diego, CA (US)

(21) Appl. No.: 18/488,731

(22) Filed: Oct. 17, 2023

Related U.S. Application Data
(63) Continuation of application No. PCT/US2022/026043, filed on Apr. 22, 2022.
(60) Provisional application No. 63/179,161, filed on Apr. 23, 2021.
(60) Provisional application No. 63/255,600, filed on Oct. 14, 2021.
(60) Provisional application No. 63/286,506, filed on Dec. 6, 2021.

(51) Int. Cl. C12N 15/10

(52) U.S. Cl. CPC C12N 15/1065

(57) ABSTRACT

Provided herein are methods, systems, compositions, and kits that can determine spatial information between a plurality of analytes in a sample by tagging such analytes with tags, where identities and/or locations of such tags are previously unknown prior to tagging. Each of a plurality of analyte sequences may be tagged with multiple spatial tags, such that each analyte sequence is associated with a set of two or more spatial tags. The sets of spatial tags may be analyzed to generate a map of analyte sequences. The map may comprise information about the respective absolute positions of each of a set of sequences with respect to a reference sequence. The map may comprise information about the respective probability cloud (or likely location) of each of a set of sequences with respect to a reference sequence.

### CROSS-REFERENCE

This application is a Continuation of PCT/US2022/026043, filed Apr. 22, 2022, which claims the benefit of U.S. Provisional Pat. App. No. 63/179,161, filed Apr. 23, 2021, U.S. Provisional Pat. App. No. 63/255,600, filed Oct. 14, 2021, and U.S. Provisional Pat. App. No. 63/286,506, filed Dec. 6, 2021, each of which is entirely incorporated by reference herein.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Despite the advance of sequencing technology, determining sequences with spatial resolution still requires laborious efforts.

### SUMMARY

Recognized herein is a need for methods, systems, compositions, and kits for the spatial mapping of a plurality of analyte sequences with respect to each other. Recognized herein is a need for methods, systems, compositions, and kits that can use tags, whose identities and/or locations are previously unknown. Provided herein are methods, systems, compositions, and kits that address at least the abovementioned needs. Beneficially, the provided systems, methods, kits, and compositions allow for the use of spatial tags which respective locations are not previously known or assayed prior to tagging the analyte sequences. This is advantageous over other systems which determine a spatial relationship between analyte sequences using an array of probes or other spatial tags with known locations, such as a system which has to deliberately generate such array with known locations and/or a system in which a pre-determination step has to be performed to determine the identities and locations of the probes or other spatial tags in the array.

Each of a plurality of analyte sequences may be tagged with multiple spatial tags, such that each analyte sequence is associated with a set of two or more spatial tags. The sets of spatial tags may be analyzed to generate a map of analyte sequences. The map may comprise information about the respective absolute positions of each of a set of sequences with respect to a reference sequence. Alternatively or in addition, the map may comprise information about the respective probability cloud (or likely location) of each of a set of sequences with respect to a reference sequence.

In an aspect, provided is a method comprising: (a) providing a substrate comprising a plurality of geolocation beads immobilized to a plurality of individually addressable locations on the substrate, wherein the plurality of geolocation beads comprises a plurality of spatial tag molecules comprising a plurality of spatial tags, and wherein each geolocation bead of the plurality of geolocation beads comprises a unique spatial tag; (b) loading a sample onto the substrate, wherein the sample comprises a plurality of analyte sequences; (c) releasing the plurality of spatial tag molecules from the plurality of geolocation beads, wherein, prior to the releasing, respective locations of the plurality of spatial tags are unknown; (d) tagging a set of at least two spatial tags of the plurality of spatial tags to each of the plurality of analyte sequences to generate a plurality of spatially tagged sequences; and (e) generating a map of the plurality of analyte sequences by identifying sets of spatial tags from the plurality of spatially tagged sequences, wherein the map comprises spatial information between at least a subset of the plurality of analyte sequences.

In some embodiments, the method further comprises, prior to (a), loading the plurality of geolocation beads onto the substrate to immobilize the plurality of geolocation beads at the plurality of individually addressable locations. In some embodiments, the method further comprises fixing the sample. In some embodiments, the method further comprises permeabilizing the sample.

In some embodiments, the sample comprises a tissue sample, wherein the plurality of analyte sequences comprises a plurality of messenger ribonucleic acid (mRNA) transcript sequences.

In some embodiments, (d) comprises contacting a plurality of bridge constructs with the plurality of spatial tag molecules and the plurality of analyte sequences, under conditions sufficient for a bridge construct of the plurality of bridge constructs to capture (1) a first spatial tag molecule from the plurality of spatial tags, wherein the first spatial tag molecule comprises a first spatial tag, (2) a second spatial tag molecule from the plurality of spatial tags, wherein the second spatial tag molecule comprises a second spatial tag, and (3) an analyte sequence of the plurality of analyte sequences, to generate a tagged complex, and generating a spatially tagged sequence of the plurality of spatially tagged sequences using the tagged complex.

In some embodiments, the bridge construct is a partially double-stranded nucleic acid molecule, comprising a capture sequence as an overhang, a first attachment binding sequence, a second attachment binding sequence, a third attachment binding sequence, and a fourth attachment binding sequence, wherein the capture sequence is configured to bind to a sequence of the analyte sequence, wherein the first spatial tag molecule comprises a first attachment sequence configured to bind to the first attachment binding sequence, the first spatial tag, and a second attachment sequence configured to bind to the second attachment binding sequence, wherein the second spatial tag molecule comprises a third attachment sequence configured to bind to the third attachment binding sequence, the second spatial tag, and a fourth attachment sequence configured to bind to the fourth attachment binding sequence, and wherein the tagged complex comprises a first nucleic acid strand comprising, in 3′ to 5′ order, (1) the capture sequence which is bound to the sequence of the analyte sequence, (2) the first attachment sequence which is bound to the first attachment binding sequence, (3) the first spatial tag which is not bound to another nucleic acid strand, (4) the second attachment sequence which is bound to the second attachment binding sequence, (5) the third attachment sequence which is bound to the third attachment binding sequence, (6) the second spatial tag which is not bound to another nucleic acid strand, and (7) the fourth attachment sequence which is bound to the fourth attachment binding sequence.

In some embodiments, the second spatial tag molecule comprises a capture entity, wherein the first nucleic acid strand of the tagged complex comprises the capture entity. In some embodiments, the capture entity comprises biotin.

In some embodiments, the bridge construct comprises a first spacer sequence disposed between the first attachment binding sequence and the second attachment binding sequence, wherein the bridge construct comprises a second spacer sequence disposed between the third attachment binding sequence and the fourth attachment binding sequence.

In some embodiments, the capture sequence comprises a polyT sequence, and wherein the sequence of the analyte sequence comprises a polyA sequence. In some embodiments, the capture sequence comprises a random n-mer sequence.

In some embodiments, a first subset of the plurality of spatial tag molecules each comprises a respective spatial tag disposed between the first attachment sequence and the second attachment sequence, and wherein a second subset of the plurality of spatial tag molecules each comprises a respective spatial tag disposed between the third attachment sequence and the fourth attachment sequence.

In some embodiments, the first attachment sequence is different from the third attachment sequence. In some embodiments, the first attachment sequence is the same as the third attachment sequence. In some embodiments, the first attachment sequence is different form the second attachment sequence. In some embodiments, the first attachment sequence is the same as the second attachment sequence.

In some embodiments, a geolocation bead of the plurality of geolocation beads comprises a set of spatial tag molecules of the plurality of spatial tag molecules, wherein each spatial tag molecule of the set of spatial tag molecules comprises a common spatial tag of the plurality of spatial tags. In some embodiments, each spatial tag molecule of the set of spatial tag molecules further comprises a unique molecular identifier (UMI) that is unique amongst the set of spatial tag molecules.

In some embodiments, each spatial tag molecule of the set of spatial tag molecules comprises a first attachment sequence and a second attachment sequence, wherein the common spatial tag is disposed between the first attachment sequence and the second attachment sequence. In some embodiments, each spatial tag molecule of the set of spatial tag molecules further comprises a UMI that is unique amongst the set of spatial tag molecules, wherein the UMI is disposed between the first attachment sequence and the second attachment sequence.

In some embodiments, the set of spatial tag molecules comprises a first subset of spatial tag molecules and a second subset of spatial tag molecules, wherein the first subset of spatial tag molecules comprises a first attachment sequence and a second attachment sequence, and wherein the second subset of spatial tag molecules comprises a third attachment sequence and a fourth attachment sequence, wherein the first attachment sequence is different from the third attachment sequence. In some embodiments, the second attachment sequence is different from the fourth attachment sequence.

In some embodiments, the set of spatial tag molecules comprises at least 100,000 spatial tag molecules.

In some embodiments, the plurality of geolocation beads comprises a first set of geolocation beads and a second set of geolocation beads, wherein the first set of geolocation beads comprises a first set of spatial tag molecules of the plurality of spatial tag molecules each comprising a first attachment sequence and a second attachment sequence, wherein the second set of geolocation beads comprises a second set of spatial tag molecules of the plurality of spatial tag molecules each comprising a third attachment sequence and a fourth attachment sequence, wherein the first attachment sequence is different from the third attachment sequence. In some embodiments, the second attachment sequence is different from the fourth attachment sequence.

In some embodiments, a geolocation bead of the plurality of geolocation beads comprises an oligonucleotide molecule, wherein the oligonucleotide molecule comprises a spatial tag molecule of the plurality of spatial tag molecules. In some embodiments, the spatial tag molecule is a first strand of the oligonucleotide molecule.

In some embodiments, the oligonucleotide molecule is a partially double-stranded nucleic acid molecule comprising a first strand and a second strand, wherein the first strand comprises the spatial tag molecule, wherein the second strand comprises a primer sequence and sequences corresponding to the spatial tag molecule. In some embodiments, (c) comprises releasing the spatial tag molecule from the second strand.

In some embodiments, a spatial tag molecule of the plurality of spatial tag molecules comprises a capture entity. In some embodiments, the capture entity comprises biotin. In some embodiments, the method further comprises recovering the plurality of spatially tagged sequences using the capture entity. In some embodiments, the method further comprises recovering a plurality of tagged complexes using the capture entity, and generating the plurality of spatially tagged sequences using the plurality of tagged complexes.

In some embodiments, a spatial tag molecule of the plurality of spatial tag molecules comprises a blocking group and a cleavage site.

In some embodiments, the plurality of geolocation beads comprises at least 1,000,000 geolocation beads. In some embodiments, the plurality of geolocation beads comprises at least 100,000,000 geolocation beads. In some embodiments, the plurality of geolocation beads comprises at least 100,000,000,000 geolocation beads.

In some embodiments, an analyte sequence comprises an mRNA sequence. In some embodiments, an analyte sequence comprises a DNA sequence.

In some embodiments, the spatial information comprises a relative position of the at least the subset of the plurality of analyte sequences with respect to a reference analyte sequence. In some embodiments, the spatial information comprises a relative probability cloud of the at least the subset of the plurality of analyte sequences with respect to a reference analyte sequence. In some embodiments, the spatial information comprises two-dimensional (2D) spatial information. In some embodiments, the spatial information comprises three-dimensional (3D) spatial information.

In some embodiments, the method further comprises, subsequent to (b) and prior to (d), (i) contacting the sample with a surface of a second substrate, wherein the second substrate comprises a second plurality of geolocation beads immobilized to a second plurality of individually addressable locations on the surface of the second substrate, wherein the second plurality of geolocation beads comprises a second plurality of spatial tag molecules comprising a second plurality of spatial tags, wherein each geolocation bead of the second plurality of geolocation beads comprises a unique spatial tag, wherein the plurality of spatial tags and the second plurality of spatial tags are mutually exclusive, and (ii) releasing the second plurality of spatial tag molecules from the second plurality of geolocation beads, wherein, prior to the releasing, respective locations or identities of the second plurality of spatial tags are unknown.

In some embodiments, the method further comprises, in (d), tagging a set of at least two spatial tags of plurality of spatial tags, at least two spatial tags of the second plurality of spatial tags, or at least two spatial tags from both the plurality of spatial tags and the second plurality of spatial tags to each of the plurality of analyte sequences to generate the plurality of spatially tagged sequences.

In another aspect, provided is a method comprising: (a) providing a substrate comprising a plurality of geolocation beads immobilized to a plurality of individually addressable locations on the substrate, wherein the plurality of geolocation beads comprises a plurality of spatial tag molecules comprising a plurality of spatial tags, and wherein each geolocation bead of the plurality of geolocation beads comprises a unique spatial tag; (b) loading a sample onto the substrate, wherein the sample comprises a plurality of analyte sequences; (c) releasing the plurality of spatial tag molecules from the plurality of geolocation beads, wherein, prior to the releasing, respective identities of the plurality of spatial tags are unknown; (d) tagging a set of at least two spatial tags of the plurality of spatial tag molecules to each of the plurality of analyte sequences to generate a plurality of spatially tagged sequences; and (e) generating a map of the plurality of analyte sequences by identifying sets of spatial tags from the plurality of spatially tagged sequences, wherein the map comprises spatial information between at least a subset of the plurality of analyte sequences.

In some embodiments, the method further comprises, prior to (a), loading the plurality of geolocation beads onto the substrate to immobilize the plurality of geolocation beads at the plurality of individually addressable locations.

In some embodiments, the method further comprises fixing the sample. In some embodiments, the method further comprises permeabilizing the sample. In some embodiments, the sample comprises a tissue sample, wherein the plurality of analyte sequences comprises a plurality of messenger ribonucleic acid (mRNA) transcript sequences.

In some embodiments, (d) comprises contacting a plurality of bridge constructs with the plurality of spatial tag molecules and the plurality of analyte sequences, under conditions sufficient for a bridge construct of the plurality of bridge constructs to capture (1) a first spatial tag molecule from the plurality of spatial tags, wherein the first spatial tag molecule comprises a first spatial tag, (2) a second spatial tag molecule from the plurality of spatial tags, wherein the second spatial tag molecule comprises a second spatial tag, and (3) an analyte sequence of the plurality of analyte sequences, to generate a tagged complex, and generating a spatially tagged sequence of the plurality of spatially tagged sequences using the tagged complex.

In some embodiments, the bridge construct is a partially double-stranded nucleic acid molecule, comprising a capture sequence as an overhang, a first attachment binding sequence, a second attachment binding sequence, a third attachment binding sequence, and a fourth attachment binding sequence, wherein the capture sequence is configured to bind to a sequence of the analyte sequence, wherein the first spatial tag molecule comprises a first attachment sequence configured to bind to the first attachment binding sequence, the first spatial tag, and a second attachment sequence configured to bind to the second attachment binding sequence, wherein the second spatial tag molecule comprises a third attachment sequence configured to bind to the third attachment binding sequence, the second spatial tag, and a fourth attachment sequence configured to bind to the fourth attachment binding sequence, and wherein the tagged complex comprises a first nucleic acid strand comprising, in 3′ to 5′ order, (1) the capture sequence which is bound to the sequence of the analyte sequence, (2) the first attachment sequence which is bound to the first attachment binding sequence, (3) the first spatial tag which is not bound to another nucleic acid strand, (4) the second attachment sequence which is bound to the second attachment binding sequence, (5) the third attachment sequence which is bound to the third attachment binding sequence, (6) the second spatial tag which is not bound to another nucleic acid strand, and (7) the fourth attachment sequence which is bound to the fourth attachment binding sequence.

In some embodiments, the second spatial tag molecule comprises a capture entity, wherein the first nucleic acid strand of the tagged complex comprises the capture entity. In some embodiments, the capture entity comprises biotin.

In some embodiments, the bridge construct comprises a first spacer sequence disposed between the first attachment binding sequence and the second attachment binding sequence, wherein the bridge construct comprises a second spacer sequence disposed between the third attachment binding sequence and the fourth attachment binding sequence.

In some embodiments, the capture sequence comprises a polyT sequence, and wherein the sequence of the analyte sequence comprises a polyA sequence. In some embodiments, the capture sequence comprises a random n-mer sequence.

In some embodiments, a first subset of the plurality of spatial tag molecules each comprises a respective spatial tag disposed between the first attachment sequence and the second attachment sequence, and wherein a second subset of the plurality of spatial tag molecules each comprises a respective spatial tag disposed between the third attachment sequence and the fourth attachment sequence.

In some embodiments, the first attachment sequence is different from the third attachment sequence. In some embodiments, the first attachment sequence is the same as the third attachment sequence. In some embodiments, the first attachment sequence is different form the second attachment sequence. In some embodiments, the first attachment sequence is the same as the second attachment sequence.

In some embodiments, a geolocation bead of the plurality of geolocation beads comprises a set of spatial tag molecules of the plurality of spatial tag molecules, wherein each spatial tag molecule of the set of spatial tag molecules comprises a common spatial tag of the plurality of spatial tags. In some embodiments, each spatial tag molecule of the set of spatial tag molecules further comprises a unique molecular identifier (UMI) that is unique amongst the set of spatial tag molecules.

In some embodiments, each spatial tag molecule of the set of spatial tag molecules comprises a first attachment sequence and a second attachment sequence, wherein the common spatial tag is disposed between the first attachment sequence and the second attachment sequence. In some embodiments, each spatial tag molecule of the set of spatial tag molecules further comprises a UMI that is unique amongst the set of spatial tag molecules, wherein the UMI is disposed between the first attachment sequence and the second attachment sequence.

In some embodiments, the set of spatial tag molecules comprises a first subset of spatial tag molecules and a second subset of spatial tag molecules, wherein the first subset of spatial tag molecules comprises a first attachment sequence and a second attachment sequence, and wherein the second subset of spatial tag molecules comprises a third attachment sequence and a fourth attachment sequence, wherein the first attachment sequence is different from the third attachment sequence. In some embodiments, the second attachment sequence is different from the fourth attachment sequence.

In some embodiments, the set of spatial tag molecules comprises at least 100,000 spatial tag molecules.

In some embodiments, the plurality of geolocation beads comprises a first set of geolocation beads and a second set of geolocation beads, wherein the first set of geolocation beads comprises a first set of spatial tag molecules of the plurality of spatial tag molecules each comprising a first attachment sequence and a second attachment sequence, wherein the second set of geolocation beads comprises a second set of spatial tag molecules of the plurality of spatial tag molecules each comprising a third attachment sequence and a fourth attachment sequence, wherein the first attachment sequence is different from the third attachment sequence. In some embodiments, the second attachment sequence is different from the fourth attachment sequence.

In some embodiments, a geolocation bead of the plurality of geolocation beads comprises an oligonucleotide molecule, wherein the oligonucleotide molecule comprises a spatial tag molecule of the plurality of spatial tag molecules. In some embodiments, the spatial tag molecule is a first strand of the oligonucleotide molecule.

In some embodiments, the oligonucleotide molecule is a partially double-stranded nucleic acid molecule comprising a first strand and a second strand, wherein the first strand comprises the spatial tag molecule, wherein the second strand comprises a primer sequence and sequences corresponding to the spatial tag molecule. In some embodiments, (c) comprises releasing the spatial tag molecule from the second strand.

In some embodiments, a spatial tag molecule of the plurality of spatial tag molecules comprises a capture entity. In some embodiments, the capture entity comprises biotin. In some embodiments, the method further comprises recovering the plurality of spatially tagged sequences using the capture entity. In some embodiments, the method further comprises recovering a plurality of tagged complexes using the capture entity, and generating the plurality of spatially tagged sequences using the plurality of tagged complexes.

In some embodiments, a spatial tag molecule of the plurality of spatial tag molecules comprises a blocking group and a cleavage site.

In some embodiments, the plurality of geolocation beads comprises at least 1,000,000 geolocation beads. In some embodiments, the plurality of geolocation beads comprises at least 100,000,000 geolocation beads. In some embodiments, the plurality of geolocation beads comprises at least 100,000,000,000 geolocation beads.

In some embodiments, an analyte sequence comprises an mRNA sequence. In some embodiments, an analyte sequence comprises a DNA sequence.

In some embodiments, the spatial information comprises a relative position of the at least the subset of the plurality of analyte sequences with respect to a reference analyte sequence. In some embodiments, the spatial information comprises a relative probability cloud of the at least the subset of the plurality of analyte sequences with respect to a reference analyte sequence. In some embodiments, the spatial information comprises two-dimensional (2D) spatial information. In some embodiments, the spatial information comprises three-dimensional (3D) spatial information.

In some embodiments, the method further comprises, subsequent to (b) and prior to (d), (i) contacting the sample with a surface of a second substrate, wherein the second substrate comprises a second plurality of geolocation beads immobilized to a second plurality of individually addressable locations on the surface of the second substrate, wherein the second plurality of geolocation beads comprises a second plurality of spatial tag molecules comprising a second plurality of spatial tags, wherein each geolocation bead of the second plurality of geolocation beads comprises a unique spatial tag, wherein the plurality of spatial tags and the second plurality of spatial tags are mutually exclusive, and (ii) releasing the second plurality of spatial tag molecules from the second plurality of geolocation beads, wherein, prior to the releasing, respective locations or identities of the second plurality of spatial tags are unknown.

In some embodiments, the method further comprises, in (d), tagging a set of at least two spatial tags of plurality of spatial tags, at least two spatial tags of the second plurality of spatial tags, or at least two spatial tags from both the plurality of spatial tags and the second plurality of spatial tags to each of the plurality of analyte sequences to generate the plurality of spatially tagged sequences.

In another aspect, provided is a method comprising: (a) partitioning cells into a plurality of partitions, wherein a partition of the plurality of partitions comprises a cell and a plurality of geolocation beads, wherein the cell comprises a plurality of analyte sequences, wherein the plurality of geolocation beads comprises a plurality of spatial tag molecules comprising a plurality of spatial tags, and wherein each geolocation bead of the plurality of geolocation beads comprises a unique spatial tag; (b) releasing the plurality of spatial tag molecules from the plurality of geolocation beads; (c) tagging a set of at least two spatial tags of the plurality of spatial tags to each of the plurality of analyte sequences to generate a plurality of spatially tagged sequences; and (d) sequencing the plurality of spatially tagged sequences, or derivatives thereof, to determine that the plurality of analyte sequences originated from the cell of the plurality of cells by identifying sets of spatial tags from the plurality of spatially tagged sequences.

In some embodiments, the partition is a droplet. In some embodiments, the partition is a well.

In some embodiments, (c) comprises contacting a plurality of bridge constructs with the plurality of spatial tag molecules and the plurality of analyte sequences, under conditions sufficient for a bridge construct of the plurality of bridge constructs to capture (1) a first spatial tag molecule from the plurality of spatial tags, wherein the first spatial tag molecule comprises a first spatial tag, (2) a second spatial tag molecule from the plurality of spatial tags, wherein the second spatial tag molecule comprises a second spatial tag, and (3) an analyte sequence of the plurality of analyte sequences, to generate a tagged complex, and generating a spatially tagged sequence of the plurality of spatially tagged sequences using the tagged complex.

In some embodiments, the bridge construct is a partially double-stranded nucleic acid molecule, comprising a capture sequence as an overhang, a first attachment binding sequence, a second attachment binding sequence, a third attachment binding sequence, and a fourth attachment binding sequence, wherein the capture sequence is configured to bind to a sequence of the analyte sequence, wherein the first spatial tag molecule comprises a first attachment sequence configured to bind to the first attachment binding sequence, the first spatial tag, and a second attachment sequence configured to bind to the second attachment binding sequence, wherein the second spatial tag molecule comprises a third attachment sequence configured to bind to the third attachment binding sequence, the second spatial tag, and a fourth attachment sequence configured to bind to the fourth attachment binding sequence, and wherein the tagged complex comprises a first nucleic acid strand comprising, in 3′ to 5′ order, (1) the capture sequence which is bound to the sequence of the analyte sequence, (2) the first attachment sequence which is bound to the first attachment binding sequence, (3) the first spatial tag which is not bound to another nucleic acid strand, (4) the second attachment sequence which is bound to the second attachment binding sequence, (5) the third attachment sequence which is bound to the third attachment binding sequence, (6) the second spatial tag which is not bound to another nucleic acid strand, and (7) the fourth attachment sequence which is bound to the fourth attachment binding sequence.

In some embodiments, the second spatial tag molecule comprises a capture entity, wherein the first nucleic acid strand of the tagged complex comprises the capture entity. In some embodiments, the capture entity comprises biotin.

In some embodiments, the bridge construct comprises a first spacer sequence disposed between the first attachment binding sequence and the second attachment binding sequence, wherein the bridge construct comprises a second spacer sequence disposed between the third attachment binding sequence and the fourth attachment binding sequence.

In some embodiments, the capture sequence comprises a polyT sequence, and wherein the sequence of the analyte sequence comprises a polyA sequence. In some embodiments, the capture sequence comprises a random n-mer sequence.

In some embodiments, a first subset of the plurality of spatial tag molecules each comprises a respective spatial tag disposed between the first attachment sequence and the second attachment sequence, and wherein a second subset of the plurality of spatial tag molecules each comprises a respective spatial tag disposed between the third attachment sequence and the fourth attachment sequence.

In some embodiments, the first attachment sequence is different from the third attachment sequence. In some embodiments, the first attachment sequence is the same as the third attachment sequence. In some embodiments, the first attachment sequence is different form the second attachment sequence. In some embodiments, the first attachment sequence is the same as the second attachment sequence.

In some embodiments, a geolocation bead of the plurality of geolocation beads comprises a set of spatial tag molecules of the plurality of spatial tag molecules, wherein each spatial tag molecule of the set of spatial tag molecules comprises a common spatial tag of the plurality of spatial tags. In some embodiments, each spatial tag molecule of the set of spatial tag molecules further comprises a unique molecular identifier (UMI) that is unique amongst the set of spatial tag molecules.

In some embodiments, each spatial tag molecule of the set of spatial tag molecules comprises a first attachment sequence and a second attachment sequence, wherein the common spatial tag is disposed between the first attachment sequence and the second attachment sequence. In some embodiments, each spatial tag molecule of the set of spatial tag molecules further comprises a UMI that is unique amongst the set of spatial tag molecules, wherein the UMI is disposed between the first attachment sequence and the second attachment sequence.

In some embodiments, the set of spatial tag molecules comprises a first subset of spatial tag molecules and a second subset of spatial tag molecules, wherein the first subset of spatial tag molecules comprises a first attachment sequence and a second attachment sequence, and wherein the second subset of spatial tag molecules comprises a third attachment sequence and a fourth attachment sequence, wherein the first attachment sequence is different from the third attachment sequence. In some embodiments, the second attachment sequence is different from the fourth attachment sequence.

In some embodiments, the set of spatial tag molecules comprises at least 100,000 spatial tag molecules.

In some embodiments, the plurality of geolocation beads comprises a first set of geolocation beads and a second set of geolocation beads, wherein the first set of geolocation beads comprises a first set of spatial tag molecules of the plurality of spatial tag molecules each comprising a first attachment sequence and a second attachment sequence, wherein the second set of geolocation beads comprises a second set of spatial tag molecules of the plurality of spatial tag molecules each comprising a third attachment sequence and a fourth attachment sequence, wherein the first attachment sequence is different from the third attachment sequence. In some embodiments, the second attachment sequence is different from the fourth attachment sequence.

In some embodiments, a geolocation bead of the plurality of geolocation beads comprises an oligonucleotide molecule, wherein the oligonucleotide molecule comprises a spatial tag molecule of the plurality of spatial tag molecules. In some embodiments, the spatial tag molecule is a first strand of the oligonucleotide molecule.

In some embodiments, oligonucleotide molecule is a partially double-stranded nucleic acid molecule comprising a first strand and a second strand, wherein the first strand comprises the spatial tag molecule, wherein the second strand comprises a primer sequence and sequences corresponding to the spatial tag molecule. In some embodiments, (b) comprises releasing the spatial tag molecule from the second strand.

In some embodiments, a spatial tag molecule of the plurality of spatial tag molecules comprises a capture entity. In some embodiments, the capture entity comprises biotin. In some embodiments, the method further comprises recovering the plurality of spatially tagged sequences using the capture entity. In some embodiments, the method further comprises recovering a plurality of tagged complexes using the capture entity, and generating the plurality of spatially tagged sequences using the plurality of tagged complexes.

In some embodiments, a spatial tag molecule of the plurality of spatial tag molecules comprises a blocking group and a cleavage site.

In some embodiments, the plurality of geolocation beads comprises at least 1,000,000 geolocation beads. In some embodiments, the plurality of geolocation beads comprises at least 100,000,000 geolocation beads. In some embodiments, the plurality of geolocation beads comprises at least 100,000,000,000 geolocation beads.

In some embodiments, an analyte sequence comprises an mRNA sequence. In some embodiments, an analyte sequence comprises a DNA sequence.

In another aspect, provided is a method comprising: (a) providing a solution comprising a plurality of cells and a plurality of geolocation beads, wherein the plurality of cells comprises a plurality of analyte sequences, wherein the plurality of geolocation beads comprises a plurality of spatial tag molecules comprising a plurality of spatial tags, and wherein each geolocation bead of the plurality of geolocation beads comprises a unique spatial tag; (b) releasing the plurality of spatial tag molecules from the plurality of geolocation beads; (c) tagging a set of at least two spatial tags of the plurality of spatial tags to each of the plurality of analyte sequences to generate a plurality of spatially tagged sequences; and (d) sequencing the plurality of spatially tagged sequences, or derivatives thereof, to determine that a subset of the plurality of analyte sequences originated from a cell of the plurality of cells by identifying sets of spatial tags from the plurality of spatially tagged sequences.

In some embodiments, (c) comprises contacting a plurality of bridge constructs with the plurality of spatial tag molecules and the plurality of analyte sequences, under conditions sufficient for a bridge construct of the plurality of bridge constructs to capture (1) a first spatial tag molecule from the plurality of spatial tags, wherein the first spatial tag molecule comprises a first spatial tag, (2) a second spatial tag molecule from the plurality of spatial tags, wherein the second spatial tag molecule comprises a second spatial tag, and (3) an analyte sequence of the plurality of analyte sequences, to generate a tagged complex, and generating a spatially tagged sequence of the plurality of spatially tagged sequences using the tagged complex.

In some embodiments, the bridge construct is a partially double-stranded nucleic acid molecule, comprising a capture sequence as an overhang, a first attachment binding sequence, a second attachment binding sequence, a third attachment binding sequence, and a fourth attachment binding sequence, wherein the capture sequence is configured to bind to a sequence of the analyte sequence, wherein the first spatial tag molecule comprises a first attachment sequence configured to bind to the first attachment binding sequence, the first spatial tag, and a second attachment sequence configured to bind to the second attachment binding sequence, wherein the second spatial tag molecule comprises a third attachment sequence configured to bind to the third attachment binding sequence, the second spatial tag, and a fourth attachment sequence configured to bind to the fourth attachment binding sequence, and wherein the tagged complex comprises a first nucleic acid strand comprising, in 3′ to 5′ order, (1) the capture sequence which is bound to the sequence of the analyte sequence, (2) the first attachment sequence which is bound to the first attachment binding sequence, (3) the first spatial tag which is not bound to another nucleic acid strand, (4) the second attachment sequence which is bound to the second attachment binding sequence, (5) the third attachment sequence which is bound to the third attachment binding sequence, (6) the second spatial tag which is not bound to another nucleic acid strand, and (7) the fourth attachment sequence which is bound to the fourth attachment binding sequence.

In some embodiments, the second spatial tag molecule comprises a capture entity, wherein the first nucleic acid strand of the tagged complex comprises the capture entity. In some embodiments, the capture entity comprises biotin.

In some embodiments, the bridge construct comprises a first spacer sequence disposed between the first attachment binding sequence and the second attachment binding sequence, wherein the bridge construct comprises a second spacer sequence disposed between the third attachment binding sequence and the fourth attachment binding sequence.

In some embodiments, the capture sequence comprises a polyT sequence, and wherein the sequence of the analyte sequence comprises a polyA sequence. In some embodiments, the capture sequence comprises a random n-mer sequence.

In some embodiments, a first subset of the plurality of spatial tag molecules each comprises a respective spatial tag disposed between the first attachment sequence and the second attachment sequence, and wherein a second subset of the plurality of spatial tag molecules each comprises a respective spatial tag disposed between the third attachment sequence and the fourth attachment sequence.

In some embodiments, the first attachment sequence is different from the third attachment sequence. In some embodiments, the first attachment sequence is the same as the third attachment sequence. In some embodiments, the first attachment sequence is different from the second attachment sequence. In some embodiments, the first attachment sequence is the same as the second attachment sequence.

In some embodiments, a geolocation bead of the plurality of geolocation beads comprises a set of spatial tag molecules of the plurality of spatial tag molecules, wherein each spatial tag molecule of the set of spatial tag molecules comprises a common spatial tag of the plurality of spatial tags. In some embodiments, each spatial tag molecule of the set of spatial tag molecules further comprises a unique molecular identifier (UMI) that is unique amongst the set of spatial tag molecules.

In some embodiments, each spatial tag molecule of the set of spatial tag molecules comprises a first attachment sequence and a second attachment sequence, wherein the common spatial tag is disposed between the first attachment sequence and the second attachment sequence. In some embodiments, each spatial tag molecule of the set of spatial tag molecules further comprises a UMI that is unique amongst the set of spatial tag molecules, wherein the UMI is disposed between the first attachment sequence and the second attachment sequence.

In some embodiments, the set of spatial tag molecules comprises a first subset of spatial tag molecules and a second subset of spatial tag molecules, wherein the first subset of spatial tag molecules comprises a first attachment sequence and a second attachment sequence, and wherein the second subset of spatial tag molecules comprises a third attachment sequence and a fourth attachment sequence, wherein the first attachment sequence is different from the third attachment sequence. In some embodiments, the second attachment sequence is different from the fourth attachment sequence.

In some embodiments, the set of spatial tag molecules comprises at least 100,000 spatial tag molecules.

In some embodiments, the plurality of geolocation beads comprises a first set of geolocation beads and a second set of geolocation beads, wherein the first set of geolocation beads comprises a first set of spatial tag molecules of the plurality of spatial tag molecules each comprising a first attachment sequence and a second attachment sequence, wherein the second set of geolocation beads comprises a second set of spatial tag molecules of the plurality of spatial tag molecules each comprising a third attachment sequence and a fourth attachment sequence, wherein the first attachment sequence is different from the third attachment sequence. In some embodiments, the second attachment sequence is different from the fourth attachment sequence.

In some embodiments, a geolocation bead of the plurality of geolocation beads comprises an oligonucleotide molecule, wherein the oligonucleotide molecule comprises a spatial tag molecule of the plurality of spatial tag molecules. In some embodiments, the spatial tag molecule is a first strand of the oligonucleotide molecule.

In some embodiments, oligonucleotide molecule is a partially double-stranded nucleic acid molecule comprising a first strand and a second strand, wherein the first strand comprises the spatial tag molecule, wherein the second strand comprises a primer sequence and sequences corresponding to the spatial tag molecule. In some embodiments, (b) comprises releasing the spatial tag molecule from the second strand.

In some embodiments, a spatial tag molecule of the plurality of spatial tag molecules comprises a capture entity. In some embodiments, the capture entity comprises biotin. In some embodiments, the method further comprises recovering the plurality of spatially tagged sequences using the capture entity. In some embodiments, the method further comprises recovering a plurality of tagged complexes using the capture entity, and generating the plurality of spatially tagged sequences using the plurality of tagged complexes.

In some embodiments, a spatial tag molecule of the plurality of spatial tag molecules comprises a blocking group and a cleavage site.

In some embodiments, the plurality of geolocation beads comprises at least 1,000,000 geolocation beads. In some embodiments, the plurality of geolocation beads comprises at least 100,000,000 geolocation beads. In some embodiments, the plurality of geolocation beads comprises at least 100,000,000,000 geolocation beads.

In some embodiments, an analyte sequence comprises an mRNA sequence. In some embodiments, an analyte sequence comprises a DNA sequence.

In another aspect, provided is a kit, comprising: a substrate comprising a plurality of geolocation beads immobilized to a plurality of individually addressable locations on the substrate, wherein the plurality of geolocation beads comprises oligonucleotide molecules coupled thereto, wherein the oligonucleotide molecules comprise spatial tag molecules, wherein each geolocation bead of said plurality of geolocation beads comprises a unique spatial tag, wherein the oligonucleotide molecules are releasable from the plurality of geolocation beads by one or more of the release mechanisms selected from the group consisting of: (a) providing an enzyme mix comprising an enzyme configured to cleave or digest a cleavage site, wherein the oligonucleotide molecules comprises the cleavage site; (b) providing biotin, wherein the oligonucleotide molecules are conjugated to a desthiobiotin moiety, wherein the geolocation bead comprises a streptavidin moiety bound to the desthiobiotin moiety; (c) providing an enzyme mix comprising an enzyme configured to cleave or digest a cleavage site and UV light, wherein the oligonucleotide molecules comprises azobenzene and the cleavage site; and (d) providing UV light, wherein the oligonucleotide molecules are conjugated azobenzene, wherein the plurality of geolocation beads comprises an alpha-cyclodextrine (a-CD) moiety bound to the azobenzene.

In some embodiments, the kit further comprises indexed data comprising a list of spatial tag sequences included in the geolocation beads.

In some embodiments, the kit further comprises a second substrate comprising a second plurality of individually addressable locations configured to immobilize a second plurality of geolocation beads. In some embodiments, the kit further comprises the second plurality of geolocation beads.

In some embodiments, the kit further comprises a reagent configured to release the oligonucleotide molecules from the plurality of geolocation beads. In some embodiments, the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of geolocation beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of geolocation beads comprises an alpha-cyclodextrine (a-CD) moiety bound to the azobenzene.

In some embodiments, the kit further comprises sequencing reagents. In some embodiments, the sequencing reagents comprise single-base nucleotide mixtures for each of the four base types. In some embodiments, a single-base nucleotide mixture of the single-base nucleotide mixtures comprises a mixture of labeled nucleotides and non-labeled nucleotides of a single base. In some embodiments, a single-base nucleotide mixture of the single-base nucleotide mixtures comprises non-terminated nucleotides.

In some embodiments, the kit further comprises amplification reagents. In some embodiments, the amplification reagents comprise a polymerase, a nucleotide mixture, a primer, a buffer, or any combination thereof.

In some embodiments, the kit further comprises a biological sample. In some embodiments, the biological sample is a tissue. In some embodiments, the biological sample is fixed. In some embodiments, the biological sample is permeabilized. In some embodiments, the biological sample is loaded on the substrate.

In some embodiments, a geolocation bead of the plurality of geolocation beads comprises at least 100,000 oligonucleotide molecules. In some embodiments, the at least 100,000 oligonucleotide molecules comprise a spatial tag sequence of the spatial tag sequences that is common and unique to the bead amongst the plurality of beads.

In some embodiments, an oligonucleotide molecule of the oligonucleotide molecules comprises a capture sequence, wherein the capture sequence is configured to hybridize with a sequence of an analyte, or derivative thereof, of a biological sample. In some embodiments, the capture sequence is selected from the group consisting of: a polyT sequence, a polyG sequence, a targeted mRNA sequence, a targeted gDNA sequence, a random n-mer sequence, and a probe sequence.

In some embodiments, the substrate comprises at least 1,000,000 individually addressable locations. In some embodiments, the substrate comprises at least 1,000,000,000 individually addressable locations.

In some embodiments, the plurality of geolocation beads is immobilized to the individually addressable locations via electrostatic interactions.

In some embodiments, the substrate is substantially planar.

In another aspect, provided is a system, comprising: a sequencing platform configured to (i) address individually addressable locations of substrates and (ii) rotate the substrates during dispensing of sequencing reagents to the substrates or during imaging of the substrates or during both; a substrate comprising a plurality of geolocation beads immobilized to a plurality of individually addressable locations on the substrate, wherein the plurality of geolocation beads comprises oligonucleotide molecules coupled thereto, wherein the oligonucleotide molecules comprise spatial tag molecules, wherein each geolocation bead of said plurality of geolocation beads comprises a unique spatial tag, wherein the oligonucleotide molecules are releasable from the plurality of geolocation beads by one or more of the release mechanisms selected from the group consisting of: (a) providing an enzyme mix comprising an enzyme configured to cleave or digest a cleavage site, wherein the oligonucleotide molecules comprise the cleavage site; (b) providing biotin, wherein the oligonucleotide molecules are conjugated to a desthiobiotin moiety, wherein the geolocation bead comprises a streptavidin moiety bound to the desthiobiotin moiety; (c) providing an enzyme mix comprising an enzyme configured to cleave or digest a cleavage site and UV light, wherein the oligonucleotide molecules comprise azobenzene and the cleavage site; and (d) providing UV light, wherein the oligonucleotide molecules are conjugated azobenzene, wherein the plurality of geolocation beads comprise an alpha-cyclodextrine (a-CD) moiety bound to the azobenzene.

In some embodiments, the system further comprises indexed data comprising a list of spatial tag sequences included in the geolocation beads.

In some embodiments, the system further comprises a second substrate comprising a second plurality of individually addressable locations configured to immobilize a second plurality of geolocation beads. In some embodiments, the system further comprises the second plurality of geolocation beads.

In some embodiments, the system further comprises a reagent configured to release the oligonucleotide molecules from the plurality of geolocation beads. In some embodiments, the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of geolocation beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of geolocation beads comprises an alpha-cyclodextrine (a-CD) moiety bound to the azobenzene.

In some embodiments, the system further comprises sequencing reagents. In some embodiments, the sequencing reagents comprise single-base nucleotide mixtures for each of the four base types. In some embodiments, a single-base nucleotide mixture of the single-base nucleotide mixtures comprises a mixture of labeled nucleotides and non-labeled nucleotides of a single base. In some embodiments, a single-base nucleotide mixture of the single-base nucleotide mixtures comprises non-terminated nucleotides.

In some embodiments, the system further comprises amplification reagents. In some embodiments, the amplification reagents comprise a polymerase, a nucleotide mixture, a primer, a buffer, or any combination thereof.

In some embodiments, the system further comprises a biological sample. In some embodiments, the biological sample is a tissue. In some embodiments, the biological sample is fixed. In some embodiments, the biological sample is permeabilized. In some embodiments, the biological sample is loaded on the first substrate.

In some embodiments, a geolocation bead of the plurality of geolocation beads comprises at least 100,000 oligonucleotide molecules. In some embodiments, the at least 100,000 oligonucleotide molecules comprise a spatial tag sequence of the spatial tag sequences that is common and unique to the bead amongst the plurality of beads.

In some embodiments, an oligonucleotide molecule of the oligonucleotide molecules comprises a capture sequence, wherein the capture sequence is configured to hybridize with a sequence of an analyte, or derivative thereof, of a biological sample. In some embodiments, the capture sequence is selected from the group consisting of: a polyT sequence, a polyG sequence, a targeted mRNA sequence, a targeted gDNA sequence, a random n-mer sequence, and a probe sequence.

In some embodiments, the substrate comprises at least 1,000,000 individually addressable locations. In some embodiments, the first substrate comprises at least 1,000,000,000 individually addressable locations.

In some embodiments, the plurality of geolocation beads is immobilized to the individually addressable locations via electrostatic interactions.

In some embodiments, the sequencing platform is configured to perform sequencing by synthesis on the substrates.

In some embodiments, the first substrate is substantially planar.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIGS. 1A-1B illustrate examples of a geolocation bead.

FIG. 1C illustrates example methods for synthesizing the geolocation bead constructs of FIGS. 1A-1B and for releasing first strands from such geolocation bead constructs.

FIGS. 2A-2B illustrate reagents and a workflow for generating a spatially tagged sequence using a bridge construct and multiple spatial tags.

FIG. 3 illustrates an example map that can be generated from a sample set of spatial tag data.

FIG. 4 illustrates a general workflow of a substrate-based spatial screening method.

FIG. 5A illustrates a spatial screening scheme using multiple substrates.

FIG. 5B illustrates an example particle comprising a plurality of oligonucleotide molecules including spatial tags.

FIG. 6 illustrates an example droplet with reagents of the present disclosure.

FIG. 7 illustrates an example solution environment with reagents of the present disclosure.

FIGS. 8A-8B illustrate examples of additional geolocations bead constructs.

FIG. 9A illustrates examples of additional bridge constructs. FIG. 9B illustrates examples of additional tagging schemes.

FIGS. 9C-9D illustrate various bead release mechanisms.

FIG. 10 illustrates an example method for generating a tagged sequence under a 5′ approach.

FIG. 11A illustrates example constructs of a geolocation bead and a capture bead. FIG. 11B provides example capture complexes comprising capture beads.

FIG. 12 illustrates an example of additional capture bead and geolocation bead constructs.

FIG. 13 illustrates example individually addressable locations on different substrates.

FIGS. 14A-14G illustrate different examples of cross-sectional surface profiles of a substrate.

FIGS. 15A-15B illustrate methods for loading beads onto a substrate. FIG. 15A illustrates a method for loading beads onto specific regions of a substrate. FIG. 15B illustrates a method for loading a subset of beads onto specific regions of a substrate.

FIG. 16 shows an example coating of a substrate with a hexagonal lattice of beads.

FIG. 17A shows an example system and method for loading a sample or a reagent onto a substrate. FIG. 17B shows another example system and method for loading a sample or a reagent onto a substrate.

FIG. 18 shows a flowchart for an example of a method for sequencing a nucleic acid molecule.

FIGS. 19A-19D illustrate an additional workflow for spatially encoding analytes using geolocation beads and bridge constructs.

FIG. 20 illustrates an additional workflow for spatially encoding analytes using geolocation beads and bridge constructs.

FIG. 21 illustrates an example of a substrate map using fiducial marker beads according to the methods described herein.

FIG. 22 illustrates a schematic for subjecting a reaction space to electrophoresis.

FIGS. 23A-23B illustrate an example method for preparing a geolocation bead library comprising two types of geolocation beads.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are systems, methods, kits, and compositions for the spatial mapping of a plurality of analyte sequences with respect to each other. Each of the plurality of analyte sequences may be tagged with multiple spatial tags, such that each analyte sequence is associated with a set of two or more spatial tags. The sets of spatial tags may be analyzed to generate a map of analyte sequences. The map may comprise information about the respective absolute positions of each of a set of sequences with respect to a reference sequence. Alternatively, or in addition, the map may comprise information about the respective probability cloud (or likely location) of each of a set of sequences with respect to a reference sequence. Beneficially, the provided systems, methods, kits, and compositions allow for the use of spatial tags which respective locations are not previously known or assayed prior to tagging the analyte sequences. This is advantageous over other systems which determine a spatial relationship between analyte sequences using an array of probes or other spatial tags with known locations, such as a system which has to deliberately generate such array with known locations and/or a system in which a pre-determination step has to be performed to determine the identities and locations of the probes or other spatial tags in the array.

A method for the spatial mapping of a plurality of analyte sequences may comprise: (a) loading a plurality of geolocation beads onto a substrate to immobilize the plurality of geolocation beads onto a plurality of individually addressable locations on the substrate, where each geolocation bead comprises a spatial tag; (b) loading a sample onto the substrate (e.g., over the plurality of geolocation beads), where the sample retains a spatial relationship between a plurality of analyte sequences; (c) releasing spatial tags from the geolocation beads; (d) subjecting the sample to conditions sufficient to tag a set of at least two spatial tags to each of the plurality of analyte sequences (e.g., mRNA) to generate a plurality of spatially tagged sequences; and (e) generating a map of the plurality of analyte sequences by identifying sets of spatial tags from the plurality of spatially tagged sequences, where the map comprises information about the respective locations or respective probability cloud (or likely location) of each of a set of analyte sequences with respect to a reference analyte sequence.

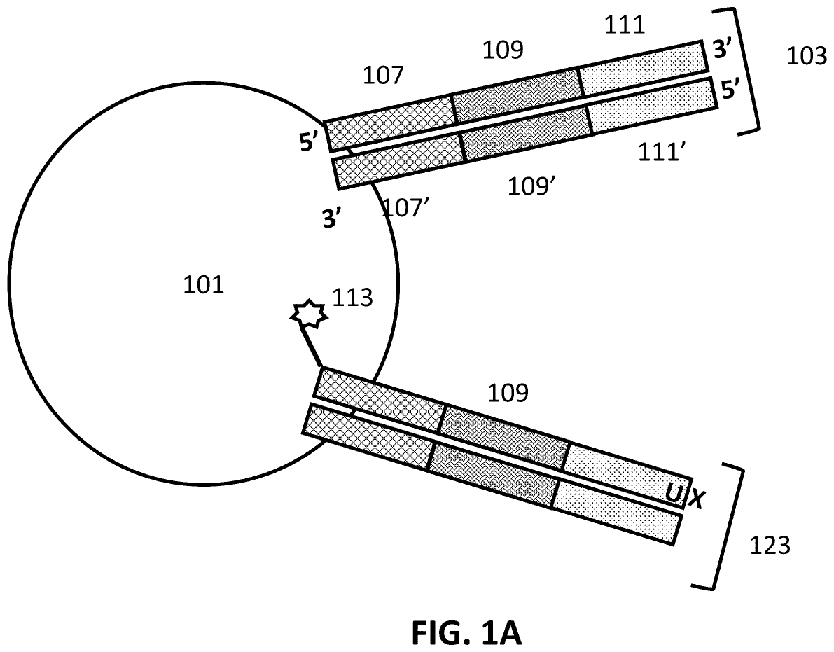

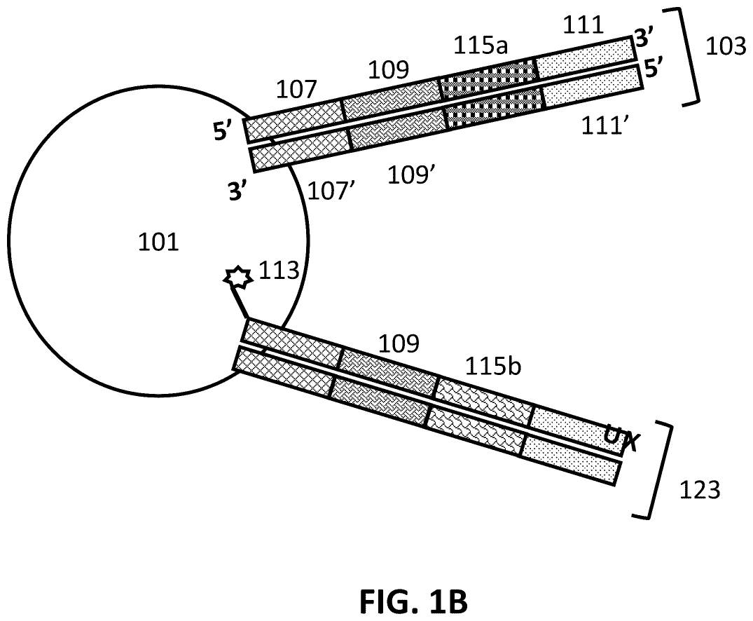

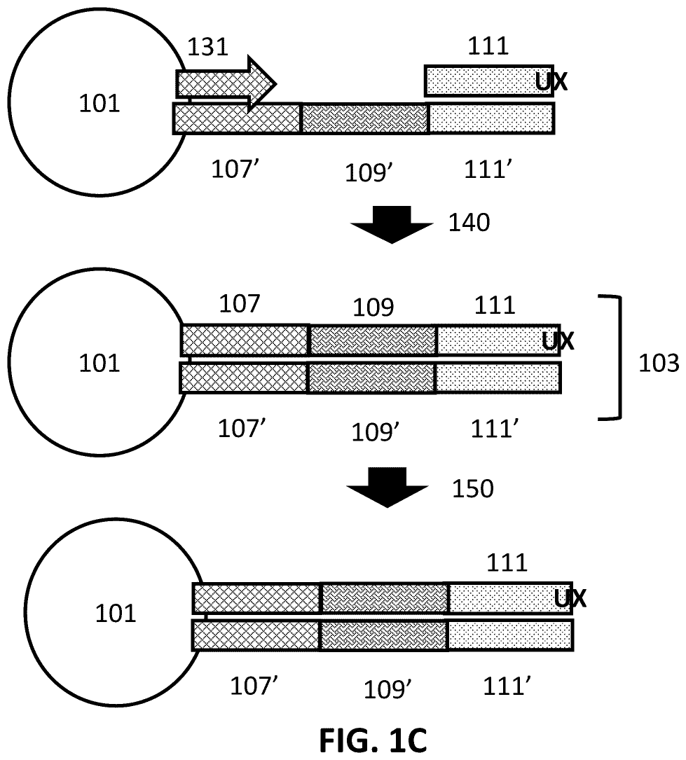

FIGS. 1A-1B illustrate examples of a geolocation bead. Referring to FIGS. 1A-1B, a geolocation bead 101 may comprise a plurality of oligonucleotide molecules (e.g., 103, 123) attached thereto. Though two oligonucleotide molecules are illustrated in these figures, any number of oligonucleotide molecules may be attached to the geolocation bead, for example on the order of 10, 10², 10³, 10⁴, 10⁵, 10⁶, 10⁷, 10⁸, or more. An oligonucleotide molecule 103 may be a partially or wholly double-stranded molecule, in which a first strand comprises a first attachment sequence 107, a spatial tag 109, and a second attachment sequence 111, and a second strand comprises complementary sequences 107′, 109′, and 111′ for the sequences in the first strand, respectively. Each oligonucleotide molecule of the plurality of oligonucleotide molecules on the geolocation bead 101 may comprise a common spatial tag 109. Different geolocation beads may comprise different spatial tags. For example, a first geolocation bead may comprise a first plurality of oligonucleotide molecules each comprising a first spatial tag and a second geolocation bead may comprise a second plurality of oligonucleotide molecules each comprising a second spatial tag that is different form the first spatial tag. Thus, two nucleic acid molecules comprising the spatial tag, when identified (e.g., sequenced), may inform an operator that the two nucleic acid molecules were tagged by oligonucleotide molecules from the same geolocation bead.

In some cases, as illustrated in FIG. 1B, an oligonucleotide molecule may comprise a unique molecular identifier (UMI) sequence (e.g., 115a, 115b). The unique molecular identifier sequence may be unique to each oligonucleotide molecule of the plurality of oligonucleotide molecules on the same geolocation bead. For example, oligonucleotide molecule 103 and oligonucleotide molecule 123 which is attached to the same geolocation bead 101 may comprise the same spatial tag 109 sequence, but different UMI sequences 115a, 115b, respectively. The UMI sequence may be disposed anywhere between the first attachment sequence 107 and the second attachment sequence 111. The UMI may be a nucleic acid sequence. The UMI may comprise at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more bases. Alternatively or in addition, the UMI may comprise at most about 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or fewer bases.

The first strands comprising the spatial tag 109 may be released from the geolocation bead 101 via various mechanisms. For example, in an example release mechanism, a strand displacing polymerase (e.g., a polymerase with relatively strong ability to strand displacement compared to polymerases that lack strand displacement activity such as T4 and T7 DNA polymerases) and a primer may be provided under conditions sufficient to displace the first strand (comprising sequences 107, 109, 111) from the oligonucleotide molecule. Examples of strand displacing polymerases include, but are not limited to, Bst DNA polymerase, large fragment polymerase, and D29 polymerase. In some cases, for strand displacement in the 5′ to 3′ direction, the strand displacing polymerase may generate or leverage a flap at the 5′ end. As described elsewhere herein, where strand displacement is in the 3′ to 5′ direction, the strand displacing polymerase may generate or leverage a flap at the 3′ end. In another example release mechanism, an enzyme may be provided to digest the second strand (e.g., at least a portion thereof) to release the first strand. The enzyme may comprise 5′ to 3′ exonuclease activity. Examples of enzymes with 5′ to 3′ exonuclease activity include, but are not limited to, Lambda Exonuclease, T7 Exonuclease, T5 Exonuclease, Exonuclease V, and Exonuclease VIII. The enzyme may comprise 3′ to 5′ exonuclease activity. Examples of enzymes with 3′ to 5′ exonuclease activity include, but are not limited to, Exonuclease III and Exonuclease V. In one example, a 5′ end of the second strand may be phosphorylated and subjected to Lambada Exonuclease.

In some cases, the oligonucleotide molecules (e.g., 103, 123) may be immobilized to the bead 101 via a cross-link or other linker. The cross-link or other link may be cleaved to release the oligonucleotide molecules, or portion thereof, such as by applying one or more stimuli, including light stimuli, heat stimuli, chemical stimuli, magnetic stimuli, electrical stimuli, and other stimuli, or combination thereof. In some cases, the oligonucleotide molecules may be immobilized to the bead via a photo-cross-link. A photo-cross-link may be generated by a photo cross-linking reaction. In some instances, an oligodeoxynucleotide (ODN) comprising 3-cyanovinylcarbazole nucleoside (^{CNV}K) can be subjected to photoirradiation conditions to photo-cross-link a target pyrimidine and the ^{CNV}K. In some instances, irradiation is provided at 366 nm for about 1 second for photo-cross-linking to thymine, and for up to about 25 seconds for photo-cross-linking to cytosine. In this example, irradiation provided at 312 nm for about 3 minutes can reverse the cross-link. Various other cross-linking reagents may be used to generate a cross-link (e.g., chemical cross-link). In some cases, heat may be applied to denature a double-stranded molecule to facilitate release of at least one of the strands (e.g., first strand) from the bead. The heat stimulus can be combined with cross-linking reactions. For example, the first strand may be released from the bead by applying a heat stimulus. In some cases, additionally, a primer may be cross-linked (e.g., photo-cross-linked) to the oligonucleotide molecule and extended, such as to prevent re-hybridization of the released strand to the remaining strand.

In some cases, the first strand may comprise one or more features (e.g., a blocking group) that prevent digestive activity by the enzyme on the first strand, such as illustrated as “X” for oligonucleotide molecule 123. In some cases, the first strand may comprise a cleavage site, such as illustrated as “U” for oligonucleotide molecule 123. In some cases, the oligonucleotide molecule may be capped by an uracil, a ribonucleotide, or other modified nucleotide to facilitate digestion of the second strand to release the first strand. In some cases, the second strand may comprise one or more nicks (or nicks may be created) to facilitate strand displacement and/or digestive activity by an enzyme. In some instances, the second strand may comprise one or more cleavable or digestible moieties (e.g., ribonucleotides, uracil, etc.) for cleavage or digestion by one or more enzymes (e.g., RNase HII).

In some examples, a USER cleavage reaction may be performed to process the cleavable or digestible moieties from the double stranded molecules and release the remaining molecule from the bead. In the USER cleavage reaction, a USER (uracil-specific excision reagent) enzyme may generate a nucleotide gap at a location of a uracil base (e.g., dU) in the molecule and facilitate cleavage.

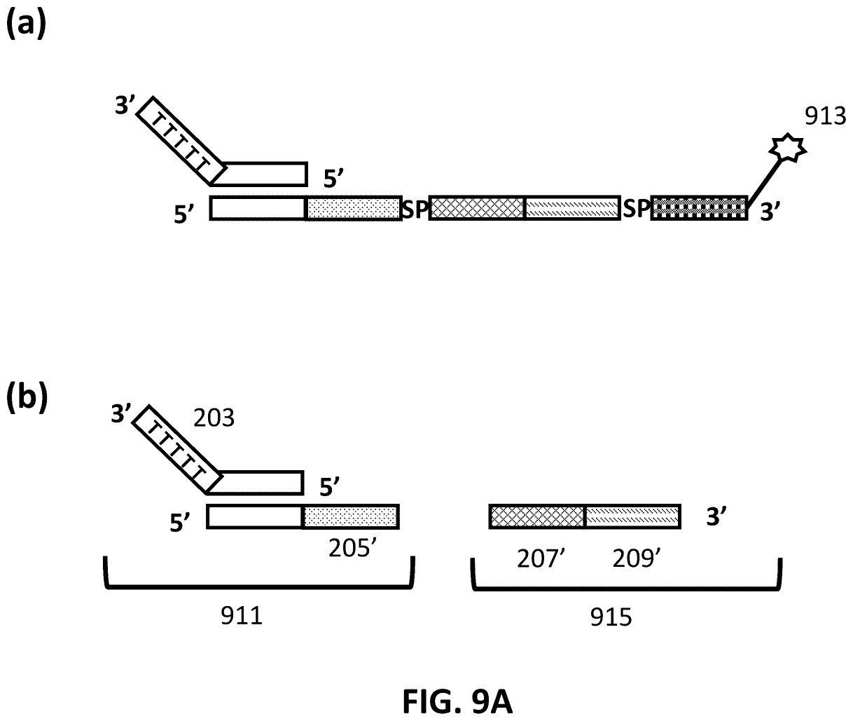

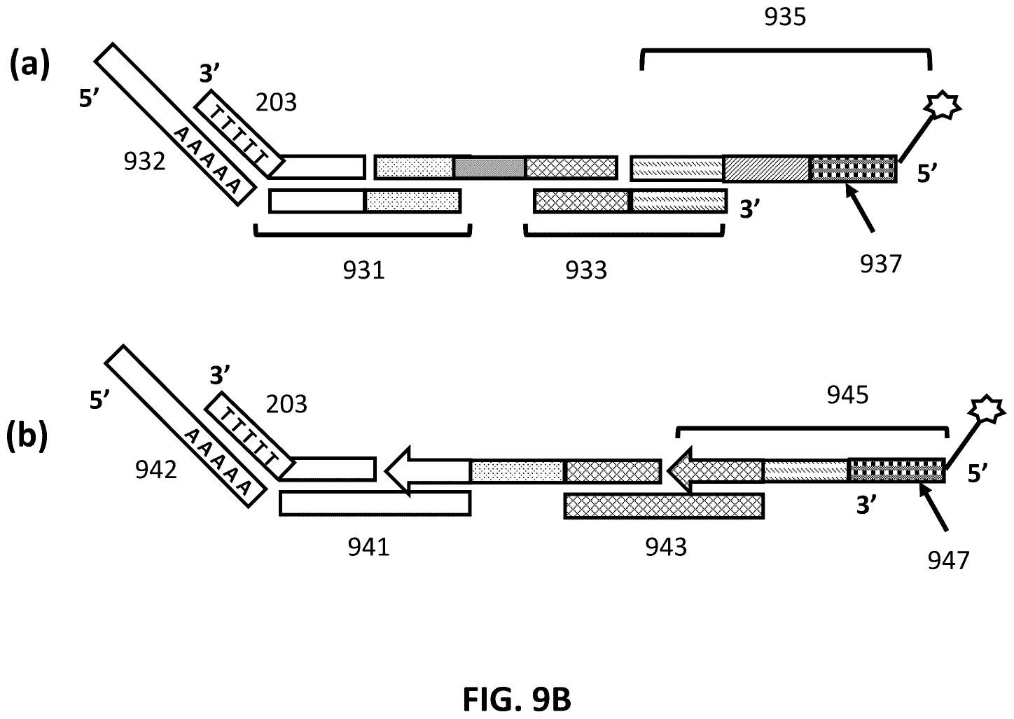

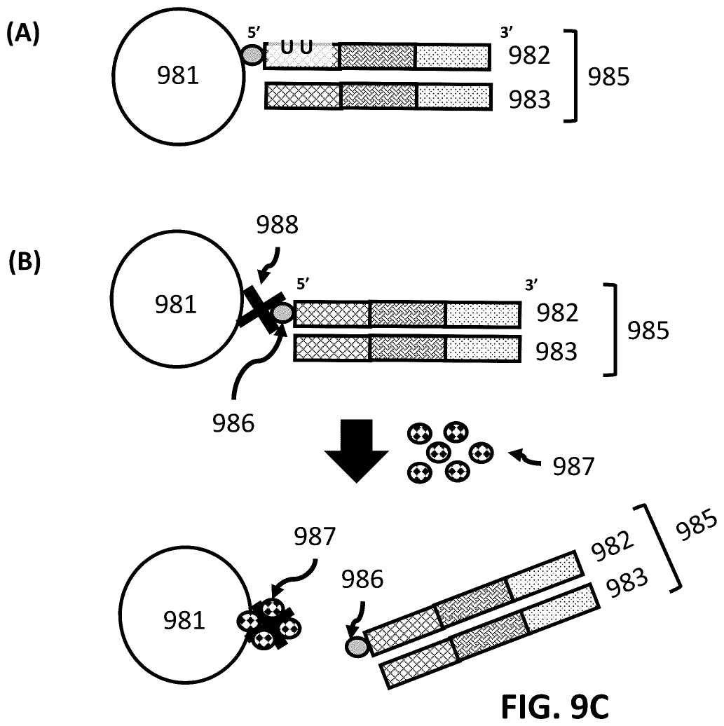

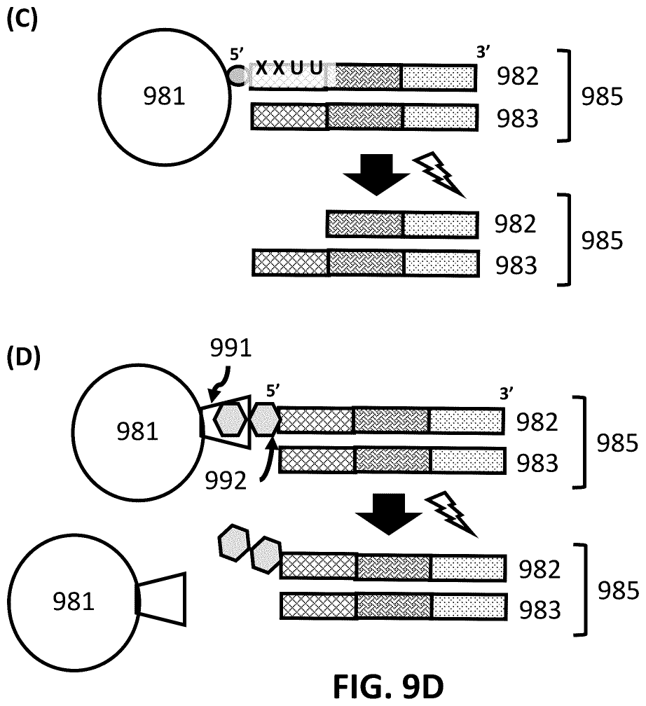

FIGS. 9C-9D illustrate various bead release mechanisms. For all panels, shown is a bead 981 comprising a double-stranded oligonucleotide molecule 985, which comprises a first strand 982 and a second strand 983.

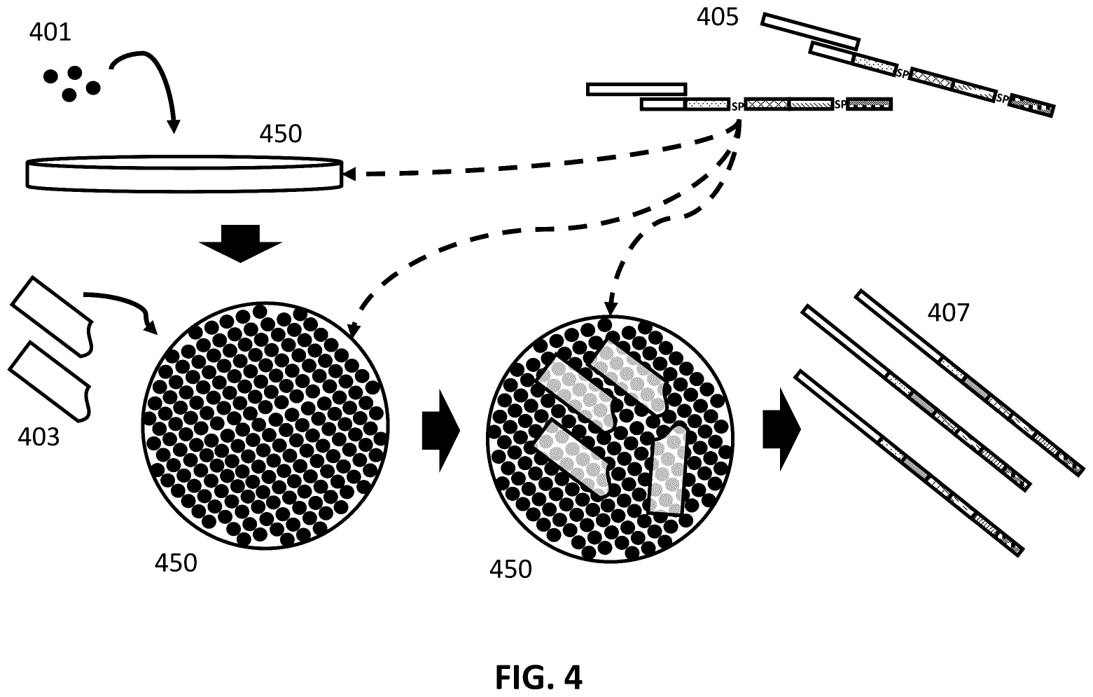

In panel (A), the first strand 982 may comprise a cleavage site, denoted “U”, at or adjacent to the 5′ terminus. The cleavage site may be cleaved to release the first strand from the bead. One or both strands may have one or more cleavage sites. In some cases, a USER enzyme mix may be used for the cleavage reaction. The USER enzyme mix may comprise uracil DNA glycosylase (UDG), which removes the sugar and creates an abasic site (AP site), and endonuclease (e.g., endonuclease VIII), which binds to the AP Site and cleaves. In some cases, to accelerate cleavage, the components of the USER enzyme mix may be divided and provided independently, and later combined. For example, with reference to the methods of FIG. 4, the UDG may be provided separately to the substrate 450 comprising a plurality of geolocation beads immobilized thereto, prior to loading of the sample (e.g., tissue), and the endonuclease may be provided separately to the sample (e.g., tissue). The cleavage reaction may take effect after the sample is loaded onto the substrate. Beneficially, the AP sites may be created as a substrate-priming step, prior to loading of the sample. In some cases, the endonuclease may be replaced with an APE1 enzyme in the USER enzyme mix, which cleaves multiple times and is Mg²⁺-dependent (as opposed to endonuclease VIII which is Mg²⁺-independent). In an example, with reference to the methods of FIG. 4, the UDG and APE1 enzyme may be provided to the substrate 450 comprising a plurality of geolocation beads immobilized thereto, without Mg²⁺, and prior to loading of the sample, to prime the substrate and form the AP sites. The APE1 enzyme may bind to the AP sites, without cleavage activity due to the lack of the Mg²⁺. Separately, Mg²⁺ may be provided separately to the sample (e.g., tissue). The cleavage reaction may take effect after the sample is loaded onto the substrate. It will be appreciated that different components of an enzyme mix may be provided to (1) the substrate, and (2) the sample, other than the specific examples (e.g., a first type of enzyme to the substrate, and a second type of enzyme to the sample; two types of enzymes to the substrate, and a catalyst or other reagent to the sample, or vice versa, etc.).

Panels (B)-(D) describe non-enzymatic release mechanisms. In panel (B), the oligonucleotide molecule 985 may be coupled to (e.g., conjugated to) a desthiobiotin moiety 986 (a biotin analog which lacks the sulfur atom), and the geolocation bead 981 may be coupled to a streptavidin moiety 988. The desthiobiotin moiety 986 and the streptavidin moiety 988 may be bound together to couple the oligonucleotide molecule 985 to the geolocation bead 981, though at less binding strength (e.g., with disassociation constant (K_{d}) on the order of 10⁻¹¹ M) than that between streptavidin and biotin moieties (e.g., with K_{d} on the order of 10⁻¹⁵ M). Upon provision of a high concentration of biotin moieties 987, the biotin-streptavidin bonds may displace the desthiobiotin-streptavidin bonds to release the oligonucleotide molecule 985 from the geolocation bead 981. Such non-enzymatic release mechanisms may be beneficial over enzymatic release mechanisms. For example, it may result in shorter release time (faster than enzymatic cleavage time, e.g., using USER cleave); it may reduce cost (biotin is cheaper compared to enzyme reagents); it may improve diffusion in hydrogel environments as biotins are much smaller in size than enzymes; it may reduce waste by permitting recycling of streptavidin-coupled beads (by extracting the biotin from the used geolocation beads, and attaching new desthiobiotin-conjugated oligonucleotide molecules).

In panel (C), the first strand 982 may comprise one or more azobenzene (denoted ‘X’) and a cleavage site, denoted “U”, at or adjacent to the 5′ terminus. For example, the cleavage site may comprise 1, 2, 3, 4, or more uracil residues. Azobenezene is a light-sensitive molecule that changes between a trans-form and a cis-form under certain light and/or heat conditions. For example, azobenzene changes from trans-form to cis-form under UV light, and changes from cis-form to trans-form under VIS light and/or heat. Azobenzene may enable fast photoswitch of hybridization states of at least a segment of two strands of nucleic acid molecules. Azobenzene may be incorporated between the nucleotides of the first strand. The melting temperature (Tₘ) between the first strand 982 and the second strand 983 may be significantly reduced when the azobenzene is in cis-form, as compared to the Tₘ between the two strands without azobenzene and as compared to the Tₘ when the azobenzene is in trans-form, as it weakens the hydrogen bonds between the two strands. The Tₘ between the first strand the second strand may not vary as much when the azobenzene is in trans-form as compared to the Tₘ between the two strands without the azobenzene. Thus, providing UV light stimulus (e.g., 365 nm) to the geolocation bead 981 may change the azobenzene to cis-form and dehybridize, destabilize, or facilitate dehybridizing or destabilizing of the two strands at the location of the azobenzene incorporations. With reference to the methods described with respect to FIG. 4, after the substrate is loaded with a plurality of geolocation beads, the geolocation beads may be subjected to a USER enzyme mix, as described elsewhere herein, to form nicked strands (e.g., on strand 982) at the cleavage sites. The geolocation bead may be subjected to UV light to trigger the azobenzene, resulting in fast release of the nicked strands from the geolocation bead.

In panel (D), the oligonucleotide molecule 985 may be coupled to (e.g., conjugated to) a azobenzene moiety 992 at a 5′ terminus, which azobenzene moiety is hydrophobic, and the bead 981 may be coupled to (e.g., conjugated to) an alpha-cyclodextrine (a-CD) moiety 991, which is a barrel protein with a hydrophilic outer shell and a hydrophobic core. The photoswitchable trans-form and cis-form of azobenzene has been described elsewhere herein. In trans-form, azobenzene may enter the a-CD core via hydrophobic interaction, and thus bind the oligonucleotide molecule 985 and the bead 981 in a non-covalent bond. In cis-form, azobenzene may exit the a-CD core and detach from the a-CD, thus releasing the oligonucleotide molecule 985 from the geolocation bead 981. The transition from trans-form to cis-form of azobenzene may be triggered by UV light.

In some cases, the first strand may comprise a capture entity 113, such as illustrated for oligonucleotide molecule 123, which is configured for capture by a capturing entity. The capture entity may comprise or be biotin, a capture sequence (e.g., nucleic acid sequence) which may be hybridized to the second strand or which may be part of another nucleic acid molecule conjugated to the oligonucleotide molecule, a magnetic particle capable of capture by application of a magnetic field, a charged particle capable of capture by application of an electric field, a combination thereof, or one or more other mechanisms configured for, or capable of, capture by a capturing entity. The capturing entity may comprise or be streptavidin when the capture moiety comprises biotin, a complementary capture sequence when the capture entity comprises a capture sequence, an apparatus, system, or device configured to apply a magnetic field when the capture entity comprises a magnetic particle, an apparatus, system, or device configured to apply an electrical field when the capture entity comprises a charged particle, a combination thereof, and/or one or more other mechanisms configured to capture the capture entity. In some instances, the capturing group may comprise a secondary capture entity, for example, for subsequent capture by a secondary capturing entity. The secondary capture entity and secondary capturing entity may comprise any one or more of the capturing mechanisms described elsewhere herein (e.g., biotin and streptavidin, complementary capture sequences, etc.). In some instances, the secondary capture entity can comprise a magnetic particle (e.g., magnetic geolocation bead) and the secondary capturing entity can comprise a magnetic system (e.g., magnet, apparatus, system, or device configured to apply a magnetic field, etc.). In some instances, the secondary capture entity can comprise a charged particle (e.g., charged geolocation bead carrying an electrical charge) and the secondary capturing entity can comprise an electrical system (e.g., magnet, apparatus, system, or device configured to apply an electric field, etc.). In some instances, the capture moiety comprises biotin, the capturing moiety comprises streptavidin coupled to a secondary capture entity, a magnetic geolocation bead, and the secondary capturing entity comprises a magnetic system.

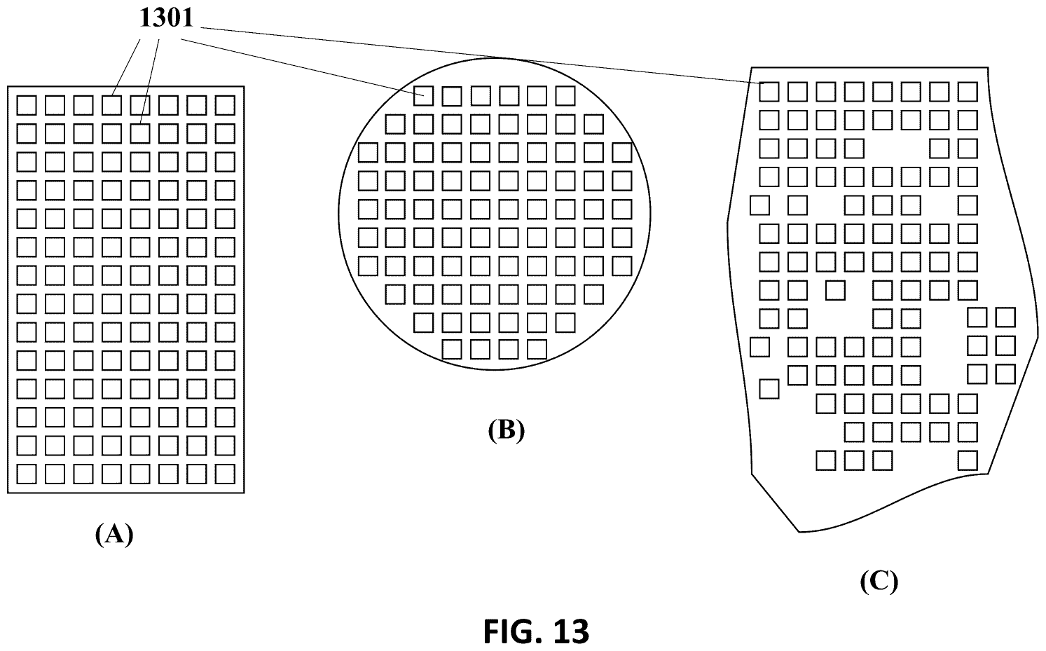

The spatial tag (e.g., 109) may be a nucleic acid sequence. The spatial tag may comprise at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more bases. Alternatively or in addition, the spatial tag may comprise at most about 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or fewer bases. The spatial tag may be unique and common to a geolocation bead amongst a plurality of geolocation beads. In some cases, the spatial tag may be substantially unique to a geolocation bead amongst a plurality of geolocation beads such that at least about 50%, 55%, 60%, 65%, 70%, 75%, 80%, 85%, 90%, 91%, 92%, 93%, 94%, 95%, 96%, 97%, 98%, 99% or more of geolocation beads in the plurality of geolocation beads comprise unique spatial tags not contained by any other geolocation bead in the plurality of geolocation beads. The methods, systems, compositions, and kits of the present disclosure may comprise any number of geolocation beads. In some cases, there may be a number of geolocation beads on the order of at least on the order of 10², 10³, 10⁴, 10⁵, 10⁶, 10⁷, 10⁸, 10⁹, 10¹⁰, 10¹¹, 10¹², 10¹³, 10¹⁴, or more beads. In some cases, there may be at least a number of geolocation beads to correspond to a number of individually addressable locations available on the substrate to which the geolocation beads are loaded. Example arrays (e.g., possible distributions) of individually addressable locations 1301 on a substrate are illustrated in FIG. 13 (e.g., from a top view). In FIG. 13, panel A shows a substantially rectangular substrate with regular linear arrays, panel B shows a substantially circular substrate with regular linear arrays, and panel C shows an arbitrarily shaped substrate with irregular arrays. Irregular or regular arrays may be disposed on any shape of substrate.

The first attachment sequence (e.g., 107) may be configured for attachment or coupling (e.g., hybridization) to a bridge construct, as described elsewhere herein. The second attachment sequence (e.g., 111) may be configured for attachment or coupling (e.g., hybridization) to the bridge construct. The first attachment sequence and the second attachment sequence may comprise different sequences. The first attachment sequence and the second attachment sequence may be or comprise the same sequence. The first attachment sequence and/or the second attachment sequence may comprise at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more bases. Alternatively or in addition, the first attachment sequence and/or the second attachment sequence may comprise at most about 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or fewer bases.

In some cases, the first attachment sequence may be common to a plurality of geolocation beads such that each geolocation bead comprises the same first attachment sequence. In some cases, the second attachment sequence may be common to a plurality of geolocation beads such that each geolocation bead comprises the same second attachment sequence. In some cases, both the first attachment sequence and the second attachment sequence may be common to a plurality of geolocation beads such that each geolocation bead comprises both the same first attachment sequence and the second attachment sequence.

In some cases, the first attachment sequence may be common only to a subset of plurality of geolocation beads such that each geolocation bead in the subset comprises the same first attachment sequence, and one or more other subsets of the geolocation beads comprises a different first attachment sequence. In some cases, the second attachment sequence may be common only to a subset of plurality of geolocation beads such that each geolocation bead in the subset comprises the same second attachment sequence, and one or more other subsets of the geolocation beads comprises a different second attachment sequence. In some cases, a pair of the first attachment sequence and the second attachment sequence may be common only to a subset of plurality of geolocation beads such that each geolocation bead in the subset comprises the same pair of the first attachment sequence and the second attachment sequence, and one or more other subsets of the geolocation beads comprises a different pair of the first attachment sequence and the second attachment sequence (where either one of or both the first attachment sequence and the second attachment sequence are different). In some cases, a plurality of geolocation beads may comprise at least 2, 3, 4, 5, 6, 7, 8, 9, 10, 20, 30, 40, 50, 60, 70, 80, 90, 100, 1000, 10,000, or more subsets of geolocation beads within a plurality of geolocation beads with the same number of different pairs of first and second attachment sequences. Alternatively or in addition, a plurality of geolocation beads may comprise at most 10,000, 1000, 100, 90, 80, 70, 60, 50, 40, 30, 20, 10, 9, 8, 7, 6, 5, 4, 3, or 2 subsets of geolocation beads within a plurality of geolocation beads with the same number of different pairs of first and second attachment sequences.

In some cases, for a geolocation bead, there may be only one pair of first attachment sequence and second attachment sequence amongst the plurality of oligonucleotide molecules on the geolocation bead. In other cases, for a geolocation bead, there may be more than one pair of first attachment sequence and second attachment sequence amongst the plurality of oligonucleotide molecules on the geolocation bead.

The plurality of geolocation beads may be loaded onto a substrate. The substrate may comprise a plurality of individually addressable locations. Upon or subsequent to loading, the plurality of geolocation beads may be immobilized to respective individually addressable locations on the substrate. All or a subset of individually addressable locations on the substrate may immobilize the plurality of geolocation beads. The identities of the spatial tags on the geolocation beads may be unknown. Alternatively, or in addition, the locations of the spatial tags on the geolocation beads may be unknown. In some cases, the identities of the spatial tags on the geolocation beads may be known, but their individual locations on the substrate may be unknown. The present disclosure may obviate the need to assay the immobilized beads to determine the identity-location information of the spatial tags prior to tagging of analyte sequences. Substrates and individually addressable locations are described in further detail elsewhere herein.

Subsequent to immobilization of the geolocation beads on the substrate, a sample may be loaded onto the substrate. The sample may retain, at least to some extent, a spatial relationship between a plurality of analyte sequences. In some instances, a tissue slice is loaded onto the substrate, where the tissue slice retains, at least to some extent, a spatial relationship between the transcripts contained therein. A sample may comprise a biological sample. The biological sample may be derived from a subject. The sample may comprise a plurality of nucleic acid molecules (e.g., comprising analyte sequences), such as messenger RNA (mRNA) molecules. Samples that can be used in the present methods, systems, compositions, and kits are described in further detail elsewhere herein.

The spatial tags may be released from the geolocation beads, by releasing the first strands as described elsewhere herein, prior to, during, or subsequent to loading of the sample on the substrate. The releasing of the first strands from the geolocation beads may be referred to herein as ‘activation’ of the geolocation beads. Geolocation beads may be activated by providing one or more enzymes, as described elsewhere herein. In some cases, geolocation beads may be activated by providing one or more stimuli, such as heat, chemical, or light stimuli, a combination thereof, or any other stimuli. In some cases, for example, both heat and enzymes may be provided to activate the geolocation beads.

FIG. 1C illustrates example methods for synthesizing the geolocation bead constructs of FIGS. 1A-1B and for releasing first strands from such geolocation bead constructs. A geolocation bead 101 may comprise a partially double-stranded nucleic acid molecule attached thereto, the partially double-stranded nucleic acid molecule including a first strand and a second strand. The second strand can comprise complementary sequences (e.g., 107′, 109′, and 111′) for the sequences to be included in the first strand of the oligonucleotide molecule (e.g., 103) on the final geolocation bead construct. For example, the complementary sequences may be complementary to a first attachment sequence 107, a spatial tag sequence 109, and a second attachment sequence 111, and optionally a UMI (e.g., 115a, 115b) of the first strand of the oligonucleotide molecule 103. The first strand of the partially double-stranded nucleic acid molecule may comprise the second attachment sequence 111 bound to the complementary sequence on the second strand, for example at the 5′ end of the second strand. The first strand can comprise one or more features (e.g., a blocking group (“X”), cleavage sites (“U”)), as described with respect to oligonucleotide molecule 123. A primer sequence 131 comprising at least a portion of the first attachment sequence 107 may bind to the complementary sequence (e.g., 107′) in the second strand, extended to fill the gap between the primer sequence 131 and the second attachment sequence 111, and ligated 140 to generate oligonucleotide molecule 103 attached to the geolocation bead 101, as described elsewhere herein. During activation of the geolocation beads 150, the first strand comprising the spatial tag 109 may be released. In some instances, an enzyme configured for strand displacement and a primer may be provided to displace the first strand, and then the cleavage site may be cleaved to make the first strand accessible or able for downstream processing (e.g., able to bind to a bridge construct).

A bridge construct may be provided to the substrate prior to, during, or subsequent to release of the spatial tags. Alternatively, or in addition, the bridge construct may be provided to the substrate prior to, during, or subsequent to loading of the sample on the substrate. Alternatively, or in addition, the bridge construct may be provided to the substrate prior to, during, or subsequent to rendering analyze sequences in the sample accessible by the bridge construct. The bridge construct may capture multiple spatial tags as well as an analyte sequence from the sample, to spatially tag the analyte sequence and generate a spatially tagged sequence. Prior to, during, or subsequent to release of the spatial tags, the analyte sequences in the sample may be rendered accessible for contact with a bridge construct, such as by allowing the bridge construct to cross a membrane of the sample (e.g., diffuse in), and/or by allowing the analyte sequences to cross a membrane of the sample (e.g., diffuse out) under conditions sufficient to retain, at least to some extent, a spatial relationship between the analyte sequences and retain, at least to some extent, a spatial relationship between the plurality of spatial tags. Upon release of the spatial tags, each of the spatial tags may begin diffusion from a respective point of origin (location of geolocation bead) towards a location of capture by the bridge construct. It will be appreciated that the distance that a spatial tag can travel between release and capture is a function of the rate of diffusion and various conditions (e.g., temperature, addition of viscous or crowding agents, concentration of various reagents, etc.). It will be appreciated that the distance that an analyte sequence (e.g., a messenger RNA (mRNA) molecule) can travel between its release from a sample, if released, and capture is a function of the rate of diffusion and various conditions (e.g., temperature, addition of viscous or crowding agents, concentration of various reagents, etc.). The systems, methods, kits, and compositions of the present disclosure provides reagents at concentrations and conditions sufficient to retain, at least to some extent, a spatial relationship between the analyte sequences and retain, at least to some extent, a spatial relationship between the plurality of spatial tags.

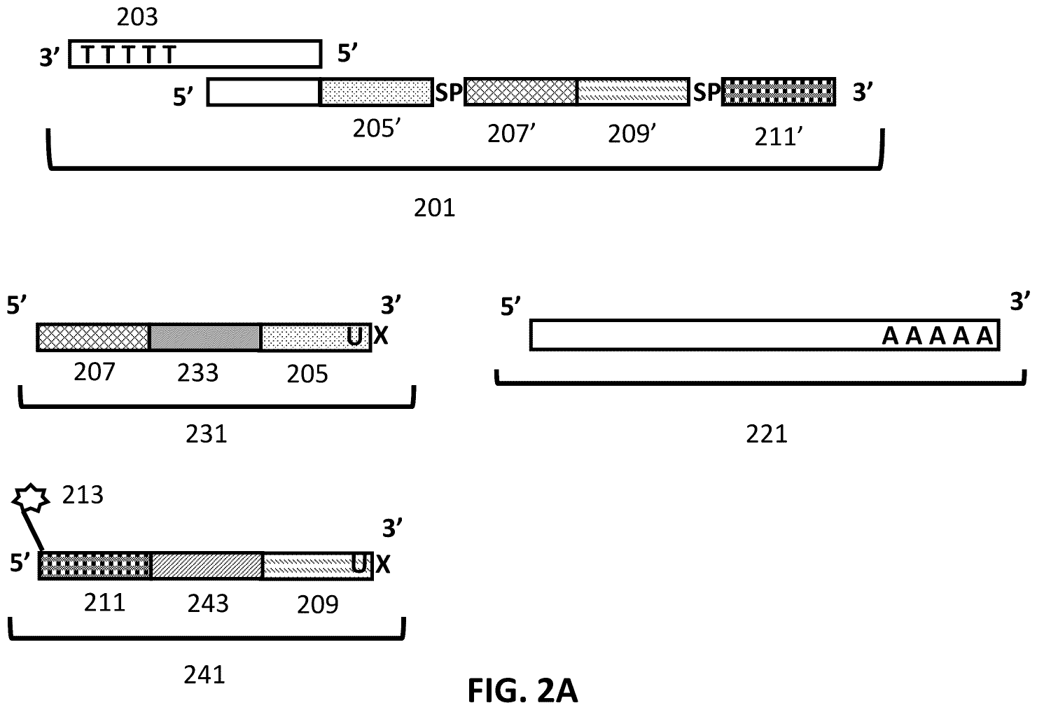

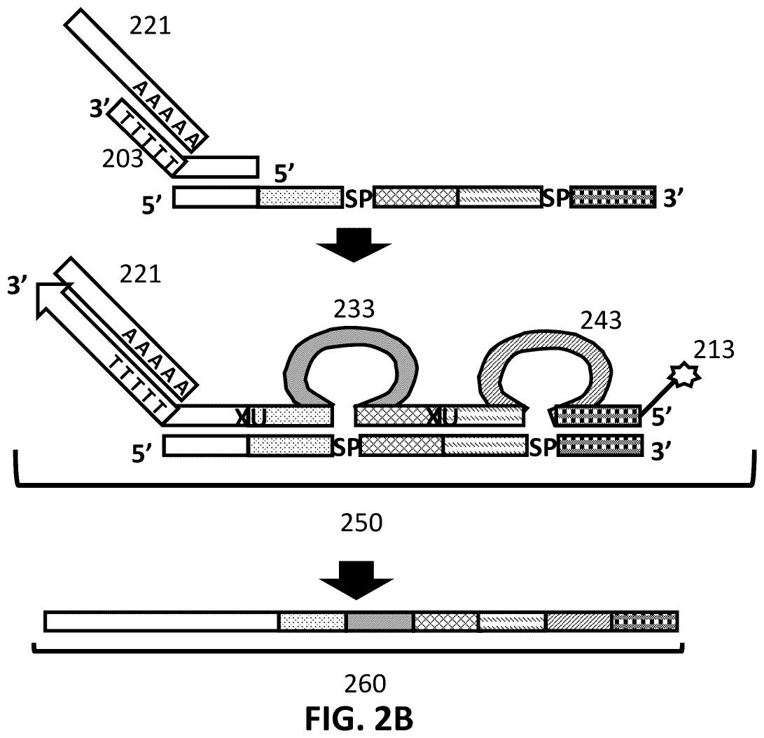

FIGS. 2A-2B illustrate reagents and a workflow for generating a spatially tagged sequence using a bridge construct and multiple spatial tags. Referring to FIG. 2A, provided and accessible for reaction are a bridge construct 201, an analyte sequence 221, and multiple spatial tags, including a first spatial tag molecule 231 and a second spatial tag molecule 241.

The first spatial tag molecule 231 may comprise a first spatial tag 233 disposed between a first pair of attachment sequences (e.g., first attachment sequence 207 and second attachment sequence 205), and the second spatial tag molecule 241 may comprise a second spatial tag 243 disposed between a second pair of attachment sequences (e.g., third attachment sequence 211 and fourth attachment sequence 209). The first and second spatial tags may be different. The first and second pairs of attachment sequences may be different. In FIG. 2A, the first spatial tag molecule 231 comprises, from 5′ to 3′, the first attachment sequence 207, the first spatial tag 233, and the second attachment sequence 205. The second spatial tag molecule 241 comprises, from 5′ to 3′, the third attachment sequence 211, the second spatial tag 243, and the fourth attachment sequence 209. The second spatial tag molecule 241 may comprise at a 5′ end a capture entity 213, as described elsewhere herein.

The bridge construct 201 may comprise a partially double-stranded molecule, where a first strand comprises a capture sequence 203 as an overhang and a binding sequence which binds to the second strand. The second strand may comprise a binding sequence which binds to the first strand, a first attachment binding sequence 207′, a second attachment binding sequence 205′, a third attachment binding sequence 211′, a fourth attachment binding sequence 209′, and spacer sequences. In FIG. 2A, the second strand comprises, from 5′ to 3′, the binding sequence, the second attachment binding sequence 205′, a spacer sequence, the first attachment binding sequence 207′, the fourth attachment binding sequence 209′, a spacer sequence, and the third attachment binding sequence 211′.

The capture sequence 203, while in FIG. 2A is denoted as a poly-T sequence (e.g., TTTTT) configured to capture a poly-A tail of the analyte sequence 221 (e.g., an mRNA sequence), may be any sequence configured to capture an analyte sequence. The analyte sequence, for example, may not have a poly-A tail. In some instances, the capture sequence may comprise a target sequence or a random sequence or any other sequence designed to capture an analyte sequence, or derivative thereof. The capture sequence may comprise a random n-mer sequence. In some examples, for targeted mRNA assays, the capture sequence may comprise a target mRNA sequence (or derivative thereof). In some examples, for targeted genomic DNA (gDNA) assays, the capture sequence may comprise a target gDNA sequence (or derivative thereof). In some examples, for antibody assays, the capture sequence may comprise a sequence configured to capture an oligonucleotide conjugated to one or more antibodies (e.g., DNA capture tags), or a derivative thereof. In some examples, for assays that utilize template switching reactions, the capture sequence may comprise a sequence configured to capture a product of a reverse transcription reaction, such as a polyG sequence. In some examples, for assays that utilize one or more probes, the capture sequence may comprise a sequence corresponding to a sequence of the probe, to a molecule associated with the probe, or derivative thereof. In some examples, the capture sequence may be part of a single strand portion, a double strand portion, or partially double-stranded complex. In some examples, the capture sequence may be part of a hybrid DNA/RNA complex.

In some examples, for a transposition assay concerning gDNA analytes, after a transposition reaction (e.g., subsequent to Tn5 transposase treatment of gDNA, where the Tn5 transposase comprises one or more barcode and/or adapter sequences), a partially double-stranded analyte may be generated. In some examples, wherein at least one end of the partially double-stranded analyte comprises an overhang comprising a barcode and/or adapter sequence. A bridge construct of the present disclosure (e.g., 201) may comprise a capture sequence (e.g., 203) that is configured to capture the overhang of the partially double-stranded analyte comprising the barcode and/or adapter sequence. One or more gap filling and/or ligation reactions may be performed to join the partially double-stranded bridge construct and transposition analyte.

Spacer sequences (e.g., “SP”) in the bridge construct may be the same sequence or different sequences. Spacer sequences may comprise a sequence of any length. For example, the spacer can be any internal spacer, e.g., C3 spacer, C12 spacer, spacer 9, spacer 18, etc. In some cases, the spacer sequence may be designed to not be complementary to any spatial tag sequence, or portion thereof.

Referring to FIG. 2B, the bridge construct 201 may capture the analyte sequence 221 using the capture sequence 203, and be extended using the analyte sequence as a template. The bridge construct 201 may capture the first spatial tag molecule 231 using the second attachment binding sequence 205′ and the first attachment binding sequence 207′ which hybridizes with the second attachment sequence 205 and the first attachment sequence 207 of the first spatial tag molecule 231, respectively. Once the first spatial tag molecule 231 (e.g., from FIG. 2A) is captured by the bridge construct 201, the first spatial tag 233 may be looped, as illustrated in FIG. 2B. The bridge construct 201 may capture the second spatial tag molecule 241 using the fourth attachment binding sequence 209′ and the third attachment binding sequence 211′ which hybridizes with the fourth attachment sequence 209 and the third attachment sequence 211 of the second spatial tag molecule 241, respectively. Once the second spatial tag molecule 241 (e.g., from FIG. 2A) is captured by the bridge construct 201, the second spatial tag 243 may be looped, as illustrated in FIG. 2B. A tagged complex 250 thus comprises the analyte sequence 221, first spatial tag 233, and second spatial tag 243. The tagged complex 250 may comprise the capture entity 213.

The sample may be incubated with the reagents (e.g., bridge constructs, spatial tags, etc.) for any period of time. For example, the sample may be incubated for at least about 1 minute (min), 2 min, 3 min, 4 min, 5 min, 6 min, 7 min, 8 min, 9 min, 10 min, 20 min, 30 min, 40 min, 50 min, 60 min, 90 min, 120 min, 150 min, 180 min, 4 hours (h), 5 h, 6 h, 7 h, 8 h, 9 h, 10 h, 11 h, 12 h or more. Alternatively or in addition, the sample may be incubated at most about 12 h, 11 h, 10 h, 9 h, 8 h, 7 h, 6 h, 5 h, 4 h, 180 min, 150 min, 120 min, 90 min, 60 min, 50 min, 40 min, 30 min 20 min, 10 min, 9 min, 8 min, 7 min, 6 min, 5 min, 4 min, 3 min, 2 min, 1 min or less. Incubation may be performed at various reaction conditions (e.g., temperature, pH, salt concentration, etc.).

After sufficient time elapses, such that multiple bridge constructs have formed tagged complexes with various spatial tags and analyte sequences, e.g., including tagged complex 250, the tagged complexes may be recovered. In some cases, the tagged complex 250 may be captured using the capture entity 213, such as via a capturing entity. In some instances, the capture entity 213 comprises biotin and the capturing entity comprises streptavidin, where the streptavidin is coupled to a magnetic bead. In some instances, magnetic beads can be captured via applying a magnetic field. It will be appreciated that any capture mechanism may be used. The tagged complexes may be collected on or off the substrate.

The tagged complexes may be processed or repaired prior to, during, or subsequent to capture or isolation, for example to perform one or more of the following operations: cleaving at a cleavage site such as to remove a blocking group, ligating, denaturing, performing extension reactions, and other operations. Spatially tagged sequences (e.g., 260) may be generated. A spatially tagged sequence may comprise a sequence corresponding to the analyte sequence 221 (or portion thereof), a sequence corresponding to the first spatial tag sequence 233 and a sequence corresponding to the second spatial tag sequence 243. For example, in some cases, a spatially tagged sequence comprises the analyte sequence 221 (or portion thereof), a complement of the first spatial tag sequence, and a complement of the second spatial tag sequence. In another example, a spatially tagged sequence comprises a complement of the analyte sequence, first spatial tag sequence, and the second spatial tag sequence. The spatially tagged sequences may subjected to library preparation, such as to attach one or more adapters, barcodes, such as to subject to amplification, etc., and sequencing to generate sequencing reads. Sequencing preparation and sequencing are described in further detail elsewhere herein.

It will be appreciated that while the workflow described herein illustrates a bridge construct that captures two spatial tags, the bridge construct may be designed to capture any number of spatial tags, for example, by including appropriate pairs of attachment binding sequences. In some instances, a bridge construct that is designed to capture five spatial tags comprises five pairs of attachment binding sequences. These pairs of attachment binding sequences may include five of the same pair of attachment binding sequences, or two different pairs, three different pairs, four different pairs, or five different pairs of attachment binding sequences. Each pair of attachment binding sequences may correspond to (e.g., be complementary to) a pair of attachment sequences known to be in at least one geolocation bead in the plurality of geolocation beads loaded on the substrate. In some cases, a bridge construct may be designed to capture at least 2, 3, 4, 5, 6, 7, 8, 9, 10 or more spatial tags. Alternatively or in addition, a bridge construct may be designed to capture at most 10, 9, 8, 7, 6, 5, 4, 3, or 2 spatial tags. A bridge construct may comprise only different pairs of attachment binding sequences, such that a first pair of attachment binding sequence is different from any other pair of attachment binding sequence in the bridge construct. Alternatively, a bridge construct may comprise a repeat of the same pair of attachment binding sequences, or a bridge construct may comprise a mixture of unique pair(s) of attachment binding sequences and overlapping pair(s) of attachment binding sequences.

Bridge constructs may be provided across the substrate such that they are available at all locations on the substrate where released spatial tags and analyte sequences are accessible. In some cases, the bridge constructs may be provided in uniform concentration across all locations. The bridge constructs may be provided in non-uniform concentrations across all locations. A solution comprising the bridge constructs may be dispensed to the substrate according to reagent dispensing mechanisms that are described in further detail elsewhere herein. All reagents, including initial loading of the geolocation beads and/or the sample may be dispensed to the substrate according to reagent dispensing mechanisms that are described elsewhere herein. For example, in some cases, the reagent may be spin-coated or otherwise dispensed to the substrate at a first location and subjected to move to a second location (e.g., radially outward) at high velocity reagent movement across the substrate to distribute the reagent across the substrate. In other cases, the reagent may be, at relatively lower velocities, be painted on the substrate such that there is minimum movement of the reagent from the location of dispensing to the location of immobilization and/or consumption of the reagent.

A map of the plurality of analyte sequences of the sample loaded to the substrate may then be generated by identifying sets of spatial tags from the spatially tagged sequences (e.g., sequencing reads thereof). The map may comprise information about the respective locations or respective probability cloud (or likely location) of each of a set of analyte sequences with respect to a reference analyte sequence. Two spatially tagged sequences comprising the same spatial tag may indicate that the two spatially tagged sequences were within a certain range of proximity of each other. Furthermore, a spatially tagged sequence comprising a set of two or more spatial tags may indicate that the two or more spatial tags are within a certain range of proximity of each other. With multiple sets of two or more spatial tags, such a map of a network of spatial tags may be generated.

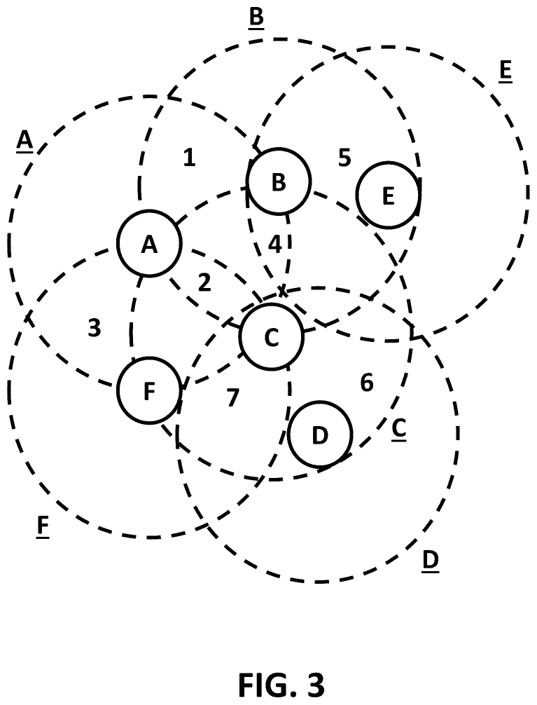

FIG. 3 illustrates an example map that can be generated from the following sets of spatial tags identified from spatially tagged sequences, where capital letters indicate different spatial tags:
Analyte sequence 1: [A, B]
Analyte sequence 2: [A, C]
Analyte sequence 3: [A, F]
Analyte sequence 4: [B, C]
Analyte sequence 5: [B, E]
Analyte sequence 6: [C, D]
Analyte sequence 7: [C, F]

For example, tag A was identified in sets with tags B, C, and F, indicating that it is likely that geolocation beads with spatial tags B, C, and F are each within a somewhat similar distance (e.g., radius) of the geolocation bead with spatial tag A. This is represented by a dotted circle around A in FIG. 3. Accordingly, analyte sequences 1, 2, and 3 are mapped to within the dotted circle. Tag B was identified in sets with tags A, C, and E, indicating that it is likely that geolocation beads attached with spatial tags A, C, and E were each within a certain distance (e.g., radius) of the geolocation bead with spatial tag B. This is represented by a dotted circle around B in FIG. 3. Accordingly, analyte sequences 1, 4, and 5 are mapped to within the dotted circle around B. Tag C was identified in sets with tags A, B, D, and F, indicating that it is likely that geolocation beads with spatial tags A, B, D, and F were each within a certain distance (e.g., radius) of the geolocation bead with spatial tag C. This is represented by a dotted circle around C in FIG. 3. Accordingly, analyte sequences 2, 4, 6, and 7 are mapped to within the dotted circle around C. In some cases, the lack of a set between two spatial tags may indicate that the geolocation beads with those two spatial tags were not located within a certain distance (e.g., radius) of each other, such as [E, F]. In some cases, for each geolocation bead, a maximum distance of diffusion of the spatial tag from the geolocation bead (from release to capture by the bridge construct) can be estimated to facilitate map generation. In some cases, a map of the plurality of geolocation beads may be generated prior to, concurrently with, or subsequent to generating the map of the plurality of analyte sequences. As will be appreciated with more data, a more accurate and/or precise map may be generated. It will be appreciated that FIG. 3 is one example of a map, which is solely provided for illustration purposes. Many different maps that can be generated from the above-provided sample data.

The map may represent an estimate of a spatial relationship between different analyte references with respect to a reference analyte sequence, reference geolocation bead, or reference location, which may be selected arbitrarily. The estimate may include a best estimated location of an analyte sequence or a probability cloud of locations of an analyze sequence with respect to a reference. In some cases the reference (e.g., reference analyte sequence, reference geolocation bead, reference location) may be selected to produce the most accurate or precise map. Computer systems may utilize one or more algorithms to generate the map. For example, in some cases, the one or more algorithms may perform triangulation or similar calculations. In some cases, the one or more algorithms may be able to solve complex problems. The one or more algorithms may include one or more machine learning algorithms.

FIG. 4 illustrates a general workflow of a substrate-based spatial screening method. A substrate 450 may be loaded with a plurality of geolocation beads 401 comprising a plurality of oligonucleotide molecules, as described elsewhere herein, such as the bead 101 described with respect to FIGS. 1A-1B. The substrate 450 may have immobilized thereto a plurality of the geolocation beads 401 on individually addressable locations. A sample 403 which retains, at least to some extent, a spatial relationship between a plurality of analyte sequences, such as tissue slices which retain a spatial relationship between transcripts, may be loaded onto the substrate 450 with the geolocation beads. At any point in time, a plurality of bridge constructs 405, as described elsewhere herein, such as bridge construct 201 described with respect to FIGS. 2A-2B, may be loaded onto the substrate. The geolocation beads may be activated to release the spatial tags (e.g., via releasing the first strands), according to various release mechanisms described elsewhere herein, such as by providing one or more enzymes and/or one or more stimuli. The sample may also be subject to conditions sufficient to render analyte sequences, or derivatives thereof (e.g., complementary DNA to (cDNA) to mRNA, ligation products, etc.) accessible to the bridge constructs. On the substrate, a plurality of bridge constructs (e.g., a subset of a population of total bridge constructs loaded to the substrate) may each capture an analyte sequence and multiple spatial tags to generate tagged complexes (e.g., 250). A plurality of spatially tagged sequences 407 may be generated from the tagged complexes on or off the substrate, recovered (e.g., via the capture entity as described elsewhere herein), and prepared for sequencing. One or more ligation reactions may be performed on the substrate. From the sequencing information, a map of the plurality of analyte sequences may be generated by identifying sets of spatial tags from the plurality of spatially tagged sequences, where the map comprises information about the respective locations or respective probability cloud (or likely location) of each of a set of analyte sequences with respect to a reference analyte sequence.

In an alternative workflow of a substrate-based spatial screening method, a sample which retains, at least to some extent, a spatial relationship between a plurality of analyte sequences, such as tissue slides which retain a spatial relationship between transcripts and/or single cells, may be loaded and immobilized to the substrate first. The sample may be immobilized on the substrate in any design or pattern. In some examples, the samples are immobilized in hydrophilic and/or hydrophobic patterns. The sample may be processed prior to being loaded on the substrate, or while on the substrate, such as to subject the sample to fixation, permeabilization, antibody treatment, probe treatment, and/or any other sample processing reactions (e.g., reverse transcription, transposition, probe reactions, capture reactions, etc.). Such sample processing reaction(s) may be performed in any sequence and/or substantially simultaneously. At any point subsequent to loading the sample on the substrate, a plurality of geolocation beads comprising a plurality of oligonucleotide molecules, as described elsewhere herein, may be provided to the sample. For example, the geolocation beads may be provided substantially simultaneously with one or more sample processing reactions. At any point subsequent to loading the sample on the substrate, a plurality of bridge constructs, as described elsewhere herein (e.g., such as bridge construct 201 described with respect to FIGS. 2A-2B) may be loaded onto the substrate. The geolocation beads may be activated to release the spatial tags (e.g., via releasing the first strands) according to various release mechanisms described elsewhere herein, such as by providing one or more enzymes and/or one or more stimuli. The sample may also be subject to conditions sufficient to render analyte sequences, or derivatives thereof (e.g., complementary DNA to (cDNA) to mRNA, ligation products, etc.) accessible to the bridge constructs. On the substrate, a plurality of bridge constructs (e.g., a subset of a population of total bridge constructs loaded to the substrate) may each capture an analyte sequence and multiple spatial tags to generate tagged complexes (e.g., 250). A plurality of spatially tagged sequences may be generated from the tagged complexes on or off the substrate, recovered (e.g., via the capture entity as described elsewhere herein), and prepared for sequencing. One or more ligation reactions maybe performed on the substrate. From the sequencing information, a map of the plurality of analyte sequences may be generated by identifying sets of spatial tags from the plurality of spatially tagged sequences, where the map comprises information about the respective locations or respective probability cloud (or likely location) of each of a set of analyte sequences with respect to a reference analyte sequence.

In some cases, a sample can be loaded onto a substrate in dilute concentrations to allow tagging of cellular contents of a plurality of cells with spatial tags from geolocation beads. The dilute concentration of the sample may allow for single cell resolution analysis. For example, a plurality of cells can be smeared onto a substrate at substantially dilute concentration. In some methods, a substrate may be loaded with a plurality of cells in a relatively dilute concentration, a plurality of geolocation beads as described elsewhere herein, and a plurality of bridge constructs as described elsewhere herein. The plurality of cells may be provided in a concentration dilute enough that they are located relatively far apart on the substrate such as to prevent or make it extremely unlikely that, between the time of release and capture of spatial tags of geolocation beads by bridge constructs, a first spatial tag of a first geolocation bead located in proximity to a first cell can diffuse and/or a cellular analyte of the first cell can diffuse to be captured by a bridge construct along with a second spatial tag of a second geolocation bead located in proximity to a second cell. After loading onto the substrate, the analyte sequences and/or other cellular content in the cells may be rendered accessible to the bridge constructs. For example, the cells can be lysed. The geolocation beads may be activated to release the spatial tags (e.g., via releasing the first strands), according to various release mechanisms described elsewhere herein, such as by providing one or more enzymes and/or one or more stimuli (e.g., heat). The bridge constructs (e.g., a subset of a population of total bridge constructs or all of the bridge constructs) may each capture an analyte sequence and multiple spatial tags from the plurality of geolocation beads to generate tagged complexes (e.g., 250), for example as described with respect to FIG. 2B. A plurality of spatially tagged sequences (e.g., 260, 407) may be generated from the tagged complexes on or off the substrate, recovered (e.g., via the capture entity as described elsewhere herein), and prepared for sequencing. From the sequencing information of the spatially tagged sequences, or derivatives thereof, an analyte sequence may be mapped to a cell by identifying sets of spatial tags from the plurality of spatially tagged sequences. For example, where a first spatially tagged sequence is identified to include a first spatial tag and a second spatial tag, where a second spatially tagged sequence is identified to include a third spatial tag and a fourth spatial tag, and where a third spatially tagged sequence is identified to include the second spatial tag and the third spatial tag, it can be inferred that all of the first spatial tag, second spatial tag, third spatial tag, and fourth spatial tag were present in proximity to each other on the substrate and therefore tagged analytes from the same cell, and thus it can also be inferred that any spatially tagged sequence found to have any one of the first, second, third, or fourth spatial tag originated from the same cell.

The methods described herein may further comprise methods for attenuation or prevention of long distance diffusion by reagents, such as by attenuating diffusion altogether or by attenuating diffusion along a certain direction(s) on the substrate, and/or in emulsion or in solution, as described elsewhere herein. It may be undesirable for reagents to diffuse too far in a direction that is along an axis or plane contained in a final spatial map generated (e.g., x-y plane) as it may confuse proximity data that is later used to reconstruct the spatial map. The methods may prevent small particles that tend to diffuse relatively fast (e.g., DNA), compared to the duration of various reactions described herein (e.g., barcode activation by USER enzyme, capture, etc.), from diffusing too far from an originating location before tagging occurs, increasing the accuracy of a final spatial map. In some cases, diffusion can be attenuated by adding viscous reagents (e.g., PEG, etc.) and/or modulating one or more other reaction conditions (e.g., temperature).

In some cases, diffusion can be attenuated by encapsulating a reaction space in a gel, hydrogel (e.g., PEG hydrogel), or other mesh or matrix (e.g., polymer mesh matrix) to hinder particle movement therethrough. The encapsulation may be reversible. For example, the mesh or matrix may be degradable, such as after a certain period of time and/or upon application of one or more stimuli (e.g., chemical stimulus to induce, e.g., hydrolysis, enzymatic stimulus, photo stimulus, etc.). In an example, the reaction space comprising the sample and geolocation beads is crosslinked with a hydrophilic polymer to create a mesh that attenuates diffusion throughout the reaction space. The mesh may be nanoscale. In an example, a 4-arm PEG-acrylate macromer and PEG-dithiolglycolate crosslinker is used to form a PEG hydrogel that is degradable. In some cases, protein (e.g., bovine serum albumin (BSA) protein) or other solutes may be embedded or entrapped within the mesh network to increase a crowding effect to further attenuate diffusion.

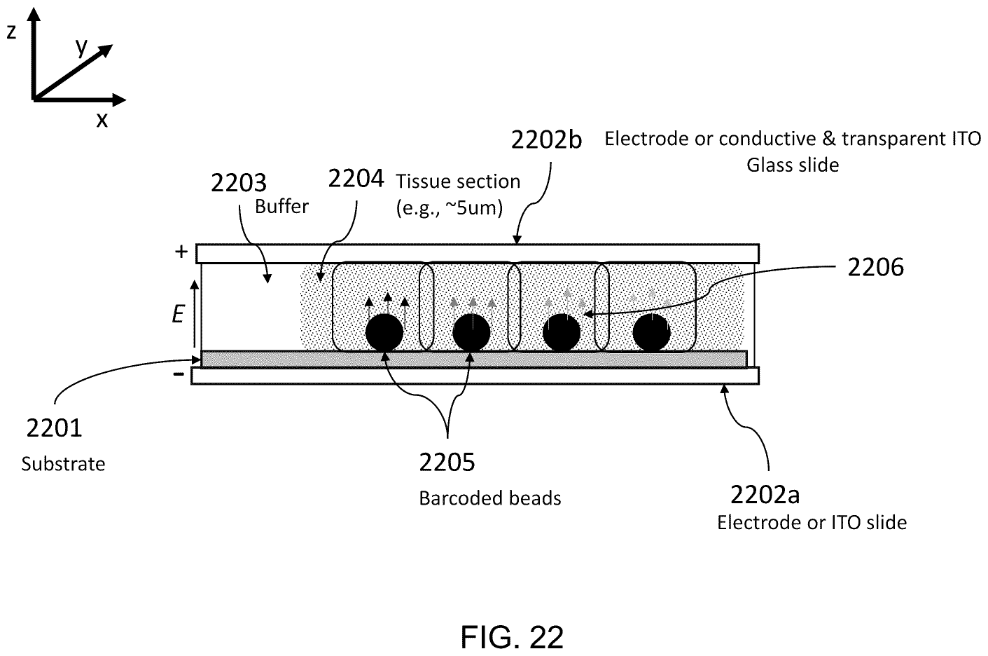

Alternatively or in addition, the reaction space may be subjected to electrophoresis to accelerate movement of charged particles (e.g., DNA, mRNA, spatial tags, etc.) along a direction of the electric field, such as along the z-axis when an x-y plane spatial map is generated, to attenuate diffusion along non-z-axis directions. FIG. 22 illustrates a schematic for subjecting the reaction space to electrophoresis. The geolocation beads 2205, each comprising the spatial tags 2206, and sample 2204 (e.g., 5 micron tissue) may be loaded onto a substrate 2201 as described elsewhere herein. An appropriate buffer 2203 (e.g., (Tris base/acetic acid/EDTA (TAE), Tris/borate/EDTA (TBE), etc.) may be added between two electrodes 2202a and 2202b that sandwich the sample-loaded substrate to facilitate electrophoresis. An electrode (conductive material) may be or comprise an indium tin oxide (ITO) slide. In some cases, the electrode is substantially transparent and conductive. The electric field may be activated with or prior to activation of the geolocation beads 2205 to release the spatial tags 2206. In some cases, the electric field may be activated after activation of the geolocation beads. The released spatial tags may be directed to diffuse primarily along a direction of the electric field (e.g., along z-axis), instead of in the x- or y-axis directions. In an example, mRNA analytes and spatial tag molecules move toward the positive electrode. The electric field may be maintained until substantial completion of all tagging reactions. FIG. 22 illustrates a box around each geolocation bead to represent the diffusion cloud of the spatial tags (in the x-z plane). A reaction space may be both encapsulated in a mesh or matrix (e.g., PEG hydrogel) as described elsewhere herein and subjected to electrophoresis.

In some cases, the methods, systems, compositions, and kits described herein may comprise fiducial marker beads, or the use thereof. A fiducial marker bead may be used as a reference bead around which a spatial map may be generated according to the methods provided herein. In some cases, an absolute position of the fiducial marker bead on a substrate may be known, predetermined, and/or detectable. In some cases, where two or more fiducial marker beads are used, a location of one fiducial marker bead may be known, predetermined, and/or detectable with respect to at least one other fiducial marker bead. One or more of the geolocation beads on the substrate may be a fiducial marker bead. For example, a substrate may have at least about 1, 2, 3, 4, 5, 6, 7, 8, 9, 10, 20, 30, 40, 50, 60, 70, 80, 90, 100, 200, 300, 400, 500 or more fiducial marker beads. Alternatively or in addition, a substrate may have at most about 500, 400, 300, 200, 100, 90, 80, 70, 60, 50, 40, 30, 20, 10, 9, 8, 7, 6, 5, 4, 3, 2, or 1 fiducial marker beads. In some cases, a ratio of the fiducial marker beads to total number of beads (including non-fiducial marker geolocation beads) may be at least about 1×10⁻¹⁰, 1×10⁻⁹, 1λ10⁻⁸, 1×10⁻⁷, 1×10⁻⁶, 1×10⁻⁵, 1×10⁻⁴, 1×10⁻³, 1λ10⁻², 1λ10⁻¹, 1 or more.

A fiducial marker bead may comprise a detectable feature, which distinguishes the fiducial marker bead from non-fiducial marker beads (e.g., remaining geolocation beads), the detectable feature being different than a spatial tag of the fiducial marker bead. In some cases, each fiducial marker bead loaded on a substrate may have a different detectable feature such that upon detection of a detectable feature, it can be identified to only one fiducial marker bead. In some cases, some or all of the fiducial marker beads loaded on a substrate may share a same detectable feature such that upon detection of that detectable feature, it can be identified to be any one of a larger group of fiducial marker beads. For example, multiple types of fiducial marker beads may be used (e.g., one group of beads that have a red fluorophore and one group of beads that have a green fluorophore, etc.). In some cases, the detectable feature is an optically detectable feature, such as a fluorescently detectable feature or other feature detectable under interrogation at one or more wavelengths. The detectable feature may be detectable upon imaging. The detectable feature may be detectable by any detector described herein. The fiducial marker bead may be and behave the same as any variation of a geolocation bead described herein, comprising a spatial tag, except for additionally comprising the detectable feature. The spatial tag of a fiducial marker bead may be known or predetermined and associated with the detectable feature. For example, a first fiducial marker bead may fluoresce in red and comprise a first spatial tag known or predetermined to originate from the fiducial marker bead which fluoresces in red, and a second fiducial marker bead may fluoresce in green and comprise a second spatial tag known or predetermined to originate from the fiducial marker bead which fluoresces in green. In some cases, the spatial tag for each type of fiducial marker bead can be the same (e.g., 100 red fluorescing fiducial marker beads all have the same spatial tag), or different (e.g., 100 red fluorescing fiducial marker beads all have different spatial tags).

In any of the methods described herein, where a plurality of geolocation beads are loaded or otherwise immobilized to a substrate, the method may comprise (A) loading or otherwise immobilizing a mixture of a plurality of geolocation beads and at least one fiducial marker bead onto the substrate, and (B) at any point subsequent to such loading or immobilizing, detecting respective location(s) of the at least one fiducial marker bead using respective detectable feature(s) of the at least one fiducial marker bead. In some instances, the methods may further comprise, prior to the loading or otherwise immobilizing, generating the fiducial marker bead, identifying a spatial tag of a fiducial marker bead (e.g., using a probe, sequencing, etc.), and/or mixing the plurality of geolocation beads and the fiducial marker bead(s) to generate the mixture. The plurality of geolocation beads and the at least one fiducial marker bead may be loaded separately or substantially simultaneously onto the substrate. The fiducial marker bead(s) may be randomly dispersed on the substrate similar to any other geolocation beads as described elsewhere herein. In some cases, the detecting of the respective detectable feature(s) can comprise imaging the substrate to generate a real image of a substrate map in which locations of each fiducial marker bead are pinned. After sequencing reads corresponding to spatially tagged sequences are generated, a sequence comprising a first spatial tag, or complement thereof, which is known or predetermined to originate from a first fiducial marker bead may be pinned or superimposed to a first location of the first fiducial marker bead as detected in (B) (e.g., such as on the substrate map). Similarly, a sequence comprising a second spatial tag, or complement thereof, which is known or predetermined to originate from a second fiducial marker bead may be pinned or superimposed to a second location of the second fiducial marker bead as detected in (B) (e.g., such as on the substrate map), and so on. A spatial map may be generated where the absolute position of at the at least one fiducial marker bead (e.g., with respect to the substrate map) is grounded to be true, and locations of other geolocation beads (or probability cloud thereof) are determined around the fiducial marker beads. Alternatively or in addition, a spatial map may be generated where the relative positions of at least two fiducial marker beads with respect to each other are grounded to be true, and locations of other geolocation beads (or probability cloud thereof) are determined around the fiducial marker beads. Alternatively or in addition, a spatial map may be generated where the position of a fiducial marker bead is determined to be one of multiple distinct positions relative to the real image of the substrate map. Without fiducial marker beads, spatial image of the data may be reconstructed based on estimating global patterns from local proximity data. Beneficially, the fiducial marker beads may significantly reduce the computational complexity of generating accurate spatial maps. In some cases, the computational complexity may reduce as the number and/or types of fiducial marker beads increases. Further, the fiducial marker beads may assist reliable recovery of spatial images that have empty spaces or disjoint features which may not be available otherwise.

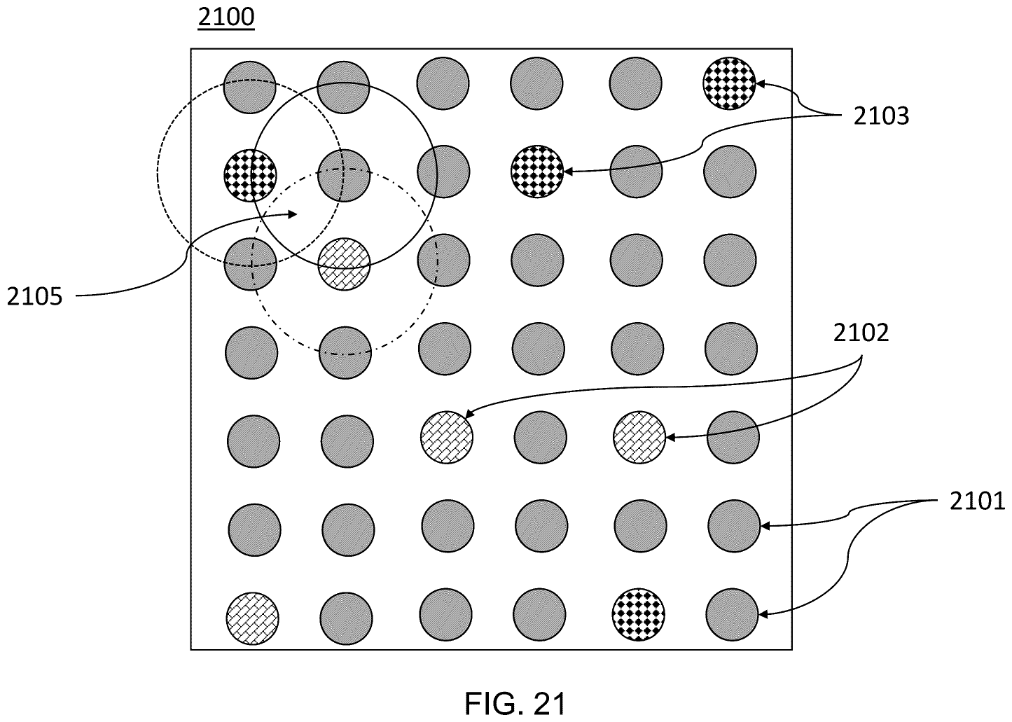

FIG. 21 illustrates an example of a substrate map using fiducial marker beads according to the methods described herein. A mixture of a first type of fiducial marker beads 2102 (e.g., red), a second type of fiducial marker beads 2103 (e.g., green), and non-fiducial marker beads 2101 are randomly loaded on a substrate 2100. The substrate 2100 is imaged to generate a real image of the substrate map. FIG. 21 illustrates a schematic of such a substrate map, where the locations of each fiducial marker beads are readily apparent. After performing the methods described herein, a sequence is identified to have been tagged by both a first type of fiducial marker bead spatial tag and a second type of fiducial marker bead spatial tag, and based on this data, and factoring in diffusion rates (e.g., see example circles in FIG. 21 marking diffusion clouds), locations where the first type of fiducial marker bead and the second type of fiducial marker bead are in close proximity are identified, and it is determined that the sequence was located in some proximity to region 2105 with respect to the substrate map.

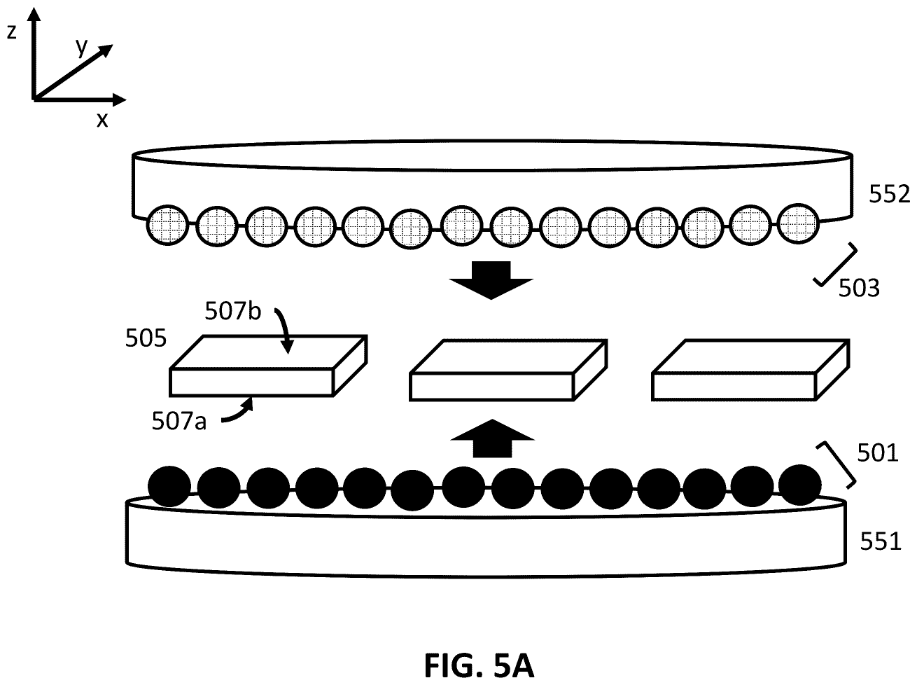

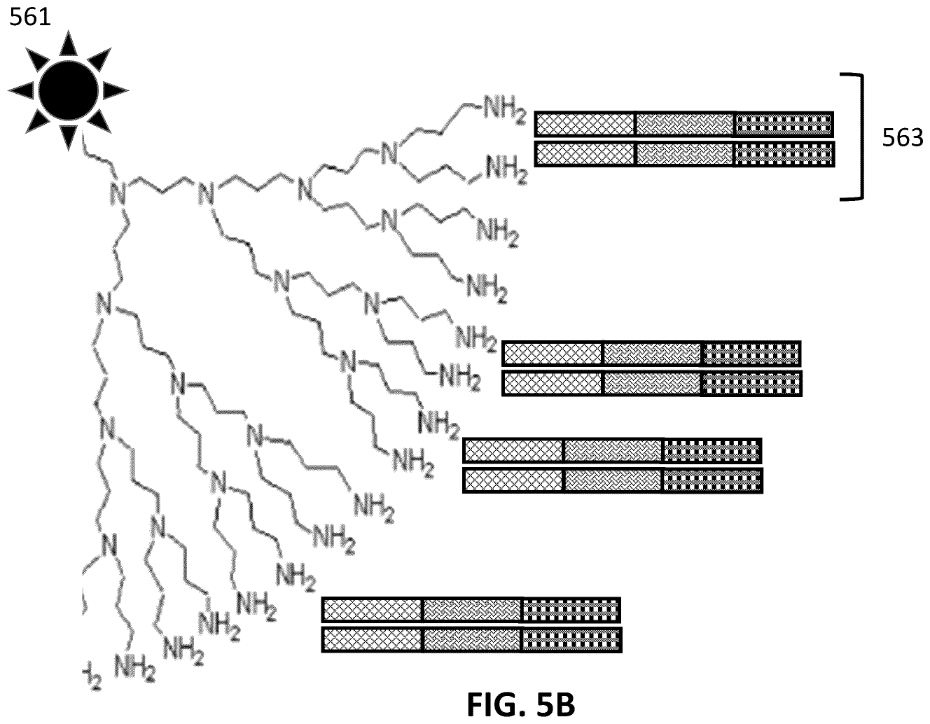

Also provided herein are methods for spatial screening with multiple substrates. FIG. 5A illustrates a spatial screening scheme using multiple substrates. Applying a sample to multiple geo-loaded substrates simultaneously may allow for mapping analyte sequences in the sample to a three-dimensional (3D) spatial resolution and/or with increased two-dimensional (2D) resolution compared to applying a sample to only one geo-loaded substrate. A ‘geo-loaded’ substrate may refer to a substrate of the present disclosure which has immobilized thereto a plurality of geolocation beads, according to methods described elsewhere herein.

A sample 505 may comprise multiple surfaces which can contact or interface a substrate, including at least a first surface 507a and a second surface 507b. In FIG. 5A, these are illustrated as a top surface and bottom surface which are opposite each other (e.g., of a tissue slice) but it will be appreciated that the multiple surfaces can have any orientation with respect to each other (e.g., sharing an edge), which allows for contact or interfacing with different substrates. A first geo-loaded substrate 551 which comprises a first plurality of geolocation beads 501 immobilized thereto may be applied to the first surface 507a and a second geo-loaded substrate 552 which comprises a second plurality of geolocation beads 503 immobilized thereto may be applied to the second surface 507b. The first plurality of geolocation beads 501 may comprise a first set of spatial tags which are completely unique from a second set of spatial tags comprised by the second plurality of geolocation beads 503, such that the two sets of spatial tags are mutually exclusive (no one spatial tag in the first plurality of geolocation beads 501 overlaps with another spatial tag from the second plurality of geolocation beads 503). Furthermore, a spatial tag may be designed and loaded to the substrates such that they are identifiable as originating from which plurality of geolocation beads and/or which substrate. The spatial tag may be identifiable as originating from one of multiple sets of plurality of geolocation beads without the full sequence or substantial portion of the full sequence of the spatial tag being known prior to loading. For example, a first set of geolocation beads may be designed such that each bead has a spatial tag that has a sequence starting with a certain base (e.g., “A”) or a certain sequence of bases (e.g., “ATA”), and a second set of geolocation beads may be designed such that each bead has a spatial tag that has a sequence starting with a different base (e.g., “T”) or a different sequence of bases (e.g., “ATT”). The first set of geolocation beads may be loaded to the first substrate (e.g., bottom substrate contacting the first surface 507a) and the second set of geolocation beads may be loaded to the second substrate (e.g., top substrate contacting the second surface 507b). Once the final tagged sequences are sequenced, an operator may be able to map a spatial tag to the top substrate or the bottom substrate based on the first base or first sequence of bases identified. In this method, one need not know the full sequences of the geolocation beads, just any characterizing features (e.g., first base, first sequence of bases, last base, last sequence of bases) of the spatial tag and to which substrate such beads with the known characterizing features were loaded.

At any point in time, a plurality of bridge constructs (not illustrated in FIG. 5A) as described elsewhere herein (e.g., 405, 201) may be loaded onto the substrate. After applying the multiple geo-loaded substrates (e.g., 551, 552) to the sample (505), the geolocation beads may be activated to release the spatial tags (e.g., via releasing the first strands), according to various release mechanisms described elsewhere herein, such as by providing one or more enzymes and/or one or more stimuli. The sample may also be subject to conditions sufficient to render analyte sequences, or derivatives thereof (e.g., complementary DNA to (cDNA) to mRNA, ligation products, etc.) accessible to the bridge constructs. On one or more of the multiple substrates, a plurality of bridge constructs (e.g., a subset of a population of total bridge constructs loaded to the substrate) may each capture an analyte sequence and multiple spatial tags to generate tagged complexes (e.g., 250). For example, in this scheme, a bridge construct may capture multiple spatial tags from multiple geolocation beads on the first substrate (e.g., 551), capture multiple spatial tags from multiple geolocation beads on the second substrate (e.g., 552), or capture multiple spatial tags from at least one geolocation bead on the first substrate and at least one geolocation bead on the second substrate. Diffusion may occur in a vector that includes a z-axis component, such as to or away from one of the substrates (see axis illustration in FIG. 5A). In some cases, the reaction space may be subjected a directed force (e.g., magnetic force, electric force), to improve or focus diffusion directionality. A plurality of spatially tagged sequences 407 may be generated from the tagged complexes on or off the substrate, recovered (e.g., via the capture entity as described elsewhere herein), and prepared for sequencing. From the sequencing information, a map of the plurality of analyte sequences may be generated by identifying sets of spatial tags from the plurality of spatially tagged sequences, where the map comprises information about the respective locations or respective probability cloud (or likely location) of each of a set of analyte sequences with respect to a reference analyte sequence. If a directed force was applied during diffusion, such conditions may be used during modeling to improve the accuracy of the spatial maps that are generated.

In some cases where a first analyte sequence has been captured with multiple spatial tags from only the first substrate, in addition to being able to map out the relative two-dimensional positions on the first substrate when combined with other data, it may be inferred that the first analyte sequence was disposed closer to the first substrate than the second substrate. In some cases where a first analyte sequence has been captured with at least one spatial tag from the first substrate and at least one spatial tag from the second substrate, it may be inferred that (1) the first analyte sequence was disposed at a particular location on the z-axis where it was possible for both spatial tags from the first substrate and the second substrate to meet (or be captured by the bridge construct) within reaction diffusion conditions, for example not too close to the first substrate and not too close to the second substrate, and that (2) the geolocation bead(s) of the spatial tag(s) identified from the first substrate and the geolocation bead(s) of the spatial tag(s) identified form the second substrate are in proximity in the x-y axis. In this way, a map generated from the data (e.g., sequencing information) collected or received from this multiple substrate scheme may have 3-D spatial resolution, such as to position an analyte sequence on a x-y-z or other 3-D coordinate system with respect to a reference point. Further, at least two 2-D maps may be generated from the data (e.g., sequencing information) collected or received from this multiple substrate scheme, one 2-D map corresponding to geolocation beads immobilized in the first substrate and one 2-D map corresponding to geolocation beads immobilized in the second substrate, to improve overall 2-D spatial resolution of the analyte sequences. When a sample provided between the two substrates are thin enough such that diffusion of reagents between the two substrates (e.g., from one substrate to the other substrate) is common throughout the layer of sample (e.g., along the z-axis), the scheme described herein may contribute more towards super 2-D resolution than the 3-D resolution.

In some cases, a subset or all of the geolocation beads may be designed to emit fluorescence to facilitate multi-channel (e.g., 2 channels, 3 channels, etc.) positional alignment, relative to tissue morphology (e.g., hematoxylin and eosin staining (H&E staining)).

While FIG. 5A illustrates an example with two substrates, it will be appreciated that more substrates may be applied where multiple surfaces are available. The different substrates may or may not be the same size and/or type. In some cases, miniature substrates may be applied to multiple surfaces of a sample which has one surface on a much larger substrate.

Also provided herein are methods for 3-D encoding of samples which may be used alternatively or in addition to the 3-D mapping workflows described with respect to FIG. 5A. A 3-D sample, such as a tissue slice, may be impregnated with a plurality of particles (e.g., nanoparticles), each comprising a plurality of spatial tags.

FIG. 5B illustrates an example of a particle 561 comprising a plurality of oligonucleotide molecules (e.g., 563). The particle may comprise any number of oligonucleotide molecules attached thereto, for example on the order of 10, 10², 10¹, 10⁴, 10⁵, or more. The oligonucleotide molecule 563 may correspond to the oligonucleotide molecule 103 described elsewhere herein, comprising the spatial tag. The particle may be a nanoparticle. In some cases, the particle may have a maximum dimension (e.g., diameter) of at least about 0.50 nanometers (nm), 0.55 nm, 0.60 nm, 0.65 nm, 0.70 nm, 0.75 nm, 0.80 nm, 0.85 nm, 0.90 nm, 0.95 nm, 1.0 nm, 1.1 nm, 1.2 nm, 1.3 nm, 1.4 nm, 1.5 nm, 1.6 nm, 1.7 nm, 1.8 nm, 1.9 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm or greater. Alternatively or in addition, a bead may have a maximum dimension of at most about 0.50 nanometers (nm), 0.55 nm, 0.60 nm, 0.65 nm, 0.70 nm, 0.75 nm, 0.80 nm, 0.85 nm, 0.90 nm, 0.95 nm, 1.0 nm, 1.1 nm, 1.2 nm, 1.3 nm, 1.4 nm, 1.5 nm, 1.6 nm, 1.7 nm, 1.8 nm, 1.9 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, or less. In one example, in a 3-dimensional space, estimating about 5 micron radius for a cell and about 0.01 micron (or 10 nm) radius for a bead, approximately 2 million beads may fit in a cell to produce useful spatial information. A nanoparticle may comprise a dendrimer architecture with any useful generation, for example G3, G4, G5, G6, G7, etc. Example dendrimer species include, without limitation, polyamidoamine (PAMAM), polypropylene imine (PPI), polylysine, poly(propyl ether imine) (PEPIM), and viologen dendrimers. The oligonucleotide molecule may be attached to a surface group of a dendrimer.

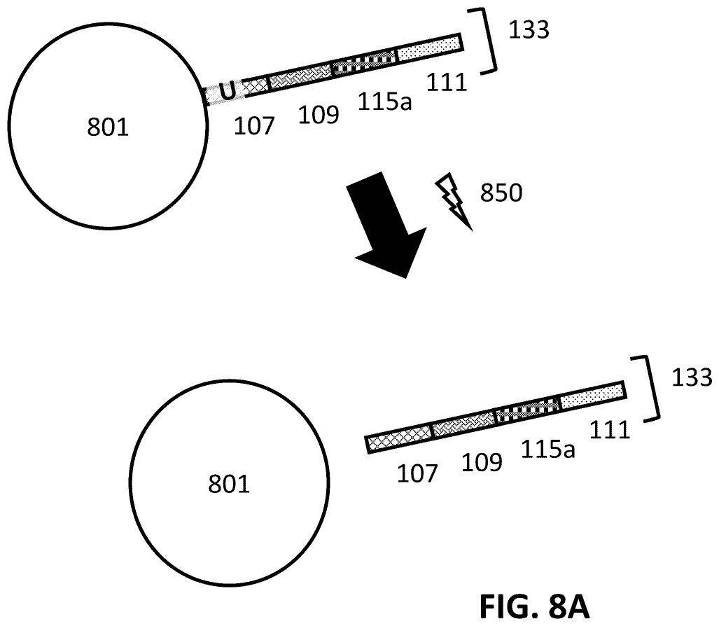

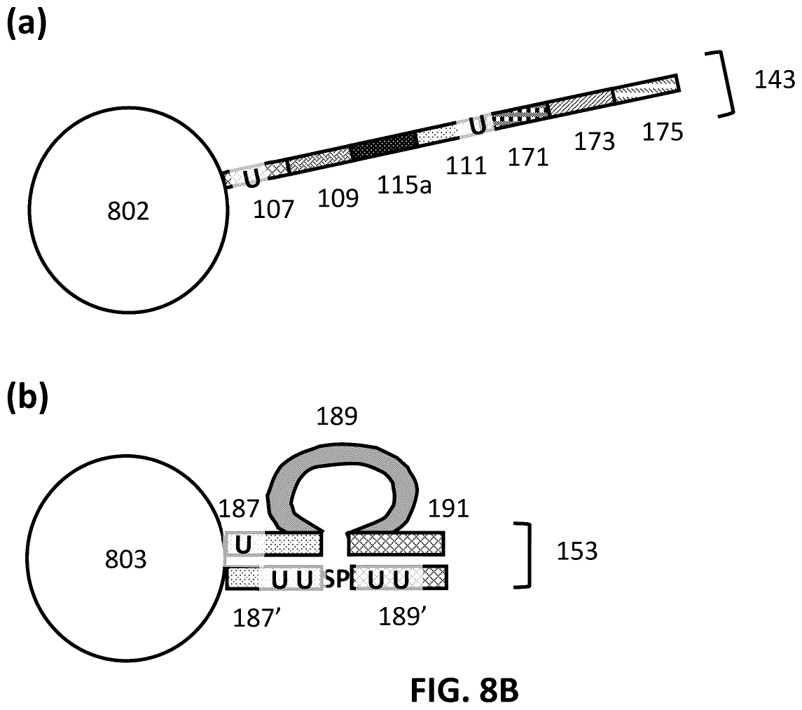

A sample may be incubated with a plurality of such particles for sufficient time to allow for sufficient penetration of the sample with the particles across the sample volume. In some cases, such penetration may be accelerated by applying one or more directed forces, such as magnetic fields, electric fields, and/or pressure. Upon penetration, the particles may be activated to release the spatial tags (e.g., via releasing the first strands), according to various release mechanisms described elsewhere herein (e.g., with respect to geolocation beads and/or capture beads), such as by providing one or more enzymes and/or one or more stimuli. In some examples, a photo or chemical stimulus is applied for the release of the spatial tags, as described with respect to FIG. 8A. At any point in time, a plurality of bridge constructs as described elsewhere herein may be provided to the sample. The sample may also be subject to conditions sufficient to render analyte sequences, or derivatives thereof (e.g., complementary DNA to (cDNA) to mRNA, ligation products, etc.) accessible to the bridge constructs. A plurality of bridge constructs may each capture an analyte sequence and multiple spatial tags to generate tagged complexes (e.g., 250). Diffusion may occur in a vector that includes a z-axis component, such as to or away from a substrate (see axis illustration in FIG. 5A). A plurality of spatially tagged sequences may be generated from the tagged complexes on or off the substrate, recovered (e.g., via the capture entity as described elsewhere herein), and prepared for sequencing. From the sequencing information, a 3-D map of the plurality of analyte sequences may be generated by identifying sets of spatial tags from the plurality of spatially tagged sequences, where the map comprises information about the respective locations or respective probability cloud (or likely location) of each of a set of analyte sequences with respect to a reference analyte sequence. If a directed force was applied, such conditions may be used during modeling to improve the accuracy of the spatial maps that are generated. Beneficially, use of such nano-dimension particles may permit significantly higher spatial resolution.

So far, methods, systems, kits, and compositions for mapping a spatial relationship between analyte sequences of a sample by loading geolocation beads on a substrate have been discussed. The geolocation beads and bridge constructs of the present disclosure may also be used in emulsion-based or solution-based reaction environments, off the substrate.

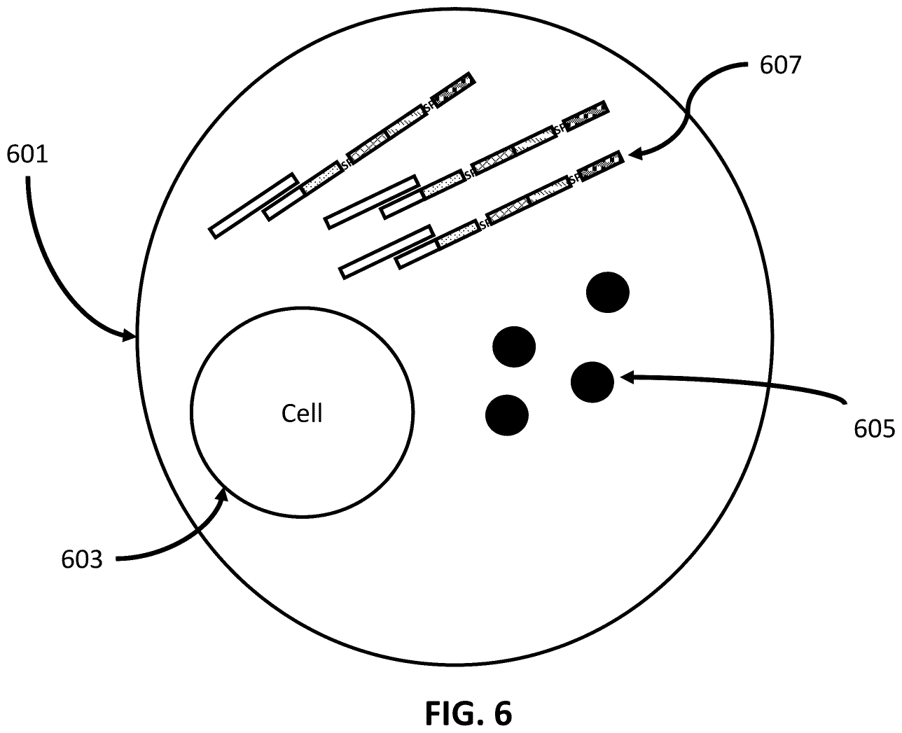

FIG. 6 illustrates an example droplet with reagents of the present disclosure. In an emulsion-based environment, a plurality of geolocation beads and bridge constructs, as described elsewhere herein, and a plurality of cells may be partitioned into a plurality of droplets to tag cellular contents of the plurality of cells with spatial tags within isolated reaction environments. The systems, methods, compositions, and kits described herein may be particularly beneficial where partitioning reagents into droplets in an emulsion are governed by the Poisson distribution. Droplet generation and partitioning systems and methods, as well as problems associated with Poisson distributions (e.g., waste of resources), are described in further detail in International Pub. No. WO2020/167656, which is entirely incorporated herein by reference for all purposes. It may be desirable to generate droplets that contain at most a single cell per droplet to provide isolated reaction environments for single cells. However, in doing so, there are many other droplets generated that contain no cell at all or in some cases more than one cell. When there are multiple analytes that each need to be singly partitioned (e.g., cells and beads), the Poisson problem becomes multiple-fold and results in a large waste of resources. Beneficially, the systems, methods, compositions, and kits permit a droplet to include multiple beads (e.g., geolocation beads). For example, previously, if a droplet contains one cell and multiple beads, each bead comprising a bead-specific tag, cellular analytes tagged by two different bead-specific tags (e.g., spatial tags) within the same droplet may be distinguished and incorrectly classified as having originated from different cells. However, systems, methods, compositions, and kits of the present disclosure allow for the relation of the different bead-specific tags within the same droplet as having originated from the same droplet (and thus same cell) by using the geolocation and bridge constructs described herein.

A plurality of cells, a plurality of geolocation beads, and a plurality of bridge constructs are partitioned to generate a plurality of partitions. The plurality of partitions may comprise a plurality of droplets. Each bead in the plurality of geolocation beads may comprise a unique spatial tag such that no spatial tag of any bead overlaps with any other spatial tag of any other bead. Referring to FIG. 6, a droplet 601 of the plurality of droplets comprises a cell 603, a plurality of geolocation beads (e.g., geolocation bead 605), and a plurality of bridge constructs (e.g., bridge construct 607). The geolocation bead 605 may correspond to any geolocation bead described herein (e.g., 101, 401). The bridge construct 607 may correspond to any bridge construct described herein (e.g., 201, 405). After partitioning, the analyte sequences and/or other cellular content in the cell 603 may be rendered accessible to the bridge constructs. For example, the cell 603 can be lysed. The geolocation beads may be activated to release the spatial tags (e.g., via releasing the first strands), according to various release mechanisms described elsewhere herein, such as by providing one or more enzymes and/or one or more stimuli (e.g., heat). Within the droplet 601, the bridge constructs (e.g., a subset of a population of total bridge constructs in the droplet or all of the bridge constructs) may each capture an analyte sequence and multiple spatial tags from the plurality of geolocation beads to generate tagged complexes (e.g., 250), for example as described with respect to FIG. 2B. A plurality of spatially tagged sequences (e.g., 260, 407) may be generated from the tagged complexes inside or outside of the droplet, recovered (e.g., via the capture entity as described elsewhere herein), and prepared for sequencing. From the sequencing information of the spatially tagged sequences, or derivatives thereof, an analyte sequence may be mapped to a cell by identifying sets of spatial tags from the plurality of spatially tagged sequences. For example, where a first spatially tagged sequence is identified to include a first spatial tag and a second spatial tag, where a second spatially tagged sequence is identified to include a third spatial tag and a fourth spatial tag, and where a third spatially tagged sequence is identified to include the second spatial tag and the third spatial tag, it can be inferred that all of the first spatial tag, second spatial tag, third spatial tag, and fourth spatial tag were present in the same droplet and therefore tagged analytes from the same cell, and thus it can also be inferred that any spatially tagged sequence found to have any one of the first, second, third, or fourth spatial tag originated from the same cell.

While FIG. 6 illustrates a droplet, the partition may be any type of partition, such as a well or micro-well. In some cases, reaction conditions may be adjusted to allow the method to be performed outside of partitions, such as in a solution-based environment. For example, the solution-based environment may comprise one or more of a diluted concentration of cells, higher viscosity, beads comprising cell affinity moieties (e.g., antibodies, lipophilic moieties, etc.), and high concentrations of beads, such that the beads are in large excess compared to other reagents (e.g., cells).

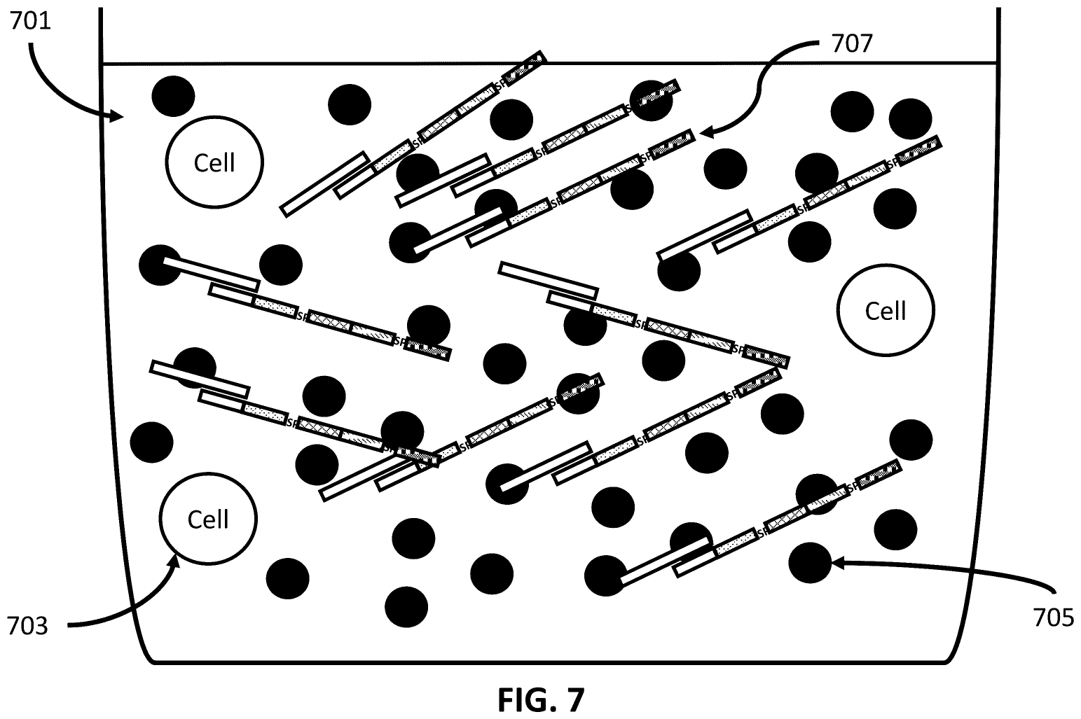

As in the emulsion-based environment described with respect to FIG. 6, a solution-based environment may allow tagging of cellular contents of a plurality of cells with spatial tags from geolocation beads. FIG. 7 illustrates an example solution environment with reagents of the present disclosure. A solution 701 may be provided to comprise a plurality of cells (e.g., 703) in a relatively dilute concentration, a plurality of geolocation beads (e.g., geolocation bead 705), and a plurality of bridge constructs (e.g., bridge construct 707). The plurality of cells may be provided in a concentration dilute enough that they are located relatively far apart within the solution such as to prevent or make it extremely unlikely that, between the time of release and capture of spatial tags of geolocation beads by bridge constructs, a first spatial tag of a first geolocation bead located in proximity to a first cell can diffuse and/or a cellular analyte of the first cell can diffuse to be captured by a bridge construct along with a second spatial tag of a second geolocation bead located in proximity to a second cell. The geolocation bead 705 may correspond to any geolocation bead described herein (e.g., 101, 401, 605). The bridge construct 707 may correspond to any bridge construct described herein (e.g., 201, 405, 607). After being provided in the solution, the analyte sequences and/or other cellular content in the cells may be rendered accessible to the bridge constructs. For example, the cells can be lysed. The geolocation beads may be activated to release the spatial tags (e.g., via releasing the first strands), according to various release mechanisms described elsewhere herein, such as by providing one or more enzymes and/or one or more stimuli (e.g., heat). Within the solution 701, the bridge constructs (e.g., a subset of a population of total bridge constructs or all of the bridge constructs) may each capture an analyte sequence and multiple spatial tags from the plurality of geolocation beads to generate tagged complexes (e.g., 250), for example as described with respect to FIG. 2B. A plurality of spatially tagged sequences (e.g., 260, 407) may be generated from the tagged complexes inside or outside of the solution, recovered (e.g., via the capture entity as described elsewhere herein), and prepared for sequencing. From the sequencing information of the spatially tagged sequences, or derivatives thereof, an analyte sequence may be mapped to a cell by identifying sets of spatial tags from the plurality of spatially tagged sequences. For example, where a first spatially tagged sequence is identified to include a first spatial tag and a second spatial tag, where a second spatially tagged sequence is identified to include a third spatial tag and a fourth spatial tag, and where a third spatially tagged sequence is identified to include the second spatial tag and the third spatial tag, it can be inferred that all of the first spatial tag, second spatial tag, third spatial tag, and fourth spatial tag were present in proximity to each other in the solution and therefore tagged analytes from the same cell, and thus it can also be inferred that any spatially tagged sequence found to have any one of the first, second, third, or fourth spatial tag originated from the same cell. Even though the different cells are provided within a same solution, because the population of cells is so dilute it is unlikely that the same spatial tag (located in proximity with one cell than another cell) will be captured along with analyte sequences from more than one cell.

In some cases, the reaction conditions in the solution may be controlled such as to prevent long distance diffusion of reagents from a first location to a second location in the solution, such as by adding viscous reagents (e.g., PEG, etc.) and/or modulating various reaction conditions (e.g., temperature).

In any of the methods described herein, where a plurality of geolocation beads are provided in bulk solution, such as described with respect to FIG. 7, the method may comprise (A) providing a mixture of a plurality of geolocation beads and at least one fiducial marker bead onto the substrate, and (B) at any point subsequent to such providing, detecting respective location(s) of the at least one fiducial marker bead using respective detectable feature(s) of the at least one fiducial marker bead. In some instances, the methods may further comprise, prior to the providing in solution, generating the fiducial marker bead, identifying a spatial tag of a fiducial marker bead (e.g., using a probe, sequencing, etc.), and/or mixing the plurality of geolocation beads and the fiducial marker bead(s) to generate the mixture. The plurality of geolocation beads and the at least one fiducial marker bead may be provided separately or substantially simultaneously in the solution. The fiducial marker bead(s) may be randomly dispersed in the solution similar to any other geolocation beads as described elsewhere herein. In some cases, the detecting of the respective detectable feature(s) can comprise imaging the solution across one or more planes. For example, various side images, top image, bottom images, etc., at various angles, may be generated of a container comprising the solution to generate one or more real images of the solution. In some cases, the one or more real images of the solution can be resolved into one or more 3D images. In some cases, the imaging may comprise 3D imaging. After sequencing reads corresponding to spatially tagged sequences are generated, a sequence comprising a first spatial tag, or complement thereof, which is known or predetermined to originate from a first fiducial marker bead may be pinned or superimposed to a first location of the first fiducial marker bead as detected in (B) (e.g., such as on the real images). Similarly, a sequence comprising a second spatial tag, or complement thereof, which is known or predetermined to originate from a second fiducial marker bead may be pinned or superimposed to a second location of the second fiducial marker bead as detected in (B), and so on.

FIGS. 8A-8B illustrate examples of additional geolocation bead constructs that can be used for any of the methods described herein. Referring to FIG. 8A, an oligonucleotide molecule 133 on geolocation bead 801 may be single-stranded, and comprise a first attachment sequence 107, a spatial tag 109, and a second attachment sequence 111. Optionally, the oligonucleotide molecule 133 may comprise a UMI sequence 115a. The different sequences are described in further detail elsewhere herein. The oligonucleotide molecule 133 may comprise a cleavage site, denoted “U” in FIG. 8. Upon application of a stimulus (e.g., heat, light) and/or providing one or more enzymes 850, the oligonucleotide molecule 133 may cleave to release a strand that comprises the first attachment sequence 107, the spatial tag 109, and the second attachment sequence 111, and optionally, the UMI sequence 115a. The cleavage site can be located to release such a strand, such as within the first attachment sequence 107 (as illustrated in FIG. 8A) or between the first attachment sequence 107 and the spatial tag 109.

Referring to FIG. 8B, panel (a), an oligonucleotide molecule 143 on geolocation bead 802 may be single-stranded and comprise two sets of a first attachment sequence, a spatial tag, and a second attachment sequence. For example, the oligonucleotide molecule 143 may comprise a third attachment sequence 171, a second spatial tag 173, and a fourth attachment sequence 175. Optionally, the oligonucleotide molecule 143 may comprise a UMI sequence 115a. There may be two cleavage sites, such as to separately or substantially simultaneously release a first strand that comprises the third attachment sequence 171, the second spatial tag 173, and the fourth attachment sequence 175, and a second strand that comprises the first attachment sequence 107, the spatial tag 109, and the second attachment sequence 111. Each of the segments (strands) may be capable of capture by the bridge constructs provided herein. In some cases, having multiple spatial tags on the same geolocation bead may increase spatial resolution. In some cases, the two cleavage sites for the two different capture-able strands may be cleaved at a controlled time to allow for further diffusion by one strand compared to the other strand. Though not illustrated in FIG. 8B, the oligonucleotide molecule may be designed to have multiple UMIs, including many as releasable strands. It will be appreciated that, similarly, the geolocation bead may have any number of sets of the first attachment sequence, spatial tag, and third attachment sequence. The different sets can include the same spatial tag or different spatial tags, or mixtures thereof. The different sets can include the same pair of attachment sequences or different attachment sequences, or mixtures thereof.

Referring to FIG. 8B, panel (b), an oligonucleotide molecule 153 on geolocation bead 803 may be partially double-stranded and partially looped. A first strand may comprise a first attachment sequence 187, a spatial tag 189 which is looped (and not hybridized to the second strand), and a second attachment sequence 191. The second strand may comprise complementary sequences 107′, 109′ for the first and second attachment sequences 187, 191 in the first strand, respectively. A spacer sequence may be disposed between the complementary sequences 107′, 109′ which region corresponds to where the spatial tag 189 loops on the first strand. Beneficially, in this configuration, a complement of the spatial tag 189, which is not used in downstream operations, need not be synthesized. Example cleavage sites are indicated as “U” in the figure. For example, multiple cleavage sites in the bottom strand may be leveraged to cleave or nick and facilitate enzyme activity to release the first strand. The cleavage site in the first strand may be cleaved to release the first strand. The geolocation bead construct may exhibit reduced non-specific interactions.

FIG. 9A illustrates examples of additional bridge constructs that can be used for any of the methods described herein. Referring to FIG. 9A, panel (a), a bridge construct may comprise the capture entity 913, alternatively to or in addition to the capture entity of the oligonucleotide molecule on the geolocation bead (e.g., capture entity 113 on oligonucleotide molecule 123 in FIG. 1A). Referring to FIG. 9A, panel (b), a bridge construct may be provided in two parts, a first part 911 and a second part 915. The first part 911 may be partially double-stranded and comprise, in a first strand, the capture sequence 203 as an overhang, and in a second strand, the second attachment binding sequence 205′. The second part 915 may comprise the first attachment binding sequence 207′ and the fourth attachment binding sequence 209′. The first part 911 of the bridge construct may capture the analyte sequence using the capture sequence and be extended using the analyte sequence as a template. The second attachment binding sequence of the first part of the bridge construct may capture a first spatial tag strand that comprises the first attachment sequence, first spatial tag, and the second attachment sequence. Then, the second part 915 of the bridge construct may hybridize to the first spatial tag strand by binding the first attachment binding sequence to the first attachment sequence. Then, the fourth attachment binding sequence of the second part of the bridge construct may capture a second spatial tag strand that comprises at least the fourth attachment sequence and the second spatial tag. Geolocation beads may be designed without the third attachment sequence, for example, as enabled by this two-part bridge construct.

FIG. 9B illustrates examples of additional tagging schemes using various bridge constructs. In FIG. 9B, panel (a), as described with respect to FIG. 9A, panel (b), a bridge construct may be provided in two parts, a first part and a second part. The first part may be partially double-stranded and comprise, in a first strand, the capture sequence 203 as an overhang, and in a second strand, a first handle 931, the first handle comprising an attachment binding sequence (e.g., second attachment binding sequence 205′) as an overhang. The second part may comprise a second handle 933 which comprises two or more attachment binding sequences (e.g., first attachment binding sequence 207′, fourth attachment binding sequence 209′). In operation, the first part of the bridge construct may capture the analyte sequence 932 using the capture sequence 203 and be extended using the analyte sequence as a template. The attachment binding sequence (e.g., second attachment binding sequence 205′) of the first part of the bridge construct may capture a first spatial tag strand (comprising a spatial tag and two or more attachment sequences) by binding to an attachment sequence (e.g., second attachment sequence 205) of the first spatial tag. The second part of the bridge construct may hybridize to the first spatial tag strand by binding to another attachment sequence (e.g., first attachment sequence 207) of the first spatial tag strand. An additional attachment binding sequence (e.g., fourth attachment binding sequence 209′) of the second handle may capture a second spatial tag strand 935 (comprising a spatial tag and two or more attachment sequences) by binding to a first attachment sequence of the second spatial tag strand (e.g., the fourth attachment sequence 209). In some cases, a second attachment sequence 937 of the second spatial tag strand (e.g., third attachment sequence 211) may remain as an overhang, and unbound to the second handle. As described elsewhere herein, the second spatial tag strand may comprise a capture moiety (e.g., biotin) at a 5′ end. In some cases, the second attachment sequence 937 of the second spatial tag strand, which remains as an overhang, may alternatively or additionally function as a capture sequence, capture binding sequence, attachment sequence, attachment binding sequence, primer sequence, primer binding sequence, or other sequence during one or more downstream processes. Any geolocation bead and spatial tag construct, as described herein, may be used (e.g., 101, 801, 802, 803, etc.).

In panel (b) of FIG. 9B, as described with respect to FIG. 9A, panel (b), a bridge construct may be provided in two parts, a first part and a second part. The first part may be partially double-stranded and comprise, in a first strand, the capture sequence 203 as an overhang, and in a second strand, a first handle 941. The, the first handle 941 may comprise an attachment binding sequence, with a portion of it being an overhanging sequence. The second part may comprise a second handle 943, where the second handle 943 may comprise an attachment binding sequence. Functionally, the first part of the bridge construct may capture the analyte sequence 942 using the capture sequence 203 and be extended using the analyte sequence as a template. The overhang portion of the attachment binding sequence of the first part of the bridge construct may capture a first spatial tag strand (comprising a spatial tag and two or more attachment sequences) by binding to a first attachment sequence of the first spatial tag. The first attachment sequence of the first spatial tag may hybridize to a subsection of the overhang of the first part and be extended through a remaining section of the overhang. Then, the second part of the bridge construct may hybridize to the first spatial tag strand by binding a portion of the attachment binding sequence of the second handle 943 to a second attachment sequence of the first spatial tag strand. Then, a remaining portion of the second handle 943 may capture a second spatial tag strand 945 (comprising a spatial tag and two or more attachment sequences) by binding to a first attachment sequence of the second spatial tag strand. The first attachment of the second spatial tag may hybridize to a subsection of the remaining portion of the second handle 943, and be extended through the remaining section of the second handle. A second attachment sequence 947 of the second spatial tag strand may remain as an overhang and unbound to the second handle. As described elsewhere herein, the second spatial tag strand may comprise a capture moiety (e.g., biotin) at a 5′ end. In some cases, the second attachment sequence 947 of the second spatial tag strand, which remains as an overhang, may alternatively or additionally function as a capture sequence, capture binding sequence, attachment sequence, attachment binding sequence, primer sequence, primer binding sequence, or other sequence during one or more downstream processes.

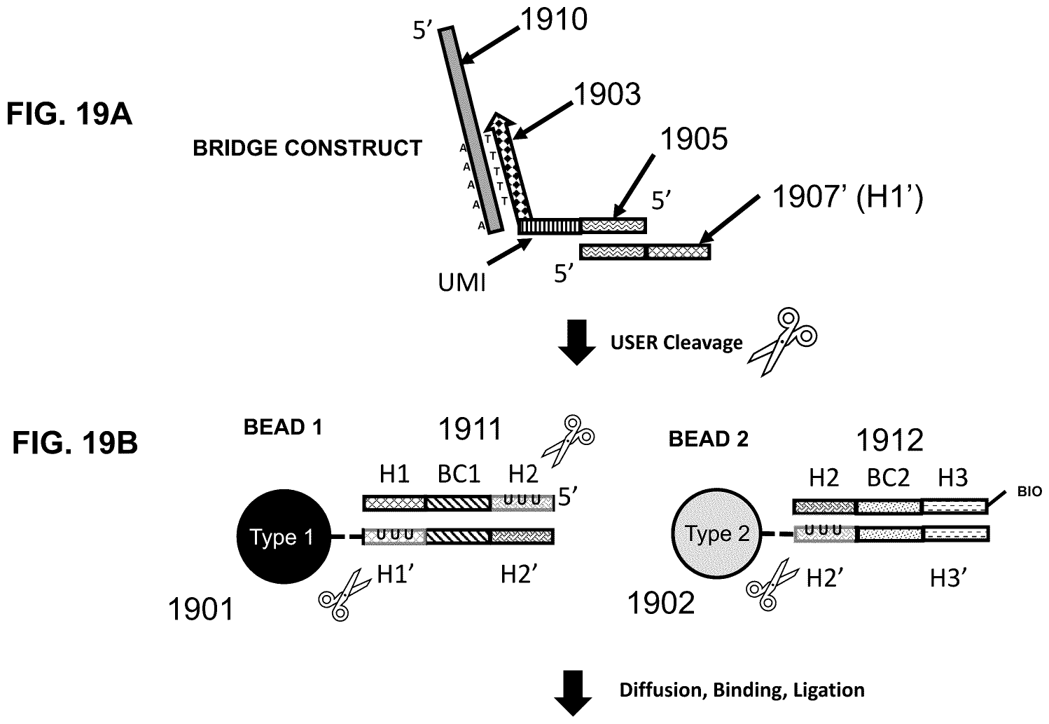

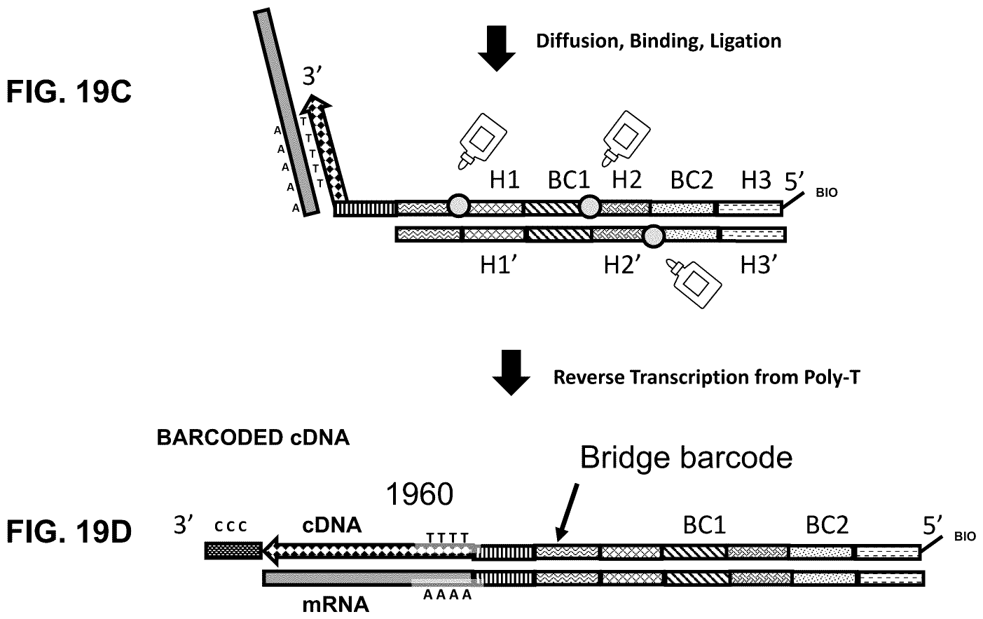

FIGS. 19A-19D illustrate an additional workflow for spatially encoding analytes using geolocation beads and bridge constructs. Referring to FIG. 19A, a bridge construct may comprise a partially double-stranded molecule, where a first strand comprises a capture sequence 1903 (e.g., polyT sequence) as an overhang and a binding sequence 1905 which binds to a second strand. Optionally, the first strand may comprise one or more additional functional sequences, such as a primer sequence, a barcode sequence, or a unique molecular identifier (UMI) sequence. In some cases, the barcode sequence may be unique to a sample (e.g., tissue), so as to be able to later attribute a tagged sequence back to the sample. The one or more additional functional sequences may be disposed between the capture sequence 1903 and the binding sequence 1905. In some cases, the binding sequence 1905 and the one or more additional functional sequences may together function as a barcode sequence for the bridge construct. In some cases, the barcode sequence for the bridge construct may be known. The second strand may comprise a binding sequence which binds to the first strand and an attachment binding sequence 1907′ (H1′). Upon contact with an analyte sequence 1910 (e.g., mRNA comprising polyA tail), the first strand of the bridge construct may capture (e.g., hybridize) the analyte sequence 1910 via the capture sequence 1903 (e.g., polyT sequence). Referring to FIG. 19B, at least two types of geolocation beads may be provided. A first type of geolocation bead 1901 may comprise a double stranded molecule, which in a first strand comprises, in a direction from proximal to distal to the bead, a first attachment sequence (H1), a first spatial tag sequence 1911 (BC1), and a second attachment sequence (H2), and in a second strand comprises, in a direction from proximal to the distal to the bead, the complement of the first strand hybridized to the first strand (i.e., first complementary attachment sequence (H1′), first spatial tag complementary sequence, and second complementary attachment sequence (H2′)). The second strand may be immobilized to the bead 1901. The first strand may comprise one or more cleavable moieties (e.g., uracil) in the second attachment sequence (H2), and the second strand may comprise one or more cleavable moieties in the first complementary attachment sequence (H1′). The first attachment sequence (H1) of the first strand may be complementary to the attachment binding sequence 1907′ (H1′) of the bridge construct. A second type of geolocation bead 1902 may comprise a double stranded molecule, which in a first strand comprises, in a direction from proximal to distal to the bead, a third attachment sequence (H2), a second spatial tag sequence 1912 (BC2), and a fourth attachment sequence (H3), and in a second strand comprises, in a direction from proximal to the distal to the bead, the complement of the first strand hybridized to the first strand (i.e., third complementary attachment sequence (H2′), first spatial tag complementary sequence, and fourth complementary attachment sequence (H3′)). The first strand may comprise, at an end (e.g., 5′ end), a capture moiety (e.g., biotin). The second strand may be immobilized to the bead 1902. The second strand may comprise one or more cleavable moieties in the third complementary attachment sequence (H2′). The third attachment sequence (H2) may correspond to the second attachment sequence (H2) of the first type of geolocation bead 1901. The two types of geolocation beads (e.g., 1901, 1902) may comprise any number of double stranded molecules, as described elsewhere herein with respect to different geolocation bead constructs.

Referring to FIG. 19C, the beads may be subjected to one or more stimuli to release the respective double stranded molecules from the beads, as described elsewhere herein. In some examples, alternatively or in addition, a USER cleavage reaction is performed to process the cleavable moieties from the double stranded molecules and release the remaining molecule from the bead. In the USER cleavage reaction, a USER (uracil-specific excision reagent) enzyme may generate a nucleotide gap at a location of the uracil base in the molecule and facilitate cleavage. Subsequent to such cleavage reaction, the double stranded molecule of the first type of geolocation bead 1901 may result in a partially double stranded molecule, where a first strand comprises the first attachment sequence (H1) as an overhang and a second strand comprises the second complementary attachment sequence (H2′) as an overhang. Subsequent to such cleavage reaction, the double stranded molecule of the second type of geolocation bead 1902 may result in a partially double stranded molecule, where a first strand comprises the third attachment sequence (H2) as an overhang. The (a) loading of the geolocation beads, sample, and the bridge construct on a substrate, and (b) releasing of the spatial tags to allow diffusion before capture by the bridge constructs, are described in further detail elsewhere herein. Still referring to FIG. 19C, the bridge construct may capture the analyte sequence 1910 via the capture sequence 1903 and capture the first spatial tag 1911 (BC1) via the attachment binding sequence 1907′ (H1′) which binds to the first attachment sequence (H1). This complex may then capture the second spatial tag 1912 (BC2) via the second complementary attachment sequence (H2′) which binds to the third attachment sequence (H2), to generate a tagged complex. The captured molecules may be ligated. Referring to FIG. 19D, a reverse transcription reaction may be performed to generate a barcoded molecule which comprises the barcode molecule for the bridge construct, the first spatial tag 1911 (BC1), the second spatial tag 1912 (BC2), and the capture moiety (e.g., biotin). In some examples, the barcoded molecule comprises a barcoded cDNA molecule. Collection of the tagged complexes, as well as processing of such tagged complexes to generate the spatially tagged sequences (e.g., 1960), are described elsewhere herein.

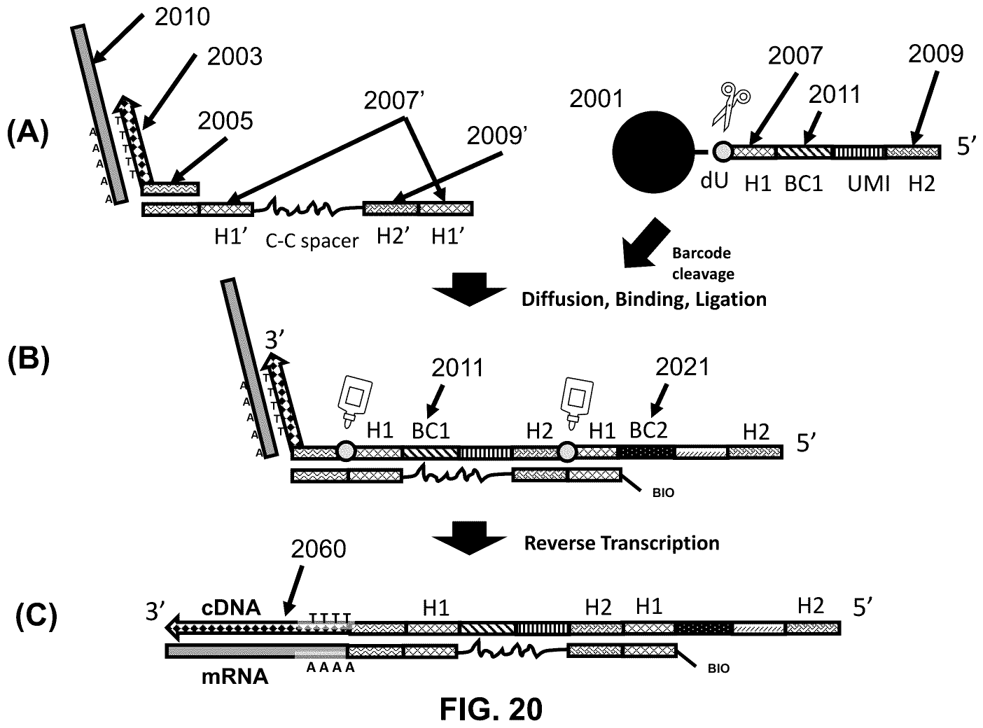

FIG. 20 illustrates an additional workflow for spatially encoding analytes using geolocation beads and bridge constructs. Referring to panel (A), a bridge construct may comprise a partially double-stranded molecule, where a first strand comprises a capture sequence 2003 (e.g., polyT sequence) as an overhang and a binding sequence 2005 which binds to a second strand. Optionally, the first strand may comprise one or more additional functional sequences, such as a primer sequence, a barcode sequence, or a unique molecular identifier (UMI) sequence. In some cases, the barcode sequence may be unique to a sample (e.g., tissue), so as to be able to later attribute a tagged sequence back to the sample. The one or more additional functional sequences may be disposed between the captures sequence 2003 and the binding sequence 2005. In some cases, the binding sequence 2005 and the one or more additional functional sequences may together function as a barcode sequence for the bridge construct. In some cases, the barcode sequence for the bridge construct may be known. The second strand may comprise, in order, a binding sequence which binds to the first strand, a first attachment binding sequence 2007′ (H1′), a spacer sequence, a second attachment binding sequence 2009′, and the first attachment binding sequence 2007′. Spacer sequences in bridge constructs are described elsewhere herein. Upon contact with an analyte sequence 2010 (e.g., mRNA comprising polyA tail), the first strand of the bridge construct may capture (e.g., hybridize) the analyte sequence 2010 via the capture sequence 2003 (e.g., polyT sequence). The method may use one type of geolocation bead 2001. The geolocation bead 2001 may comprise a nucleic acid molecule which comprises, win a direction from proximal to distal to the bead, a first attachment sequence 2007 (H1), a first spatial tag sequence 2011 (BC1), and a second attachment sequence 2009 (H2). In some cases, the nucleic acid molecule may comprise one or more additional functional sequences, such as a primer sequence, a barcode sequence or a UMI sequence. In some cases, the one or more additional sequences may be disposed between the first spatial tag sequence 2011 and the second attachment sequence 2009 (H2). The nucleic acid molecule may be single stranded. The strand may comprise one or more cleavable moieties (e.g., uracil) or cleavage sites proximal to the bead. The first attachment sequence 2007 (H1) of the bead may be complementary to the first attachment binding sequence 2007′ (H1′) of the bridge construct. The second attachment sequence 2009 (H2) of the bead may be complementary to the second attachment binding sequence 2009′ of the bridge construct. The geolocation bead 2001 may comprise any number of oligonucleotide molecules, as described elsewhere herein with respect to different geolocation bead constructs.

Referring to panel (B), the beads may be subjected to one or more stimuli to release the respective oligonucleotide molecules from the beads, as described elsewhere herein. For example, the cleavage sites in the nucleic acid molecule may be cleaved to release the strands. The (a) loading of the geolocation beads, sample, and the bridge construct on a substrate, and (b) releasing of the spatial tags to allow diffusion before capture by the bridge constructs, are described in further detail elsewhere herein. The bridge construct may capture the analyte sequence 2010 via the capture sequence 2003 and capture the first spatial tag 2011 (BC1) via the first of the first attachment binding sequences 2007′ (H1′) which binds to the first attachment sequence 2007 (H1) and the second attachment binding sequence 2009′ which binds to the second attachment sequence 2009 (H2). When bound to the bridge construct, the first spatial tag 2011 (BC1) segment may correspond to spacer sequence region of the bridge construct. This complex may then capture a second spatial tag 2021 (BC2) via the second of the first attachment binding sequences 2007′ (H1′) of the bridge construct which binds to the first attachment binding sequences 2007 of another spatial tag strand, to generate a tagged complex. The captured molecules may be ligated. Referring to panel (C), a reverse transcription reaction may be performed to generate a barcoded molecule which comprises at least the first spatial tag 2011 (BC1) and the second spatial tag 2021 (BC2). In some examples, the barcoded molecule comprises a barcoded cDNA molecule. Collection of the tagged complexes, as well as processing of such tagged complexes to generate the spatially tagged sequences (e.g., 2060), are described elsewhere herein.

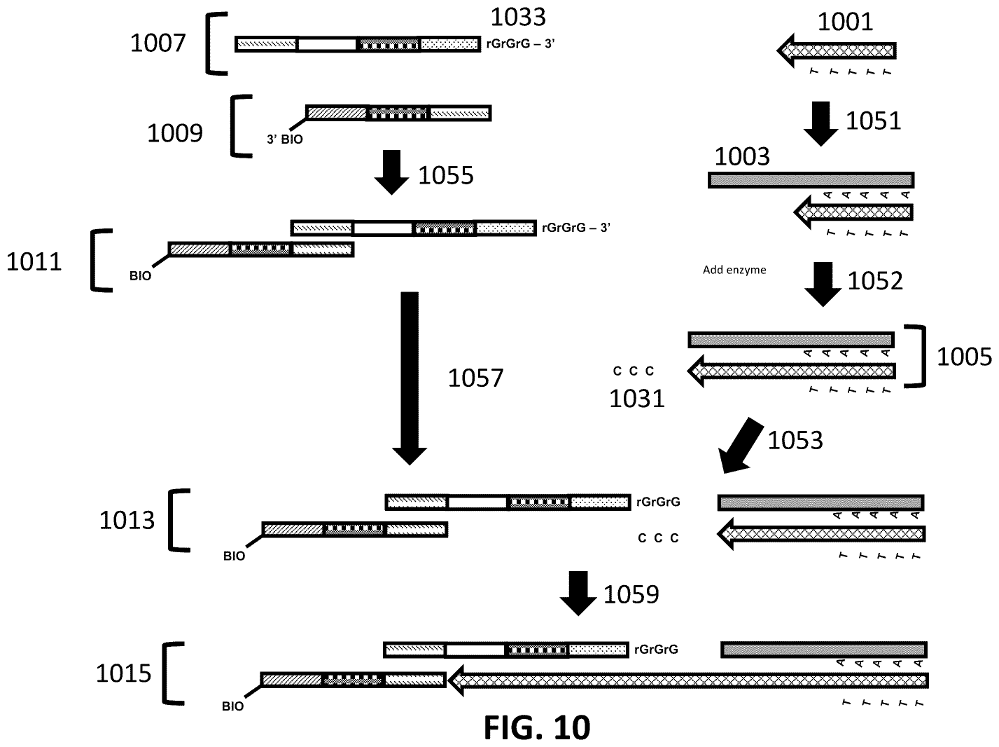

Also provided herein are methods, systems, kits, and compositions that allow for a 5′ approach, as illustrated in FIG. 10. A primer sequence 1001 is provided to couple to an analyte sequence 1003 (e.g., mRNA sequence) (1051). For example, a primer sequence comprising a poly-T sequence captures a poly-A sequence at the 3′ end of a mRNA molecule. The primer sequence 1001 can be extended in a reverse transcription reaction to generate a cDNA transcript 1005 comprising an additional sequence 1031 (e.g., polyC sequence) (1052). For example, the additional sequence 1031 may comprise bases that are added as a result of terminal transferase activity.

A 5′ template switching spatial tag oligonucleotide 1007 may be provided along with a spatial tag strand 1009. The template switching spatial tag oligonucleotide 1007 may comprise a switch sequence 1033 at the 3′ end, which is configured to capture the additional sequence 1031. For example, the switch sequence 1033 comprises a polyG sequence or poly rG sequence. The template switching spatial tag oligonucleotide 1007 may comprise a first spatial tag. The spatial tag strand 1009 may comprise a second spatial tag. The template switching spatial tag oligonucleotide 1007 may attach to the spatial tag strand 1009 via complementary sequences on the 5′ end of the template switching spatial tag oligonucleotide 1007 and the 5′ end of the spatial tag strand 1009, respectively, to form complex 1011 (1055). The spatial tag strand 1009 may comprise a capture entity at the 3′ end such that an end of the complex 1011 comprises the capture entity, and the other end of the complex 1011 comprises the switch sequence 1033. The complex 1011 and the cDNA transcript 1005 may be provided to contact each other (1057, 1053), such that the switch sequence 1033 hybridizes to the additional sequence 1031 to form complex 1013. The cDNA transcript may then be extended to form complex 1015 (1059). The extended complex may be processed to generate a spatially tagged sequence which comprises the cDNA transcript, a complement of the first spatial tag, and the second spatial tag. Alternatively, the spatially tagged sequence may comprise a complement thereof. The complex 1015, or derivative thereof may be captured via the capture entity. The sets of spatial tags identified in the spatially tagged sequences may be analyzed to generate a map of analyte sequences, in accordance with systems, methods, compositions, and kits described herein.

The spatial tag strand 1009 and the 5′ template switching spatial tag oligonucleotide 1007 may be provided on geolocation beads in any of the different types of bead constructs described herein and configured for release of strands 1009 and 1007 instead of other spatial tag strands which are described herein (e.g., 231, 241 comprising spatial tags disposed between a pair of attachment sequences). For example, the spatial tag strand 1009 and the 5′ template switching spatial tag oligonucleotide 1007 may each be provided on different geolocation beads. Alternatively, they may be provided on the same bead. The primer sequence 1001 may be provided to the sample instead of bridge constructs which are described herein to facilitate the reverse transcription reactions prior to capture of the cDNA transcripts.

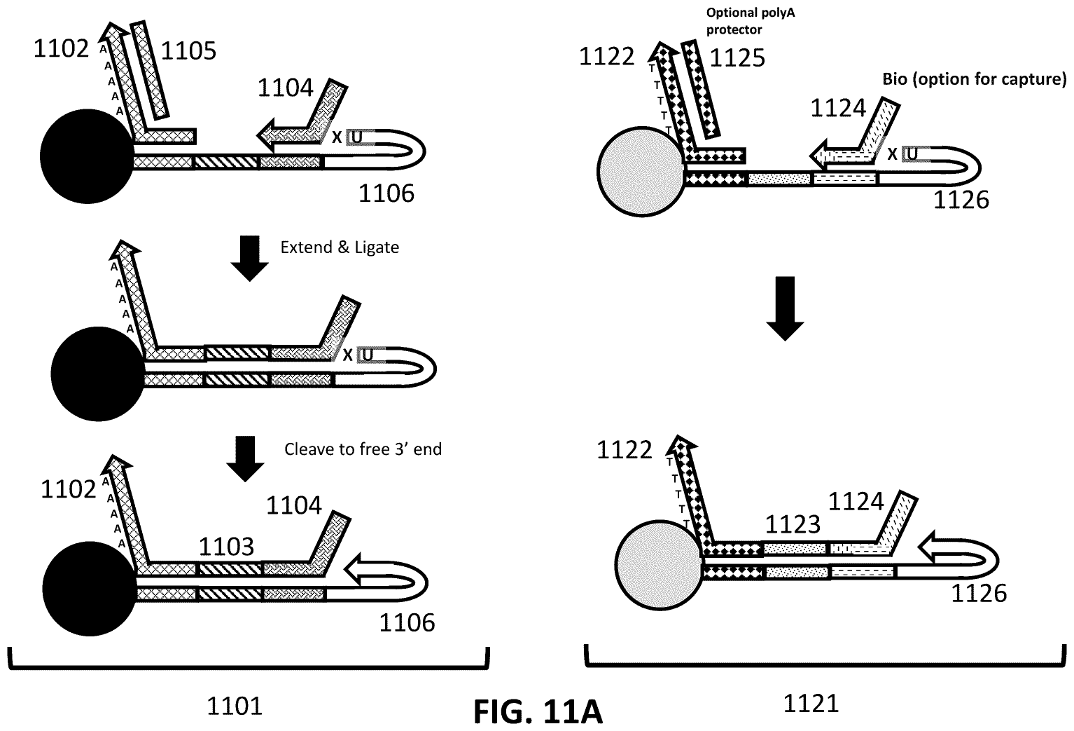

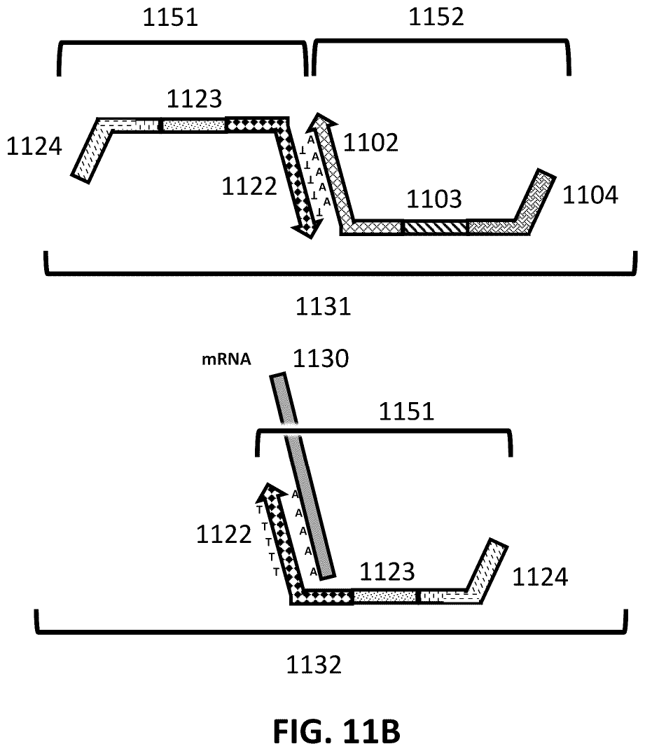

Also provided herein are alternative methods, systems, compositions, and kits for spatial screening. A plurality of geolocation beads and capture beads may be used. FIG. 11A illustrates example constructs of a geolocation bead 1101 and a capture bead 1121.

A geolocation bead 1101 may comprise a plurality of oligonucleotide molecules. An oligonucleotide molecule of the plurality of oligonucleotide molecules, illustrated in FIG. 11A, may comprise a first strand and a second strand, where one end 1106 of the oligonucleotide molecule is looped. The first strand may comprise a capture sequence 1102 (e.g., polyA), a spatial tag 1103, and a first primer sequence 1104. The first strand may be partially bound to a second strand which comprises sequences complementary to a portion of the capture sequence, the spatial tag, and a portion of the first primer sequence, respectively. A loop or hairpin may form at one end 1106 of the oligonucleotide, which does not form part of the first strand. Optionally, the capture sequence 1102 may be bound to a protecting sequence 1105 which is complementary to the capture sequence. Optionally, the oligonucleotide molecule may comprise a UMI sequence. Each of the plurality of oligonucleotide molecules may comprise a common spatial tag, and where there are UMI sequences, each of the plurality of oligonucleotide molecules may comprise a unique UMI sequence (different amongst the plurality of oligonucleotide molecules). The geolocation bead may comprise any number of oligonucleotide molecules, for example on the order of 10, 10², 10³, 10⁴, 10⁵, 10⁶, 10⁷, 10⁸, or more. The spatial tag may correspond to any spatial tag described herein.

To generate this oligonucleotide molecule on the geolocation bead 1101, an oligonucleotide molecule comprising the second strand and the loop, where the loop terminates in a blocking group (“X”) and a cleavage site (“U”), can be provided. At least a portion of the capture sequence 1102 may be bound to the second strand. At least a portion of the first primer sequence 1104 may be bound to the second strand, extended using the second strand as a template until reaching the portion of the capture sequence 1102 bound to the second strand, and ligated. Then, the cleavage site can be cleaved to free the 3′ end.

A capture bead 1121 may comprise a plurality of oligonucleotide molecules. An oligonucleotide molecule of the plurality of oligonucleotide molecules, illustrated in FIG. 11A, may comprise a first strand and a second strand, where one end 1126 of the oligonucleotide molecule is looped. The first strand may comprise a capture sequence 1122 (e.g., polyT), a barcode sequence 1123, and a second primer sequence 1124. The first strand may be partially bound to a second strand which comprises sequences complementary to a portion of the capture sequence, the barcode sequence, and a portion of the second primer sequence, respectively. A loop or hairpin may form at one end 1126 of the oligonucleotide, which does not form part of the first strand. Optionally, the capture sequence 1122 may be bound to a protecting sequence 1125 which is complementary to the capture sequence. Optionally, the oligonucleotide molecule may comprise a UMI sequence. Each of the plurality of oligonucleotide molecules may comprise a common barcode sequence, and where there are UMI sequences, each of the plurality of oligonucleotide molecules may comprise a unique UMI sequence (different amongst the plurality of oligonucleotide molecules). The capture bead may comprise any number of oligonucleotide molecules, for example on the order of 10, 10², 10³, 10⁴, 10⁵, 10⁶, 10⁷, 10⁸, or more. The barcode sequence may be a nucleic acid sequence. The barcode sequence may comprise at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more bases. Alternatively or in addition, the barcode sequence may comprise at most about 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or fewer bases.

To generate this oligonucleotide molecule on the capture bead 1121, an oligonucleotide molecule comprising the second strand and the loop, where the loop terminates in a blocking group (“X”) and a cleavage site (“U”), can be provided. At least a portion of the capture sequence 1122 may be bound to the second strand. At least a portion of the second primer sequence 1124 may be bound to the second strand, extended using the second strand as a template until reaching the portion of the capture sequence 1122 bound to the second strand, and ligated. Then, the cleavage site can be cleaved to free the 3′ end.

The oligonucleotide molecule on the geolocation bead and/or the oligonucleotide molecule on the capture bead may comprise a capture entity, as described elsewhere herein. The first strands on the oligonucleotide molecules on the beads may be released by release mechanisms described elsewhere herein, such as by providing an enzyme configured for strand displacement, digestion, and/or providing one or more stimuli.

In a substrate-based approach, a substrate may be loaded with a plurality of geolocation beads (e.g., 1101) and a plurality of capture beads (e.g., 1121). Loading of beads are described elsewhere herein. The substrate may have immobilized thereto a plurality of geolocation beads and a plurality of capture beads on individually addressable locations. The two types of beads may be loaded onto the substrate in any ratio. In some cases, the ratio between the geolocation beads to the capture beads is about 1:1. In some cases, the ratio of the geolocation beads to the capture beads is at least about 0.00001, 0.0001, 0.001, 0.01, 0.1, 1, 10, 100, 1000, 10000 or more. In some cases, the ratio of the geolocation beads to the capture beads is at most about 10000, 1000, 100, 10, 1, 0.1, 0.01, 0.001, 0.0001, 0.00001, or less.

A sample which retains, at least to some extent, a spatial relationship between a plurality of analyte sequences, such as tissue slices which retain a spatial relationship between transcripts, may be loaded onto the substrate. The geolocation beads and the capture beads may be activated to release the spatial tags and the barcode sequences, respectively (e.g., via releasing the first strands), according to various release mechanisms described elsewhere herein, such as by providing one or more enzymes and/or one or more stimuli. The geolocation beads and the capture beads may be activated simultaneously or substantially simultaneously. The geolocation beads and the capture beads may be activated at different times, for example the capture beads first and then the geolocation beads next, or the geolocation beads first and the capture beads next. The same or different stimuli and/or enzymes may activate the two beads. The sample may also be subject to conditions sufficient to render analyte sequences, or derivatives thereof (e.g., complementary DNA to (cDNA) to mRNA, ligation products, etc.) accessible to the barcode sequences released from the capture beads.

On the substrate, a subset of a plurality of barcode sequences may each capture an analyte sequence from the sample, and another subset of a plurality of barcode sequences may each capture a spatial tag sequence from the geolocation beads. FIG. 11B provides example capture complexes. A first capture complex 1131 comprises a first barcode strand 1151, released from a capture bead, hybridized to a first spatial tag strand 1152, released from a geolocation bead, and hybridized via capture sequence 1122 and 1102 of the first barcode strand the first spatial tag strand, respectively. The complex thus comprises the first primer sequence 1104, the spatial tag 1103, the barcode sequence 1123, and the second primer sequence 1124. A second capture complex 1132 comprises a first barcode strand 1151, released from a capture bead, hybridized to an analyte sequence 1130, released from the sample, and hybridized via capture sequence 1122 of the first barcode strand to a target sequence (e.g., polyA tail) of the analyte sequence 1130. The complex thus comprises the second primer sequence 1124, the analyte sequence 1130, and the barcode sequence 1123. A plurality of spatially tagged sequences may be generated from the two different types of capture complexes, which capture a spatial tag strand or which capture an analyte sequence, such as by extending one or more strands, on or off the substrate, recovered (e.g., via the capture entity as described elsewhere herein), and prepared for sequencing. From the sequencing information, a map of the plurality of analyte sequences may be generated by identifying sets of a spatial tag and barcode sequence from the plurality of spatially tagged sequences, where the map comprises information about the respective locations or respective probability cloud (or likely location) of each of a set of analyte sequences with respect to a reference analyte sequence.

For example, where the sequence information yields a set of [analyte sequence 1, barcode sequence 1]; [spatial tag 5, barcode sequence 1]; [spatial tag 13, barcode sequence 1]; [analyte sequence 16, barcode sequence 1], it can be inferred that (1) the geolocation bead with spatial tag 5 and the geolocation bead with spatial tag 13 are in proximity to each other, that (2) each of the analyte sequence 1 and analyte sequence 16 is in proximity to both the geolocation bead with spatial tag 5 and the geolocation bead with spatial tag 13, and therefore that (3) analyte sequence 1 and analyte sequence 16 are in proximity to each other. More sets of data may be used to map out different analyte sequences and their relative locations.

In an emulsion-based approach, a plurality of geolocation beads and a plurality of capture beads, as described elsewhere herein, and a plurality of cells may be partitioned into a plurality of droplets to barcode cellular contents of the plurality of cells within isolated reaction environments. Beneficially, the systems, methods, compositions, and kits permit a droplet to include multiple beads (e.g., geolocation beads), to allow for the relation of the different bead-specific tags within the same droplet as having originated from the same droplet (and thus same cell).

A plurality of cells, a plurality of geolocation beads, and a plurality of capture beads are partitioned to generate a plurality of partitions. The plurality of partitions may comprise a plurality of droplets. Each bead in the plurality of geolocation beads may comprise a unique spatial tag such that no spatial tag of any bead overlaps with any other spatial tag of any other bead. Each bead in the plurality of capture beads may comprise a unique barcode sequence such that no barcode sequence of any bead overlaps with any other barcode sequence of any other bead. In some instances, a droplet comprises a cell, a plurality of geolocation beads, and a plurality of capture beads. After partitioning, the analyte sequences and/or other cellular content in the cell may be rendered accessible to the barcode sequences of the capture beads. For example, the cell can be lysed. The geolocation beads and the capture beads may be activated to release the spatial tags and the barcode sequences, respectively (e.g., via releasing the first strands), according to various release mechanisms described elsewhere herein, such as by providing one or more enzymes and/or one or more stimuli (e.g., heat). The release can be simultaneous or spaced for the two different types of beads. Within the droplet, a subset of a plurality of barcode sequences may each capture an analyte sequence from the sample, and another subset of a plurality of barcode sequences may each capture a spatial tag sequence from the geolocation beads, for example as described with respect to FIG. 11B, to generate barcoded complexes. A plurality of spatially tagged sequences may be generated from the barcoded complexes inside or outside of the droplet, recovered (e.g., via the capture entity as described elsewhere herein), and prepared for sequencing. From the sequencing information of the spatially tagged sequences, or derivatives thereof, an analyte sequence may be mapped to a cell by identifying sets of a spatial tag and barcode sequence from the plurality of spatially tagged sequences. For example, where a first spatially tagged sequence is identified to include a first spatial tag and a first barcode sequence, where a second spatially tagged sequence is identified to include the first spatial tag and a second barcode sequence, where a third spatially tagged sequence is identified to include a first analyte sequence and the first barcode sequence, and where a fourth spatially tagged sequence is identified to include a second analyte sequence and the second barcode sequence, it can be inferred that all of the first spatial tag, first barcode sequence, second barcode sequence, first analyte sequence, and second analyte sequence were present in the same droplet and therefore the first and second analyte sequences are from the same cell, and thus it can also be inferred that any spatially tagged sequence found to have any one of the first and second barcode sequence originated from the same cell. While this example is specific to a droplet, the partition may be any type of partition, such as a well or micro-well.

As in the emulsion-based environment, a solution-based environment may allow barcoding of cellular contents of a plurality of cells. A solution may be provided to comprise a plurality of cells in a relatively dilute concentration, a plurality of geolocation beads, and a plurality of capture beads. The plurality of cells may be provided in a concentration dilute enough that they are located relatively far apart within the solution such as to prevent or make it extremely unlikely that, between the time of release and capture of spatial tags of geolocation beads by capture beads, a first spatial tag of a first geolocation bead located in proximity to a first cell can diffuse and/or a cellular analyte of the first cell can diffuse to be captured by a barcode sequence of a capture bead located in proximity to a second cell. The geolocation beads and the capture beads may be activated to release the spatial tags and the barcode sequences, respectively (e.g., via releasing the first strands), according to various release mechanisms described elsewhere herein, such as by providing one or more enzymes and/or one or more stimuli (e.g., heat). The release can be simultaneous or spaced for the two different types of beads. Within the solution, a subset of a plurality of barcode sequences may each capture an analyte sequence from the sample, and another subset of a plurality of barcode sequences may each capture a spatial tag sequence from the geolocation beads, for example as described with respect to FIG. 11B, to generate barcoded complexes. A plurality of spatially tagged sequences may be generated from the barcoded complexes inside or outside of the solution, recovered (e.g., via the capture entity as described elsewhere herein), and prepared for sequencing. From the sequencing information of the spatially tagged sequences, or derivatives thereof, an analyte sequence may be mapped to a cell by identifying sets of a spatial tag and barcode sequence from the plurality of spatially tagged sequences. For example, where a first spatially tagged sequence is identified to include a first spatial tag and a first barcode sequence, where a second spatially tagged sequence is identified to include the first spatial tag and a second barcode sequence, where a third spatially tagged sequence is identified to include a first analyte sequence and the first barcode sequence, and where a fourth spatially tagged sequence is identified to include a second analyte sequence and the second barcode sequence, it can be inferred that all of the first spatial tag, first barcode sequence, second barcode sequence, first analyte sequence, and second analyte sequence were present in proximity to each other in the solution and therefore the first and second analyte sequences are from the same cell, and thus it can also be inferred that any spatially tagged sequence found to have any one of the first and second barcode sequence originated from the same cell.

In some cases, the reaction conditions in the solution may be controlled such as to prevent long distance diffusion of reagents from a first location to a second location in the solution, such as by adding viscous reagents (e.g., PEG, etc.) and/or modulating various reaction conditions (e.g., temperature).

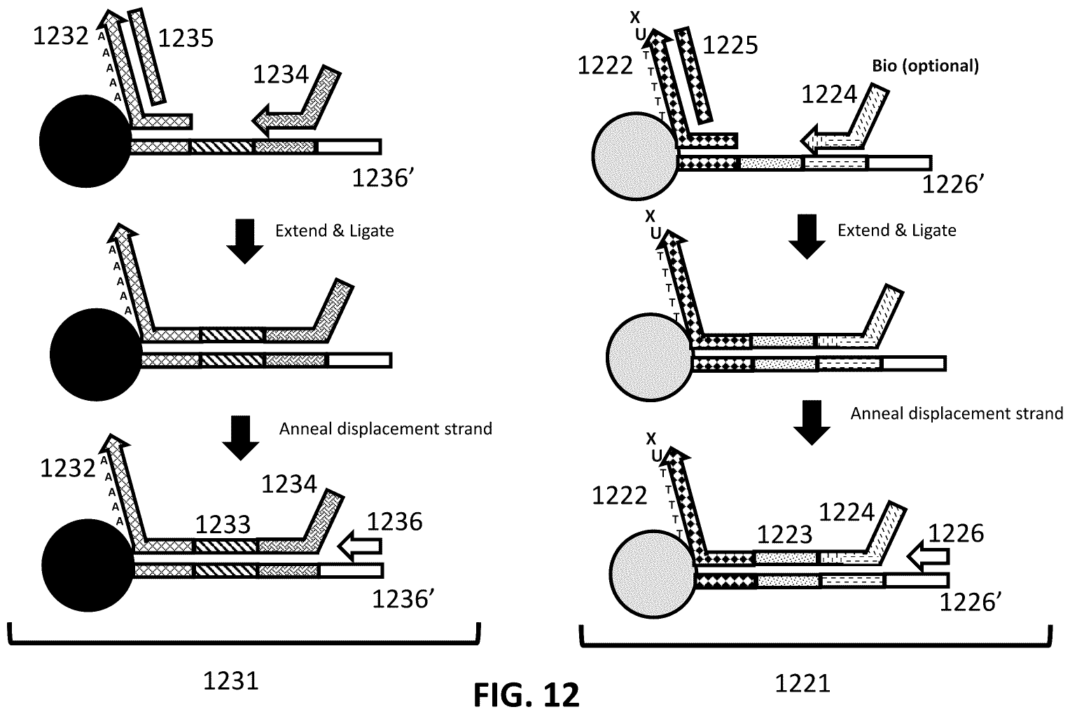

FIG. 12 illustrates an example of additional capture bead and geolocation bead constructs. A capture bead 1221 may comprise a plurality of oligonucleotide molecules. An oligonucleotide molecule of the plurality of oligonucleotide molecules, illustrated in FIG. 12, may comprise a first strand and a second strand. The first strand may comprise a capture sequence 1222 (e.g., polyT), a barcode sequence 1223, and a second primer sequence 1224. The first strand may be partially bound to a second strand which comprises sequences complementary to a portion of the capture sequence, the barcode sequence, and a portion of the second primer sequence, respectively, and a primer binding sequence 1226′. Optionally, the capture sequence 1222 may be bound to a protecting sequence 1225 which is complementary to the capture sequence. Optionally, the oligonucleotide molecule may comprise a UMI sequence. In some cases, the capture sequence 1222 may be capped by a blocking group, denoted “X” and a cleavage site “U.” Such beads may be beneficial for suppressing artefacts from forming during extension. During activation of the beads, an enzyme configured for strand displacement and primer 1226 (e.g., displacement strand) may be provided to displace the first strand (e.g., in the 3′ to 5′ direction), and then the cleavage site may be cleaved to make the first strand accessible or able to bind to the analyte sequence or the spatial tag strand. The cleavage site “U” may be activatable by any one or more stimuli (e.g., light, heat, etc.) and/or one or more enzymes described herein. The oligonucleotide molecule may comprise a capture entity, as described elsewhere herein.

To generate this oligonucleotide molecule on the capture bead 1221, an oligonucleotide molecule comprising the second strand can be provided. At least a portion of the capture sequence 1222 may be bound to the second strand. At least a portion of the second primer sequence 1224 may be bound to the second strand, extended using the second strand as a template until reaching the portion of the capture sequence 1222 bound to the second strand, and ligated.

A geolocation bead 1231 may comprise a plurality of oligonucleotide molecules. An oligonucleotide molecule of the plurality of oligonucleotide molecules, illustrated in FIG. 12, may comprise a first strand and a second strand. The first strand may comprise a capture sequence 1232 (e.g., polyA), a spatial tag 1233, and a first primer sequence 1234. The first strand may be partially bound to a second strand which comprises sequences complementary to a portion of the capture sequence, the spatial tag, and a portion of the first primer sequence, respectively, and a primer binding sequence 1236′. Optionally, the capture sequence 1232 may be bound to a protecting sequence 1235, which is complementary to the capture sequence. Optionally, the oligonucleotide molecule may comprise a UMI sequence. In some cases, the capture sequence 1232 may be capped by a blocking group, denoted “X” and a cleavage site “U.” During activation of the beads, an enzyme configured for strand displacement and primer 1236 (e.g., displacement strand) may be provided to displace the first strand (e.g., in the 3′ to 5′ direction), and then the cleavage site may be cleaved to make the first strand accessible or able to bind to a nucleic acid released from the capture bead. The cleavage site “U” may be activatable by any one or more stimuli (e.g., light, heat, etc.) and/or one or more enzymes described herein. The oligonucleotide molecule may comprise a capture entity, as described elsewhere herein.

To generate this oligonucleotide molecule on the capture bead 1231, an oligonucleotide molecule comprising the second strand can be provided. At least a portion of the capture sequence 1232 may be bound to the second strand. At least a portion of the first primer sequence 1234 may be bound to the second strand, extended using the second strand as a template until reaching the portion of the capture sequence 1232 bound to the second strand, and ligated.

A geolocation bead or capture bead may correspond to any bead described elsewhere herein. In some cases, a bead may have a maximum dimension (e.g., diameter) of at least about 0.05 μm, 0.10 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.40 μm, 0.45 μm, 0.50 μm, 0.55 μm, 0.60 μm, 0.65 μm, 0.70 μm, 0.75 μm, 0.80 μm, 0.85 μm, 0.90 μm, 0.95 μm, 1.0 μm, 1.1 μm, 1.2 μm, 1.3 μm, 1.4 μm, 1.5 μm, 1.6 μm, 1.7 μm, 1.8 μm, 1.9 μm, 2 μm, 3 μm, or greater. Alternatively or in addition, a bead may have a maximum dimension of at most about 0.05 μm, 0.10 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.40 μm, 0.45 μm, 0.50 μm, 0.55 μm, 0.60 μm, 0.65 μm, 0.70 μm, 0.75 μm, 0.80 μm, 0.85 μm, 0.90 μm, 0.95 μm, 1.0 μm, 1.1 μm, 1.2 μm, 1.3 μm, 1.4 μm, 1.5 μm, 1.6 μm, 1.7 μm, 1.8 μm, 1.9 μm, 2 μm, 3 μm, or less. In one example, in a 2-dimensional space, estimating about 5 micron radius for a cell and about 0.25 micron radius for a bead, approximately 100 beads may fit in a cell to produce useful spatial information.

A geolocation bead and/or capture bead, such as described with respect to FIGS. 11A-12, may be a fiducial marker bead, as described elsewhere herein.

### Systems & Kits

Systems, kits, and compositions may comprise any or a combination of the reagents, such as the geolocation beads, capture beads, bridge constructs, enzymes, and primers described herein. In some cases, an index comprising a list of spatial tag sequences included in the geolocation beads may be provided. In some cases, an index comprising a list of barcode sequences included in the capture beads may be provided. The systems, kits, and compositions may include any reagent described herein, such as enzymes, viscosity or crowing agents, sequencing reagents, amplification reagents, and other reagents. A system may comprise any kit and/or reagent described herein. A system may comprise a state in which the provided kit has not been used, has been used, or is being used.

For example, a kit may comprise substrates, geolocation beads, capture beads, bridge constructs, primers and/or enzymes. A kit may comprise any substrate described herein, such as (i) a substrate that does not have any geolocation beads immobilized thereto, or (ii) a substrate comprising geolocation beads immobilized thereto, the geolocation beads comprising spatial tags. The kit may comprise indexed data comprising a list of spatial tag sequences included in the geolocation beads. A kit may comprise a plurality of geolocation beads comprising spatial tag sequences. A kit may comprise a plurality of oligonucleotide molecules comprising spatial tag sequences. A kit may comprise a plurality of oligonucleotide molecules comprising capture sequences. A kit may comprise a plurality of oligonucleotide molecules each comprising both a spatial tag sequence and a capture sequence. A kit may comprise any sequencing reagent described herein. A kit may comprise any amplification reagent described herein.

In one example, a kit comprises (1) a substrate comprising a plurality of geolocation beads immobilized to a plurality of individually addressable locations on the substrate, wherein the plurality of geolocation beads comprises oligonucleotide molecules coupled thereto, wherein the oligonucleotide molecules comprise spatial tag molecules, wherein each geolocation bead of said plurality of geolocation beads comprises a unique spatial tag; (2) indexed data comprising a list of spatial tag sequences included in the geolocation beads; and (3) a second substrate comprising a second plurality of individually addressable locations configured to immobilize a second plurality of geolocation beads. In some cases, the kit further comprises the second plurality of geolocation beads that are not immobilized to the second substrate. In some cases, the kit further comprises a reagent configured to release the oligonucleotide molecules from the plurality of geolocation beads. The reagent can comprise one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of geolocation beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of geolocation beads comprises an alpha-cyclodextrine (a-CD) moiety bound to the azobenzene. In some cases, the kit further comprises sequencing reagents, such as single-base nucleotide mixtures for each of the four base types (e.g., A, C, G, T or U) or multi-base nucleotide mixtures (e.g., A&C, A&T, A&C&G, etc.). A single-base or multi-base nucleotide mixture may comprise a mixture of labeled and unlabeled nucleotides. A single-base or multi-base nucleotide mixture may comprise non-terminated nucleotides, in some cases comprising only non-terminated nucleotides (vs. terminated nucleotides). In some cases, the kit further comprises amplification reagents. Amplification reagents may comprise a polymerase, a nucleotide mixture, a primer, a buffer, or any combination thereof. In some cases, the kit further comprises a biological sample. In some cases, the biological sample may comprise a tissue. In some cases, the biological sample may be fixed and/or permeabilized. In some cases, the biological sample may be loaded on the substrate. In some cases, the kit further comprises fixing and/or permeabilizing reagents. In some cases, a bead of the plurality of geolocation beads may comprise at least 100,000 oligonucleotide molecules. In some cases, the at least 100,000 oligonucleotide molecules may comprise a spatial tag sequence of the spatial tag sequences that is common and unique to the geolocation bead amongst the plurality of geolocation beads. In some cases, an oligonucleotide molecule of the oligonucleotide molecules may comprise a capture sequence, wherein the capture sequence is configured to hybridize with a sequence of an analyte, or derivative thereof, of a biological sample. The capture sequence may be selected from, for example, a polyT sequence, a polyG sequence, a targeted mRNA sequence, a targeted gDNA sequence, a random n-mer sequence, and a probe sequence. The first and second substrate may be substantially identical in size, shape, and/or material.

In another example, a kit comprises a substrate comprising a plurality of geolocation beads immobilized to a plurality of individually addressable locations on the substrate, wherein the plurality of geolocation beads comprises oligonucleotide molecules coupled thereto, wherein the oligonucleotide molecules comprise spatial tag molecules, wherein each geolocation bead of said plurality of geolocation beads comprises a unique spatial tag, wherein the oligonucleotide molecules are releasable from the plurality of geolocation beads by one or more of the release mechanisms selected from the group consisting of: (a) providing an enzyme mix comprising an enzyme configured to cleave or digest a cleavage site, wherein the oligonucleotide molecules comprises the cleavage site; (b) providing biotin, wherein the oligonucleotide molecules are conjugated to a desthiobiotin moiety, wherein the geolocation bead comprises a streptavidin moiety bound to the desthiobiotin moiety; (c) providing an enzyme mix comprising an enzyme configured to cleave or digest a cleavage site and UV light, wherein the oligonucleotide molecules comprises azobenzene and the cleavage site; and (d) providing UV light, wherein the oligonucleotide molecules are conjugated azobenzene, wherein the plurality of geolocation beads comprises an alpha-cyclodextrine (a-CD) moiety bound to the azobenzene. In some cases, the kit may further comprise indexed data comprising a list of spatial tag sequences included in the geolocation beads. In some cases, the kit may further comprise a second substrate comprising a second plurality of individually addressable locations configured to immobilize a second plurality of geolocation beads. In some cases, the kit may further comprise the second plurality of geolocation beads. In some cases, the kit further comprises a reagent configured to release the oligonucleotide molecules from the plurality of geolocation beads. The reagent can comprise one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of geolocation beads comprises an alpha-cyclodextrine (a-CD) moiety bound to the azobenzene. In some cases, the kit further comprises sequencing reagents, such as single-base nucleotide mixtures for each of the four base types (e.g., A, C, G, T or U) or multi-base nucleotide mixtures (e.g., A&C, A&T, A&C&G, etc.). A single-base or multi-base nucleotide mixture may comprise a mixture of labeled and unlabeled nucleotides. A single-base or multi-base nucleotide mixture may comprise non-terminated nucleotides, in some cases comprising only non-terminated nucleotides (vs terminated nucleotides). In some cases, the kit further comprises amplification reagents. Amplification reagents may comprise a polymerase, a nucleotide mixture, a primer, a buffer, or any combination thereof. In some cases, the kit further comprises a biological sample. In some cases, the biological sample may comprise a tissue. In some cases, the biological sample may be fixed and/or permeabilized. In some cases, the biological sample may be loaded on the substrate. In some cases, the kit further comprises fixing and/or permeabilizing reagents. In some cases, a geolocation bead of the plurality of geolocation beads may comprise at least 100,000 oligonucleotide molecules. In some cases, the at least 100,000 oligonucleotide molecules may comprise a spatial tag sequence of the spatial tag sequences that is common and unique to the geolocation bead amongst the plurality of geolocation beads. In some cases, an oligonucleotide molecule of the oligonucleotide molecules may comprise a capture sequence, wherein the capture sequence is configured hybridize with a sequence of an analyte, or derivative thereof, of a biological sample. The capture sequence may be selected from, for example, a polyT sequence, a polyG sequence, a targeted mRNA sequence, a targeted gDNA sequence, a random n-mer sequence, and a probe sequence. The first and second substrate may be substantially identical in size, shape, and/or material.

In one example, a system comprises a sequencing platform configured to (i) address individually addressable locations of substrates and (ii) rotate the substrates during dispensing of sequencing reagents to the substrates or during imaging of the substrates or during both. The sequencing platform may be any sequencing platform described herein. The system may further comprise any kit and/or reagent described herein (e.g., substrate, geolocation beads, bridge constructs, indexed data, reagent configured to release oligonucleotide molecules from a plurality of beads, sequencing reagent, amplification reagent, fixing and/or permeabilizing reagent, etc.). In some cases, the system may comprise a light source configured to provide light at desired frequencies (e.g., UV light, fluorescent light, etc.).

In some instances, a sample is treated by a fixative or fixated, before it is loaded onto the substrate or provided in a reaction environment with one or more reagents of the present disclosure (e.g., geolocation beads, capture beads, etc.). Fixation, in some cases, may render the location of an analyte invariable. For example, an mRNA molecule may not diffuse away from its location in a tissue after fixation. In some case, permeabilization of a fixed biological sample may facilitate the contacting between the reagents and the analyte(s). In some cases, permeabilization of a sample may release an analyte. For example, permeabilization of a fixed tissue may release an mRNA vertically downward, e.g., via gravity, so that it can contact the reagents on the substrate. Once captured, the endogenous mRNA may be tagged with spatial tags and/or barcode sequences, as described elsewhere herein, which can be decoded or processed to determine spatial information. In some instances, a biological sample may be dissected, dissociated, digested, or degraded after an analyte is tagged, according to methods described herein. Such dissection, dissociation, digestion, or degradation of a biological sample may facilitate the processing of the processing of an analyte. Spatial information of an analyte may be retained and decoded via the tags disclosed herein, afterwards. For example, dissection, dissociation, digestion, or degradation of a biological sample may not remove the encoded location information of an analyte. The location of each analyte may be reconstructed digitally by decoding the tags and/or barcode sequences of the plurality of tagged analytes.

A plurality of spatially tagged sequences may be generated from the tagged and/or barcoded analytes on or off the substrate, such as by extending one or more strands of the tagged and/or barcoded analytes. For example, one or more extension reactions may be performed. For example, one or more ligation reactions may be performed. The plurality of spatially tagged sequences may be prepared for sequencing. In some cases, one or more adapters may be attached to one or both ends of the spatially tagged sequences. The adapter-containing spatially tagged sequences may be subjected to amplification reactions. In some cases, subsequent to amplification reactions, a plurality of beads may be collected, wherein each bead of at least a subset of beads (positive beads) comprises a colony of amplification products. For example, each positive bead may comprise, attached thereto, a plurality of nucleic acid molecules having sequence homology or sequence identity and comprising a sequence corresponding to a spatially tagged sequence. The plurality of beads may also comprise negative beads, or beads that do not have nucleic acid molecules comprising a sequence corresponding to a spatially tagged sequence. Optionally, the positive beads may be isolated from the negative beads. The plurality of beads or isolated positive beads may be loaded onto a substrate, as described elsewhere herein, and subjected to a method of sequencing nucleic acid molecules, as described elsewhere herein. Methods for processing analytes or templates (e.g., spatially tagged sequences) for sequencing to generate input material for substrates and sequencing systems described herein are described in International Patent Publication No. 2020/167656, which is entirely incorporated herein by reference for all purposes.

It will be appreciated that various methods for sample preparation or library preparation may be applied. It will be appreciated that various methods for barcoding the spatially tagged sequences may be applied. It will be appreciated that various method for amplification of the processed spatially tagged sequences may be applied. For example, an amplification may comprise a reverse transcription, primer extension, PCR, LCR, helicase-dependent amplification, asymmetric amplification, RCA, RPA, LAMP, NASBA, 3SR, HCR, MDA, derivatives herein and thereof, or any combination herein and thereof. Amplification may comprise emulsion PCR (ePCR or emPCR). During downstream processing, such as during sample preparation or library preparation, any useful sequence may be appended to the spatially tagged sequence, or derivative thereof, such as flow cell attachment sequences, primer sequences, index sequences, barcode sequences, capture sequences, target sequences, etc. In some examples, a strand of a tagged complex 250 or a derivative thereof, may be captured via a primer molecule comprising a capture sequence (e.g., polyT), and the primer molecule extended. In another example, a strand of a tagged complex 250, or derivative thereof, may be subject to reverse transcription, and the transcript captured via a primer molecule comprising a capture sequence (e.g., polyG). In another example, a strand of a tagged complex 250, or derivative thereof, may be captured via a primer molecule comprising a capture sequence (e.g., polyT), the primer molecule extended to generate a transcript comprising a polyC sequence at one end, and the transcript captured via a template switching oligonucleotide comprising a capture sequence (e.g., polyG). In another example, template switching reactions using one or more template switching sequences. In another example, a strand of a tagged complex 250, or derivative thereof, can be contacted with a single Tn5 adapter, reverse transcription performed, and PCR performed. In another example, a strand of a tagged complex 250, or derivative thereof, may be contacted with an enzyme to shear the nucleic acid molecule, a splint adapter may be ligated, and PCR performed. One or more downstream processes may comprise multiple rounds of PCR. One or more downstream processes may comprise enzymatic fragmentation. One or more downstream processes may comprise end repair of the A-tail.

### Sample Processing Systems

Despite the prevalence of sample processing systems and methods, such systems and methods may have low efficiency that can be time-intensive and wasteful of valuable resources, such as reagents. For example, prior microfluidic systems have utilized substrates containing numerous long, narrow channels. The typical flow cell geometry for such substrates introduces a need to compromise between two competing requirements: 1) minimizing volume to minimize reagent usage; and 2) maximizing effective hydraulic diameter to minimize flow time. This trade-off may be especially important for washing operations, which may require large wash volumes and thus long amounts of time to complete. The tradeoff is illustrated by the Poiseuille equation that dictates flow in the laminar regime and is thus inherent to microfluidic systems that utilize such flow cell geometries. Such flow cell geometries may also be susceptible to contamination. Because such flow cell geometries allow for a finite, limited number of channels in the microfluidic systems, such finite number of channels may be shared between a plurality of different mixtures comprising different analytes, reagents, agents, and/or buffers. Contents of fluids flowing through the same channels may be contaminated. Thus, recognized herein is a need for methods and systems for sample processing and/or analysis with high efficiency.

Described herein are devices, systems, and methods for processing analytes using open substrates or open flow cell geometries that can address at least the abovementioned problems. The devices, systems and methods may be used to facilitate any application or process involving a reaction or interaction between an analyte and a fluid (e.g., a fluid comprising reagents, agents, buffers, other analytes, etc.). Such reaction or interaction may be chemical (e.g., polymerase reaction) or physical (e.g., displacement). The systems and methods described herein may benefit from higher efficiency, such as from faster reagent delivery and lower volumes of reagents required per surface area. The systems and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate, which permit highly efficient dispensing of reagents onto the substrate, and highly efficient imaging of one or more analytes, or signals corresponding thereto, on the substrate, among other operations. The substrate may be an open substrate. The substrate may be substantially planar. The substrate may be textured and/or patterned. In some cases, the texture and/or pattern can distinguish individually addressable locations as described elsewhere herein. The sample processing system may comprise an imaging system comprising a detector. Substrates and detectors that can be used in the sample processing system are described in further detail in U.S. Patent Pub. No. 2021/0079464, which is entirely incorporated herein by reference for all purposes.

The term “biological sample,” as used herein, generally refers to any sample from a subject or specimen. The biological sample can be a fluid or tissue from the subject or specimen. The fluid can be blood (e.g., whole blood), saliva, urine, or sweat. The tissue can be from an organ (e.g., liver, lung, or thyroid), or a mass of cellular material, such as, for example, a tumor. The biological sample can be a feces sample, collection of cells (e.g., cheek swab), or hair sample. The biological sample can be a cell-free or cellular sample. Examples of biological samples include nucleic acid molecules, amino acids, polypeptides, proteins, carbohydrates, fats, or viruses. For example, a biological sample may be a nucleic acid sample including one or more nucleic acid molecules, such as deoxyribonucleic acid (DNA) and/or ribonucleic acid (RNA). The nucleic acid molecules may be cell-free or cell-free nucleic acid molecules, such as cell free DNA or cell free RNA. The nucleic acid molecules may be derived from a variety of sources including human, mammal, non-human mammal, ape, monkey, chimpanzee, reptilian, amphibian, avian, or plant sources. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself.

The term “subject,” as used herein, generally refers to an individual from whom a biological sample is obtained. The subject may be a mammal or non-mammal. The subject may be an animal, such as a monkey, dog, cat, bird, or rodent. The subject may be a human. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or deoxyribonucleic acids (DNA) or ribonucleotides or ribonucleic acids (RNA), or analogs thereof. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid molecule can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), or more. A nucleic acid molecule (e.g., polynucleotide) can comprise a sequence of four natural nucleotide bases: adenine (A); cytosine (C); guanine (G); and thymine (T) (uracil (U) for thymine (T) when the polynucleotide is RNA). A nucleic acid molecule may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide analog may be a modified, synthesized or engineered nucleotide. The nucleotide analog may not be naturally occurring or may include a non-canonical base. The naturally occurring nucleotide may include a canonical base. The nucleotide analog may include a modified polyphosphate chain (e.g., triphosphate coupled to a fluorophore). The nucleotide analog may comprise a label. The nucleotide analog may be terminated (e.g., reversibly terminated). The nucleotide analog may comprise an alternative base.

Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotide analogs may be capable of reacting or bonding with detectable moieties for nucleotide detection.

The term “analyte” may refer to molecules, cells, biological particles, or organisms. In some instances, a molecule may be a nucleic acid molecule, antibody, antigen, peptide, protein, or other biological molecule obtained from or derived from a biological sample. An analyte may originate from, and/or be derived from, a sample, such as a biological sample, such as from a cell or organism. An analyte may be synthetic. An analyte may be a biological analyte. For instance, the biological analyte may be a macromolecule, e.g., a nucleic acid, a carbohydrate, a protein, a lipid, etc. The biological analyte may comprise multiple macromolecular groups, e.g., glycoproteins, proteoglycans, ribozymes, liposomes, etc. The biological analyte may be an antibody, antibody fragment, or engineered variant thereof, an antigen, a cell, a peptide, a polypeptide, etc. In some cases, the biological analyte comprises a nucleic acid molecule. The nucleic acid molecule may comprise at least about 10, 100, 1000, 10,000, 100,000, 1,000,000, 10,000,000, 100,000,000, 1,000,000,000 or more nucleotides. Alternatively or in addition, the nucleic acid molecule may comprise at most about 1,000,000,000, 100,000,000, 10,000,000, 1,000,000, 100,000, 10,000, 1000, 100, 10 or fewer nucleotides. The nucleic acid molecule may have a number of nucleotides that is within a range defined by any two of the preceding values. In some cases, the nucleic acid molecule may also comprise a common sequence, to which an N-mer may bind. An N-mer may comprise 1, 2, 3, 4, 5, or 6 nucleotides and may bind the common sequence. In some cases, the nucleic acid molecules may be amplified to produce a colony of nucleic acid molecules attached to the substrate or attached to beads that may associate with or be immobilized to the substrate. In some instances, the nucleic acid molecules may be attached to beads and subjected to a nucleic acid reaction, e.g., amplification, to produce a clonal population of nucleic acid molecules attached to the beads.

The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The term “sequencing,” as used herein, generally refers to a process for generating or identifying a sequence of a biological molecule, such as a nucleic molecule. Such sequence may be a nucleic acid sequence, which may include a sequence of nucleic acid bases. Sequencing may be single molecule sequencing or sequencing by synthesis, for example. Sequencing may be performed using analyte nucleic acid molecules immobilized on a support, such as a flow cell or one or more beads. In some cases, sequencing may comprise generating sequencing signals and/or sequencing reads from the analyte nucleic acid molecules.

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Moreover, amplification of a nucleic acid may be linear, exponential, or a combination thereof. Amplification may be emulsion based or may be non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR and touchdown PCR. Moreover, amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety.

Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference).

The terms “dispense” and “disperse” may be used interchangeably herein. In some cases, dispensing may comprise dispersing and/or dispersing may comprise dispensing. Dispensing generally refers to distributing, depositing, providing, or supplying a reagent, solution, or other object, etc. Dispensing may comprise dispersing, which may generally refer to spreading.

The term “detector,” as used herein, generally refers to a device that is capable of detecting a signal, including a signal indicative of the presence or absence of one or more incorporated nucleotides or fluorescent labels. The detector may detect multiple signals. The signal or multiple signals may be detected in real-time during, substantially during a biological reaction, such as a sequencing reaction (e.g., sequencing during a primer extension reaction), or subsequent to a biological reaction. In some cases, a detector can include optical and/or electronic components that can detect signals. The term “detector” may be used in detection methods. Non-limiting examples of detection methods include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. A detector may be a continuous area scanning detector. For example, the detector may comprise an imaging array sensor capable of continuous integration over a scanning area wherein the scanning is electronically synchronized to the image of an object in relative motion. A continuous area scanning detector may comprise a time delay and integration (TDI) charge coupled device (CCD), Hybrid TDI, or complementary metal oxide semiconductor (CMOS) pseudo TDI device. For example, a continuous area scanning detector may comprise a TDI line-scan camera.

The term “open substrate,” as used herein, generally refers to a substrate in which any point on an active surface of the substrate is physically accessible from a direction normal to the substrate.

The systems and methods may utilize a substrate comprising a plurality of individually addressable locations. The plurality of individually addressable locations may be arranged as an array on the substrate. The plurality of individually addressable locations may be otherwise arranged, such as randomly or in any order, on the substrate. Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). For example, an analyte or reagent may be immobilized to an individually addressable location via a support, such as a bead. In some instances, a bead is immobilized to the individually addressable location, and the analyte or reagent is immobilized to the bead. In some cases, an individually addressable location may immobilize thereto a plurality of analytes or a plurality of reagents. The plurality of analytes may be copies of a template analyte. For example, the plurality of analytes may have sequence homology or sequence identity. For example, the plurality of analytes may be a clonal amplification colony. In other instances, the plurality of analytes may be different (e.g., comprise different sequences). In some examples, the plurality of analytes is immobilized to the individually addressable location via a support, such as a bead. In some examples, a bead comprises a plurality of amplification products, as analytes, immobilized thereto, and the bead is immobilized to an individually addressable location on the substrate. In another example, the bead is immobilized to an individually addressable location on the substrate and is configured to capture or bind to a plurality of analytes. In another example, a plurality of reagents are immobilized to an individually addressable location on the substrate via a support, such as a bead. The plurality of reagents may be configured for capturing or binding an analyte or another reagent. The plurality of reagents may be configured for release from the bead. The plurality of reagents bound to the bead may be releasable prior to, during, or subsequent to capturing or binding, or otherwise interacting with, an analyte or another reagent. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.).

One or more surfaces of the substrate may be exposed to a surrounding open environment, and accessible from such surrounding open environment. For example, the array may be exposed and accessible from such surrounding open environment. In some cases, as described elsewhere herein, the surrounding open environment may be controlled and/or confined in a larger controlled environment.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used in reference to a reagent or a sample herein, may refer to dispensing of the reagent or the sample to a surface of the substrate in accordance with any reagent dispensing mechanism described herein. In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In some instances, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations). In some instances, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In some instances, the method for dispensing reagents may comprise vibration. In some such instances, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents. In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some cases, the substrate may be rotatable about an axis. Analytes or reagents may be immobilized to the substrate during rotation. During one or more downstream processing operations, reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. This mode of directing reagents across a substrate may be herein referred to as centrifugal or inertial pumping. In some cases, the substrate may be rotated at relatively low velocities such that reagents dispensed to a certain location do not move to another location, or moves minimally, because of the rotation, to permit controlled dispensing of reagents to desired locations. Reagents dispensed on the substrate may or may not interact with analytes immobilized on the substrate. For example, when the analytes are nucleic acid molecules and when the reagents comprise nucleotides, the nucleic acid molecules may incorporate or otherwise react with one or more nucleotides. In another example, when the analytes are protein molecules and when the reagents comprise antibodies, the protein molecules may bind to or otherwise react with one or more antibodies. In another example, when the reagents comprise washing reagents, the substrate (and/or analytes on the substrate) may be washed of any unreacted (and/or unbound) reagents, agents, buffers, and/or other particles.

In some cases, the substrate may be movable in any vector or direction, as described elsewhere herein. For example, such motion may be non-linear (e.g., in rotation about an axis). In another example, such motion may be linear. In other examples, the motion may be a hybrid of linear and non-linear motion. The analytes may be immobilized to the substrate during any such motion. Reagents may be dispensed onto the substrate prior to, during, or subsequent to motion of the substrate. In some cases, inertial forces may direct unconstrained reagents across the substrate in any direction during any type of motion (e.g., rotational motion, non-rotational motion, linear motion, non-linear motion, accelerated motion, etc.) of the substrate.

One or more signals (such as optical signals) may be detected from a detection area on the substrate prior to, during, or subsequent to, the dispensing of reagents to generate an output. For example, the output may be an intermediate or final result obtained from processing of the analyte. Signals may be detected in multiple instances. The dispensing, rotating (or other motion), and/or detecting operations, in any order (independently or simultaneously), may be repeated any number of times to process an analyte. In some instances, the substrate may be washed (e.g., via dispensing washing reagents) between consecutive dispensing of the reagents. One or more detection operations can be performed within a desired time frame. For example, the detection operation can be performed within about 1 minute, 50 seconds, 40 seconds, 30 seconds, 20 seconds, 10 seconds or less than 10 seconds. In some instances, at least two detection operations can be performed within 1 minute, 50 seconds, 40 seconds, 30 seconds, 20 seconds, 10 seconds or less than 10 seconds etc. In some instances, at least three detection operations can be performed within 1 minute, 50 seconds, 40 seconds, 30 seconds, 20 seconds, 10 seconds or less than 10 seconds.

One or more dispensing operations can be performed within a desired time frame. For example, the dispensing operation can be performed within 1 minute, 50 seconds, 40 seconds, 30 seconds, 20 seconds, 10 seconds or less than 10 seconds. In some instances, at least two dispensing operations can be performed within 1 minute, 50 seconds, 40 seconds, 30 seconds, 20 seconds, 10 seconds or less than 10 seconds etc. In some instances, at least three dispensing operations can be performed within 1 minute, 50 seconds, 40 seconds, 30 seconds, 20 seconds, 10 seconds or less than 10 seconds. Any operation or process of one or more methods disclosed herein may be performed within a desired time frame. In some instances, a combination of two or more operations or processes disclosed herein may be performed within a desired time frame. For example, the dispensing operation and the detection method may both be performed within 1 minute, 50 seconds, 40 seconds, 30 seconds, 20 seconds, 10 seconds or less than 10 seconds. In some instances, at least two dispensing and detection operations can be performed within 1 minute, 50 seconds, 40 seconds, 30 seconds, 20 seconds, 10 seconds or less than 10 seconds etc. In some instances, at least three dispensing and detection operations can be performed within 1 minute, 50 seconds, 40 seconds, 30 seconds, 20 seconds, 10 seconds or less than 10 seconds.

Systems and methods disclosed herein may obviate the need for barcoding of analytes (e.g., nucleic acid molecules), which may be time-consuming and expensive. For example, alternative or in addition to barcoding, the substrate and/or array of individually addressable locations may be spatially indexed to identify the analytes. Systems and methods disclosed herein may obviate the need for unique barcoding of individual analytes (e.g., individual nucleic acid molecules) or individual samples. Systems and methods for spatial indexing are described in further detail in U.S. Patent Pub. No. 2021/0079464, which is entirely incorporated herein by reference for all purposes.

A substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. A substrate may be fully or partially opaque to visible light. In some cases, a substrate may be at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, or 100% opaque to visible light. The substrate may have an opacity that is within a range defined by any two of the preceding values. A substrate may be fully or partially transparent to visible light. In some cases, a substrate may be at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, or 100% transparent to visible light. The substrate may have a transparency that is within a range defined by any two of the preceding values. In some cases, an illumination power (e.g., a laser power), during detection of a detection area of the substrate, may be adjusted based on the opacity or transparency of the substrate. The one or more layers of the substrate may have a thickness of at least 1 nanometer (nm), 2 nm, 5 nm, 10 nm, 20 nm, 50 nm, 100 nm, 200 nm, 500 nm, 1 micrometer (μm), 2 μm, 5 μm, 10 μm, 20 μm, 50 μm, 100 μm, 200 μm, 500 μm, or 1 millimeter (mm). The one or more layers may have a thickness that is within a range defined by any two of the preceding values. A surface of the substrate may be modified to comprise any of the binders or linkers described herein. A surface of the substrate may be modified to comprise active chemical groups, such as amines, esters, hydroxyls, epoxides, and the like, or a combination thereof. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 μm, 200 μm, 500 μm, 1 mm, 2 mm, 5 mm, or 10 mm. The substrate may have a thickness that is within a range defined by any two of the preceding values. The substrate may have a first lateral dimension (such as a width for a substrate having the general form of a rectangular prism or a radius for a substrate having the general form of a cylinder) of at least 1 mm, 2 mm, 5 mm, 10 mm, 20 mm, 50 mm, 100 mm, 200 mm, 500 mm, or 1,000 mm. The substrate may have a first lateral dimension that is within a range defined by any two of the preceding values. The substrate may have a second lateral dimension (such as a length for a substrate having the general form of a rectangular prism) or at least 1 mm, 2 mm, 5 mm, 10 mm, 20 mm, 50 mm, 100 mm, 200 mm, 500 mm, or 1,000 mm. The substrate may have a second lateral dimension that is within a range defined by any two of the preceding values.

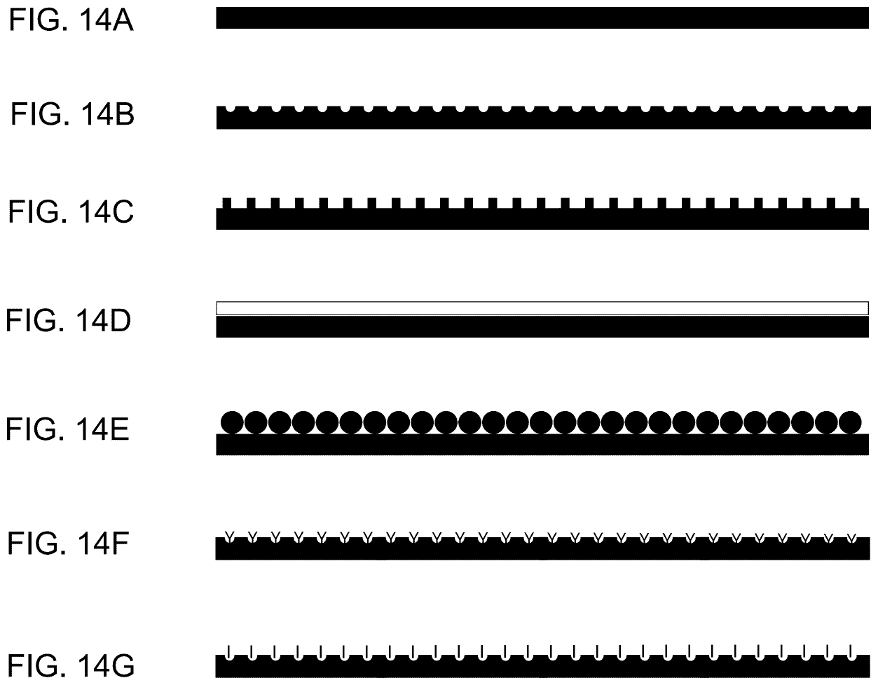

A surface of the substrate may be planar. The surface of the substrate may be substantially planar. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). A surface of the substrate may be uncovered and may be exposed to an atmosphere. Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. For example, the substrate may have any arbitrary structure above or below a reference level of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 14A-14G illustrate different examples of cross-sectional surface profiles of a substrate. FIG. 14A illustrates a cross-sectional surface profile of a substrate having a completely planar surface. FIG. 14B illustrates a cross-sectional surface profile of a substrate having semi-spherical troughs or grooves. FIG. 14C illustrates a cross-sectional surface profile of a substrate having pillars, or alternatively or in conjunction, wells. FIG. 14D illustrates a cross-sectional surface profile of a substrate having a coating. FIG. 14E illustrates a cross-sectional surface profile of a substrate having spherical particles. FIG. 14F illustrates a cross-sectional surface profile of FIG. 14B, with a first type of binders seeded or associated with the respective grooves. FIG. 14G illustrates a cross-sectional surface profile of FIG. 14B, with a second type of binders seeded or associated with the respective grooves.

The substrate may comprise an array. For instance, the array may be located on a lateral surface of the substrate. The array may be a planar array. The array may have the general shape of a circle, annulus, rectangle, or any other shape. The array may comprise linear and/or non-linear rows. The array may be evenly spaced or distributed. The array may be arbitrarily spaced or distributed. The array may have regular spacing. The array may have irregular spacing. The array may be a textured array. The array may be a patterned array. The array may comprise a plurality of individually addressable locations. The individually addressable locations may be arranged in any convenient pattern. For example, the individually addressable locations may be randomly oriented on the array. The plurality of individually addressable locations may form separate radial regions around a disk-shaped substrate. The plurality of individually addressable locations may form a square, rectangle, disc, circular, annulus, pentagonal, hexagonal, heptagonal, octagonal, array, or any other pattern. One or more types of individually addressable locations may be generated. The types of individually addressable locations may be arrayed in any useful pattern, such as a square, rectangle, disc, annulus, pentagon, hexagon, radial pattern, etc. In some cases, the two types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

The analyte to be processed may be immobilized to the array. The array may comprise one or more binders described herein, such as one or more physical or chemical linkers or adaptors, that are coupled to a biological analyte. For instance, the array may comprise a linker or adaptor that is coupled to a nucleic acid molecule. Alternatively or in addition, the biological analyte may be coupled to a bead, which bead may be immobilized to the array. In some cases, a subset of the array may not be coupled to a sample or analyte. In some cases, the array may be coupled to a sample or an analyte, but not all of the array may be processed. For example, the substrate may be coupled to a sample or analyte (e.g., comprising nucleic acid molecules), but the region of the array that is in proximity to the border of the array may not be subjected to further processing (e.g., detection). Similarly, other reagents may be immobilized to the array.

The individually addressable locations may comprise locations of analytes or groups of analytes that are accessible for manipulation. The manipulation may comprise placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings.

In some cases, the individually addressable locations may be indexed, e.g., spatially, such that the analyte immobilized or coupled to each individually addressable location may be identified from a plurality of analytes immobilized to other individually addressable locations. For example, data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the substrate. In some embodiments, the surface of the substrate is demarcated using etching. In some embodiments, the surface of the substrate is demarcated using a notch in the surface. In some embodiments, the surface of the substrate is demarcated using a dye or ink. In some embodiments, the surface of the substrate is demarcated by depositing a topographical mark on the surface. In some embodiments, a sample, such as a control nucleic acid sample, may be used to demarcate the surface of the substrate. As will be appreciated, a combination of positive demarcations and negative demarcations (lack thereof) may be used to index the individually addressable locations. In some embodiments, one or more reference objects (e.g., a reference bead that always emits a detectable signal during detection) are immobilized to any location(s) on the substrate, and the individually addressable locations are indexed with reference to the reference object. In some instances, a single reference point or axis (e.g., single demarcation) may be used to index all individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

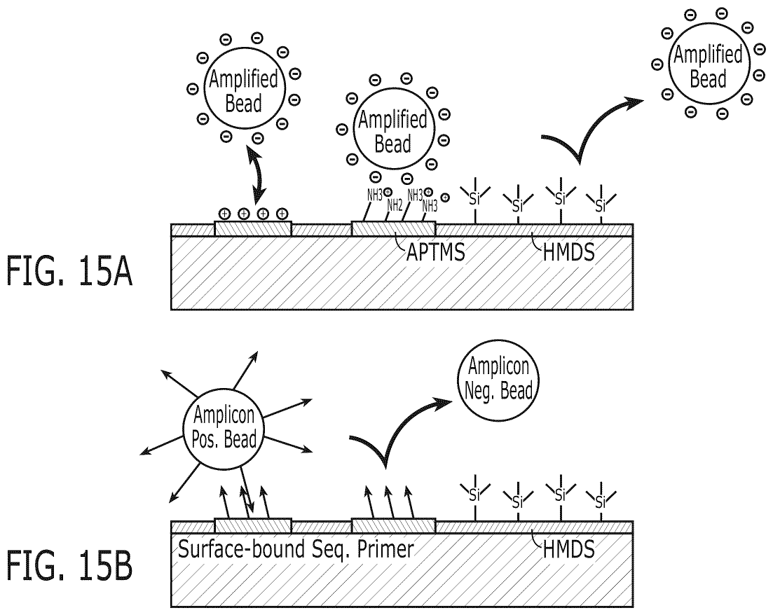

In some cases, an individually addressable location may comprise a distinct surface chemistry. The distinct surface chemistry may distinguish between different addressable locations. The distinct surface chemistry may distinguish between different regions on the substrate. For example, a first location has a first affinity towards an object (e.g., a bead comprising nucleic acid molecules, e.g., amplicons, immobilized thereto) and a second location has a second, different affinity towards the object due to the distinct surface chemistries. The first location and the second location may or may not be located in the same region. The first location and the second location may or may not be disposed on the surface in alternating fashion. In another example, a first region (e.g., comprising a plurality of individually addressable locations) has a first affinity towards an object and a second region has a second, different affinity towards the object due to the distinct surface chemistries. A first location type or region type may comprise a first surface chemistry, and a second location type or region type may comprise a second surface chemistry. In some cases, a third location type or region type may comprise a third surface chemistry. For example, a first location type or region type may comprise a positively charged surface chemistry and/or a hydrophobic surface chemistry, and a second location type or region type may comprise a negatively charged surface chemistry and/or a hydrophilic surface chemistry, as shown in FIG. 15A. The same object (e.g., a bead comprising nucleic acid molecules, e.g., amplicons, immobilized thereto) may have higher affinity towards a first location type or region type compared to a second location type or region type. The same object may be attracted towards a first location type or region type and repelled from a second location type or region type. In other examples, a first location type or region type comprising a first surface chemistry (e.g., a positively charged surface chemistry or a negatively charged surface chemistry) may interact with (e.g., have an affinity towards) a first sample type (e.g., a bead comprising nucleic acid molecules, e.g., amplicons, immobilized thereto) and exclude a second sample type (e.g., a bead lacking nucleic acid molecules, e.g., amplicons, immobilized thereto, e.g., entirely or in substantial volume), for example as illustrated in FIG. 15B. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule.

Each individually addressable location may have the general shape or form of a circle, pit, bump, rectangle, or any other shape or form. An individually addressable location of a plurality of locations (e.g., alternating locations) may have an area. In some cases, a location may have an area of about 0.1 square micron (μm²), 0.2 μm², 0.25 μm², 0.3 μm², 0.4 μm², 0.5 μm², 0.6 μm², 0.7 μm², 0.8 μm², 0.9 μm², 1 μm², 1.1 μm², 1.2 μm², 1.25 μm², 1.3 μm², 1.4 μm², 1.5 μm², 1.6 μm², 1.7 μm², 1.75 μm², 1.8 μm², 1.9 μm², 2 μm², 2.25 μm², 2.5 μm², 2.75 μm², 3 μm², 3.25 μm², 3.5 μm², 3.75 μm², 4 μm², 4.25 μm², 4.5 μm², 4.75 μm², 5 μm², 5.5 μm², or 6 μm². A location may have an area that is within a range defined by any two of the preceding values. A location may have an area that is less than about 0.1 μm² or greater than about 6 μm². In some cases, a location may have a width of about 0.1 micron (μm), 0.2 μm, 0.25 μm, 0.3 μm, 0.4 μm, 0.5 μm, 0.6 μm, 0.7 μm, 0.8 μm, 0.9 μm, 1 μm, 1.1 μm, 1.2 μm, 1.25 μm, 1.3 μm, 1.4 μm, 1.5 μm, 1.6 μm, 1.7 μm, 1.75 μm, 1.8 μm, 1.9 μm, 2 μm, 2.25 μm, 2.5 μm, 2.75 μm, 3 μm, 3.25 μm, 3.5 μm, 3.75 μm, 4 μm, 4.25 μm, 4.5 μm, 4.75 μm, 5 μm, 5.5 μm, or 6 μm. In some cases, a location may have a width that is within a range defined by any two of the preceding values. A location may have a width that is less than about 0.1 μm or greater than about 6 μm. Each individually addressable location may have a first lateral dimension (such as a radius for individually addressable locations having the general shape of a circle or a width for individually addressable locations having the general shape of a rectangle). In some cases, a first lateral dimension of a location may be at least 1 nanometer (nm), 2 nm, 5 nm, 10 nm, 20 nm, 50 nm, 100 nm, 200 nm, 500 nm, 1,000 nm, 2,000 nm, 5,000 nm, or 10,000 nm. The first lateral dimension may be within a range defined by any two of the preceding values. Each individually addressable location may have a second lateral dimension (such as a length for individually addressable locations having the general shape of a rectangle). The second lateral dimension may be at least 1 nanometer (nm), 2 nm, 5 nm, 10 nm, 20 nm, 50 nm, 100 nm, 200 nm, 500 nm, 1,000 nm, 2,000 nm, 5,000 nm, or 10,000 nm. The second lateral dimension may be within a range defined by any two of the preceding values.

In some cases, the locations (e.g., of a same type) may be distributed on a substrate with a pitch determined by the distance between the center of a first location and the center of the closest or neighboring location (e.g., of the same type). Locations may be spaced with a pitch of about 0.1 micron (μm), 0.2 μm, 0.25 μm, 0.3 μm, 0.4 μm, 0.5 μm, 0.6 μm, 0.7 μm, 0.8 μm, 0.9 μm, 1 μm, 1.1 μm, 1.2 μm, 1.25 μm, 1.3 μm, 1.4 μm, 1.5 μm, 1.6 μm, 1.7 μm, 1.75 μm, 1.8 μm, 1.9 μm, 2 μm, 2.25 μm, 2.5 μm, 2.75 μm, 3 μm, 3.25 μm, 3.5 μm, 3.75 μm, 4 μm, 4.25 μm, 4.5 μm, 4.75 μm, 5 μm, 5.5 μm, 6 μm, 6.5 μm, 7 μm, 7.5 μm, 8 μm, 8.5 μm, 9 μm, 9.5 μm, or 10 μm. In some case the locations may be positioned with a pitch that is within a range defined by any two of the preceding values. The locations may be positioned with a pitch of less than about 0.1 μm or greater than about 10 μm. In some cases, the pitch between any two locations of the same type may be determined as a function of a size of a loading object (e.g., bead). For example, where the loading object is a bead having a maximum diameter, the pitch may be at least about the maximum diameter of the loading object.

Indexing may be performed using a detection method and may be performed at any convenient or useful step. A substrate that is indexed, e.g., demarcated, may be subjected to detection, such as optical imaging, to locate the indexed locations, individually addressable locations, and/or the biological analyte. Imaging may be performed using a detection unit. Imaging may be performed using one or more sensors. Imaging may not be performed using the naked eye. The substrate that is indexed may be imaged prior to loading of the biological analyte. Following loading of the biological analyte onto the individually addressable locations, the substrate may be imaged again, e.g. to determine occupancy or to determine the positioning of the biological analyte relative to the substrate. In some cases, the substrate may be imaged after iterative cycles of nucleotide addition (or other probe or other reagent), as described elsewhere herein. The indexing of the substrate and known initial position (individually addressable location) of the biological analyte may allow for analysis and identification of the sequence information for each individually addressable location and/or position. Additionally, spatial indexing may allow for identification of errors that may occur, e.g., sample contamination, sample loss, etc.

The array may be coated with binders. For instance, the array may be randomly coated with binders. Alternatively, the array may be coated with binders arranged in a regular pattern (e.g., in linear arrays, radial arrays, hexagonal arrays etc.). The array may be coated with binders on at least 10%, 20%, 30%, 40%, 50%, 60%, 70%, 80%, 90%, 91%, 92%, 93%, 94%, 95%, 96%, 97%, 98%, or 99% of the number of individually addressable locations, or of the surface area of the substrate. The array may be coated with binders on a fraction of individually addressable locations, or of the surface areas of the substrate, that is within a range defined by any two of the preceding values. The binders may be integral to the array. The binders may be added to the array. For instance, the binders may be added to the array as one or more coating layers on the array.

The binders may be configured to immobilize analytes or reagents, such as through non-specific interactions, such as one or more of hydrophilic interactions, hydrophobic interactions, electrostatic interactions, physical interactions (for instance, adhesion to pillars or settling within wells), and the like. The binders may immobilize analytes or reagents through specific interactions. For instance, where the analyte or reagent is a nucleic acid molecule, the binders may comprise oligonucleotide adaptors configured to bind to the nucleic acid molecule. Alternatively or in addition, such as to bind other types of analytes or reagents, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. The array may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the array may comprise an order of magnitude of at most about 10¹¹, 10¹⁰, 10⁹, 10⁸, 10⁷, 10⁶, 10⁵, 10⁴, 10³, 100, 10 or fewer binders. The array may have a number of binders that is within a range defined by any two of the preceding values. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents.

In some instances, each location, or a subset of such locations, may have immobilized thereto an analyte (e.g., a nucleic acid molecule, a protein molecule, a carbohydrate molecule, etc.) or reagent. In other instances, a fraction of the plurality of individually addressable location may have immobilized thereto an analyte or reagent. A plurality of analytes or reagents immobilized to the substrate may be copies of a template analyte or template reagent. For example, the plurality of analytes (e.g., nucleic acid molecules) or reagents may have sequence homology. In other instances, the plurality of analytes or reagents immobilized to the substrate may not be copies. The plurality of analytes may be of the same type of analyte (e.g., a nucleic acid molecule) or reagent or may be a combination of different types of analytes or reagents (e.g., nucleic acid molecules, protein molecules, etc.).

In some instances, the array may comprise a plurality of types of binders. For example, the array may comprise different types of binders to bind different types of analytes or reagents. For example, the array may comprise a first type of binders (e.g., oligonucleotides) configured to bind a first type of analyte (e.g., nucleic acid molecules) or reagent, and a second type of binders (e.g., antibodies) configured to bind a second type of analyte (e.g., proteins) or reagent, and the like. In another example, the array may comprise a first type of binders (e.g., first type of oligonucleotide molecules) to bind a first type of nucleic acid molecules and a second type of binders (e.g., second type of oligonucleotide molecules) to bind a second type of nucleic acid molecules, and the like. For example, the substrate may be configured to bind different types of analytes or reagents in certain fractions or specific locations on the substrate by having the different types of binders in the certain fractions or specific locations on the substrate.

An array may have any number of individually addressable locations. For instance, the array may have at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, 1,000,000,000, 2,000,000,000, 5,000,000,000, 10,000,000,000, 20,000,000,000, 50,000,000,000, or 100,000,000,000 individually addressable locations. The array may have a number of individually addressable locations that is within a range defined by any two of the preceding values. Each individually addressable location may be digitally and/or physically accessible individually (from the plurality of individually addressable locations). For example, each individually addressable location may be located, identified, and/or accessed electronically or digitally for mapping, sensing, associating with a device (e.g., detector, processor, dispenser, etc.), or otherwise processing. As described elsewhere herein, each individually addressable location may be indexed. Alternatively, the substrate may be indexed such that each individually addressable location may be identified during at least one step of the process. Alternatively or in addition, each individually addressable location may be located, identified, and/or accessed physically, such as for physical manipulation or extraction of an analyte, reagent, particle, or other component located at an individually addressable location. In some instances, each individually addressable locations may have or be coupled to a binder, as described herein, to immobilize an analyte thereto. In some instances, only a fraction of the individually addressable locations may have or be coupled to a binder. In some instances, an individually addressable location may have or be coupled to a plurality of binders to immobilize an analyte or reagent thereto.

The analytes bound to the individually addressable locations may include, but are not limited to, molecules, cells, tissues, organisms, nucleic acid molecules, nucleic acid colonies, beads, clusters, polonies, DNA nanoballs, or any combination thereof (e.g., bead having attached thereto one or more nucleic acid molecules, e.g., one or more clonal populations of nucleic acid molecules). The analytes bound to the individually addressable locations may include any analyte described herein. The bound analytes may be immobilized to the array in a regular, patterned, periodic, random, or pseudo-random configuration, or any other spatial arrangement. In some embodiments, the analytes are bound to bead(s) which may then associate with or be immobilized to the substrate or regions of the substrate (e.g., individually addressable locations). In some embodiments, the analytes comprise a bead or a plurality of beads. In some cases, the bead or plurality of beads may comprise another analyte (e.g., nucleic acid molecule) or a clonal population of other analytes (e.g., a nucleic acid molecule that has been amplified on the bead). Such other analytes may be attached or otherwise coupled to the bead. For example, an analyte may comprise a plurality of beads, each bead having a clonal population of nucleic acid molecules attached thereto. In some cases, the bead is magnetic, and application of a magnetic field or using a magnet may be used to direct the analytes or beads comprising the analytes to the individually addressable locations. In some cases, the bead is electrically charged, and application of an electric field may be used to direct the analytes or beads comprising the analytes to the individually addressable locations. In some cases, a fluid may be used to direct the analyte to the individually addressable locations. The fluid may be a ferrofluid, and a magnet may be used to direct the fluid to the individually addressable locations. The individually addressable locations may alternatively or in conjunction comprise a material that is sensitive to a stimulus, e.g., thermal, chemical, or electrical or magnetic stimulus. For example, the individually addressable location may comprise a photo-sensitive polymer or reagent that is activated when exposed to electromagnetic radiation. In some cases, a caged molecule may be used to reveal binding (e.g., biotin) moieties (e.g., binders) on the substrate. Subsequent exposure to a particular wavelength of light may result in un-caging of the binding moieties. A bead, e.g., with streptavidin, comprising the analyte may then associate with the uncaged binding moieties. In some cases, a subset of the individually addressable locations may not contain beads. In such cases, blank beads may be added to the substrate. The blank beads may then occupy the regions that are unoccupied by an analyte. In some cases, the blank beads have a higher binding affinity or avidity for the individually addressable locations than the beads comprising the analyte. In some cases, unoccupied locations, or binders at such locations, may be destroyed or rendered inactive. In some cases, unoccupied locations may be subjected to a process to remove any unbound analyte, e.g., aspiration, washing, air blasting etc. In some cases, the sample comprising the analyte may be loaded onto the substrate using a device, e.g., a microfluidic device, closed flow cell, etc. The loaded analyte may then associate with or be immobilized to the substrate or the individually addressable locations of the substrate. In such cases, the device may be removed following loading of the sample. Though examples herein describe immobilization of analytes to the substrate, similar mechanisms may immobilize reagents to the substrate. For example, reagents may comprise or be coupled to bead(s).

An analyte may be bound to any number of beads. Different analytes may be bound to any number of beads. The beads may be unique (i.e., distinct from each other). Any number of unique beads may be used. For instance, an order of magnitude of at least about 10, 100, 1000, 10,000, 100,000, 1,000,000, 10,000,000, 100,000,000, 1,000,000,000, 10,000,000,000, 100,000,000,000 or more different beads may be used. Alternatively or in addition, an order of magnitude of at most about 100,000,000,000, 10,000,000,000, 1,000,000,000, 100,000,000, 10,000,000, 1,000,000, 100,000, 10,000, 1000, 100, or 10 different beads may be used. A number of different beads can be within a range defined by any two of the preceding values. The beads may be distinguishable from one another using a property of the beads, such as color, reflectance, anisotropy, brightness, fluorescence, etc. As described elsewhere herein, in some cases, different beads may comprise different tags (e.g., nucleic acid sequences) coupled thereto. For example, a bead may comprise an oligonucleotide molecule comprising a tag that identifies a bead amongst a plurality of beads.

A sample may be diluted such that the approximate occupancy of the individually addressable locations is controlled. A sample may be diluted at least to a dilution of 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900, 1:1000, 1:10000, 1:100000, 1:1000000, 1:10000000, 1:100000000. Alternatively, a sample may be diluted at most to a dilution of 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900, 1:1000, 1:10000, 1:100000, 1:1000000, 1:10000000, 1:100000000. A dilution between any of these dilution values may also be used.

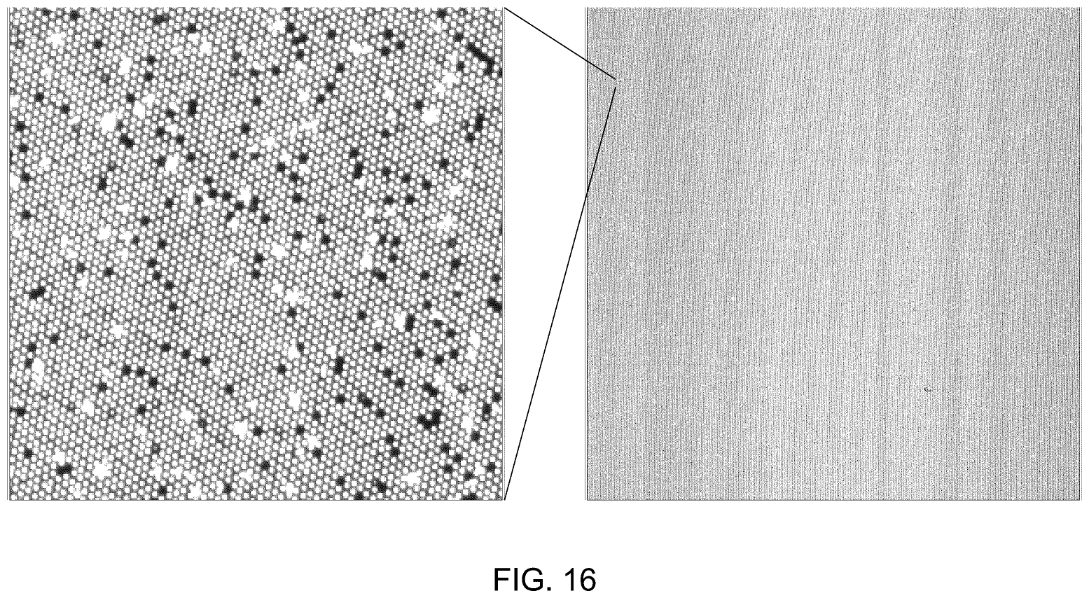

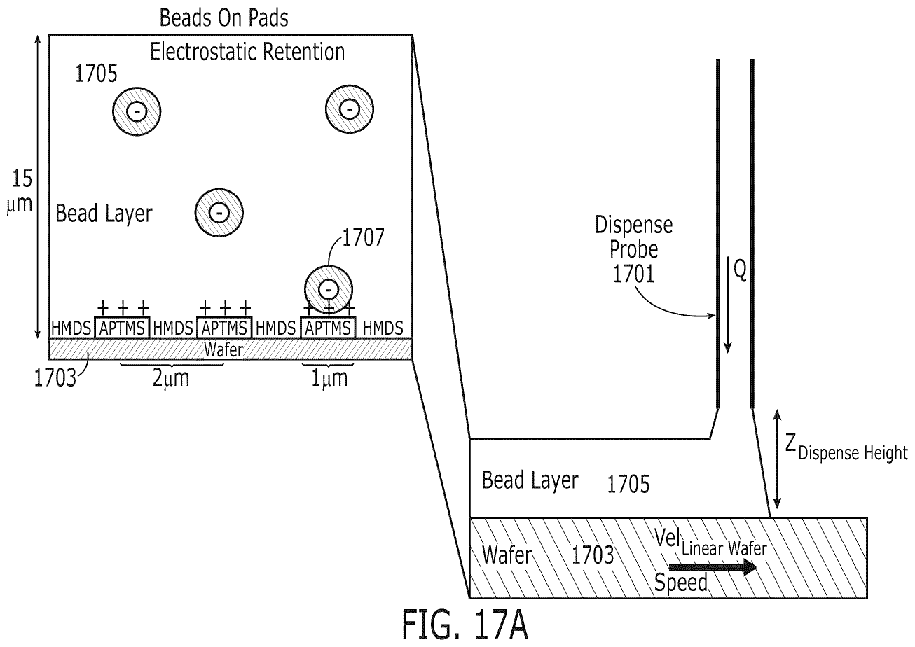

In some instances, a sample may comprise beads. Beads may be dispersed on a surface in any pattern, or randomly. Beads may be dispersed on one or more regions (e.g., a region having a particular surface chemistry) of a surface. In some cases, beads may be dispersed on a surface or a region of a surface in a hexagonal lattice, as shown in FIG. 16, which illustrates in the right panel a zoomed out image of a portion of a surface, and in the left panel a zoomed in image of a section of the portion of the surface. In some instances, a sample comprising beads may be dispersed on a surface comprising distinct locations/regions differentiated by surface chemistry (e.g., as illustrated in FIG. 15A and FIG. 15B). For example, a sample comprising beads may be dispensed on a surface comprising positively charged locations/regions and/or hydrophobic locations/regions. The beads may have a high affinity for a first location type or region type (e.g., positively charged). The beads may have a low affinity for a second location type or region type (e.g., hydrophobic). A location may comprise no more than 1, 2, 3, 4, 5, 6, 7, 8, 9, or 10 beads per location. In some embodiments, a bead may be substantially centered within an individually addressable location when immobilized. A location may have a width that is up to about 0.5 times, 0.6 times, 0.7 times, 0.8 times, 0.9 times, 1 times, 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, or 3 times the diameter (e.g., maximum diameter) of the bead. In some embodiments, a region may be spaced with a pitch determined by the distance between the center of a first location and the center of the closest or neighboring location of the same type. A location may be spaced with a pitch that is at least about 1 times, 1.2 times, 1.4 times, 1.6 times, 1.8 times, 2 times, 2.2 times, 2.4 times, 2.6 times, 2.8 times, 3 times, 3.2 times, 3.4 times, 3.6 times, 3.8 times, 4 times, 4.2 times, 4.4 times, 4.6 times, 4.8 times, or 5 times the diameter (e.g., maximum diameter) of the bead. In some cases, one or more of a location size, a location spacing, a bead affinity, a location surface chemistry may be adjusted to reduce a deviation of a bead contact point from the center of a region. Though examples herein describe a sample comprising beads, similarly, a reagent dispensed to the substrate may comprise beads.

A surface comprising a plurality of individually addressable locations may be loaded with beads. The beads may be loaded onto the surface at an occupancy determined by the number of locations of a given location type comprising at least one bead out of the total number of locations of the same location type. A surface comprising a plurality of locations may have occupancy of at least about 50%, 60%, 70%, 80%, 85%, 86%, 87%, 88%, 89%, 90%, 91%, 92%, 93%, 94%, 95%, 96%, 97%, 98%, 99.5%, or 100%. For example, a surface may have at least about 90% of the locations of a given location type loaded with at least one bead. Beads may land on the surface with a landing efficiency determined by the number of beads that bind to the surface out of the total number of beads dispensed on the surface. Beads may be dispensed onto a surface with a landing efficiency of at least about 10%, 20%, 30%, 40%, 50%, 55%, 60%, 65%, 70%, 75%, 80%, 90%, or 100%. In some embodiments, one or more of a temperature, an incubation time, a surfactant, or a salt concentration of a solution comprising beads may be adjusted to increase bead occupancy. In some embodiments, one or more of a temperature, an incubation time, a surfactant, or a salt concentration of a solution comprising beads may be adjusted to increase bead loading efficiency.

In some cases, at least about 5%, 10%, 15%, 20%, 25%, 30%, 35%, 40%, 45%, 50%, 55%, 60%, 65%, 70%, 75%, 80%, 85%, 90%, or 95% of the available surface area of a substrate may be configured to accept a bead. In some instances, where less than 100% of the available surface area has loaded thereon a bead (e.g., has a bead immobilized thereto), negative space (e.g., locations with no bead immobilized thereto) may be used as a reference to identify and/or index different individually addressable locations of the positive space (e.g., locations in which there is a bead). In some instances, a single individually addressable location in negative space is sufficient to index the entire substrate. The single individually addressable location in negative space will always remain ‘dark’ during imaging (e.g., during sequencing). In contrast, individually addressable locations in positive space will light up (e.g., be detectable, e.g., fluoresce) at different points in time (e.g., in more than 1 point in time) due to the present of analyte or reagent in the positive space. Thus, the single individually addressable location which is always ‘dark’ may act as a reference against all other individually addressable locations. In other examples, multiple individually addressable locations in negative space may facilitate indexing of the substrate (e.g., serve as reference points). Alternatively or in addition, a reference bead which is always ‘bright’ (e.g., always fluorescing regardless of time point) may be used as a reference to identify and/or index different individually addressable locations of the positive space. In such cases, even with 100% or substantially 100% of the available surface area loaded with beads, including the reference bead, the different individually addressable locations may be identified and/or indexed.

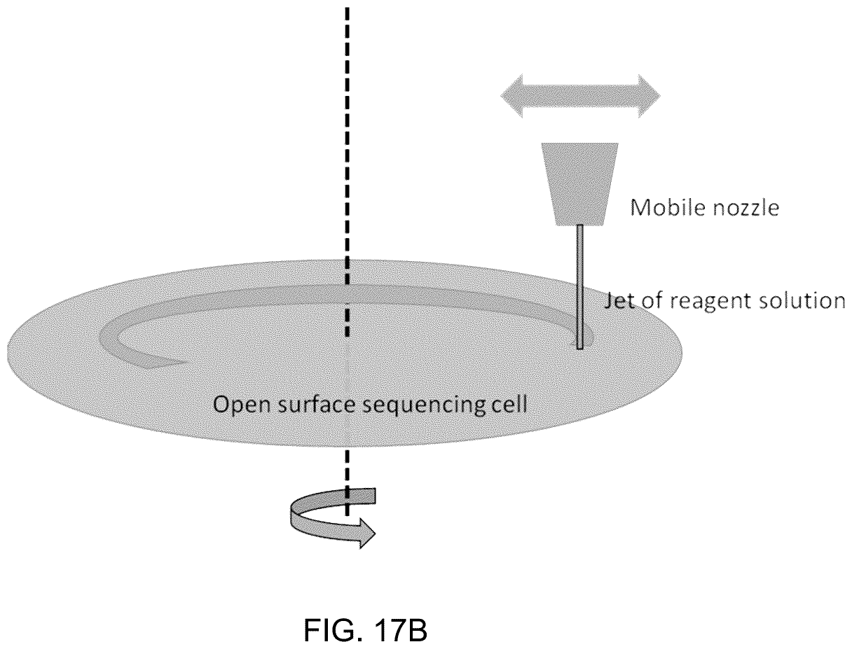

In some cases, beads may be dispensed to the substrate according to one or more systems and methods shown in FIGS. 17A-17B. As shown in FIG. 17A, a solution comprising beads may be dispensed from a dispense probe 1701 (e.g., a nozzle) to a substrate 1703 (e.g., a wafer) to form a layer 1705. The dispense probe may be positioned at a fixed height (“Z”) above the substrate. In the illustrated example, the beads are retained in the layer 1705 by electrostatic retention, and may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 1705 comprising the dispensed bead may successfully land on a first location of the first location type (as in 1707). In the illustrated example, the location size is 1 micron, the pitch between the different locations of the same location type (e.g., first location type) is 2 microns, and the layer has a depth of 15 micron.

FIG. 17B illustrates a reagent (e.g., beads) being dispensed along a path on an open surface of the substrate. As shown in FIG. 17B, a reagent solution may be dispensed from a dispense probe (e.g., a nozzle). In some embodiments, a solution may be dispensed from a plurality of dispense probes. For example, a first reagent in a solution may be dispensed from a first dispense probe, a second reagent in a solution may be dispensed from a second dispense probe, and a third reagent in a solution may be dispensed from a third dispense probe. In some cases, the reagents dispensed from different dispense probes may combine on the substrate to form a homogenous or substantially homogenous solution. The dispense probe may be positioned at a fixed height above a substrate (e.g., a wafer). The reagent may be dispensed on the surface in any desired pattern or path. This may be achieved by moving one or both of the substrate and the dispense nozzle. In some cases, the reagent (e.g., beads) may be dispensed to obtain a substantially spiral pattern. In order to achieve this the substrate and/or the dispense probe may have angular and/or linear velocity with respect to each other. In some instances, beads may be dispersed in a spiral pattern moving radially inward toward an axis of rotation of the surface. In some instances, beads may be dispersed in a spiral pattern moving radially outward from an axis of rotation of the surface. The substrate and the dispense probe may move in any configuration with respect to each other to achieve any pattern (e.g., linear pattern, substantially spiral pattern, etc.).

The substrate may be configured to move in any vector with respect to a reference point. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Such components may be mechanically connected to the substrate directly or indirectly via intermediary components (e.g., gears, stages, actuators, discs, pulleys, etc.). The motion unit may be automated. Alternatively or in addition, the motion unit may receive manual input. The substrate may be configured to move with any velocity. In some instances, the substrate may be configured to move with different velocities during different operations described herein. The substrate may be configured to move with a velocity that varies according to a time-dependent function, such as a ramp, sinusoid, pulse, or other function or combination of functions. The time-varying function may be periodic or aperiodic. In some examples, the substrate may be configured to move along an x-axis, a y-axis, a z-axis, or any combination thereof, where an x-axis and y-axis are substantially parallel to a surface plane of the substrate and a z-axis is substantially normal to the surface plane of the substrate.

A solution may be provided to the substrate prior to or during motion of the substrate to inertially direct the solution across the array on the substrate. The surface-reagent exchange may comprise washing, in which each subsequent pulse comprises a reduced concentration of the surface reagent. The solution may have a temperature different than that of the substrate, thereby providing a source or sink of thermal energy to the substrate or to an analyte or reagent located on the substrate. The thermal energy may provide a temperature change to the substrate or to the analyte or reagent. The temperature change may be transient. The temperature change may enable, disable, enhance, or inhibit a chemical reaction, such as a chemical reaction to be carried out upon the analyte. For example, the chemical reaction may comprise denaturation, hybridization, or annealing of nucleic acid molecules. The chemical reaction may comprise a step in a polymerase chain reaction (PCR), bridge amplification, or other nucleic acid amplification reaction. The temperature change may modulate, increase, or decrease a signal detected from the analyte (or from probes in the solution).

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an airgap. The nozzle may be configured to direct a solution to the array. The outlet may be configured to receive a solution from the array. The solution may be directed to the array using one or more dispensing nozzles. For example, the solution may be directed to the array using at least 1, 2, 3, 4, 5, 6, 7, 8, 9, 10, 11, 12, 13, 14, 15, 16, 17, 18, 19, or 20 dispensing nozzles. The solution may be directed to the array using a number of nozzles that is within a range defined by any two of the preceding values. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some instances, one or more factors such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. In some cases, a surfactant may be added to the solution, or a surfactant may be added to the surface to facilitate uniform coating or to facilitate sample loading efficiency. Such optimization may prevent the solution from exiting the substrate along a relatively focused stream or travel path such that the fluid only contacts the substrate at partial surface areas (as opposed to the entire surface area)—in such cases, a significantly larger volume of reagents may have to be dispensed to achieve uniform and full coating of the substrate. Such optimization may also prevent the solution from scattering or otherwise reflecting or bouncing off the substrate upon contact and disturbing the surface fluid. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

In some cases, the solution may be heated prior to being dispensed on the substrate. The solution may be at a higher temperature than the ambient temperature. The solution may be heated to about 25° C., 26° C., 27° C., 28° C., 29° C., 30° C., 31° C., 32° C., 33° C., 34° C., 35° C., 36° C., 37° C., 38° C., 39° C., 40° C., 45° C., 50° C., 55° C., 60° C., 65° C., 70° C., 75° C., 80° C., 85° C., 90° C., 95° C. prior to dispensing. In some cases, a solution may be heated to a temperature that is within a range defined by any two of the preceding values.

One or more solutions dispensed on a surface may undergo a reaction on the surface. For example, a first solution (e.g., comprising a reactant) dispensed on the surface may react with a second solution (e.g., comprising an enzyme) dispensed on the surface on top of the first solution. One or more solutions dispensed on a surface may deactivate or quench a chemical reaction. For example, a quenching solution (e.g., comprising EDTA or an acid) may be added to the substrate on top of a reaction to quench the reaction. A solution (e.g., a solution comprising a reactant, a solution comprising an enzyme, or a quenching solution) may be dispensed on the surface in a pattern (e.g., a spiral pattern). In some embodiments, a quenching solution is dispensed on the surface in the same pattern as a solution comprising a reactant, thereby maintaining a substantially constant reaction time at each point on the surface to which a solution is dispensed. In some embodiments, a quenching solution is dispensed on the surface in the same pattern as a solution comprising an enzyme, thereby maintaining a substantially constant reaction time at each point on the surface to which a solution is dispensed. Alternatively or in addition, similarly, one or more solutions dispensed on a surface may activate or catalyze a chemical reaction. For example, an activating solution (e.g., comprising catalysts, enzymes, primers, etc.) may be added to the substrate on top of a reaction (e.g., in the same dispense pattern) to activate or catalyze a reaction.

A variety of methods may be employed to dispense one or more solutions onto a substrate to ensure a substantially similar reaction time across an area of the substrate in contact with the one or more solutions. In some embodiments, a solution may be spin-coated onto a surface by dispensing the solution at or near the axis of rotation of a rotating substrate such that the centrifugal force of the rotating substrate facilitates the outward spread of the solution away from the axis of rotation. Spin-coating may be well-suited for dispensing one or more solutions that initiate or quench a reaction that occurs on a time scale that is slow relative to the dispensing time. In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. The pattern may be a spiral pattern. The pattern may be a circular pattern. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. The pattern may be a spiral pattern. The pattern may be a circular pattern. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. The pattern may be a spiral pattern. The pattern may be a circular pattern. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet.

A solution may be incubated on the substrate. In some embodiments, the solution may be incubated on the substrate under conditions that maintain a layer of fluid on the surface. The solution may be incubated for at least about 5 min, 10 min, 15 min, 20 min, 25 min, 30 min, 35 min, 40 min, 45 min, 50 min, 55 min, 60 min, 65 min, 70 min, 75 min, 80 min, 85 min, or 90 min. In some cases the incubation time may be within a range defined by any two of the preceding values. In some cases, the incubation may be for more than 90 minutes. In some instances, the layer of fluid may maintain a film thickness of at least 10 nanometers (nm), 20 nm, 50 nm, 100 nm, 200 nm, 500 nm, 1 micrometer (μm), 2 μm, 5 μm, 10 μm, 20 μm, 50 μm, 100 μm. 200 μm, 500 μm, or 1 mm during incubation. One or more of the temperature of the chamber, the humidity of the chamber, the motion of the substrate, or the composition of the fluid may be adjusted such that the layer of fluid is maintained during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The solution may comprise any sample or any analyte disclosed herein. The solution may comprise any reagent disclosed herein. In some cases, the solution may be a reaction mixture comprising a variety of components. For example, the solution may comprise a plurality of probes configured to interact with the analyte. For example, the probes may have binding specificity to the analyte. In another example, the probes may not have binding specificity to the analyte. A probe may be configured to permanently couple to the analyte. A probe may be configured to transiently couple to the analyte. For example, a nucleotide probe may be permanently incorporated into a growing strand hybridized to a nucleic acid molecule analyte. Alternatively, a nucleotide probe may transiently bind to the nucleic acid molecule analyte. Transiently coupled probes may be subsequently removed from the analyte. Subsequent removal of the transiently coupled probes from an analyte may or may not leave a residue (e.g., chemical residue) on the analyte. A type of probe in the solution may depend on the type of analyte. A probe may comprise a functional group or moiety configured to perform specific functions. For example, a probe may comprise a label (e.g., dye). A probe may be configured to generate a detectable signal (e.g., optical signal), such as via the label, upon coupling or otherwise interacting with the analyte. In some instances, a probe may be configured to generate a detectable signal upon activation (e.g., application of a stimulus). In another example, a nucleotide probe may comprise reversible terminators (e.g., blocking groups) configured to terminate polymerase reactions (until unblocked). The solution may comprise other components to aid, accelerate, or decelerate a reaction between the probe and the analyte (e.g., enzymes, catalysts, buffers, saline solutions, chelating agents, reducing agents, other agents, etc.). In some instances, the solution may be a washing solution. In some instances, a washing solution may be directed to the substrate to bring the washing solution in contact with the array after a reaction or interaction between reagents (e.g., a probe) in a reaction mixture solution with an analyte immobilized on the array. The washing solution may wash away any free reagents from the previous reaction mixture solution. In some instances, the solution may comprise a cleaving agent, such as to cleave a label and/or a blocking group, and/or otherwise act on a cleavage site (e.g., to cleave a sequence). Though examples herein describe interaction between a probe and an analyte, the probe may be configured to interact with any other reagent described herein, for example a reagent immobilized to an individually addressable location. In some examples, an analyte in one processing experiment may be used as a reagent for another processing experiment. The different processing experiments may be performed on the same substrate or different substrates. In some instances, a bead comprising an oligonucleotide molecule comprising a barcode sequence may be immobilized to an individually addressable location on the substrate, the oligonucleotide molecule may be interrogated as the analyte by one or more probes (e.g., so as to identify the barcode sequence, such as to index the individually addressable location with the barcode sequence), a sample may be loaded onto the substrate (e.g., such as over the bead), and then the bead comprising the oligonucleotide molecule immobilized to the individually addressable location may be used to capture another analyte (e.g., nucleic acid molecule, e.g., mRNA transcript) at the individually addressable location (e.g., so as to tag the other analyte with the barcode sequence). The tagged analyte may be collected from the substrate, processed (e.g., released from the bead and amplified on another bead such that the other bead comprises an amplification product of the tagged analyte), and it or its derivative reloaded onto another substrate for interrogation by one or more probes such as to determine a sequence of the tagged analyte or its derivative.

A detectable signal, such as an optical signal (e.g., fluorescent signal), may be generated upon reaction between a probe in the solution and the analyte. For example, the signal may originate from the probe and/or the analyte. The detectable signal may be indicative of a reaction or interaction between the probe and the analyte. The detectable signal may be a non-optical signal. For example, the detectable signal may be an electronic signal. The detectable signal may be detected by one or more sensors. For example, an optical signal may be detected via one or more optical detectors in an optical detection scheme described elsewhere herein. The signal may be detected during motion of the substrate. The signal may be detected following termination of the motion of the substrate. The signal may be detected while the analyte is in fluid contact with the solution. The signal may be detected following washing of the solution. In some instances, after the detection, the signal may be muted, such as by cleaving a label from the probe and/or the analyte, and/or modifying the probe and/or the analyte. Such cleaving and/or modification may be affected by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of directing a solution to the substrate and detection of one or more signals indicative of a reaction between a probe in the solution and an analyte in the array may be repeated one or more times. Such operations may be repeated in an iterative manner. For example, the same analyte immobilized to a given location in the array may interact with multiple solutions in the multiple repetition cycles. For each iteration, the additional signals detected may provide incremental, or final, data about the analyte during the processing. For example, where the analyte is a nucleic acid molecule and the processing is sequencing, additional signals detected for each iteration may be indicative of a base in the nucleic acid sequence of the nucleic acid molecule. The operations may be repeated at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 cycles to process the analyte. In some instances, a different solution may be directed to the substrate for each cycle. For example, at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 solutions may be directed to the substrate.

In some instances, a washing solution may be directed to the substrate between each cycle (or at least once during each cycle). For instance, a washing solution may be directed to the substrate after each type of reaction mixture solution is directed to the substrate. The washing solutions may be distinct. The washing solutions may be identical. For example, at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 washing solutions may be directed to the substrate.

In some instances, a subset or an entirety of the solution(s) may be recycled after the solution(s) have contacted the substrate. Recycling may comprise collecting, filtering, and reusing the subset or entirety of the solution. The filtering may be molecule filtering.

### Sample Processing Using an Array

In some instances, a method for sequencing may employ sequencing by synthesis schemes wherein a nucleic acid molecule is sequenced base-by-base with primer extension reactions. For example, a method for sequencing a nucleic acid molecule may comprise providing a substrate comprising an array having immobilized thereto the nucleic acid molecule. The array may be a planar array. The substrate may be configured to move with respect to a reference point. The method may comprise directing a solution comprising a plurality of nucleotides across the array prior to or during motion of the substrate. The nucleic acid molecule may be subjected to a primer extension reaction under conditions sufficient to incorporate or specifically bind at least one nucleotide from the plurality of nucleotides into a growing strand that is complementary to the nucleic acid molecule. A signal indicative of incorporation or binding of at least one nucleotide may be detected, thereby sequencing the nucleic acid molecule.

In some instances, the method may comprise, prior to providing the substrate having immobilized thereto the nucleic acid molecule, immobilizing the nucleic acid molecule to the substrate. For example, a solution comprising a plurality of nucleic acid molecules comprising the nucleic acid molecule may be directed to the substrate prior to, during, or subsequent to motion of the substrate, and the substrate may be subject to conditions sufficient to immobilize at least a subset of the plurality of nucleic acid molecules as an array on the substrate.

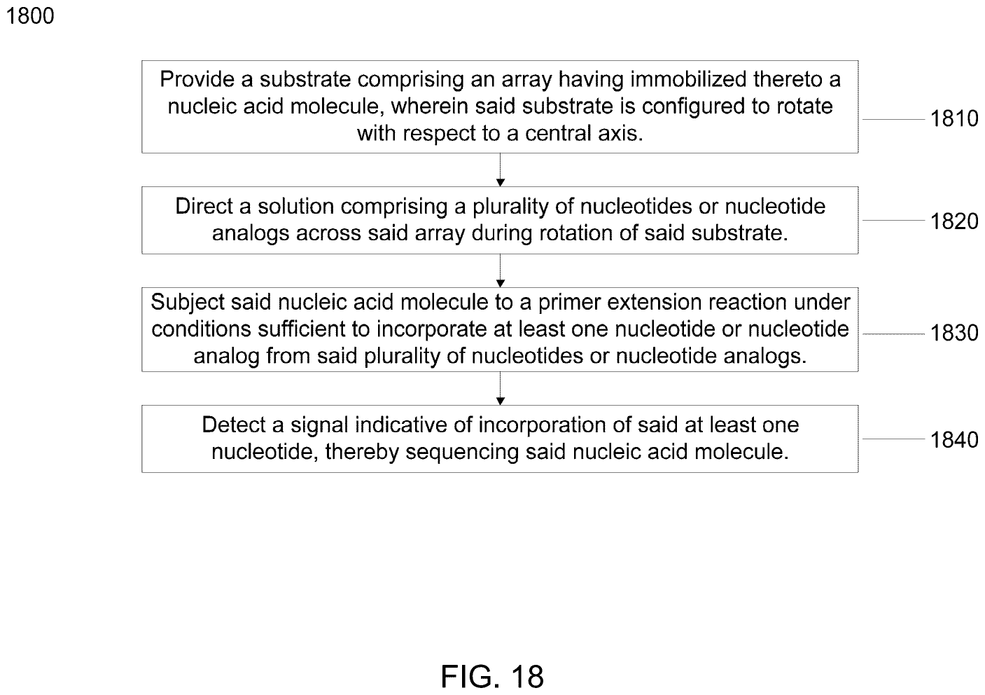

FIG. 18 shows a flowchart for an example of a method 1800 for sequencing a nucleic acid molecule. In a first operation 1810, the method may comprise providing a substrate, as described elsewhere herein. The substrate may comprise an array of a plurality of individually addressable locations. The array may be a planar array. The array may be a textured array. The array may be a patterned array. For example, the array may define individually addressable locations with wells and/or pillars. A plurality of nucleic acid molecules, which may or may not be copies of the same nucleic acid molecule, may be immobilized to the array. Each nucleic acid molecule from the plurality of nucleic acid molecules may be immobilized to the array at a given individually addressable location of the plurality of individually addressable locations. The substrate may be configured to move with respect to a reference point. The substrate may be configured to move with different velocities during different operations described herein. The substrate may be configured to move with a velocity that varies according to a time-dependent function, such as a ramp, sinusoid, pulse, or other function or combination of functions. The time-varying function may be periodic or aperiodic.

In a second operation 1820, the method may comprise directing a solution across the array prior to or during motion of the substrate. The solution may be centrifugally directed across the array. In some instances, the solution may be directed to the array during motion of the substrate in a continuous stream while the stream moves radially with respect to a reference point of the substrate. In some cases, a solution may comprise beads, as described elsewhere herein. The beads may be coated with a nucleic acid molecule to be sequenced. The solution comprising beads may be dispensed onto the substrate using the methods described herein. For example, the solution comprising beads may be dispensed onto the substrate, as illustrated in FIGS. 17A-17B. The beads may be dispensed according to any pattern (e.g., spiral pattern). In some cases, the beads may preferentially interact with a first region type of the substrate (e.g., a positively charged region), as illustrated in FIG. 17A. In some cases, a bead may not interact with a second region type of the substrate (e.g., a hydrophobic region). In some cases, a bead coated with a nucleic acid molecule may interact with a first region of the substrate (e.g., a positively charged region), and a bead that is not coated with a nucleic acid molecule may not interact with the first region type of the substrate, as shown in FIG. 17B.

In some instances, the solution may comprise probes configured to interact with nucleic acid molecules. For example, in some instances, such as for performing sequencing by synthesis, the solution may comprise a plurality of nucleotides (in single bases). The plurality of nucleotides may include nucleotide analogs, naturally occurring nucleotides, and/or non-naturally occurring nucleotides, collectively referred to herein as “nucleotides.” The plurality of nucleotides may or may not be bases of the same canonical base type (e.g., A, T, G, C, etc.). For example, the solution may or may not comprise bases of only one type. The solution may comprise at least 1 type of base or bases of at least 2, 3, 4, 5, 6, 7, 8, 9, or 10 types. For instance, the solution may comprise any possible mixture of A, T, C, and G, or subset thereof. In some instances, the solution may comprise a plurality of natural nucleotides and non-natural nucleotides. The plurality of natural nucleotides and non-natural nucleotides may or may not be bases of the same type (e.g., A, T, G, C). In some cases, the solution may comprise probes that are oligomeric (e.g., oligonucleotide primers). For example, in some instances, such as for performing sequencing by synthesis, the solution may comprise a plurality of nucleic acid molecules, e.g., primers, that comprise 1, 2, 3, 4, 5, 6, 7, 8, 9, 10, 20, 30, 40, 50, 60, 70, 80, 90, 100 or more nucleotide bases. The plurality of nucleic acid molecules may comprise nucleotide analogs, naturally occurring nucleotides, and/or non-naturally occurring nucleotides, collectively referred to herein as “nucleotides.”

In some cases, the plurality of nucleotides in the solution may be non-terminated. In some cases, none of the nucleotides in the solution may be terminated. Following incorporation of a non-terminated nucleotide into a nucleic acid strand, the nucleic acid strand may be able to incorporate another nucleotide. For example, where a solution of non-terminated A-base nucleotides are provided to a template that comprises a poly-T sequence, at the poly-T sequence locations the nucleic acid strand may incorporate multiple non-terminated A-base nucleotides in consecution.

Alternatively, one or more nucleotides of the plurality of nucleotides may be terminated (e.g., reversibly terminated). For example, a nucleotide may comprise a reversible terminator, or a moiety that is capable of terminating primer extension reversibly. Nucleotides comprising reversible terminators may be accepted by polymerases and incorporated into growing nucleic acid sequences analogously to non-reversibly terminated nucleotides. A polymerase may be any naturally occurring (i.e., native or wild-type) or engineered variant of a polymerase (e.g., DNA polymerase, Taq polymerase, etc.). Following incorporation of a nucleotide analog comprising a reversible terminator into a nucleic acid strand, the reversible terminator may be removed to permit further extension of the nucleic acid strand. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH₂ reversible terminators, 3′-O-allyl reversible terminators, and 3′-O-aziomethyl reversible terminators. Alternatively, a reversible terminator may comprise a blocking group in a linker (e.g., a cleavable linker) and/or dye moiety of a nucleotide analog. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al. Cleavage of a reversible terminator may be achieved by, for example, irradiating a nucleic acid molecule including the reversible terminator. In some instances the plurality of nucleotides may not comprise a terminated nucleotide.

One or more nucleotides of the plurality of nucleotides may be labeled with a dye, fluorophore, or quantum dot. For example, the solution may comprise labeled nucleotides. In another example, the solution may comprise unlabeled nucleotides. In another example, the solution may comprise a mixture of labeled and unlabeled nucleotides. Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, Atto 390, 425, 465, 488, 495, 532, 565, 594, 633, 647, 647N, 665, 680 and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q. In some cases, the label may be one with linkers. For instance, a label may have a disulfide linker attached to the label. Non-limiting examples of such labels include Cy5-azide, Cy-2-azide, Cy-3-azide, Cy-3.5-azide, Cy5.5-azide and Cy-7-azide. In some cases, a linker may be a cleavable linker. In some cases, the label may be a type that does not self-quench or exhibit proximity quenching. Non-limiting examples of a label type that does not self-quench or exhibit proximity quenching include Bimane derivatives such as Monobromobimane. Alternatively, the label may be a type that self-quenches or exhibits proximity quenching. Non-limiting examples of such labels include Cy5-azide, Cy-2-azide, Cy-3-azide, Cy-3.5-azide, Cy5.5-azide and Cy-7-azide. In some instances, a blocking group of a reversible terminator may comprise the dye.

The solution may be directed to the array using one or more nozzles. In some cases, different reagents (e.g., nucleotide solutions of different types, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Two or more nozzles may be operated in combination to deliver fluids to the substrate more efficiently.

The solution may be dispensed on the substrate while the substrate is stationary; the substrate may then be subjected to motion following the dispensing of the solution. Alternatively, the substrate may be subjected to motion prior to the dispensing of the solution; the solution may then be dispensed on the substrate while the substrate is moving.

In a third operation 1830, the method may comprise subjecting the nucleic acid molecule to a primer extension reaction. The primer extension reaction may be conducted under conditions sufficient to incorporate at least one nucleotide from the plurality of nucleotides into a growing strand that is complementary to the nucleic acid molecule. The nucleotide incorporated may or may not be labeled. In some cases, the operation 1830 may further comprise modifying at least one nucleotide. Modifying the nucleotide may comprise labeling the nucleotide. For instance, the nucleotide may be labeled, such as with a dye, fluorophore, or quantum dot. The nucleotide may be cleavably labeled. In some instances, modifying the nucleotide may comprise activating (e.g., stimulating) a label of the nucleotide.

In a fourth operation 1840, the method may comprise detecting a signal indicative of incorporation of the at least one nucleotide. The signal may be an optical signal. The signal may be a fluorescence signal. The signal may be detected during motion of the substrate. The signal may be detected following termination of the motion of the substrate. The signal may be detected while the nucleic acid molecule to be sequenced is in fluid contact with the solution. The signal may be detected following fluid contact of the nucleic acid molecule with the solution. The operation 1840 may further comprise modifying a label of the at least one nucleotide. For instance, the operation 1840 may further comprise cleaving the label of the nucleotide (e.g., after detection). The nucleotide may be cleaved by one or more stimuli, such as exposure to a chemical, an enzyme, light (e.g., ultraviolet light), or heat. Once the label is cleaved, a signal indicative of the incorporated nucleotide may not be detectable with one or more detectors.

The method 1800 may further comprise repeating operations 1820, 1830, and/or 1840 one or more times to identify one or more additional signals indicative of incorporation of one or more additional nucleotides, thereby sequencing the nucleic acid molecule. The method 1800 may comprise repeating operations 1820, 1830, and/or 1840 in an iterative manner. For each iteration, an additional signal may indicate incorporation of an additional nucleotide. The additional nucleotide may be the same nucleotide as detected in the previous iteration. The additional nucleotide may be a different nucleotide from the nucleotide detected in the previous iteration. In some instances, at least one nucleotide may be modified (e.g., labeled and/or cleaved) between each iteration of the operations 1820, 1830, or 1840. For instance, the method may comprise repeating the operations 1820, 1830, and/or 1840 at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 times. The method may comprise repeating the operations 1820, 1830, and/or 1840 a number of times that is within a range defined by any two of the preceding values. The method 1800 may thus result in the sequencing of a nucleic acid molecule of any size.

The method may comprise directing different solutions to the array during rotation of the substrate in a cyclical manner. For instance, the method may comprise directing a first solution containing a first type of nucleotide (e.g., in a plurality of nucleotides of the first type) to the array, followed by a second solution containing a second type of nucleotide, followed by a third type of nucleotide, followed by a fourth type of nucleotide, etc. In another example, different solutions may comprise different combinations of types of nucleotides. For example, a first solution may comprise a first canonical type of nucleotide (e.g., A), a second solution may comprise a second canonical type of nucleotide (e.g., C), a third solution may comprise a third canonical type of nucleotide (e.g., T), and a fourth solution may comprise a fourth canonical type (e.g., G) of nucleotide. In another example, a first solution may comprise a first canonical type of nucleotide (e.g., A) and a second canonical type of nucleotide (e.g., C), and a second solution may comprise the first canonical type of nucleotide (e.g., A) and a third canonical type of nucleotide (e.g., T), and a third solution may comprise the first canonical type, second canonical type, third canonical type, and a fourth canonical type (e.g., G) of nucleotide. In another example, a first solution may comprise a mixture of labeled and unlabeled nucleotides, and a second solution may comprise unlabeled nucleotides. In another example, a first solution may comprise labeled nucleotides, and a second solution may comprise unlabeled nucleotides, and a third solution may comprise a mixture of labeled and unlabeled nucleotides. In another example, a first solution may comprise a mixture of labeled and unlabeled nucleotides of a first canonical base type (e.g., A), a second solution may comprise unlabeled nucleotides of the first canonical base type (e.g., A), a third solution may comprise a mixture of labeled and unlabeled nucleotides of a second canonical base type (e.g., C), a fourth solution may comprise unlabeled nucleotides of the second canonical base type (e.g., C), a fifth solution may comprise a mixture of labeled and unlabeled nucleotides of a third canonical base type (e.g., T), a sixth solution may comprise unlabeled nucleotides of the third canonical base type (e.g., T), a seventh solution may comprise a mixture of labeled and unlabeled nucleotides of a fourth canonical base type (e.g., G), and an eighth solution may comprise unlabeled nucleotides of the fourth canonical base type (e.g., G). The method may comprise directing at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, at least 10,000,000, 20,000,000, 50,000,000, 100,000,000, 200,000,000, 500,000,000, or 1,000,000,000 types of solutions to the array. The method may comprise directing a number of types of solutions that is within a range defined by any two of the preceding values to the array.

The method may comprise directing at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 washing solutions to the substrate. For instance, a washing solution may be directed to the substrate after each type of nucleotide is directed to the substrate. The washing solutions may be distinct. The washing solutions may be identical.

The method may further comprise recycling a subset or an entirety of the solution(s) after the solution(s) have contacted the substrate. Recycling may comprise collecting, filtering, and reusing the subset or entirety of the solution. The filtering may be molecule filtering.

In some cases, the operations 1820 and 1830 may occur at a first location and the operation 1840 may occur at a second location. The first and second locations may comprise first and second processing stations, respectively, as described herein. Alternatively, the operation 1820 may occur at a first location and the operations 1830 and 1840 may occur at the second location.

The method may further comprise transferring the substrate between the first and second locations. Operations 1820 and 1830 may occur while the substrate is moved at a first velocity and operation 1840 may occur while the substrate is moved at a second velocity. The first velocity may be less than the second velocity. Alternatively, the operation 1820 may occur while the substrate is moved at the first velocity and the operations 1830 and 1840 may occur while the substrate is moved at the second velocity.

Many variations, alterations, and adaptations based on the method 1800 provided herein are possible. For example, the order of the operations of the method 1800 may be changed, some of the operations removed, some of the operations duplicated, and additional operations added as appropriate. Some of the operations may be performed in succession. Some of the operations may be performed in parallel. Some of the operations may be performed once. Some of the operations may be performed more than once. Some of the operations may comprise sub-operations. Some of the operations may be automated. Some of the operations may be manual. Some of the operations may be performed separately, e.g., in different locations or during different steps and/or processes. For example, directing a solution comprising a plurality of probes to the substrate may occur separately from the reaction and detection processes.

For example, in some cases, in the third operation 1830, instead of facilitating a primer extension reaction, the nucleic acid molecule may be subject to conditions to allow transient binding of a nucleotide from the plurality of nucleotides to the nucleic acid molecule. The transiently bound nucleotide may be labeled. The transiently bound nucleotide may be removed, such as after detection (e.g., see operation 1840). Then, a second solution may be directed to the substrate, this time under conditions to facilitate the primer extension reaction, such that a nucleotide of the second solution is incorporated (e.g., into a growing strand hybridized to the nucleic acid molecule). The incorporated nucleotide may be unlabeled. After washing, and without detecting, another solution of labeled nucleotides may be directed to the substrate, such as for another cycle of transient binding.

In some instances, such as for performing sequencing by ligation, the solution may comprise different probes. For example, the solution may comprise a plurality of oligonucleotide molecules. For example, the oligonucleotide molecules may have a length of about 2 bases, 3 bases, 4 bases, 5 bases, 6 bases, 7 bases, 8 bases, 9 bases, 10 bases or more. The oligonucleotide molecules may be labeled with a dye (e.g., fluorescent dye), as described elsewhere herein. In some instances, such as for detecting repeated sequences in nucleic acid molecules, such as homopolymer repeated sequences, dinucleotide repeated sequences, and trinucleotide repeated sequences, the solution may comprise targeted probes (e.g., homopolymer probe) configured to bind to the repeated sequences. The solution may comprise one type of probe (e.g., nucleotides). The solution may comprise different types of probes (e.g., nucleotides, oligonucleotide molecules, etc.). The solution may comprise different types of probes (e.g., oligonucleotide molecules, antibodies, etc.) for interacting with different types of analytes (e.g., nucleic acid molecules, proteins, etc.). Different solutions comprising different types of probes may be directed to the substrate any number of times, with or without detection between consecutive cycles (e.g., detection may be performed between some consecutive cycles, but not between some others), to sequence or otherwise process the nucleic acid molecule, depending on the type of processing.

### EXAMPLES

### Example 1. Single Bead Type, Using Single-Stranded DNA

Provided is an example method for barcoding analytes using a single bead type, according to the methods illustrated by and described with respect to FIG. 20 and FIG. 8A. A solid surface is covered by a single type of barcoded bead (e.g., 2001, 801) to form a bead array. A bead comprises single-stranded DNA with spatial tags and one or more uracils as a cleavage site, as described elsewhere herein. Tissues are sectioned and permeabilized. Tissues can be sectioned and/or permeabilized while on top of the bead array. On the tissue, a solution of barcoded bridge constructs is pipetted to bind targets (e.g., mRNA with poly-A tail). The barcoded bridge constructs can comprise a random UMI and a known bridge barcode that is unique to the tissue. The known bridge barcode can be used to attribute an mRNA to a certain tissue even after analytes, or their derivates, from many tissues are pooled during one or more downstream operations. A USER enzyme mix is added to the tissue. The tissue is flipped and placed on the bead array. The USER enzyme cleaves the uracils at the cleavage sites, which releases the spatial tag strands. Diffusion carries the spatial tags to the targets, and/or vice versa. The spatial tags bind to the bridge construct-target complex, such that two spatial tags form a complex per target mRNA. The complex is ligated. Reverse transcription is performed to generate cDNA. As described elsewhere herein, the cDNA can comprise a capture moiety (e.g., biotin), such as from the bridge construct and/or the spatial tag strands. The cDNA is enriched using the capture moiety (e.g., biotin captured by streptavidin), subjected to library preparation where one or more sequencing adaptors are ligated, and subjected to sequencing. In some cases, template switching is performed.

### Example 2. Two Bead Types, on Substrate

Provided is an example method for barcoding analytes using two bead types, according to the methods illustrated by and described with respect to FIGS. 19A-19D. A solid surface is covered by two types of barcoded beads (e.g., 1901, 1902) to form a bead array. The beads may be immobilized or bound to the solid surface. One of the bead types includes spatial tag oligonucleotides that have a biotin at the 5′ end of a spatial tag-containing strand. Tissues are sectioned on glass slides or coverslips and permeabilized. On the tissue, a solution of barcoded bridge constructs is pipetted to bind targets (e.g., mRNA with poly-A tail). The barcoded bridge constructs comprise a random UMI and a known bridge barcode that is unique to the tissue. The known bridge barcode can be used to attribute an mRNA to a certain tissue even after analytes, or their derivates, from many tissues are pooled during one or more downstream operations. A USER enzyme mix is added to the tissue. The tissue is flipped and placed on the bead array. The USER enzyme cleaves the dUs in H1′ and H2 (first bead type, e.g., 1901) and in H2′ (second bead type, e.g., 1902), which releases the spatial tag-containing partially double-stranded DNAs (with overhangs). Diffusion carries the spatial tags to the targets, and/or vice versa. The spatial tags bind to the bridge construct-target complex, such that two spatial tags form a complex per target mRNA. The bound molecules are ligated. Reverse transcription is performed to generate cDNA. The reverse transcription reaction appends a polyC sequence at the 3′ end of the first synthesized strand upon reaching the 5′ end of the mRNA template, which is used for template switching downstream. The cDNA comprises a biotin at the 5′ end of the first synthesized strand, which is used for target enrichment (e.g., capture by a streptavidin bead).

### Example 3. Two Bead Types, Off Substrate

Provided is another example method for barcoding analytes using two bead types, according to the methods illustrated by and described with respect to FIGS. 19A-19D. Different from Example 2, the two types of barcoded beads barcoded beads (e.g., 1901, 1902) are not bound to a solid surface but as a solution pipetted on the tissue section. The tissue section is sandwiched by one or more other glass slides. The USER enzyme can be added to the tissue before or after the beads are added. The remaining operations provided in Example 2 are performed.

### Example 4. Geolocation Bead Library Preparation

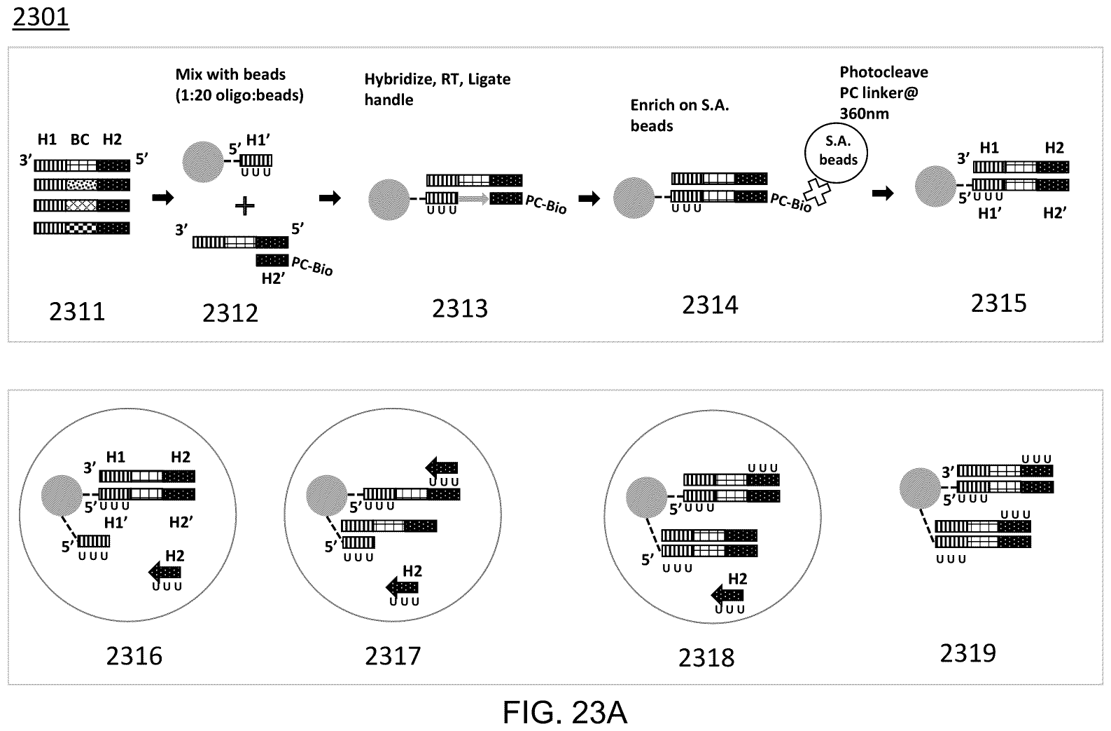

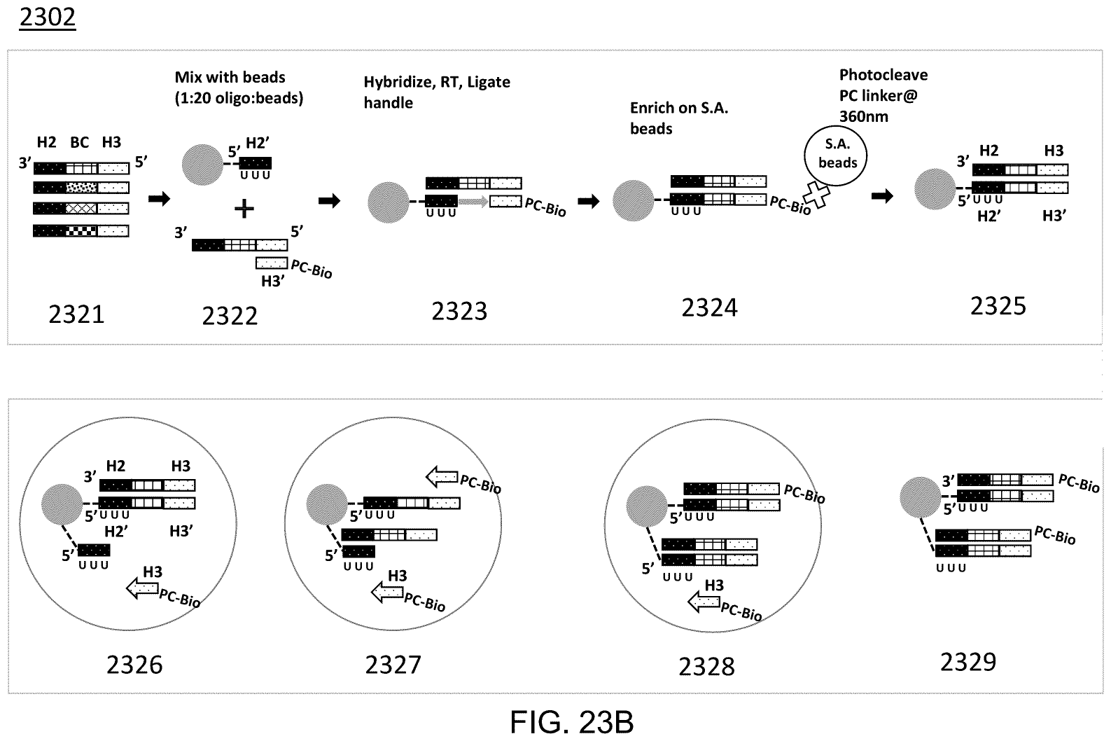

FIGS. 23A-23B illustrate an example method for preparing a geolocation bead library comprising two types of geolocation beads. The geolocation bead library generated may be used, for example, in the methods described with respect to FIGS. 19A-19D or other methods that use two types of geolocation beads.

For generation of a library of a first type of beads (2301), referring to FIG. 23A, a first barcode library is provided (2311), where each barcode library molecule has, from 3′ to 5′, a first handle (H1), a spatial tag (BC), and a second handle (H2). The spatial tag varies across different barcode library molecules. The first handle (H1) and the second handle (H2) are common to each barcode library molecule. In some cases, the number of molecules in the first barcode library has 10⁶˜10⁷ orders of magnitude. In some cases, the first handle (H1) has 12 bases and the second handle (H2) has 15 bases. Each of the library molecules is hybridized to a strand comprising a complement of the second handle (H2′) and photocleavable biotin at the 5′ end to form an intermediary complex. A plurality of starting beads is provided, at a ratio of approximately 1:20 intermediary complex to starting bead (2312). Other ratios may be used where the starting beads are provided in abundance of the intermediary complexes. Each starting bead is coupled to a plurality of oligonucleotide molecules. Each oligonucleotide molecule is coupled to the starting bead at the 5′ end and comprises a complement of the first handle (H1′) and one or more cleavage sites (e.g., uracils, marked as ‘U in FIG. 23A). The intermediary complexes are hybridized to the starting beads via the first handle (H1) of the intermediary complexes and the complement of the first handle (H1′) of the starting beads, reverse transcription is performed to extend the oligonucleotide coupled to the bead to fill in the gap corresponding to the spatial tag, and ligated, to generate a barcoded bead (2313). As there are many more starting beads than intermediary complexes, it is likely that only one intermediary complex binds to a starting bead (instead of multiple intermediary complex binding to a same starting bead). The mixture now has barcoded beads (bound to at least one spatial tag-containing intermediary complex) and starting beads (unbound to a spatial tag-containing intermediary complex). The barcoded beads in the mixture are pre-enriched (2314) by capturing the photocleavable biotin with streptavidin beads. After pre-enrichment, the barcoded beads are subjected to photo stimulus at 360 nm to cleave the photocleavable biotin (2315). ePCR is performed (bottom panel of FIG. 23A) using the pre-enriched barcoded beads. The barcoded beads are partitioned in droplets along with primers comprising the second handle (H2) and one or more cleavage sites (e.g., uracils, marked as ‘U in FIG. 23A) (2316). In some cases, the partitioning is controlled to ensure the presence of singularly occupied droplets, where a droplet has a single barcoded bead. In some cases, the partitioning is controlled to ensure that most occupied droplets are singularly occupied. A resulting droplet mixture may include a number of unoccupied droplets, a number of singularly occupied droplets, and rare or few multiply occupied droplets. In the droplet, the double strand on the barcoded bead is melted, and the primers annealed (2317). Reverse transcription cycles in the droplet generate double stranded oligonucleotide copies coupled to the bead (2318), a first strand comprising the first handle (H1), a spatial tag (BC), and the second handle (H2) with cleavage sites, and a second strand comprising a complement of the first handle (H1′) with cleavage sites, a complement of the spatial tag, and a complement of the second handle (H2′). The emulsion is broken and a library of the first type of beads is collected (2319).

For generation of a library of a second type of beads (2302), referring to FIG. 23B a second barcode library is provided (2321), where each barcode library molecule has, from 3′ to 5′, the second handle (H2), a spatial tag (BC), and a third handle (H3). The spatial tag varies across different barcode library molecules. The second handle (H2) and the third handle (H3) are common to each barcode library molecule. In some cases, the number of molecules in the second barcode library has 10⁶˜10⁷ orders of magnitude. Each of the library molecules is hybridized to a strand comprising a complement of the third handle (H3′) and photocleavable biotin at the 5′ end to form an intermediary complex. A plurality of starting beads is provided, at a ratio of approximately 1:20 intermediary complex to starting bead (2322). Other ratios may be used where the starting beads are provided in abundance of the intermediary complexes. Each starting bead is coupled to a plurality of oligonucleotide molecules. Each oligonucleotide molecule is coupled to the starting bead at the 5′ end and comprises a complement of the second handle (H2′) and one or more cleavage sites (e.g., uracils, marked as ‘U in FIG. 23B). The intermediary complexes are hybridized to the starting beads via the second handle (H2) of the intermediary complexes and the complement of the second handle (H2′) of the starting beads, reverse transcription is performed to extend the oligonucleotide coupled to the bead to fill in the gap corresponding to the spatial tag, and ligated, to generate a barcoded bead (2323). As there are many more starting beads than intermediary complexes, it is likely that only one intermediary complex binds to a starting bead (instead of multiple intermediary complex binding to a same starting bead). The mixture now has barcoded beads (bound to at least one spatial tag-containing intermediary complex) and starting beads (unbound to a spatial tag-containing intermediary complex). The barcoded beads in the mixture are pre-enriched (2324) by capturing the photocleavable biotin with streptavidin beads. After pre-enrichment, the barcoded beads are subjected to photo stimulus at 360 nm to cleave the photocleavable biotin (2325). ePCR is performed (bottom panel of FIG. 23B) using the pre-enriched barcoded beads. The barcoded beads are partitioned in droplets along with primers comprising the third handle (H3) and photocleavable biotin (or other capture moiety) (2326). In some cases, the partitioning is controlled to ensure the presence of singularly occupied droplets, where a droplet has a single barcoded bead. In some cases, the partitioning is controlled to ensure that most occupied droplets are singularly occupied. A resulting droplet mixture may include a number of unoccupied droplets, a number of singularly occupied droplets, and rare or few multiply occupied droplets. In the droplet, the double strand on the barcoded bead is melted, and the primers annealed (2327). Reverse transcription cycles in the droplet generate double stranded oligonucleotide copies coupled to the bead (2328), a first strand comprising the second handle (H2), a spatial tag (BC), and the third handle (H2) with the photocleavable biotin (or other capture moiety), and a second strand comprising a complement of the second handle (H2′) with cleavage sites, a complement of the spatial tag, and a complement of the third handle (H3′). The emulsion is broken and a library of the second type of beads is collected (2329).

The library of the first type of beads and the library of the second type of beads may be mixed before use (e.g., contacting tissue), or separately added during use.

In some cases, the same methods can be performed without the pre-enrichment operations (e.g., 2314-2315, 2324-2325), in which the barcoded beads may be subjected directly to ePCR without pre-enrichment. As such, in these methods, the intermediary complex may not require the photocleavable biotin or other capture moiety used for such pre-enrichment operations.

The method for generating a library for either type of bead may be used to generate a library of geolocation beads that is used in methods use a single type of bead.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

1. A method comprising: (a) providing a substrate comprising a plurality of geolocation beads immobilized to a plurality of individually addressable locations on said substrate, wherein said plurality of geolocation beads comprises a plurality of spatial tag molecules comprising a plurality of spatial tags, and wherein each geolocation bead of said plurality of geolocation beads comprises a unique spatial tag; (b) loading a sample onto said substrate, wherein said sample comprises a plurality of analyte sequences; (c) releasing said plurality of spatial tag molecules from said plurality of geolocation beads, wherein, prior to said releasing, respective locations of said plurality of spatial tags are unknown; (d) tagging a set of at least two spatial tags of said plurality of spatial tags to each of said plurality of analyte sequences to generate a plurality of spatially tagged sequences; and (e) generating a map of said plurality of analyte sequences by identifying sets of spatial tags from said plurality of spatially tagged sequences, wherein said map comprises spatial information between at least a subset of said plurality of analyte sequences. 2. The method of embodiment 1, further comprising, prior to (a), loading said plurality of geolocation beads onto said substrate to immobilize said plurality of geolocation beads at said plurality of individually addressable locations. 3. The method of any one of embodiments 1-2, further comprising fixing said sample. 4. The method of any one of embodiments 1-3, further comprising permeabilizing said sample. 5. The method of any one of embodiments 1-4, wherein said sample comprises a tissue sample, wherein said plurality of analyte sequences comprises a plurality of messenger ribonucleic acid (mRNA) transcript sequences. 6. The method of any one of embodiments 1-5, wherein (d) comprises contacting a plurality of bridge constructs with said plurality of spatial tag molecules and said plurality of analyte sequences, under conditions sufficient for a bridge construct of said plurality of bridge constructs to capture (1) a first spatial tag molecule from said plurality of spatial tags, wherein said first spatial tag molecule comprises a first spatial tag, (2) a second spatial tag molecule from said plurality of spatial tags, wherein said second spatial tag molecule comprises a second spatial tag, and (3) an analyte sequence of said plurality of analyte sequences, to generate a tagged complex, and generating a spatially tagged sequence of said plurality of spatially tagged sequences using said tagged complex. 7. The method of embodiment 6, wherein said bridge construct is a partially double-stranded nucleic acid molecule, comprising a capture sequence as an overhang, a first attachment binding sequence, a second attachment binding sequence, a third attachment binding sequence, and a fourth attachment binding sequence, wherein said capture sequence is configured to bind to a sequence of said analyte sequence, wherein said first spatial tag molecule comprises a first attachment sequence configured to bind to said first attachment binding sequence, said first spatial tag, and a second attachment sequence configured to bind to said second attachment binding sequence, wherein said second spatial tag molecule comprises a third attachment sequence configured to bind to said third attachment binding sequence, said second spatial tag, and a fourth attachment sequence configured to bind to said fourth attachment binding sequence, and wherein said tagged complex comprises a first nucleic acid strand comprising, in 3′ to 5′ order, (1) said capture sequence which is bound to said sequence of said analyte sequence, (2) said first attachment sequence which is bound to said first attachment binding sequence, (3) said first spatial tag which is not bound to another nucleic acid strand, (4) said second attachment sequence which is bound to said second attachment binding sequence, (5) said third attachment sequence which is bound to said third attachment binding sequence, (6) said second spatial tag which is not bound to another nucleic acid strand, and (7) said fourth attachment sequence which is bound to said fourth attachment binding sequence. 8. The method of embodiment 7, wherein said second spatial tag molecule comprises a capture entity, wherein said first nucleic acid strand of said tagged complex comprises said capture entity. 9. The method of embodiment 8, wherein said capture entity comprises biotin. 10. The method of any one of embodiments 7-9, wherein said bridge construct comprises a first spacer sequence disposed between said first attachment binding sequence and said second attachment binding sequence, wherein said bridge construct comprises a second spacer sequence disposed between said third attachment binding sequence and said fourth attachment binding sequence. 11. The method of any one of embodiments 7-10, wherein said capture sequence comprises a polyT sequence, and wherein said sequence of said analyte sequence comprises a polyA sequence. 12. The method of any one of embodiments 7-10, wherein said capture sequence comprises a random n-mer sequence. 13. The method of any one of embodiments 7-12, wherein a first subset of said plurality of spatial tag molecules each comprises a respective spatial tag disposed between said first attachment sequence and said second attachment sequence, and wherein a second subset of said plurality of spatial tag molecules each comprises a respective spatial tag disposed between said third attachment sequence and said fourth attachment sequence. 14. The method of any one of embodiments 7-13, wherein said first attachment sequence is different from said third attachment sequence. 15. The method of any one of embodiments 7-13, wherein said first attachment sequence is the same as said third attachment sequence. 16. The method of any one of embodiments 7-15, wherein said first attachment sequence is different from said second attachment sequence. 17. The method of any one of embodiments 7-15, wherein said first attachment sequence is the same as said second attachment sequence. 18. The method of any one of embodiments 1-17, wherein a geolocation bead of said plurality of geolocation beads comprises a set of spatial tag molecules of said plurality of spatial tag molecules, wherein each spatial tag molecule of said set of spatial tag molecules comprises a common spatial tag of said plurality of spatial tags. 19. The method of embodiment 18, wherein each spatial tag molecule of said set of spatial tag molecules further comprises a unique molecular identifier (UMI) that is unique amongst said set of spatial tag molecules. 20. The method of any one of embodiments 18-19, wherein each spatial tag molecule of said set of spatial tag molecules comprises a first attachment sequence and a second attachment sequence, wherein said common spatial tag is disposed between said first attachment sequence and said second attachment sequence. 21. The method of embodiment 20, wherein each spatial tag molecule of said set of spatial tag molecules further comprises a UMI that is unique amongst said set of spatial tag molecules, wherein said UMI is disposed between said first attachment sequence and said second attachment sequence. 22. The method of any one of embodiments 18-21, wherein said set of spatial tag molecules comprises a first subset of spatial tag molecules and a second subset of spatial tag molecules, wherein said first subset of spatial tag molecules comprises a first attachment sequence and a second attachment sequence, and wherein said second subset of spatial tag molecules comprises a third attachment sequence and a fourth attachment sequence, wherein said first attachment sequence is different from said third attachment sequence. 23. The method of embodiment 22, wherein said second attachment sequence is different from said fourth attachment sequence. 24. The method of any one of embodiments 18-23, wherein said set of spatial tag molecules comprises at least 100,000 spatial tag molecules. 25. The method of any one of embodiments 1-24, wherein said plurality of geolocation beads comprises a first set of geolocation beads and a second set of geolocation beads, wherein said first set of geolocation beads comprises a first set of spatial tag molecules of said plurality of spatial tag molecules each comprising a first attachment sequence and a second attachment sequence, wherein said second set of geolocation beads comprises a second set of spatial tag molecules of said plurality of spatial tag molecules each comprising a third attachment sequence and a fourth attachment sequence, wherein said first attachment sequence is different from said third attachment sequence. 26. The method of embodiment 25, wherein said second attachment sequence is different from said fourth attachment sequence. 27. The method of any one of embodiments 1-26, wherein a geolocation bead of said plurality of geolocation beads comprises an oligonucleotide molecule, wherein said oligonucleotide molecule comprises a spatial tag molecule of said plurality of spatial tag molecules. 28. The method of embodiment 27, wherein the releasing said plurality of spatial tag molecules from said plurality of geolocation beads comprises releasing oligonucleotide molecules from the plurality of geolocation beads. 29. The method of embodiment 28, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of geolocation beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of geolocation beads comprises an alpha-cyclodextrine (a-CD) moiety bound to the azobenzene. 30. The method of any one of embodiments 27-29, wherein said spatial tag molecule is a first strand of said oligonucleotide molecule. 31. The method of any one of embodiments 27-30, wherein said oligonucleotide molecule is a partially double-stranded nucleic acid molecule comprising a first strand a second strand, wherein said first strand comprises said spatial tag molecule, wherein said second strand comprises a primer sequence and sequences corresponding to said spatial tag molecule. 32. The method of embodiment 31, wherein (c) comprises releasing said spatial tag molecule from said second strand. 33. The method of any one of embodiments 1-32, wherein a spatial tag molecule of said plurality of spatial tag molecules comprises a capture entity. 34. The method of embodiment 33, wherein said capture entity comprises biotin. 35. The method of any one of embodiments 33-35, further comprising recovering said plurality of spatially tagged sequences using said capture entity. 36. The method of any one of embodiments 33-36, further comprising recovering a plurality of tagged complexes using said capture entity, and generating said plurality of spatially tagged sequences using said plurality of tagged complexes. 37. The method of any one of embodiments 1-36, wherein a spatial tag molecule of said plurality of spatial tag molecules comprises a blocking group and a cleavage site. 38. The method of any one of embodiments 1-37, wherein said plurality of geolocation beads comprises at least 1,000,000 geolocation beads. 39. The method of any one of embodiments 1-38, wherein said plurality of geolocation beads comprises at least 100,000,000 geolocation beads. 40. The method of any one of embodiments 1-39, wherein said plurality of geolocation beads comprises at least 100,000,000,000 geolocation beads. 41. The method of any one of embodiments 1-40, wherein an analyte sequence comprises an mRNA sequence. 42. The method of any one of embodiments 1-40, wherein an analyte sequence comprises a DNA sequence. 43. The method of any one of embodiments 1-42, wherein said spatial information comprises a relative position of said at least said subset of said plurality of analyte sequences with respect to a reference analyte sequence. 44. The method of any one of embodiments 1-43, wherein said spatial information comprises a relative probability cloud of said at least said subset of said plurality of analyte sequences with respect to a reference analyte sequence. 45. The method of any one of embodiments 1-44, wherein said spatial information comprises two-dimensional (2D) spatial information. 46. The method of any one of embodiments 1-45, wherein said spatial information comprises three-dimensional (3D) spatial information. 47. The method of any one of embodiments 1-46, further comprising, subsequent to (b) and prior to (d), (i) contacting said sample with a surface of a second substrate, wherein said second substrate comprises a second plurality of geolocation beads immobilized to a second plurality of individually addressable locations on said surface of said second substrate, wherein said second plurality of geolocation beads comprises a second plurality of spatial tag molecules comprising a second plurality of spatial tags, wherein each geolocation bead of said second plurality of geolocation beads comprises a unique spatial tag, wherein said plurality of spatial tags and said second plurality of spatial tags are mutually exclusive, and (ii) releasing said second plurality of spatial tag molecules from said second plurality of geolocation beads, wherein, prior to said releasing, respective locations or identities of said second plurality of spatial tags are unknown. 48. The method of embodiment 47, further comprising, in (d), tagging a set of at least two spatial tags of plurality of spatial tags, at least two spatial tags of said second plurality of spatial tags, or at least two spatial tags from both said plurality of spatial tags and said second plurality of spatial tags to each of said plurality of analyte sequences to generate said plurality of spatially tagged sequences.

49. A method comprising: (a) providing a substrate comprising a plurality of geolocation beads immobilized to a plurality of individually addressable locations on said substrate, wherein said plurality of geolocation beads comprises a plurality of spatial tag molecules comprising a plurality of spatial tags, and wherein each geolocation bead of said plurality of geolocation beads comprises a unique spatial tag; (b) loading a sample onto said substrate, wherein said sample comprises a plurality of analyte sequences; (c) releasing said plurality of spatial tag molecules from said plurality of geolocation beads, wherein, prior to said releasing, respective identities of said plurality of spatial tags are unknown; (d) tagging a set of at least two spatial tags of said plurality of spatial tag molecules to each of said plurality of analyte sequences to generate a plurality of spatially tagged sequences; and (e) generating a map of said plurality of analyte sequences by identifying sets of spatial tags from said plurality of spatially tagged sequences, wherein said map comprises spatial information between at least a subset of said plurality of analyte sequences. 50. The method of embodiment 49, further comprising, prior to (a), loading said plurality of geolocation beads onto said substrate to immobilize said plurality of geolocation beads at said plurality of individually addressable locations. 51. The method of any one of embodiments 49-50, further comprising fixing said sample. 52. The method of any one of embodiments 49-51, further comprising permeabilizing said sample. 53. The method of any one of embodiments 49-52, wherein said sample comprises a tissue sample, wherein said plurality of analyte sequences comprises a plurality of messenger ribonucleic acid (mRNA) transcript sequences. 54. The method of any one of embodiments 49-53, wherein (d) comprises contacting a plurality of bridge constructs with said plurality of spatial tag molecules and said plurality of analyte sequences, under conditions sufficient for a bridge construct of said plurality of bridge constructs to capture (1) a first spatial tag molecule from said plurality of spatial tags, wherein said first spatial tag molecule comprises a first spatial tag, (2) a second spatial tag molecule from said plurality of spatial tags, wherein said second spatial tag molecule comprises a second spatial tag, and (3) an analyte sequence of said plurality of analyte sequences, to generate a tagged complex, and generating a spatially tagged sequence of said plurality of spatially tagged sequences using said tagged complex. 55. The method of embodiment 54, wherein said bridge construct is a partially double-stranded nucleic acid molecule, comprising a capture sequence as an overhang, a first attachment binding sequence, a second attachment binding sequence, a third attachment binding sequence, and a fourth attachment binding sequence, wherein said capture sequence is configured to bind to a sequence of said analyte sequence, wherein said first spatial tag molecule comprises a first attachment sequence configured to bind to said first attachment binding sequence, said first spatial tag, and a second attachment sequence configured to bind to said second attachment binding sequence, wherein said second spatial tag molecule comprises a third attachment sequence configured to bind to said third attachment binding sequence, said second spatial tag, and a fourth attachment sequence configured to bind to said fourth attachment binding sequence, and wherein said tagged complex comprises a first nucleic acid strand comprising, in 3′ to 5′ order, (1) said capture sequence which is bound to said sequence of said analyte sequence, (2) said first attachment sequence which is bound to said first attachment binding sequence, (3) said first spatial tag which is not bound to another nucleic acid strand, (4) said second attachment sequence which is bound to said second attachment binding sequence, (5) said third attachment sequence which is bound to said third attachment binding sequence, (6) said second spatial tag which is not bound to another nucleic acid strand, and (7) said fourth attachment sequence which is bound to said fourth attachment binding sequence. 56. The method of embodiment 55, wherein said second spatial tag molecule comprises a capture entity, wherein said first nucleic acid strand of said tagged complex comprises said capture entity. 57. The method of embodiment 56, wherein said capture entity comprises biotin. 58. The method of any one of embodiments 55-57, wherein said bridge construct comprises a first spacer sequence disposed between said first attachment binding sequence and said second attachment binding sequence, wherein said bridge construct comprises a second spacer sequence disposed between said third attachment binding sequence and said fourth attachment binding sequence. 59. The method of any one of embodiments 55-58, wherein said capture sequence comprises a polyT sequence, and wherein said sequence of said analyte sequence comprises a polyA sequence. 60. The method of any one of embodiments 55-58, wherein said capture sequence comprises a random n-mer sequence. 61. The method of any one of embodiments 55-60, wherein a first subset of said plurality of spatial tag molecules each comprises a respective spatial tag disposed between said first attachment sequence and said second attachment sequence, and wherein a second subset of said plurality of spatial tag molecules each comprises a respective spatial tag disposed between said third attachment sequence and said fourth attachment sequence. 62. The method of any one of embodiments 55-61, wherein said first attachment sequence is different from said third attachment sequence. 63. The method of any one of embodiments 55-61, wherein said first attachment sequence is the same as said third attachment sequence. 64. The method of any one of embodiments 55-63, wherein said first attachment sequence is different from said second attachment sequence. 65. The method of any one of embodiments 55-63, wherein said first attachment sequence is the same as said second attachment sequence. 66. The method of any one of embodiments 49-65, wherein a geolocation bead of said plurality of geolocation beads comprises a set of spatial tag molecules of said plurality of spatial tag molecules, wherein each spatial tag molecule of said set of spatial tag molecules comprises a common spatial tag of said plurality of spatial tags. 67. The method of embodiment 66, wherein each spatial tag molecule of said set of spatial tag molecules further comprises a unique molecular identifier (UMI) that is unique amongst said set of spatial tag molecules. 68. The method of any one of embodiments 66-67, wherein each spatial tag molecule of said set of spatial tag molecules comprises a first attachment sequence and a second attachment sequence, wherein said common spatial tag is disposed between said first attachment sequence and said second attachment sequence. 69. The method of embodiment 68, wherein each spatial tag molecule of said set of spatial tag molecules further comprises a UMI that is unique amongst said set of spatial tag molecules, wherein said UMI is disposed between said first attachment sequence and said second attachment sequence. 70. The method of any one of embodiments 66-69, wherein said set of spatial tag molecules comprises a first subset of spatial tag molecules and a second subset of spatial tag molecules, wherein said first subset of spatial tag molecules comprises a first attachment sequence and a second attachment sequence, and wherein said second subset of spatial tag molecules comprises a third attachment sequence and a fourth attachment sequence, wherein said first attachment sequence is different from said third attachment sequence. 71. The method of embodiment 70, wherein said second attachment sequence is different from said fourth attachment sequence. 72. The method of any one of embodiments 66-71, wherein said set of spatial tag molecules comprises at least 100,000 spatial tag molecules. 73. The method of any one of embodiments 49-72, wherein said plurality of geolocation beads comprises a first set of geolocation beads and a second set of geolocation beads, wherein said first set of geolocation beads comprises a first set of spatial tag molecules of said plurality of spatial tag molecules each comprising a first attachment sequence and a second attachment sequence, wherein said second set of geolocation beads comprises a second set of spatial tag molecules of said plurality of spatial tag molecules each comprising a third attachment sequence and a fourth attachment sequence, wherein said first attachment sequence is different from said third attachment sequence. 74. The method of embodiment 73, wherein said second attachment sequence is different from said fourth attachment sequence. 75. The method of any one of embodiments 49-74, wherein a geolocation bead of said plurality of geolocation beads comprises an oligonucleotide molecule, wherein said oligonucleotide molecule comprises a spatial tag molecule of said plurality of spatial tag molecules. 76. The method of embodiment 75, wherein the releasing said plurality of spatial tag molecules from said plurality of geolocation beads comprises releasing oligonucleotide molecules from the plurality of geolocation beads. 77. The method of embodiment 76, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of geolocation beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of geolocation beads comprises an alpha-cyclodextrine (a-CD) moiety bound to the azobenzene. 78. The method of any one of embodiments 75-77, wherein said spatial tag molecule is a first strand of said oligonucleotide molecule. 79. The method of any one of embodiments 75-78, wherein said oligonucleotide molecule is a partially double-stranded nucleic acid molecule comprising a first strand a second strand, wherein said first strand comprises said spatial tag molecule, wherein said second strand comprises a primer sequence and sequences corresponding to said spatial tag molecule. 80. The method of embodiment 79, wherein (c) comprises releasing said spatial tag molecule from said second strand. 81. The method of any one of embodiments 49-80, wherein a spatial tag molecule of said plurality of spatial tag molecules comprises a capture entity. 82. The method of embodiment 81, wherein said capture entity comprises biotin. 83. The method of any one of embodiments 81-82, further comprising recovering said plurality of spatially tagged sequences using said capture entity. 84. The method of any one of embodiments 81-83, further comprising recovering a plurality of tagged complexes using said capture entity, and generating said plurality of spatially tagged sequences using said plurality of tagged complexes. 85. The method of any one of embodiments 49-84, wherein a spatial tag molecule of said plurality of spatial tag molecules comprises a blocking group and a cleavage site. 86. The method of any one of embodiments 49-85, wherein said plurality of geolocation beads comprises at least 1,000,000 geolocation beads. 87. The method of any one of embodiments 49-86, wherein said plurality of geolocation beads comprises at least 100,000,000 geolocation beads. 88. The method of any one of embodiments 49-87, wherein said plurality of geolocation beads comprises at least 100,000,000,000 geolocation beads. 89. The method of any one of embodiments 49-88, wherein an analyte sequence comprises an mRNA sequence. 90. The method of any one of embodiments 49-88, wherein an analyte sequence comprises a DNA sequence. 91. The method of any one of embodiments 49-90, wherein said spatial information comprises a relative position of said at least said subset of said plurality of analyte sequences with respect to a reference analyte sequence. 92. The method of any one of embodiments 49-91, wherein said spatial information comprises a relative probability cloud of said at least said subset of said plurality of analyte sequences with respect to a reference analyte sequence. 93. The method of any one of embodiments 49-92, wherein said spatial information comprises two-dimensional (2D) spatial information. 94. The method of any one of embodiments 49-93, wherein said spatial information comprises three-dimensional (3D) spatial information. 95. The method of any one of embodiments 49-94, further comprising, subsequent to (b) and prior to (d), (i) contacting said sample with a surface of a second substrate, wherein said second substrate comprises a second plurality of geolocation beads immobilized to a second plurality of individually addressable locations on said surface of said second substrate, wherein said second plurality of geolocation beads comprises a second plurality of spatial tag molecules comprising a second plurality of spatial tags, wherein each geolocation bead of said second plurality of geolocation beads comprises a unique spatial tag, wherein said plurality of spatial tags and said second plurality of spatial tags are mutually exclusive, and (ii) releasing said second plurality of spatial tag molecules from said second plurality of geolocation beads, wherein, prior to said releasing, respective locations or identities of said second plurality of spatial tags are unknown. 96. The method of embodiment 95, further comprising, in (d), tagging a set of at least two spatial tags of plurality of spatial tags, at least two spatial tags of said second plurality of spatial tags, or at least two spatial tags from both said plurality of spatial tags and said second plurality of spatial tags to each of said plurality of analyte sequences to generate said plurality of spatially tagged sequences.

97. A method comprising: (a) partitioning cells from a plurality of cells into a plurality of partitions, wherein a partition of said plurality of partitions comprises a cell and a plurality of geolocation beads, wherein said cell comprises a plurality of analyte sequences, wherein said plurality of geolocation beads comprises a plurality of spatial tag molecules comprising a plurality of spatial tags, and wherein each geolocation bead of said plurality of geolocation beads comprises a unique spatial tag; (b) releasing said plurality of spatial tag molecules from said plurality of geolocation beads; (c) tagging a set of at least two spatial tags of said plurality of spatial tags to each of said plurality of analyte sequences to generate a plurality of spatially tagged sequences; and (d) sequencing said plurality of spatially tagged sequences, or derivatives thereof, to determine that said plurality of analyte sequences originated from said cell of said plurality of cells by identifying sets of spatial tags from said plurality of spatially tagged sequences. 98. The method of embodiment 97, wherein said partition is a droplet. 99. The method of embodiment 97, wherein said partition is a well. 100. The method of any one of embodiments 97-99, wherein (c) comprises contacting a plurality of bridge constructs with said plurality of spatial tag molecules and said plurality of analyte sequences, under conditions sufficient for a bridge construct of said plurality of bridge constructs to capture (1) a first spatial tag molecule from said plurality of spatial tags, wherein said first spatial tag molecule comprises a first spatial tag, (2) a second spatial tag molecule from said plurality of spatial tags, wherein said second spatial tag molecule comprises a second spatial tag, and (3) an analyte sequence of said plurality of analyte sequences, to generate a tagged complex, and generating a spatially tagged sequence of said plurality of spatially tagged sequences using said tagged complex. 101. The method of embodiment 100, wherein said bridge construct is a partially double-stranded nucleic acid molecule, comprising a capture sequence as an overhang, a first attachment binding sequence, a second attachment binding sequence, a third attachment binding sequence, and a fourth attachment binding sequence, wherein said capture sequence is configured to bind to a sequence of said analyte sequence, wherein said first spatial tag molecule comprises a first attachment sequence configured to bind to said first attachment binding sequence, said first spatial tag, and a second attachment sequence configured to bind to said second attachment binding sequence, wherein said second spatial tag molecule comprises a third attachment sequence configured to bind to said third attachment binding sequence, said second spatial tag, and a fourth attachment sequence configured to bind to said fourth attachment binding sequence, and wherein said tagged complex comprises a first nucleic acid strand comprising, in 3′ to 5′ order, (1) said capture sequence which is bound to said sequence of said analyte sequence, (2) said first attachment sequence which is bound to said first attachment binding sequence, (3) said first spatial tag which is not bound to another nucleic acid strand, (4) said second attachment sequence which is bound to said second attachment binding sequence, (5) said third attachment sequence which is bound to said third attachment binding sequence, (6) said second spatial tag which is not bound to another nucleic acid strand, and (7) said fourth attachment sequence which is bound to said fourth attachment binding sequence. 102. The method of embodiment 101, wherein said second spatial tag molecule comprises a capture entity, wherein said first nucleic acid strand of said tagged complex comprises said capture entity. 103. The method of embodiment 102, wherein said capture entity comprises biotin. 104. The method of any one of embodiments 101-103, wherein said bridge construct comprises a first spacer sequence disposed between said first attachment binding sequence and said second attachment binding sequence, wherein said bridge construct comprises a second spacer sequence disposed between said third attachment binding sequence and said fourth attachment binding sequence. 105. The method of any one of embodiments 101-104, wherein said capture sequence comprises a polyT sequence, and wherein said sequence of said analyte sequence comprises a polyA sequence. 106. The method of any one of embodiments 101-104, wherein said capture sequence comprises a random n-mer sequence. 107. The method of any one of embodiments 101-106, wherein a first subset of said plurality of spatial tag molecules each comprises a respective spatial tag disposed between said first attachment sequence and said second attachment sequence, and wherein a second subset of said plurality of spatial tag molecules each comprises a respective spatial tag disposed between said third attachment sequence and said fourth attachment sequence. 108. The method of any one of embodiments 101-107, wherein said first attachment sequence is different from said third attachment sequence. 109. The method of any one of embodiments 101-107, wherein said first attachment sequence is the same as said third attachment sequence. 110. The method of any one of embodiments 101-109, wherein said first attachment sequence is different from said second attachment sequence. 111. The method of any one of embodiments 101-109, wherein said first attachment sequence is the same as said second attachment sequence. 112. The method of any one of embodiments 97-111, wherein a geolocation bead of said plurality of geolocation beads comprises a set of spatial tag molecules of said plurality of spatial tag molecules, wherein each spatial tag molecule of said set of spatial tag molecules comprises a common spatial tag of said plurality of spatial tags. 113. The method of embodiment 112, wherein each spatial tag molecule of said set of spatial tag molecules further comprises a unique molecular identifier (UMI) that is unique amongst said set of spatial tag molecules. 114. The method of any one of embodiments 112-113, wherein each spatial tag molecule of said set of spatial tag molecules comprises a first attachment sequence and a second attachment sequence, wherein said common spatial tag is disposed between said first attachment sequence and said second attachment sequence. 115. The method of embodiment 114, wherein each spatial tag molecule of said set of spatial tag molecules further comprises a UMI that is unique amongst said set of spatial tag molecules, wherein said UMI is disposed between said first attachment sequence and said second attachment sequence. 116. The method of any one of embodiments 112-115, wherein said set of spatial tag molecules comprises a first subset of spatial tag molecules and a second subset of spatial tag molecules, wherein said first subset of spatial tag molecules comprises a first attachment sequence and a second attachment sequence, and wherein said second subset of spatial tag molecules comprises a third attachment sequence and a fourth attachment sequence, wherein said first attachment sequence is different from said third attachment sequence. 117. The method of embodiment 116, wherein said second attachment sequence is different from said fourth attachment sequence. 118. The method of any one of embodiments 112-117, wherein said set of spatial tag molecules comprises at least 100,000 spatial tag molecules. 119. The method of any one of embodiments 97-118, wherein said plurality of geolocation beads comprises a first set of geolocation beads and a second set of geolocation beads, wherein said first set of geolocation beads comprises a first set of spatial tag molecules of said plurality of spatial tag molecules each comprising a first attachment sequence and a second attachment sequence, wherein said second set of geolocation beads comprises a second set of spatial tag molecules of said plurality of spatial tag molecules each comprising a third attachment sequence and a fourth attachment sequence, wherein said first attachment sequence is different from said third attachment sequence. 120. The method of embodiment 119, wherein said second attachment sequence is different from said fourth attachment sequence. 121. The method of any one of embodiments 97-120, wherein a geolocation bead of said plurality of geolocation beads comprises an oligonucleotide molecule, wherein said oligonucleotide molecule comprises a spatial tag molecule of said plurality of spatial tag molecules. 122. The method of embodiment 121, wherein the releasing said plurality of spatial tag molecules from said plurality of geolocation beads comprises releasing oligonucleotide molecules from the plurality of geolocation beads. 123. The method of embodiment 122, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of geolocation beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of geolocation beads comprises an alpha-cyclodextrine (a-CD) moiety bound to the azobenzene. 124. The method of embodiment 123, wherein said spatial tag molecule is a first strand of said oligonucleotide molecule. 125. The method of any one of embodiments 123-124, wherein said oligonucleotide molecule is a partially double-stranded nucleic acid molecule comprising a first strand a second strand, wherein said first strand comprises said spatial tag molecule, wherein said second strand comprises a primer sequence and sequences corresponding to said spatial tag molecule. 126. The method of embodiment 125, wherein (b) comprises releasing said spatial tag molecule from said second strand. 127. The method of any one of embodiments 97-126, wherein a spatial tag molecule of said plurality of spatial tag molecules comprises a capture entity. 128. The method of embodiment 127, wherein said capture entity comprises biotin. 129. The method of any one of embodiments 127-128, further comprising recovering said plurality of spatially tagged sequences using said capture entity. 130. The method of any one of embodiments 127-129, further comprising recovering a plurality of tagged complexes using said capture entity, and generating said plurality of spatially tagged sequences using said plurality of tagged complexes. 131. The method of any one of embodiments 97-130, wherein a spatial tag molecule of said plurality of spatial tag molecules comprises a blocking group and a cleavage site. 132. The method of any one of embodiments 97-131, wherein said plurality of geolocation beads comprises at least 1,000,000 geolocation beads. 133. The method of any one of embodiments 97-132, wherein said plurality of geolocation beads comprises at least 100,000,000 geolocation beads. 134. The method of any one of embodiments 97-133, wherein said plurality of geolocation beads comprises at least 100,000,000,000 geolocation beads. 135. The method of any one of embodiments 97-134, wherein an analyte sequence comprises an mRNA sequence. 136. The method of any one of embodiments 97-134, wherein an analyte sequence comprises a DNA sequence.

137. A method comprising: (a) providing a solution comprising a plurality of cells and a plurality of geolocation beads, wherein said plurality of cells comprises a plurality of analyte sequences, wherein said plurality of geolocation beads comprises a plurality of spatial tag molecules comprising a plurality of spatial tags, and wherein each geolocation bead of said plurality of geolocation beads comprises a unique spatial tag; (b) releasing said plurality of spatial tag molecules from said plurality of geolocation beads; (c) tagging a set of at least two spatial tags of said plurality of spatial tags to each of said plurality of analyte sequences to generate a plurality of spatially tagged sequences; and (d) sequencing said plurality of spatially tagged sequences, or derivatives thereof, to determine that a subset of said plurality of analyte sequences originated from a cell of said plurality of cells by identifying sets of spatial tags from said plurality of spatially tagged sequences. 138. The method of embodiment 137, wherein (c) comprises contacting a plurality of bridge constructs with said plurality of spatial tag molecules and said plurality of analyte sequences, under conditions sufficient for a bridge construct of said plurality of bridge constructs to capture (1) a first spatial tag molecule from said plurality of spatial tags, wherein said first spatial tag molecule comprises a first spatial tag, (2) a second spatial tag molecule from said plurality of spatial tags, wherein said second spatial tag molecule comprises a second spatial tag, and (3) an analyte sequence of said plurality of analyte sequences, to generate a tagged complex, and generating a spatially tagged sequence of said plurality of spatially tagged sequences using said tagged complex. 139. The method of embodiment 138, wherein said bridge construct is a partially double-stranded nucleic acid molecule, comprising a capture sequence as an overhang, a first attachment binding sequence, a second attachment binding sequence, a third attachment binding sequence, and a fourth attachment binding sequence, wherein said capture sequence is configured to bind to a sequence of said analyte sequence, wherein said first spatial tag molecule comprises a first attachment sequence configured to bind to said first attachment binding sequence, said first spatial tag, and a second attachment sequence configured to bind to said second attachment binding sequence, wherein said second spatial tag molecule comprises a third attachment sequence configured to bind to said third attachment binding sequence, said second spatial tag, and a fourth attachment sequence configured to bind to said fourth attachment binding sequence, and wherein said tagged complex comprises a first nucleic acid strand comprising, in 3′ to 5′ order, (1) said capture sequence which is bound to said sequence of said analyte sequence, (2) said first attachment sequence which is bound to said first attachment binding sequence, (3) said first spatial tag which is not bound to another nucleic acid strand, (4) said second attachment sequence which is bound to said second attachment binding sequence, (5) said third attachment sequence which is bound to said third attachment binding sequence, (6) said second spatial tag which is not bound to another nucleic acid strand, and (7) said fourth attachment sequence which is bound to said fourth attachment binding sequence. 140. The method of embodiment 139, wherein said second spatial tag molecule comprises a capture entity, wherein said first nucleic acid strand of said tagged complex comprises said capture entity. 141. The method of embodiment 140, wherein said capture entity comprises biotin. 142. The method of any one of embodiments 139-141, wherein said bridge construct comprises a first spacer sequence disposed between said first attachment binding sequence and said second attachment binding sequence, wherein said bridge construct comprises a second spacer sequence disposed between said third attachment binding sequence and said fourth attachment binding sequence. 143. The method of any one of embodiments 139-142, wherein said capture sequence comprises a polyT sequence, and wherein said sequence of said analyte sequence comprises a polyA sequence. 144. The method of any one of embodiments 139-142, wherein said capture sequence comprises a random n-mer sequence. 145. The method of any one of embodiments 139-144, wherein a first subset of said plurality of spatial tag molecules each comprises a respective spatial tag disposed between said first attachment sequence and said second attachment sequence, and wherein a second subset of said plurality of spatial tag molecules each comprises a respective spatial tag disposed between said third attachment sequence and said fourth attachment sequence. 146. The method of any one of embodiments 139-145, wherein said first attachment sequence is different from said third attachment sequence. 147. The method of any one of embodiments 139-145, wherein said first attachment sequence is the same as said third attachment sequence. 148. The method of any one of embodiments 139-147, wherein said first attachment sequence is different from said second attachment sequence. 149. The method of any one of embodiments 139-147, wherein said first attachment sequence is the same as said second attachment sequence. 150. The method of any one of embodiments 139-149, wherein a geolocation bead of said plurality of geolocation beads comprises a set of spatial tag molecules of said plurality of spatial tag molecules, wherein each spatial tag molecule of said set of spatial tag molecules comprises a common spatial tag of said plurality of spatial tags. 151. The method of embodiment 150, wherein each spatial tag molecule of said set of spatial tag molecules further comprises a unique molecular identifier (UMI) that is unique amongst said set of spatial tag molecules. 152. The method of any one of embodiments 150-151, wherein each spatial tag molecule of said set of spatial tag molecules comprises a first attachment sequence and a second attachment sequence, wherein said common spatial tag is disposed between said first attachment sequence and said second attachment sequence. 153. The method of embodiment 152, wherein each spatial tag molecule of said set of spatial tag molecules further comprises a UMI that is unique amongst said set of spatial tag molecules, wherein said UMI is disposed between said first attachment sequence and said second attachment sequence. 154. The method of any one of embodiments 150-153, wherein said set of spatial tag molecules comprises a first subset of spatial tag molecules and a second subset of spatial tag molecules, wherein said first subset of spatial tag molecules comprises a first attachment sequence and a second attachment sequence, and wherein said second subset of spatial tag molecules comprises a third attachment sequence and a fourth attachment sequence, wherein said first attachment sequence is different from said third attachment sequence. 155. The method of embodiment 154, wherein said second attachment sequence is different from said fourth attachment sequence. 156. The method of any one of embodiments 150-155, wherein said set of spatial tag molecules comprises at least 100,000 spatial tag molecules. 157. The method any one of embodiments 137-156, wherein said plurality of geolocation beads comprises a first set of geolocation beads and a second set of geolocation beads, wherein said first set of geolocation beads comprises a first set of spatial tag molecules of said plurality of spatial tag molecules each comprising a first attachment sequence and a second attachment sequence, wherein said second set of geolocation beads comprises a second set of spatial tag molecules of said plurality of spatial tag molecules each comprising a third attachment sequence and a fourth attachment sequence, wherein said first attachment sequence is different from said third attachment sequence. 158. The method of embodiment 157, wherein said second attachment sequence is different from said fourth attachment sequence. 159. The method of any one of embodiments 137-158, wherein a geolocation bead of said plurality of geolocation beads comprises an oligonucleotide molecule, wherein said oligonucleotide molecule comprises a spatial tag molecule of said plurality of spatial tag molecules. 160. The method of embodiment 159, wherein the releasing said plurality of spatial tag molecules from said plurality of geolocation beads comprises releasing oligonucleotide molecules from the plurality of geolocation beads. 161. The method of embodiment 160, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of geolocation beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of geolocation beads comprises an alpha-cyclodextrine (a-CD) moiety bound to the azobenzene. 162. The method of any one of embodiments 159-161, wherein said spatial tag molecule is a first strand of said oligonucleotide molecule. 163. The method of any one of embodiments 159-162, wherein said oligonucleotide molecule is a partially double-stranded nucleic acid molecule comprising a first strand a second strand, wherein said first strand comprises said spatial tag molecule, wherein said second strand comprises a primer sequence and sequences corresponding to said spatial tag molecule. 164. The method of embodiment 163, wherein (c) comprises releasing said spatial tag molecule from said second strand. 165. The method of any one of embodiments 137-164, wherein a spatial tag molecule of said plurality of spatial tag molecules comprises a capture entity. 166. The method of embodiment 165, wherein said capture entity comprises biotin. 167. The method of any one of embodiments 165-166, further comprising recovering said plurality of spatially tagged sequences using said capture entity. 168. The method of any one of embodiments 165-167, further comprising recovering a plurality of tagged complexes using said capture entity, and generating said plurality of spatially tagged sequences using said plurality of tagged complexes. 169. The method of any one of embodiments 137-168, wherein a spatial tag molecule of said plurality of spatial tag molecules comprises a blocking group and a cleavage site. 170. The method of any one of embodiments 137-169, wherein said plurality of geolocation beads comprises at least 1,000,000 geolocation beads. 171. The method of any one of embodiments 137-170, wherein said plurality of geolocation beads comprises at least 100,000,000 geolocation beads. 172. The method of any one of embodiments 137-171, wherein said plurality of geolocation beads comprises at least 100,000,000,000 geolocation beads. 173. The method of any one of embodiments 137-172, wherein an analyte sequence comprises an mRNA sequence. 174. The method of any one of embodiments 137-172, wherein an analyte sequence comprises a DNA sequence.

175. A kit, comprising: a substrate comprising a plurality of geolocation beads immobilized to a plurality of individually addressable locations on the substrate, wherein the plurality of geolocation beads comprises oligonucleotide molecules coupled thereto, wherein the oligonucleotide molecules comprise spatial tag molecules, wherein each geolocation bead of said plurality of geolocation beads comprises a unique spatial tag, wherein the oligonucleotide molecules are releasable from the plurality of geolocation beads by one or more of the release mechanisms selected from the group consisting of: (a) providing an enzyme mix comprising an enzyme configured to cleave or digest a cleavage site, wherein the oligonucleotide molecules comprise the cleavage site; (b) providing biotin, wherein the oligonucleotide molecules are conjugated to a desthiobiotin moiety, wherein the geolocation bead comprises a streptavidin moiety bound to the desthiobiotin moiety; (c) providing an enzyme mix comprising an enzyme configured to cleave or digest a cleavage site and UV light, wherein the oligonucleotide molecules comprise azobenzene and the cleavage site; and (d) providing UV light, wherein the oligonucleotide molecules are conjugated azobenzene, wherein the plurality of geolocation beads comprise an alpha-cyclodextrine (a-CD) moiety bound to the azobenzene. 176. The kit of embodiment 175, further comprising indexed data comprising a list of spatial tag sequences included in the plurality of geolocation beads. 177. The kit of embodiment 175 or 176, further comprising a second substrate comprising a second plurality of individually addressable locations configured to immobilize a second plurality of geolocation beads. 178. The kit of embodiment 177, further comprising the second plurality of geolocation beads. 179. The kit of any one of embodiments 175-178, further comprising a reagent configured to release the oligonucleotide molecules from the plurality of geolocation beads. 180. The kit of embodiment 179, wherein the reagent comprises one or more of: (i) an enzyme mix comprising an enzyme configured to cleave or digest a cleavage site, wherein the oligonucleotide molecules comprise the cleavage site; (ii) biotin, wherein the oligonucleotide molecules are conjugated to a desthiobiotin moiety, wherein the plurality of geolocation beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of geolocation beads comprises an alpha-cyclodextrine (a-CD) moiety bound to the azobenzene. 181. The kit of any one of embodiments 175-180, further comprising sequencing reagents. 182. The kit of embodiment 181, wherein the sequencing reagents comprise single-base nucleotide mixtures for each of the four base types. 183. The kit of embodiment 182, wherein a single-base nucleotide mixture of the single-base nucleotide mixtures comprises a mixture of labeled nucleotides and non-labeled nucleotides of a single base. 184. The kit of embodiment 182 or 183, wherein a single-base nucleotide mixture of the single-base nucleotide mixtures comprises non-terminated nucleotides. 185. The kit of any one of embodiments 175-184, further comprising amplification reagents. 186. The kit of embodiment 185, wherein the amplification reagents comprise a polymerase, a nucleotide mixture, a primer, a buffer, or any combination thereof. 187. The kit of any one of embodiments 175-186, further comprising a biological sample. 188. The kit of embodiment 187, wherein the biological sample is a tissue. 189. The kit of embodiment 187 or 188, wherein the biological sample is fixed. 190. The kit of any one of embodiments 187-189, wherein the biological sample is permeabilized. 191. The kit of any one of embodiments 187-190, wherein the biological sample is loaded on the substrate. 192. The kit of any one of embodiments 175-191, wherein a geolocation bead of the plurality of geolocation beads comprises at least 100,000 oligonucleotides molecules. 193. The kit of embodiment 192, wherein the at least 100,000 oligonucleotides molecules comprise a spatial tag of the spatial tags that is common and unique to the geolocation bead amongst the plurality of geolocation beads. 194. The kit of any one of embodiments 175-193, wherein an oligonucleotide molecule of the oligonucleotide molecules comprises a capture sequence, wherein the capture sequence is configured to hybridize with a sequence of an analyte, or derivative thereof, of a biological sample. 195. The kit of embodiment 194, wherein the capture sequence is selected from the group consisting of: a polyT sequence, a polyG sequence, a targeted mRNA sequence, a targeted gDNA sequence, a random n-mer sequence, and a probe sequence. 196. The kit of any one of embodiments 175-195, wherein the substrate comprises at least 1,000,000 individually addressable locations. 197. The kit of embodiment 196 wherein the substrate comprises at least 1,000,000,000 individually addressable locations. 198. The kit of any one of embodiments 175-197, wherein the plurality of geolocation beads is immobilized to the individually addressable locations via electrostatic interactions. 199. The kit of any one of embodiments 175-198, wherein the substrate is substantially planar.

200. A system, comprising: a sequencing platform configured to (i) address individually addressable locations of substrates and (ii) rotate the substrates during dispensing of sequencing reagents to the substrates or during imaging of the substrates or during both; and a substrate comprising a plurality of geolocation beads immobilized to a plurality of individually addressable locations on the substrate, wherein the plurality of geolocation beads comprises oligonucleotide molecules coupled thereto, wherein the oligonucleotide molecules comprise spatial tag molecules, wherein each geolocation bead of said plurality of geolocation beads comprises a unique spatial tag, wherein the oligonucleotide molecules are releasable from the plurality of geolocation beads by one or more of the release mechanisms selected from the group consisting of: (a) providing an enzyme mix comprising an enzyme configured to cleave or digest a cleavage site, wherein the oligonucleotide molecules comprise the cleavage site; (b) providing biotin, wherein the oligonucleotide molecules are conjugated to a desthiobiotin moiety, wherein the geolocation bead comprises a streptavidin moiety bound to the desthiobiotin moiety; (c) providing an enzyme mix comprising an enzyme configured to cleave or digest a cleavage site and UV light, wherein the oligonucleotide molecules comprise azobenzene and the cleavage site; and (d) providing UV light, wherein the oligonucleotide molecules are conjugated azobenzene, wherein the plurality of geolocation beads comprise an alpha-cyclodextrine (a-CD) moiety bound to the azobenzene. 201. The system of embodiment 200, further comprising indexed data comprising a list of spatial tag sequences included in the plurality of geolocation beads. 202. The system of embodiment 200 or 201, further comprising a second substrate comprising a second plurality of individually addressable locations configured to immobilize a second plurality of geolocation beads. 203. The system of embodiment 202, further comprising the second plurality of geolocation beads. 204. The system of any one of embodiments 200-203, further comprising a reagent configured to release the oligonucleotide molecules from the plurality of geolocation beads. 205. The system of embodiment 204, wherein the reagent comprises one or more of: (i) an enzyme mix comprising an enzyme configured to cleave or digest a cleavage site, wherein the oligonucleotide molecules comprise the cleavage site; (ii) biotin, wherein the oligonucleotide molecules are conjugated to a desthiobiotin moiety, wherein the plurality of geolocation beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of geolocation beads comprises an alpha-cyclodextrine (a-CD) moiety bound to the azobenzene. 206. The system of any one of embodiments 200-205, further comprising sequencing reagents. 207. The system of embodiment 206, wherein the sequencing reagents comprise single-base nucleotide mixtures for each of the four base types. 208. The system of embodiment 207, wherein a single-base nucleotide mixture of the single-base nucleotide mixtures comprises a mixture of labeled nucleotides and non-labeled nucleotides of a single base. 209. The system of embodiment 207 or 208, wherein a single-base nucleotide mixture of the single-base nucleotide mixtures comprises non-terminated nucleotides. 210. The system of any one of embodiments 200-209, further comprising amplification reagents. 211. The system of embodiment 210, wherein the amplification reagents comprise a polymerase, a nucleotide mixture, a primer, a buffer, or any combination thereof. 212. The system of any one of embodiments 200-211, further comprising a biological sample. 213. The system of embodiment 212, wherein the biological sample is a tissue. 214. The system of embodiment 212 or 213, wherein the biological sample is fixed. 215. The system of any one of embodiments 212-214, wherein the biological sample is permeabilized. 216. The system of any one of embodiments 212-215, wherein the biological sample is loaded on the substrate. 217. The system of any one of embodiments 200-216, wherein a geolocation bead of the plurality of geolocation beads comprises at least 100,000 oligonucleotides molecules. 218. The system of embodiment 217, wherein the at least 100,000 oligonucleotides molecules comprise a spatial tag of the spatial tags that is common and unique to the geolocation bead amongst the plurality of geolocation beads. 219. The system of any one of embodiments 200-218, wherein an oligonucleotide molecule of the oligonucleotide molecules comprises a capture sequence, wherein the capture sequence is configured to hybridize with a sequence of an analyte, or derivative thereof, of a biological sample. 220. The system of embodiment 219, wherein the capture sequence is selected from the group consisting of: a polyT sequence, a polyG sequence, a targeted mRNA sequence, a targeted gDNA sequence, a random n-mer sequence, and a probe sequence. 221. The system of any one of embodiments 200-220, wherein the substrate comprises at least 1,000,000 individually addressable locations. 222. The system of embodiment 221, wherein the substrate comprises at least 1,000,000,000 individually addressable locations. 223. The system of any one of embodiments 200-222, wherein the plurality of geolocation beads is immobilized to the individually addressable locations via electrostatic interactions. 224. The system of any one of embodiments 200-223, wherein the sequencing platform is configured to perform sequencing by synthesis on the substrates. 225. The system of any one of embodiments 200-224, wherein the substrate is substantially planar.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/179,161, filed Apr. 23, 2021, U.S. Provisional Application No. 63/255,600, filed Oct. 14, 2021, and U.S. Provisional Application No. 63/286,506, filed Dec. 6, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method comprising:
(a) providing a substrate comprising a plurality of geolocation beads immobilized to a plurality of individually addressable locations on said substrate, wherein said plurality of geolocation beads comprises a plurality of spatial tag molecules comprising a plurality of spatial tags, and wherein each geolocation bead of said plurality of geolocation beads comprises a unique spatial tag;
(b) loading a sample onto said substrate, wherein said sample comprises a plurality of analyte sequences;
(c) releasing said plurality of spatial tag molecules from said plurality of geolocation beads, wherein, prior to said releasing, respective locations of said plurality of spatial tags are unknown;
(d) tagging a set of at least two spatial tags of said plurality of spatial tags to each of said plurality of analyte sequences to generate a plurality of spatially tagged sequences; and
(e) generating a map of said plurality of analyte sequences by identifying sets of spatial tags from said plurality of spatially tagged sequences, wherein said map comprises spatial information between at least a subset of said plurality of analyte sequences.

2. The method of claim 1, further comprising, prior to (a), loading said plurality of geolocation beads onto said substrate to immobilize said plurality of geolocation beads at said plurality of individually addressable locations.

3. The method of any one of claims 1-2, further comprising fixing said sample.

4. The method of any one of claims 1-3, further comprising permeabilizing said sample.

5. The method of any one of claims 1-4, wherein said sample comprises a tissue sample, wherein said plurality of analyte sequences comprises a plurality of messenger ribonucleic acid (mRNA) transcript sequences.

6. The method of any one of claims 1-5, wherein (d) comprises contacting a plurality of bridge constructs with said plurality of spatial tag molecules and said plurality of analyte sequences, under conditions sufficient for a bridge construct of said plurality of bridge constructs to capture (1) a first spatial tag molecule from said plurality of spatial tags, wherein said first spatial tag molecule comprises a first spatial tag, (2) a second spatial tag molecule from said plurality of spatial tags, wherein said second spatial tag molecule comprises a second spatial tag, and (3) an analyte sequence of said plurality of analyte sequences, to generate a tagged complex, and generating a spatially tagged sequence of said plurality of spatially tagged sequences using said tagged complex.

7. The method of claim 6, wherein said bridge construct is a partially double-stranded nucleic acid molecule, comprising a capture sequence as an overhang, a first attachment binding sequence, a second attachment binding sequence, a third attachment binding sequence, and a fourth attachment binding sequence, wherein said capture sequence is configured to bind to a sequence of said analyte sequence,
wherein said first spatial tag molecule comprises a first attachment sequence configured to bind to said first attachment binding sequence, said first spatial tag, and a second attachment sequence configured to bind to said second attachment binding sequence,
wherein said second spatial tag molecule comprises a third attachment sequence configured to bind to said third attachment binding sequence, said second spatial tag, and a fourth attachment sequence configured to bind to said fourth attachment binding sequence, and
wherein said tagged complex comprises a first nucleic acid strand comprising, in 3′ to 5′ order, (1) said capture sequence which is bound to said sequence of said analyte sequence, (2) said first attachment sequence which is bound to said first attachment binding sequence, (3) said first spatial tag which is not bound to another nucleic acid strand, (4) said second attachment sequence which is bound to said second attachment binding sequence, (5) said third attachment sequence which is bound to said third attachment binding sequence, (6) said second spatial tag which is not bound to another nucleic acid strand, and (7) said fourth attachment sequence which is bound to said fourth attachment binding sequence.

8. The method of claim 7, wherein said second spatial tag molecule comprises a capture entity, wherein said first nucleic acid strand of said tagged complex comprises said capture entity.

9. The method of claim 8, wherein said capture entity comprises biotin.

10. The method of any one of claims 7-9, wherein said bridge construct comprises a first spacer sequence disposed between said first attachment binding sequence and said second attachment binding sequence, wherein said bridge construct comprises a second spacer sequence disposed between said third attachment binding sequence and said fourth attachment binding sequence.

11. The method of any one of claims 7-10, wherein said capture sequence comprises a polyT sequence, and wherein said sequence of said analyte sequence comprises a polyA sequence.

12. The method of any one of claims 7-10, wherein said capture sequence comprises a random n-mer sequence.

13. The method of any one of claims 7-12, wherein a first subset of said plurality of spatial tag molecules each comprises a respective spatial tag disposed between said first attachment sequence and said second attachment sequence, and wherein a second subset of said plurality of spatial tag molecules each comprises a respective spatial tag disposed between said third attachment sequence and said fourth attachment sequence.

14. The method of any one of claims 7-13, wherein said first attachment sequence is different from said third attachment sequence.

15. The method of any one of claims 7-13, wherein said first attachment sequence is the same as said third attachment sequence.

16. The method of any one of claims 7-15, wherein said first attachment sequence is different from said second attachment sequence.

17. The method of any one of claims 7-15, wherein said first attachment sequence is the same as said second attachment sequence.

18. The method of any one of claims 1-17, wherein a geolocation bead of said plurality of geolocation beads comprises a set of spatial tag molecules of said plurality of spatial tag molecules, wherein each spatial tag molecule of said set of spatial tag molecules comprises a common spatial tag of said plurality of spatial tags.

19. The method of claim 18, wherein each spatial tag molecule of said set of spatial tag molecules further comprises a unique molecular identifier (UMI) that is unique amongst said set of spatial tag molecules.

20. The method of any one of claims 18-19, wherein each spatial tag molecule of said set of spatial tag molecules comprises a first attachment sequence and a second attachment sequence, wherein said common spatial tag is disposed between said first attachment sequence and said second attachment sequence.

21. The method of claim 20, wherein each spatial tag molecule of said set of spatial tag molecules further comprises a UMI that is unique amongst said set of spatial tag molecules, wherein said UMI is disposed between said first attachment sequence and said second attachment sequence.

22. The method of any one of claims 18-21, wherein said set of spatial tag molecules comprises a first subset of spatial tag molecules and a second subset of spatial tag molecules, wherein said first subset of spatial tag molecules comprises a first attachment sequence and a second attachment sequence, and wherein said second subset of spatial tag molecules comprises a third attachment sequence and a fourth attachment sequence, wherein said first attachment sequence is different from said third attachment sequence.

23. The method of claim 22, wherein said second attachment sequence is different from said fourth attachment sequence.

24. The method of any one of claims 18-23, wherein said set of spatial tag molecules comprises at least 100,000 spatial tag molecules.

25. The method of any one of claims 1-24, wherein said plurality of geolocation beads comprises a first set of geolocation beads and a second set of geolocation beads, wherein said first set of geolocation beads comprises a first set of spatial tag molecules of said plurality of spatial tag molecules each comprising a first attachment sequence and a second attachment sequence, wherein said second set of geolocation beads comprises a second set of spatial tag molecules of said plurality of spatial tag molecules each comprising a third attachment sequence and a fourth attachment sequence, wherein said first attachment sequence is different from said third attachment sequence.

26. The method of claim 25, wherein said second attachment sequence is different from said fourth attachment sequence.

27. The method of any one of claims 1-26, wherein a geolocation bead of said plurality of geolocation beads comprises an oligonucleotide molecule, wherein said oligonucleotide molecule comprises a spatial tag molecule of said plurality of spatial tag molecules.

28. The method of claim 27, wherein the releasing said plurality of spatial tag molecules from said plurality of geolocation beads comprises releasing oligonucleotide molecules from the plurality of geolocation beads.

29. The method of claim 28, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of geolocation beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of geolocation beads comprises an alpha-cyclodextrine (a-CD) moiety bound to the azobenzene.

30. The method of any one of claims 27-29, wherein said spatial tag molecule is a first strand of said oligonucleotide molecule.

31. The method of any one of claims 27-30, wherein said oligonucleotide molecule is a partially double-stranded nucleic acid molecule comprising a first strand a second strand, wherein said first strand comprises said spatial tag molecule, wherein said second strand comprises a primer sequence and sequences corresponding to said spatial tag molecule.

32. The method of claim 31, wherein (c) comprises releasing said spatial tag molecule from said second strand.

33. The method of any one of claims 1-32, wherein a spatial tag molecule of said plurality of spatial tag molecules comprises a capture entity.

34. The method of claim 33, wherein said capture entity comprises biotin.

35. The method of any one of claims 33-34, further comprising recovering said plurality of spatially tagged sequences using said capture entity.

36. The method of any one of claims 33-35, further comprising recovering a plurality of tagged complexes using said capture entity, and generating said plurality of spatially tagged sequences using said plurality of tagged complexes.

37. The method of any one of claims 1-36, wherein a spatial tag molecule of said plurality of spatial tag molecules comprises a blocking group and a cleavage site.

38. The method of any one of claims 1-37, wherein said plurality of geolocation beads comprises at least 1,000,000 geolocation beads.

39. The method of any one of claims 1-38, wherein said plurality of geolocation beads comprises at least 100,000,000 geolocation beads.

40. The method of any one of claims 1-39, wherein said plurality of geolocation beads comprises at least 100,000,000,000 geolocation beads.

41. The method of any one of claims 1-40, wherein an analyte sequence comprises an mRNA sequence.

42. The method of any one of claims 1-40, wherein an analyte sequence comprises a DNA sequence.

43. The method of any one of claims 1-42, wherein said spatial information comprises a relative position of said at least said subset of said plurality of analyte sequences with respect to a reference analyte sequence.

44. The method of any one of claims 1-43, wherein said spatial information comprises a relative probability cloud of said at least said subset of said plurality of analyte sequences with respect to a reference analyte sequence.

45. The method of any one of claims 1-44, wherein said spatial information comprises two-dimensional (2D) spatial information.

46. The method of any one of claims 1-45, wherein said spatial information comprises three-dimensional (3D) spatial information.

47. The method of any one of claims 1-46, further comprising, subsequent to (b) and prior to (d), (i) contacting said sample with a surface of a second substrate, wherein said second substrate comprises a second plurality of geolocation beads immobilized to a second plurality of individually addressable locations on said surface of said second substrate, wherein said second plurality of geolocation beads comprises a second plurality of spatial tag molecules comprising a second plurality of spatial tags, wherein each geolocation bead of said second plurality of geolocation beads comprises a unique spatial tag, wherein said plurality of spatial tags and said second plurality of spatial tags are mutually exclusive, and (ii) releasing said second plurality of spatial tag molecules from said second plurality of geolocation beads, wherein, prior to said releasing, respective locations or identities of said second plurality of spatial tags are unknown.

48. The method of claim 47, further comprising, in (d), tagging a set of at least two spatial tags of plurality of spatial tags, at least two spatial tags of said second plurality of spatial tags, or at least two spatial tags from both said plurality of spatial tags and said second plurality of spatial tags to each of said plurality of analyte sequences to generate said plurality of spatially tagged sequences.

49. A method comprising:
(a) providing a substrate comprising a plurality of geolocation beads immobilized to a plurality of individually addressable locations on said substrate, wherein said plurality of geolocation beads comprises a plurality of spatial tag molecules comprising a plurality of spatial tags, and wherein each geolocation bead of said plurality of geolocation beads comprises a unique spatial tag;
(b) loading a sample onto said substrate, wherein said sample comprises a plurality of analyte sequences;
(c) releasing said plurality of spatial tag molecules from said plurality of geolocation beads, wherein, prior to said releasing, respective identities of said plurality of spatial tags are unknown;
(d) tagging a set of at least two spatial tags of said plurality of spatial tag molecules to each of said plurality of analyte sequences to generate a plurality of spatially tagged sequences; and
(e) generating a map of said plurality of analyte sequences by identifying sets of spatial tags from said plurality of spatially tagged sequences, wherein said map comprises spatial information between at least a subset of said plurality of analyte sequences.

50. The method of claim 49, further comprising, prior to (a), loading said plurality of geolocation beads onto said substrate to immobilize said plurality of geolocation beads at said plurality of individually addressable locations.

51. The method of any one of claims 49-50, further comprising fixing said sample.

52. The method of any one of claims 49-51, further comprising permeabilizing said sample.

53. The method of any one of claims 49-52, wherein said sample comprises a tissue sample, wherein said plurality of analyte sequences comprises a plurality of messenger ribonucleic acid (mRNA) transcript sequences.

54. The method of any one of claims 49-53, wherein (d) comprises contacting a plurality of bridge constructs with said plurality of spatial tag molecules and said plurality of analyte sequences, under conditions sufficient for a bridge construct of said plurality of bridge constructs to capture (1) a first spatial tag molecule from said plurality of spatial tags, wherein said first spatial tag molecule comprises a first spatial tag, (2) a second spatial tag molecule from said plurality of spatial tags, wherein said second spatial tag molecule comprises a second spatial tag, and (3) an analyte sequence of said plurality of analyte sequences, to generate a tagged complex, and generating a spatially tagged sequence of said plurality of spatially tagged sequences using said tagged complex.

55. The method of claim 54, wherein said bridge construct is a partially double-stranded nucleic acid molecule, comprising a capture sequence as an overhang, a first attachment binding sequence, a second attachment binding sequence, a third attachment binding sequence, and a fourth attachment binding sequence, wherein said capture sequence is configured to bind to a sequence of said analyte sequence,
wherein said first spatial tag molecule comprises a first attachment sequence configured to bind to said first attachment binding sequence, said first spatial tag, and a second attachment sequence configured to bind to said second attachment binding sequence,
wherein said second spatial tag molecule comprises a third attachment sequence configured to bind to said third attachment binding sequence, said second spatial tag, and a fourth attachment sequence configured to bind to said fourth attachment binding sequence, and
wherein said tagged complex comprises a first nucleic acid strand comprising, in 3′ to 5′ order, (1) said capture sequence which is bound to said sequence of said analyte sequence, (2) said first attachment sequence which is bound to said first attachment binding sequence, (3) said first spatial tag which is not bound to another nucleic acid strand, (4) said second attachment sequence which is bound to said second attachment binding sequence, (5) said third attachment sequence which is bound to said third attachment binding sequence, (6) said second spatial tag which is not bound to another nucleic acid strand, and (7) said fourth attachment sequence which is bound to said fourth attachment binding sequence.

56. The method of claim 55, wherein said second spatial tag molecule comprises a capture entity, wherein said first nucleic acid strand of said tagged complex comprises said capture entity.

57. The method of claim 56, wherein said capture entity comprises biotin.

58. The method of any one of claims 55-57, wherein said bridge construct comprises a first spacer sequence disposed between said first attachment binding sequence and said second attachment binding sequence, wherein said bridge construct comprises a second spacer sequence disposed between said third attachment binding sequence and said fourth attachment binding sequence.

59. The method of any one of claims 55-58, wherein said capture sequence comprises a polyT sequence, and wherein said sequence of said analyte sequence comprises a polyA sequence.

60. The method of any one of claims 55-58, wherein said capture sequence comprises a random n-mer sequence.

61. The method of any one of claims 55-60, wherein a first subset of said plurality of spatial tag molecules each comprises a respective spatial tag disposed between said first attachment sequence and said second attachment sequence, and wherein a second subset of said plurality of spatial tag molecules each comprises a respective spatial tag disposed between said third attachment sequence and said fourth attachment sequence.

62. The method of any one of claims 55-61, wherein said first attachment sequence is different from said third attachment sequence.

63. The method of any one of claims 55-61, wherein said first attachment sequence is the same as said third attachment sequence.

64. The method of any one of claims 55-63, wherein said first attachment sequence is different from said second attachment sequence.

65. The method of any one of claims 55-63, wherein said first attachment sequence is the same as said second attachment sequence.

66. The method of any one of claims 49-65, wherein a geolocation bead of said plurality of geolocation beads comprises a set of spatial tag molecules of said plurality of spatial tag molecules, wherein each spatial tag molecule of said set of spatial tag molecules comprises a common spatial tag of said plurality of spatial tags.

67. The method of claim 66, wherein each spatial tag molecule of said set of spatial tag molecules further comprises a unique molecular identifier (UMI) that is unique amongst said set of spatial tag molecules.

68. The method of any one of claims 66-67, wherein each spatial tag molecule of said set of spatial tag molecules comprises a first attachment sequence and a second attachment sequence, wherein said common spatial tag is disposed between said first attachment sequence and said second attachment sequence.

69. The method of claim 68, wherein each spatial tag molecule of said set of spatial tag molecules further comprises a UMI that is unique amongst said set of spatial tag molecules, wherein said UMI is disposed between said first attachment sequence and said second attachment sequence.

70. The method of any one of claims 66-69, wherein said set of spatial tag molecules comprises a first subset of spatial tag molecules and a second subset of spatial tag molecules, wherein said first subset of spatial tag molecules comprises a first attachment sequence and a second attachment sequence, and wherein said second subset of spatial tag molecules comprises a third attachment sequence and a fourth attachment sequence, wherein said first attachment sequence is different from said third attachment sequence.

71. The method of claim 70, wherein said second attachment sequence is different from said fourth attachment sequence.

72. The method of any one of claims 66-71, wherein said set of spatial tag molecules comprises at least 100,000 spatial tag molecules.

73. The method of any one of claims 49-72, wherein said plurality of geolocation beads comprises a first set of geolocation beads and a second set of geolocation beads, wherein said first set of geolocation beads comprises a first set of spatial tag molecules of said plurality of spatial tag molecules each comprising a first attachment sequence and a second attachment sequence, wherein said second set of geolocation beads comprises a second set of spatial tag molecules of said plurality of spatial tag molecules each comprising a third attachment sequence and a fourth attachment sequence, wherein said first attachment sequence is different from said third attachment sequence.

74. The method of claim 73, wherein said second attachment sequence is different from said fourth attachment sequence.

75. The method of any one of claims 49-74, wherein a geolocation bead of said plurality of geolocation beads comprises an oligonucleotide molecule, wherein said oligonucleotide molecule comprises a spatial tag molecule of said plurality of spatial tag molecules.

76. The method of claim 75, wherein the releasing said plurality of spatial tag molecules from said plurality of geolocation beads comprises releasing oligonucleotide molecules from the plurality of geolocation beads.

77. The method of claim 76, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of geolocation beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of geolocation beads comprises an alpha-cyclodextrine (a-CD) moiety bound to the azobenzene.

78. The method of any one of claims 75-77, wherein said spatial tag molecule is a first strand of said oligonucleotide molecule.

79. The method of any one of claims 75-78, wherein said oligonucleotide molecule is a partially double-stranded nucleic acid molecule comprising a first strand a second strand, wherein said first strand comprises said spatial tag molecule, wherein said second strand comprises a primer sequence and sequences corresponding to said spatial tag molecule.

80. The method of claim 79, wherein (c) comprises releasing said spatial tag molecule from said second strand.

81. The method of any one of claims 49-80, wherein a spatial tag molecule of said plurality of spatial tag molecules comprises a capture entity.

82. The method of claim 81, wherein said capture entity comprises biotin.

83. The method of any one of claims 81-82, further comprising recovering said plurality of spatially tagged sequences using said capture entity.

84. The method of any one of claims 81-83, further comprising recovering a plurality of tagged complexes using said capture entity, and generating said plurality of spatially tagged sequences using said plurality of tagged complexes.

85. The method of any one of claims 49-84, wherein a spatial tag molecule of said plurality of spatial tag molecules comprises a blocking group and a cleavage site.

86. The method of any one of claims 49-85, wherein said plurality of geolocation beads comprises at least 1,000,000 geolocation beads.

87. The method of any one of claims 49-86, wherein said plurality of geolocation beads comprises at least 100,000,000 geolocation beads.

88. The method of any one of claims 49-87, wherein said plurality of geolocation beads comprises at least 100,000,000,000 geolocation beads.

89. The method of any one of claims 49-88, wherein an analyte sequence comprises an mRNA sequence.

90. The method of any one of claims 49-88, wherein an analyte sequence comprises a DNA sequence.

91. The method of any one of claims 49-90, wherein said spatial information comprises a relative position of said at least said subset of said plurality of analyte sequences with respect to a reference analyte sequence.

92. The method of any one of claims 49-91, wherein said spatial information comprises a relative probability cloud of said at least said subset of said plurality of analyte sequences with respect to a reference analyte sequence.

93. The method of any one of claims 49-92, wherein said spatial information comprises two-dimensional (2D) spatial information.

94. The method of any one of claims 49-93, wherein said spatial information comprises three-dimensional (3D) spatial information.

95. The method of any one of claims 49-94, further comprising, subsequent to (b) and prior to (d), (i) contacting said sample with a surface of a second substrate, wherein said second substrate comprises a second plurality of geolocation beads immobilized to a second plurality of individually addressable locations on said surface of said second substrate, wherein said second plurality of geolocation beads comprises a second plurality of spatial tag molecules comprising a second plurality of spatial tags, wherein each geolocation bead of said second plurality of geolocation beads comprises a unique spatial tag, wherein said plurality of spatial tags and said second plurality of spatial tags are mutually exclusive, and (ii) releasing said second plurality of spatial tag molecules from said second plurality of geolocation beads, wherein, prior to said releasing, respective locations or identities of said second plurality of spatial tags are unknown.

96. The method of claim 95, further comprising, in (d), tagging a set of at least two spatial tags of plurality of spatial tags, at least two spatial tags of said second plurality of spatial tags, or at least two spatial tags from both said plurality of spatial tags and said second plurality of spatial tags to each of said plurality of analyte sequences to generate said plurality of spatially tagged sequences.

97. A method comprising:
(a) partitioning cells from a plurality of cells into a plurality of partitions, wherein a partition of said plurality of partitions comprises a cell and a plurality of geolocation beads, wherein said cell comprises a plurality of analyte sequences, wherein said plurality of geolocation beads comprises a plurality of spatial tag molecules comprising a plurality of spatial tags, and wherein each geolocation bead of said plurality of geolocation beads comprises a unique spatial tag;
(b) releasing said plurality of spatial tag molecules from said plurality of geolocation beads;
(c) tagging a set of at least two spatial tags of said plurality of spatial tags to each of said plurality of analyte sequences to generate a plurality of spatially tagged sequences; and
(d) sequencing said plurality of spatially tagged sequences, or derivatives thereof, to determine that said plurality of analyte sequences originated from said cell of said plurality of cells by identifying sets of spatial tags from said plurality of spatially tagged sequences.

98. The method of claim 97, wherein said partition is a droplet.

99. The method of claim 97, wherein said partition is a well.

100. The method of any one of claims 97-99, wherein (c) comprises contacting a plurality of bridge constructs with said plurality of spatial tag molecules and said plurality of analyte sequences, under conditions sufficient for a bridge construct of said plurality of bridge constructs to capture (1) a first spatial tag molecule from said plurality of spatial tags, wherein said first spatial tag molecule comprises a first spatial tag, (2) a second spatial tag molecule from said plurality of spatial tags, wherein said second spatial tag molecule comprises a second spatial tag, and (3) an analyte sequence of said plurality of analyte sequences, to generate a tagged complex, and generating a spatially tagged sequence of said plurality of spatially tagged sequences using said tagged complex.

101. The method of claim 100, wherein said bridge construct is a partially double-stranded nucleic acid molecule, comprising a capture sequence as an overhang, a first attachment binding sequence, a second attachment binding sequence, a third attachment binding sequence, and a fourth attachment binding sequence, wherein said capture sequence is configured to bind to a sequence of said analyte sequence,
wherein said first spatial tag molecule comprises a first attachment sequence configured to bind to said first attachment binding sequence, said first spatial tag, and a second attachment sequence configured to bind to said second attachment binding sequence,
wherein said second spatial tag molecule comprises a third attachment sequence configured to bind to said third attachment binding sequence, said second spatial tag, and a fourth attachment sequence configured to bind to said fourth attachment binding sequence, and
wherein said tagged complex comprises a first nucleic acid strand comprising, in 3′ to 5′ order, (1) said capture sequence which is bound to said sequence of said analyte sequence, (2) said first attachment sequence which is bound to said first attachment binding sequence, (3) said first spatial tag which is not bound to another nucleic acid strand, (4) said second attachment sequence which is bound to said second attachment binding sequence, (5) said third attachment sequence which is bound to said third attachment binding sequence, (6) said second spatial tag which is not bound to another nucleic acid strand, and (7) said fourth attachment sequence which is bound to said fourth attachment binding sequence.

102. The method of claim 101, wherein said second spatial tag molecule comprises a capture entity, wherein said first nucleic acid strand of said tagged complex comprises said capture entity.

103. The method of claim 102, wherein said capture entity comprises biotin.

104. The method of any one of claims 101-103, wherein said bridge construct comprises a first spacer sequence disposed between said first attachment binding sequence and said second attachment binding sequence, wherein said bridge construct comprises a second spacer sequence disposed between said third attachment binding sequence and said fourth attachment binding sequence.

105. The method of any one of claims 101-104, wherein said capture sequence comprises a polyT sequence, and wherein said sequence of said analyte sequence comprises a polyA sequence.

106. The method of any one of claims 101-104, wherein said capture sequence comprises a random n-mer sequence.

107. The method of any one of claims 101-106, wherein a first subset of said plurality of spatial tag molecules each comprises a respective spatial tag disposed between said first attachment sequence and said second attachment sequence, and wherein a second subset of said plurality of spatial tag molecules each comprises a respective spatial tag disposed between said third attachment sequence and said fourth attachment sequence.

108. The method of any one of claims 101-107, wherein said first attachment sequence is different from said third attachment sequence.

109. The method of any one of claims 101-107, wherein said first attachment sequence is the same as said third attachment sequence.

110. The method of any one of claims 101-109, wherein said first attachment sequence is different from said second attachment sequence.

111. The method of any one of claims 101-109, wherein said first attachment sequence is the same as said second attachment sequence.

112. The method of any one of claims 97-111, wherein a geolocation bead of said plurality of geolocation beads comprises a set of spatial tag molecules of said plurality of spatial tag molecules, wherein each spatial tag molecule of said set of spatial tag molecules comprises a common spatial tag of said plurality of spatial tags.

113. The method of claim 112, wherein each spatial tag molecule of said set of spatial tag molecules further comprises a unique molecular identifier (UMI) that is unique amongst said set of spatial tag molecules.

114. The method of any one of claims 112-113, wherein each spatial tag molecule of said set of spatial tag molecules comprises a first attachment sequence and a second attachment sequence, wherein said common spatial tag is disposed between said first attachment sequence and said second attachment sequence.

115. The method of claim 114, wherein each spatial tag molecule of said set of spatial tag molecules further comprises a UMI that is unique amongst said set of spatial tag molecules, wherein said UMI is disposed between said first attachment sequence and said second attachment sequence.

116. The method of any one of claims 112-115, wherein said set of spatial tag molecules comprises a first subset of spatial tag molecules and a second subset of spatial tag molecules, wherein said first subset of spatial tag molecules comprises a first attachment sequence and a second attachment sequence, and wherein said second subset of spatial tag molecules comprises a third attachment sequence and a fourth attachment sequence, wherein said first attachment sequence is different from said third attachment sequence.

117. The method of claim 116, wherein said second attachment sequence is different from said fourth attachment sequence.

118. The method of any one of claims 112-117, wherein said set of spatial tag molecules comprises at least 100,000 spatial tag molecules.

119. The method of any one of claims 97-118, wherein said plurality of geolocation beads comprises a first set of geolocation beads and a second set of geolocation beads, wherein said first set of geolocation beads comprises a first set of spatial tag molecules of said plurality of spatial tag molecules each comprising a first attachment sequence and a second attachment sequence, wherein said second set of geolocation beads comprises a second set of spatial tag molecules of said plurality of spatial tag molecules each comprising a third attachment sequence and a fourth attachment sequence, wherein said first attachment sequence is different from said third attachment sequence.

120. The method of claim 119, wherein said second attachment sequence is different from said fourth attachment sequence.

121. The method of any one of claims 97-120, wherein a geolocation bead of said plurality of geolocation beads comprises an oligonucleotide molecule, wherein said oligonucleotide molecule comprises a spatial tag molecule of said plurality of spatial tag molecules.

122. The method of claim 121, wherein the releasing said plurality of spatial tag molecules from said plurality of geolocation beads comprises releasing oligonucleotide molecules from the plurality of geolocation beads.

123. The method of claim 122, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of geolocation beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of geolocation beads comprises an alpha-cyclodextrine (a-CD) moiety bound to the azobenzene.

124. The method of claim 123, wherein said spatial tag molecule is a first strand of said oligonucleotide molecule.

125. The method of any one of claims 123-124, wherein said oligonucleotide molecule is a partially double-stranded nucleic acid molecule comprising a first strand a second strand, wherein said first strand comprises said spatial tag molecule, wherein said second strand comprises a primer sequence and sequences corresponding to said spatial tag molecule.

126. The method of claim 125, wherein (b) comprises releasing said spatial tag molecule from said second strand.

127. The method of any one of claims 97-126, wherein a spatial tag molecule of said plurality of spatial tag molecules comprises a capture entity.

128. The method of claim 127, wherein said capture entity comprises biotin.

129. The method of any one of claims 127-128, further comprising recovering said plurality of spatially tagged sequences using said capture entity.

130. The method of any one of claims 127-129, further comprising recovering a plurality of tagged complexes using said capture entity, and generating said plurality of spatially tagged sequences using said plurality of tagged complexes.

131. The method of any one of claims 97-130, wherein a spatial tag molecule of said plurality of spatial tag molecules comprises a blocking group and a cleavage site.

132. The method of any one of claims 97-131, wherein said plurality of geolocation beads comprises at least 1,000,000 geolocation beads.

133. The method of any one of claims 97-132, wherein said plurality of geolocation beads comprises at least 100,000,000 geolocation beads.

134. The method of any one of claims 97-133, wherein said plurality of geolocation beads comprises at least 100,000,000,000 geolocation beads.

135. The method of any one of claims 97-134, wherein an analyte sequence comprises an mRNA sequence.

136. The method of any one of claims 97-134, wherein an analyte sequence comprises a DNA sequence.

137. A method comprising:
(a) providing a solution comprising a plurality of cells and a plurality of geolocation beads, wherein said plurality of cells comprises a plurality of analyte sequences, wherein said plurality of geolocation beads comprises a plurality of spatial tag molecules comprising a plurality of spatial tags, and wherein each geolocation bead of said plurality of geolocation beads comprises a unique spatial tag;
(b) releasing said plurality of spatial tag molecules from said plurality of geolocation beads;
(c) tagging a set of at least two spatial tags of said plurality of spatial tags to each of said plurality of analyte sequences to generate a plurality of spatially tagged sequences; and
(d) sequencing said plurality of spatially tagged sequences, or derivatives thereof, to determine that a subset of said plurality of analyte sequences originated from a cell of said plurality of cells by identifying sets of spatial tags from said plurality of spatially tagged sequences.

138. The method of claim 137, wherein (c) comprises contacting a plurality of bridge constructs with said plurality of spatial tag molecules and said plurality of analyte sequences, under conditions sufficient for a bridge construct of said plurality of bridge constructs to capture (1) a first spatial tag molecule from said plurality of spatial tags, wherein said first spatial tag molecule comprises a first spatial tag, (2) a second spatial tag molecule from said plurality of spatial tags, wherein said second spatial tag molecule comprises a second spatial tag, and (3) an analyte sequence of said plurality of analyte sequences, to generate a tagged complex, and generating a spatially tagged sequence of said plurality of spatially tagged sequences using said tagged complex.

139. The method of claim 138, wherein said bridge construct is a partially double-stranded nucleic acid molecule, comprising a capture sequence as an overhang, a first attachment binding sequence, a second attachment binding sequence, a third attachment binding sequence, and a fourth attachment binding sequence, wherein said capture sequence is configured to bind to a sequence of said analyte sequence,
wherein said first spatial tag molecule comprises a first attachment sequence configured to bind to said first attachment binding sequence, said first spatial tag, and a second attachment sequence configured to bind to said second attachment binding sequence,
wherein said second spatial tag molecule comprises a third attachment sequence configured to bind to said third attachment binding sequence, said second spatial tag, and a fourth attachment sequence configured to bind to said fourth attachment binding sequence, and
wherein said tagged complex comprises a first nucleic acid strand comprising, in 3′ to 5′ order, (1) said capture sequence which is bound to said sequence of said analyte sequence, (2) said first attachment sequence which is bound to said first attachment binding sequence, (3) said first spatial tag which is not bound to another nucleic acid strand, (4) said second attachment sequence which is bound to said second attachment binding sequence, (5) said third attachment sequence which is bound to said third attachment binding sequence, (6) said second spatial tag which is not bound to another nucleic acid strand, and (7) said fourth attachment sequence which is bound to said fourth attachment binding sequence.

140. The method of claim 139, wherein said second spatial tag molecule comprises a capture entity, wherein said first nucleic acid strand of said tagged complex comprises said capture entity.

141. The method of claim 140, wherein said capture entity comprises biotin.

142. The method of any one of claims 139-141, wherein said bridge construct comprises a first spacer sequence disposed between said first attachment binding sequence and said second attachment binding sequence, wherein said bridge construct comprises a second spacer sequence disposed between said third attachment binding sequence and said fourth attachment binding sequence.

143. The method of any one of claims 139-142, wherein said capture sequence comprises a polyT sequence, and wherein said sequence of said analyte sequence comprises a polyA sequence.

144. The method of any one of claims 139-142, wherein said capture sequence comprises a random n-mer sequence.

145. The method of any one of claims 139-144, wherein a first subset of said plurality of spatial tag molecules each comprises a respective spatial tag disposed between said first attachment sequence and said second attachment sequence, and wherein a second subset of said plurality of spatial tag molecules each comprises a respective spatial tag disposed between said third attachment sequence and said fourth attachment sequence.

146. The method of any one of claims 139-145, wherein said first attachment sequence is different from said third attachment sequence.

147. The method of any one of claims 139-145, wherein said first attachment sequence is the same as said third attachment sequence.

148. The method of any one of claims 139-147, wherein said first attachment sequence is different from said second attachment sequence.

149. The method of any one of claims 139-147, wherein said first attachment sequence is the same as said second attachment sequence.

150. The method of any one of claims 139-149, wherein a geolocation bead of said plurality of geolocation beads comprises a set of spatial tag molecules of said plurality of spatial tag molecules, wherein each spatial tag molecule of said set of spatial tag molecules comprises a common spatial tag of said plurality of spatial tags.

151. The method of claim 150, wherein each spatial tag molecule of said set of spatial tag molecules further comprises a unique molecular identifier (UMI) that is unique amongst said set of spatial tag molecules.

152. The method of any one of claims 150-151, wherein each spatial tag molecule of said set of spatial tag molecules comprises a first attachment sequence and a second attachment sequence, wherein said common spatial tag is disposed between said first attachment sequence and said second attachment sequence.

153. The method of claim 152, wherein each spatial tag molecule of said set of spatial tag molecules further comprises a UMI that is unique amongst said set of spatial tag molecules, wherein said UMI is disposed between said first attachment sequence and said second attachment sequence.

154. The method of any one of claims 150-153, wherein said set of spatial tag molecules comprises a first subset of spatial tag molecules and a second subset of spatial tag molecules, wherein said first subset of spatial tag molecules comprises a first attachment sequence and a second attachment sequence, and wherein said second subset of spatial tag molecules comprises a third attachment sequence and a fourth attachment sequence, wherein said first attachment sequence is different from said third attachment sequence.

155. The method of claim 154, wherein said second attachment sequence is different from said fourth attachment sequence.

156. The method of any one of claims 150-155, wherein said set of spatial tag molecules comprises at least 100,000 spatial tag molecules.

157. The method any one of claims 137-156, wherein said plurality of geolocation beads comprises a first set of geolocation beads and a second set of geolocation beads, wherein said first set of geolocation beads comprises a first set of spatial tag molecules of said plurality of spatial tag molecules each comprising a first attachment sequence and a second attachment sequence, wherein said second set of geolocation beads comprises a second set of spatial tag molecules of said plurality of spatial tag molecules each comprising a third attachment sequence and a fourth attachment sequence, wherein said first attachment sequence is different from said third attachment sequence.

158. The method of claim 157, wherein said second attachment sequence is different from said fourth attachment sequence.

159. The method of any one of claims 137-158, wherein a geolocation bead of said plurality of geolocation beads comprises an oligonucleotide molecule, wherein said oligonucleotide molecule comprises a spatial tag molecule of said plurality of spatial tag molecules.

160. The method of claim 159, wherein the releasing said plurality of spatial tag molecules from said plurality of geolocation beads comprises releasing oligonucleotide molecules from the plurality of geolocation beads.

161. The method of claim 160, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of geolocation beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of geolocation beads comprises an alpha-cyclodextrine (a-CD) moiety bound to the azobenzene.

162. The method of any one of claims 159-161, wherein said spatial tag molecule is a first strand of said oligonucleotide molecule.

163. The method of any one of claims 159-162, wherein said oligonucleotide molecule is a partially double-stranded nucleic acid molecule comprising a first strand a second strand, wherein said first strand comprises said spatial tag molecule, wherein said second strand comprises a primer sequence and sequences corresponding to said spatial tag molecule.

164. The method of claim 163, wherein (c) comprises releasing said spatial tag molecule from said second strand.

165. The method of any one of claims 137-164, wherein a spatial tag molecule of said plurality of spatial tag molecules comprises a capture entity.

166. The method of claim 165, wherein said capture entity comprises biotin.

167. The method of any one of claims 165-166, further comprising recovering said plurality of spatially tagged sequences using said capture entity.

168. The method of any one of claims 165-167, further comprising recovering a plurality of tagged complexes using said capture entity, and generating said plurality of spatially tagged sequences using said plurality of tagged complexes.

169. The method of any one of claims 137-168, wherein a spatial tag molecule of said plurality of spatial tag molecules comprises a blocking group and a cleavage site.

170. The method of any one of claims 137-169, wherein said plurality of geolocation beads comprises at least 1,000,000 geolocation beads.

171. The method of any one of claims 137-170, wherein said plurality of geolocation beads comprises at least 100,000,000 geolocation beads.

172. The method of any one of claims 137-171, wherein said plurality of geolocation beads comprises at least 100,000,000,000 geolocation beads.

173. The method of any one of claims 137-172, wherein an analyte sequence comprises an mRNA sequence.

174. The method of any one of claims 137-172, wherein an analyte sequence comprises a DNA sequence.

175. A kit, comprising:
a substrate comprising a plurality of geolocation beads immobilized to a plurality of individually addressable locations on the substrate, wherein the plurality of geolocation beads comprises oligonucleotide molecules coupled thereto, wherein the oligonucleotide molecules comprise spatial tag molecules, wherein each geolocation bead of said plurality of geolocation beads comprises a unique spatial tag,
wherein the oligonucleotide molecules are releasable from the plurality of geolocation beads by one or more of the release mechanisms selected from the group consisting of:
(a) providing an enzyme mix comprising an enzyme configured to cleave or digest a cleavage site, wherein the oligonucleotide molecules comprise the cleavage site;
(b) providing biotin, wherein the oligonucleotide molecules are conjugated to a desthiobiotin moiety, wherein the geolocation bead comprises a streptavidin moiety bound to the desthiobiotin moiety;
(c) providing an enzyme mix comprising an enzyme configured to cleave or digest a cleavage site and UV light, wherein the oligonucleotide molecules comprise azobenzene and the cleavage site; and
(d) providing UV light, wherein the oligonucleotide molecules are conjugated azobenzene, wherein the plurality of geolocation beads comprise an alpha-cyclodextrine (a-CD) moiety bound to the azobenzene.

176. The kit of claim 175, further comprising indexed data comprising a list of spatial tag sequences included in the plurality of geolocation beads.

177. The kit of claim 175 or 176, further comprising a second substrate comprising a second plurality of individually addressable locations configured to immobilize a second plurality of geolocation beads.

178. The kit of claim 177, further comprising the second plurality of geolocation beads.

179. The kit of any one of claims 175-178, further comprising a reagent configured to release the oligonucleotide molecules from the plurality of geolocation beads.

180. The kit of claim 179, wherein the reagent comprises one or more of:
(i) an enzyme mix comprising an enzyme configured to cleave or digest a cleavage site, wherein the oligonucleotide molecules comprise the cleavage site; (ii) biotin, wherein the oligonucleotide molecules are conjugated to a desthiobiotin moiety, wherein the plurality of geolocation beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of geolocation beads comprises an alpha-cyclodextrine (a-CD) moiety bound to the azobenzene.

181. The kit of any one of claims 175-180, further comprising sequencing reagents.

182. The kit of claim 181, wherein the sequencing reagents comprise single-base nucleotide mixtures for each of the four base types.

183. The kit of claim 182, wherein a single-base nucleotide mixture of the single-base nucleotide mixtures comprises a mixture of labeled nucleotides and non-labeled nucleotides of a single base.

184. The kit of claim 182 or 183, wherein a single-base nucleotide mixture of the single-base nucleotide mixtures comprises non-terminated nucleotides.

185. The kit of any one of claims 175-184, further comprising amplification reagents.

186. The kit of claim 185, wherein the amplification reagents comprise a polymerase, a nucleotide mixture, a primer, a buffer, or any combination thereof.

187. The kit of any one of claims 175-186, further comprising a biological sample.

188. The kit of claim 187, wherein the biological sample is a tissue.

189. The kit of claim 187 or 188, wherein the biological sample is fixed.

190. The kit of any one of claims 187-189, wherein the biological sample is permeabilized.

191. The kit of any one of claims 187-190, wherein the biological sample is loaded on the substrate.

192. The kit of any one of claims 175-191, wherein a geolocation bead of the plurality of geolocation beads comprises at least 100,000 oligonucleotides molecules.

193. The kit of claim 192, wherein the at least 100,000 oligonucleotides molecules comprise a spatial tag of the spatial tags that is common and unique to the geolocation bead amongst the plurality of geolocation beads.

194. The kit of any one of claims 175-193, wherein an oligonucleotide molecule of the oligonucleotide molecules comprises a capture sequence, wherein the capture sequence is configured to hybridize with a sequence of an analyte, or derivative thereof, of a biological sample.

195. The kit of claim 194, wherein the capture sequence is selected from the group consisting of: a polyT sequence, a polyG sequence, a targeted mRNA sequence, a targeted gDNA sequence, a random n-mer sequence, and a probe sequence.

196. The kit of any one of claims 175-195, wherein the substrate comprises at least 1,000,000 individually addressable locations.

197. The kit of claim 196 wherein the substrate comprises at least 1,000,000,000 individually addressable locations.

198. The kit of any one of claims 175-197, wherein the plurality of geolocation beads is immobilized to the individually addressable locations via electrostatic interactions.

199. The kit of any one of claims 175-198, wherein the substrate is substantially planar.

200. A system, comprising:
a sequencing platform configured to (i) address individually addressable locations of substrates and (ii) rotate the substrates during dispensing of sequencing reagents to the substrates or during imaging of the substrates or during both; and
a substrate comprising a plurality of geolocation beads immobilized to a plurality of individually addressable locations on the substrate, wherein the plurality of geolocation beads comprises oligonucleotide molecules coupled thereto, wherein the oligonucleotide molecules comprise spatial tag molecules, wherein each geolocation bead of said plurality of geolocation beads comprises a unique spatial tag,
wherein the oligonucleotide molecules are releasable from the plurality of geolocation beads by one or more of the release mechanisms selected from the group consisting of:
(a) providing an enzyme mix comprising an enzyme configured to cleave or digest a cleavage site, wherein the oligonucleotide molecules comprise the cleavage site;
(b) providing biotin, wherein the oligonucleotide molecules are conjugated to a desthiobiotin moiety, wherein the geolocation bead comprises a streptavidin moiety bound to the desthiobiotin moiety;
(c) providing an enzyme mix comprising an enzyme configured to cleave or digest a cleavage site and UV light, wherein the oligonucleotide molecules comprise azobenzene and the cleavage site; and
(d) providing UV light, wherein the oligonucleotide molecules are conjugated azobenzene, wherein the plurality of geolocation beads comprise an alpha-cyclodextrine (a-CD) moiety bound to the azobenzene.

201. The system of claim 200, further comprising indexed data comprising a list of spatial tag sequences included in the plurality of geolocation beads.

202. The system of claim 200 or 201, further comprising a second substrate comprising a second plurality of individually addressable locations configured to immobilize a second plurality of geolocation beads.

203. The system of claim 202, further comprising the second plurality of geolocation beads.

204. The system of any one of claims 200-203, further comprising a reagent configured to release the oligonucleotide molecules from the plurality of geolocation beads.

205. The system of claim 204, wherein the reagent comprises one or more of:
(i) an enzyme mix comprising an enzyme configured to cleave or digest a cleavage site, wherein the oligonucleotide molecules comprise the cleavage site; (ii) biotin, wherein the oligonucleotide molecules are conjugated to a desthiobiotin moiety, wherein the plurality of geolocation beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of geolocation beads comprises an alpha-cyclodextrine (a-CD) moiety bound to the azobenzene.

206. The system of any one of claims 200-205, further comprising sequencing reagents.

207. The system of claim 206, wherein the sequencing reagents comprise single-base nucleotide mixtures for each of the four base types.

208. The system of claim 207, wherein a single-base nucleotide mixture of the single-base nucleotide mixtures comprises a mixture of labeled nucleotides and non-labeled nucleotides of a single base.

209. The system of claim 207 or 208, wherein a single-base nucleotide mixture of the single-base nucleotide mixtures comprises non-terminated nucleotides.

210. The system of any one of claims 200-209, further comprising amplification reagents.

211. The system of claim 210, wherein the amplification reagents comprise a polymerase, a nucleotide mixture, a primer, a buffer, or any combination thereof.

212. The system of any one of claims 200-211, further comprising a biological sample.

213. The system of claim 212, wherein the biological sample is a tissue.

214. The system of claim 212 or 213, wherein the biological sample is fixed.

215. The system of any one of claims 212-214, wherein the biological sample is permeabilized.

216. The system of any one of claims 212-215, wherein the biological sample is loaded on the substrate.

217. The system of any one of claims 200-216, wherein a geolocation bead of the plurality of geolocation beads comprises at least 100,000 oligonucleotides molecules.

218. The system of claim 217, wherein the at least 100,000 oligonucleotides molecules comprise a spatial tag of the spatial tags that is common and unique to the geolocation bead amongst the plurality of geolocation beads.

219. The system of any one of claims 200-218, wherein an oligonucleotide molecule of the oligonucleotide molecules comprises a capture sequence, wherein the capture sequence is configured to hybridize with a sequence of an analyte, or derivative thereof, of a biological sample.

220. The system of claim 219, wherein the capture sequence is selected from the group consisting of: a polyT sequence, a polyG sequence, a targeted mRNA sequence, a targeted gDNA sequence, a random n-mer sequence, and a probe sequence.

221. The system of any one of claims 200-220, wherein the substrate comprises at least 1,000,000 individually addressable locations.

222. The system of claim 221, wherein the substrate comprises at least 1,000,000,000 individually addressable locations.

223. The system of any one of claims 200-222, wherein the plurality of geolocation beads is immobilized to the individually addressable locations via electrostatic interactions.

224. The system of any one of claims 200-223, wherein the sequencing platform is configured to perform sequencing by synthesis on the substrates.

225. The system of any one of claims 200-224, wherein the substrate is substantially planar.
